(19) United States
(12) Patent Application Publication
(10) Pub. No.: US 2018/0044343 A1
(43) Pub. Date: Feb. 15, 2018

(54) BICYCLIC IMIDAZOLO DERIVATIVE

(71) Applicant: Sumitomo Dainippon Pharma Co., Ltd., Osaka (JP)

(72) Inventors: Yuki FUJII, Osaka-shi, Osaka (JP); Hiroaki FUJIWARA, Chuo-ku, Tokyo (JP); Muneo KAWASUMI, Kanazawa-shi, Ishikawa (JP); Seiji IWAMA, Osaka-shi, Osaka (JP); Tomoko IKEDA, Osaka-shi, Osaka (JP); Saori KIYOSHIGE, Osaka-shi, Osaka (JP)

(73) Assignee: Sumitomo Dainippon Pharma Co., Ltd., Osaka (JP)

(21) Appl. No.: 15/557,580

(22) PCT Filed: Mar. 15, 2016
(86) PCT No.: PCT/JP2016/001482

§ 371 Date: Sep. 12, 2017

(30) Foreign Application Priority Data
Mar. 16, 2015 (JP) 2015-052588
Jan. 25, 2016 (JP) 2016-011511

(51) Int. Cl. C07D 487/04

(52) U.S. Cl. CPC C07D 487/04

(57) ABSTRACT

Disclosed are compounds, having the following structure, useful as inhibitors of Phosphodiesterase 1 (PDE1), compositions comprising the compounds, and methods of using the same.

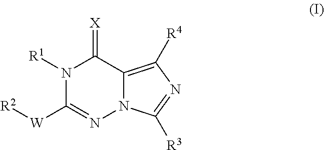

### TECHNICAL FIELD

The present invention relates to bicyclic imidazolo derivative compounds and pharmaceutical compositions comprising the same, which may be useful as inhibitors of Phosphodiesterase 1 (PDE1) enzymes.

### BACKGROUND ART

The prevalence of neurological and psychiatric disorders is increasing worldwide. Up to one billion people suffer from debilitating neurological conditions such as Alzheimer's disease and Parkinson's disease, with almost seven million people dying every year. “Neurological disorders: public health challenges” World Health Organization, 2006. Neurological and psychiatric disorders are prevalent in all countries, often without regard to age, sex, education or income. However, as many neurological disorders are correlated with increased age, as the global population ages, the impact of these disorders becomes more evident.

Despite the availability of treatments for some of these diseases, first-line therapies (such as L-DOPA for Parkinson's) are often burdened by unfavorable side effects, or may lack efficacy. For instance, there is currently no approved treatment for the cognitive deficits in schizophrenia despite high unmet medical needs.

### SUMMARY OF INVENTION

### Technical Problem

The continuing and increasing problem of neurological and psychiatric disorders, and the current lack of safe and effective drugs for treating them highlight overwhelming needs for new drugs to treat these conditions and their underlying causes.

### Solution to Problem

It has now been found that compounds disclosed herein and pharmaceutically acceptable compositions comprising the same may be effective as inhibitors of Phosphodiesterase 1 (PDE1) enzymes. Such compounds have a structure represented by the general formula I:

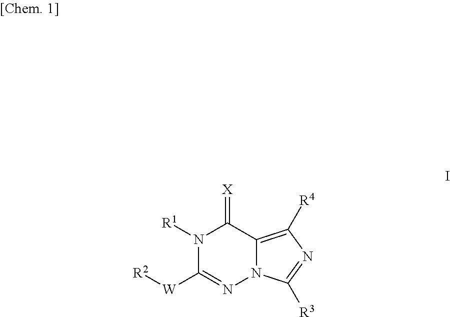

or a pharmaceutically acceptable salt thereof, wherein each variable is as defined and described herein.

Compounds disclosed herein and pharmaceutically acceptable compositions comprising the same may be useful for treating a variety of diseases, disorders or conditions, associated with regulation of PDE1 enzymes. Such diseases, disorders, or conditions include those described herein.

Compounds disclosed herein may be also useful for the study of PDE1 enzymes in biological and pathological phenomena, the study of intracellular signal transduction pathways occurring in PDE1-expressing tissues, and the comparative evaluation of new PDE1 inhibitors or other regulators neuronal activity in vitro or in vivo.

### BRIEF DESCRIPTION OF DRAWINGS

FIG. 1 shows discrimination index of Example 1 in Test Example 5. ## means P is <0.01 compared to the vehicle+vehicle-treated group in the unpaired t-test. ** means P is <0.01 compared to the PCP+vehicle-treated group in Dunnett's test. SE of the Mean is n=14-17.

FIG. 2 shows discrimination index of Example 115 in Test Example 5. ## means P is <0.01 compared to the vehicle+vehicle-treated group in the unpaired t-test. * means P is <0.05 compared to the PCP+vehicle-treated group in Dunnett's test. ** means P is <0.01 compared to the PCP+vehicle-treated group in Dunnett's test. SE of the Mean is n=11-15.

FIG. 3 shows discrimination index of Example 149 in Test Example 5. ## means P is <0.01 compared to the vehicle+vehicle-treated group in the unpaired t-test. ** means P is <0.01 compared to the PCP+vehicle-treated group in Dunnett's test. SE of the Mean is n=10-14.

FIG. 4 shows discrimination index of Example 245 in Test Example 5. ## means P is <0.01 compared to the vehicle+vehicle-treated group in the unpaired t-test. ** means P is <0.01 compared to the PCP+vehicle-treated group in Dunnett's test. SE of the Mean is n=11-14.

FIG. 5 shows discrimination index of Example 288 in Test Example 5. ## means P is <0.01 compared to the vehicle+vehicle-treated group in the unpaired t-test. ** means P is <0.01 compared to the PCP+vehicle-treated group in Dunnett's test. SE of the Mean is n=7-11.

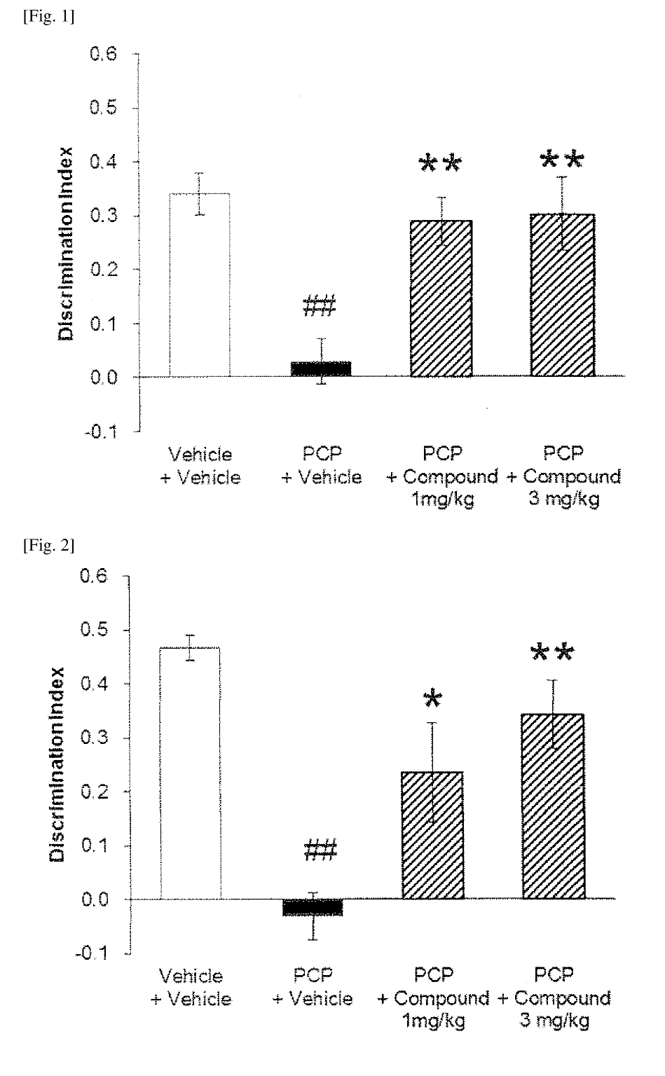

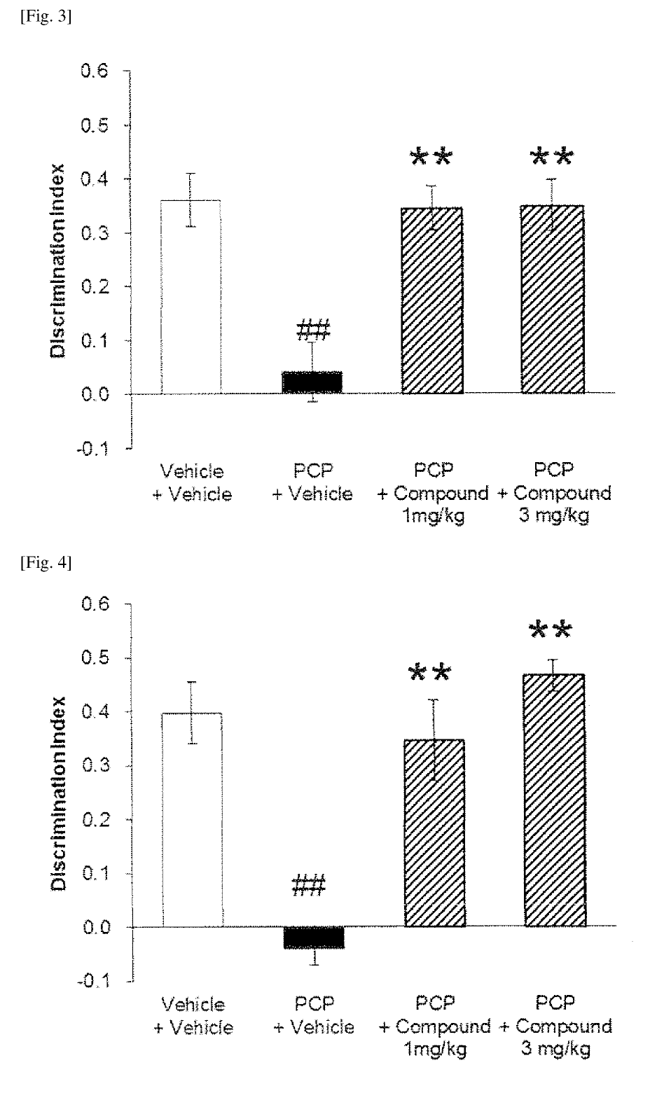

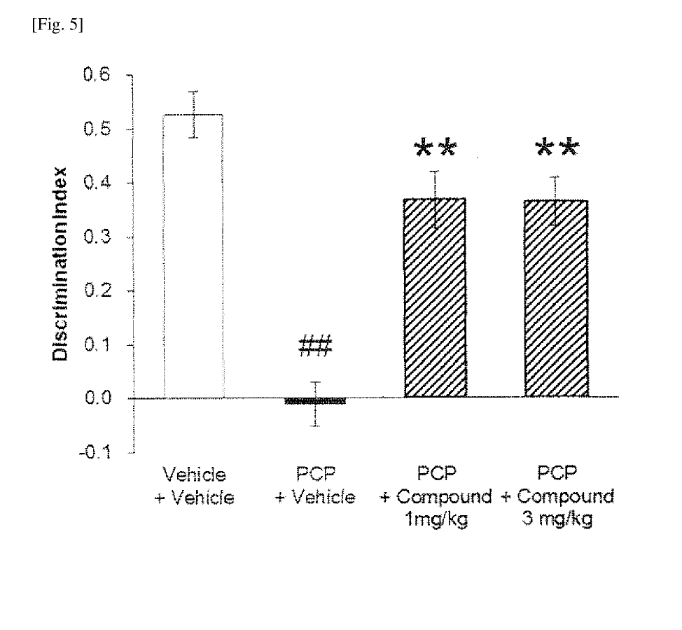

### DESCRIPTION OF EMBODIMENTS

### 1. General Description of Compounds of the Invention

In certain embodiments, the present invention provides inhibitors of PDE1. In one embodiment, the compounds provided by the present invention include the compound of formula I:

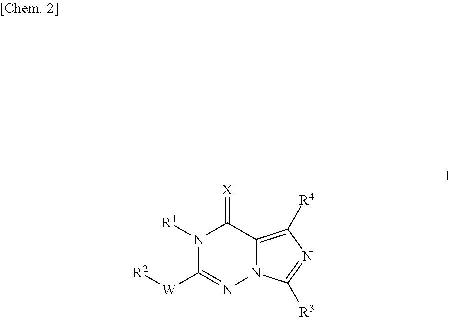

or a pharmaceutically acceptable salt thereof, wherein:

R¹ is selected from

(i) a hydrogen,

(ii) a C₁₋₆ alkyl (said group being optionally substituted with the same or different 1 to 3 group(s) selected from

(a) a halogen,

(b) a C₁₋₆ alkoxy (said group being optionally substituted with the same or different 1 to 3 halogen or hydroxy),

(c) a hydroxyl,

(d) a phenoxy (said group being optionally substituted with the same or different 1 to 4 halogen, C₁₋₆ alkyl, or C₁₋₆ alkoxy), and

(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and

(iii) a C₃₋₁₀ cycloalkyl; a phenyl; a 5-6 membered monocyclic heteroaryl; a 4-8 membered saturated or partially unsaturated monocyclic heterocyclyl; a C₃₋₁₀ cycloalkyl-C₁₋₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;

wherein each of said groups in the aforesaid (iii) is optionally substituted with the same or different 1 to 4 group(s) selected from

(a) a halogen,

(b) a C₁₋₆ alkyl (said group being optionally substituted with the same or different 1 to 3 halogen, C₁₋₆ alkoxy, or hydroxy),

(c) a C₁₋₆ alkoxy (said group being optionally substituted with the same or different 1 to 3 halogen, C₁₋₆ alkoxy, or hydroxy),

(d) a hydroxy,

(e) a cyano,

(f) a phenyl (said group being optionally substituted with the same or different 1 to 4 halogen, cyano, C₁₋₆ alkyl, or C₁₋₆ alkoxy),

(g) a 5-6 membered monocyclic heteroaryl (said group being optionally substituted with the same or different 1 to 4 halogen, cyano, C₁₋₆ alkyl, or C₁₋₆ alkoxy), and

(h) an aminocarbonyl (said amino being optionally substituted with the same or different 1 to 2 C₁₋₆ alkyl);

X is O or S;

W is a covalent bond, —C≡C—, —CH═CH—, —O—, or —N(R⁵)—;

R⁵ is a hydrogen or a C₁₋₆ alkyl;

R² is selected from

(i) a C₁₋₆ alkyl (said group being optionally substituted with the same or different 1 to 3 group(s) selected from

(a) a halogen,

(b) a C₁₋₆ alkoxy (said group being optionally substituted with the same or different 1 to 3 halogen or hydroxy),

(c) a hydroxyl,

(d) a phenoxy (said group being optionally substituted with the same or different 1 to 4 halogen, C₁₋₆ alkyl, or C₁₋₆ alkoxy), and

(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and

(ii) a C₃₋₁₀ cycloalkyl; a phenyl; a 5-6 membered heteroaryl; a 4-8 membered saturated or partially unsaturated heterocyclyl; a C₃₋₁₀ cycloalkyl-C₁₋₄ alkyl; a phenyl-C₁₋₄ alkyl; a 5 or 6-membered heteroaryl-C₁₋₄ alkyl; or a 4-8 membered saturated or partially unsaturated heterocyclyl-C₁₋₄ alkyl;

wherein each of said groups in the aforesaid (ii) is optionally substituted with the same or different 1 to 4 group(s) selected from

(a) a halogen,

(b) a C₁₋₆ alkyl (said group being optionally substituted with the same or different 1 to 3 halogen, C₁₋₆ alkoxy, or hydroxy),

(c) a C₁₋₆ alkoxy (said group being optionally substituted with the same or different 1 to 3 halogen, C₁₋₆ alkoxy, or hydroxy),

(d) a hydroxy,

(e) a cyano,

(f) a phenyl (said group being optionally substituted with the same or different 1 to 4 halogen, cyano, C₁₋₆ alkyl, or C₁₋₆ alkoxy),

(g) a 5-6 membered monocyclic heteroaryl (said group being optionally substituted with the same or different 1 to 4 halogen, cyano, C₁₋₆ alkyl, or C₁₋₆ alkoxy), and

(h) an aminocarbonyl (said amino being optionally substituted with the same or different 1 to 2 C₁₋₆ alkyl);

alternatively, R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from

(a) a halogen,

(b) a C₁₋₆ alkyl (said group being optionally substituted with the same or different 1 to 3 halogen, C₁₋₆ alkoxy, hydroxy, or aminocarbonyl (said amino being optionally substituted with the same or different 1 to 2 C₁₋₆ alkyl)),

(c) a C₁₋₆ alkoxy (said group being optionally substituted with the same or different 1 to 3 halogen, C₁₋₆ alkoxy, or hydroxy),

(d) a phenyl (said group being optionally substituted with the same or different 1 to 4 halogen, cyano, C₁₋₆ alkyl, or C₁₋₆ alkoxy),

(e) a 5-6 membered monocyclic heteroaryl (said group being optionally substituted with the same or different 1 to 4 halogen, cyano, C₁₋₆ alkyl, or C₁₋₆ alkoxy),

(f) a 5-6 membered monocyclic heteroaryloxy (said group being optionally substituted with the same or different 1 to 4 halogen, cyano, C₁₋₆ alkyl, or C₁₋₆ alkoxy),

(g) a C₁₋₆ alkylcarbonyl (said alkyl being optionally substituted with the same or different 1 to 3 halogen, C₁₋₆ alkoxy, or amino (said amino being optionally substituted with the same or different 1 to 2 C₁₋₆ alkyl)), and

(h) an aminocarbonyl (said amino being optionally substituted with the same or different 1 to 2 C₁₋₆ alkyl);

R³ is selected from

(i) a hydrogen,

(ii) a halogen,

(iii) a C₁₋₆ alkyl (said group being optionally substituted with the same or different 1 to 3 group(s) selected from

(a) a halogen,

(b) a C₁₋₆ alkoxy (said group being optionally substituted with the same or different 1 to 3 halogen),

(c) a hydroxy, and

(d) an oxo), or

(iv) a C₃₋₈ cycloalkyl (said group being optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) an oxo),

(v) a C₂₋₆ alkenyl (said group being optionally substituted with the same or different 1 to 4 halogen),

(vi) a C₃₋₈ cycloalkenyl (said group being optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) an oxo), and

(vii) 4-8 membered saturated or partially unsaturated monocyclic heterocyclyl (said group being optionally substituted with the same or different 1 to 4 group(s) selected from

(a) a halogen,

(b) a C₁₋₆ alkyl (said group being optionally substituted with the same or different 1 to 3 halogen),

(c) a C₁₋₆ alkoxy (said group being optionally substituted with the same or different 1 to 3 halogen),

(d) a hydroxy, and

(e) a cyano);

R⁴ is selected from

(i) a hydrogen,

(ii) a halogen,

(iii) a C₁₋₆ alkyl (said group being optionally substituted with the same or different 1 to 3 halogen), or

(iv) a cyano;

provided that when R¹ is a hydrogen, then W is —O— or —N(R⁵)— and R⁴ is a hydrogen.

### 2. Compounds and Definitions

Compounds of the present invention include those generally described above, and are further illustrated by the classes, subclasses, and species disclosed herein. As used herein, the following definitions shall apply unless otherwise indicated. For purposes of the present invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001; the entire contents of which are herein incorporated by reference.

The term “C₁₋₆ alkyl” used alone or as part of a larger moiety, e.g. “C₁₋₆ alkylcarbonyl,” refers to a straight or branched alkyl group with 1 to 6 carbon atoms. Exemplary groups for this group are methyl, ethyl, propyl, isopropyl, butyl, isobutyl, and tert-butyl.

The term “C₂₋₆ alkenyl” refers to a straight or branched alkenyl group having 2 to 6 carbon atoms and at least one carbon-carbon double bond. Exemplary groups for this group are ethenyl, propenyl, butenyl, 3-methyl-1-butenyl, pentenyl, and hexenyl.

The term “C₁₋₆ alkoxy” refers to a straight or branched alkoxy group with 1 to 6 carbon atoms. Exemplary groups for this group are methoxy, ethoxy, propoxy, isopropoxy, butoxy, and tert-butyloxy.

The term “C₃₋₁₀ cycloalkyl” refers to a 3 to 10-membered, preferably 5 to 6-membered, cycloalkyl group. The term “cycloalkyl” used herein includes a mono- or bi-cyclic hydrocarbon ring which may be optionally fused with another cycloalkyl or aryl group. Exemplary groups for this group include, for example, cyclopropyl, cyclobutyl, cyclopentyl, cyclohepyl, cyclohepyl, cyclooctyl, adamantyl, indanyl, and tetrahydronaphthyl.

The term “C₃₋₈ cycloalkenyl” refers to a 3 to 8-membered, preferably 5 to 6-membered, cycloalkenyl group comprising at least one unsaturated bond between carbon atoms that constitute the ring. The term “cycloalkenyl” used herein includes a mono- or bi-cyclic hydrocarbon ring which may be optionally fused with another cycloalkenyl or aryl group. Exemplary groups for this group include, for example, cyclopropenyl, cyclobutenyl, cyclopentenyl, cyclohexenyl, and dihydronaphthyl.

The term “halogen” means F, Cl, Br, or I.

The term “aryl”, used alone or as part of a larger moiety as in “aryloxy,” refers to a monocyclic or bicyclic ring system having a total of five to fourteen ring members, wherein at least one ring in the system is a carbocyclic aromatic ring and wherein each ring in the system contains 3 to 7 ring members. The term “aryl” may be used interchangeably with the term “aryl ring.” In certain embodiments of the present invention, “aryl” refers to a carbocyclic aromatic ring system which, for example, includes, but not limited to, phenyl, naphthyl, anthracyl and the like, which may be optionally substituted.

The term “heteroatom” means one or more of oxygen, sulfur, nitrogen, phosphorus, or silicon (including, any of oxidized forms of nitrogen, sulfur, phosphorus, boron, or silicon; a quaternized form of any basic nitrogen; or a substitutable nitrogen of a heterocyclic ring, for example, N (as in 3,4-dihydro-2H-pyrrolyl), NH (as in pyrrolidinyl) or NR+ (as in N-substituted pyrrolidinyl)).

The terms “heteroaryl” and “heteroar-,” used alone or as part of a larger moiety, e.g. “heteroaryloxy,” refer to groups having 5 to 10 ring atoms, preferably 5, 6, 9 or 10 ring atoms; having 6, 10, or 14π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl ring, to which a group attaches via any of members on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted.

As used herein, the terms “heterocycle,” “heterocyclyl,” “heterocyclic radical,” and “heterocyclic ring” are used interchangeably with each other and refer to a 5- to 7-membered monocyclic or 7- to 10-membered bicyclic heterocyclic moiety that is either saturated or partially unsaturated and has, in addition to carbon atoms, one or more, preferably one to four, heteroatom. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

The heterocyclic ring can attach to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl tetrahydropyranyl, dihydropyranyl, pyrrolidinyl, piperidinyl, pyrrolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are herein used interchangeably with each other, and also include groups in which the heterocyclyl ring is fused to one or more aryl, heteroaryl, or cycloaliphatic ring, such as indolinyl, 3H-indolyl, isoindolinyl, chromanyl, phenanthridinyl, tetrahydroquinolinyl, tetrahydroisoquinolinyl, or decahydroquinolinyl, to which a group attaches via any of members on the heterocyclyl ring. The heterocyclyl group may be optionally further substituted with one or more oxo group, which includes, for example, 1-oxo-2H-isoindolyl, 1,3-dioxoisoindolyl. The heterocyclyl group may be mono- or bicyclic group.

The term “C₃₋₁₀ cycloalkyl-C₁₋₄ alkyl” refers to a group wherein “C₃₋₁₀ cycloalkyl” as defined above is substituted on “C₁₋₄ alkyl”. “C₁₋₄ alkyl” includes, for example, a straight chain or branched chain C₁₋₄ alkyl and a C₃₋₄ alkyl having a cyclic structure. The straight chain or branched chain C₁₋₄ alkyl includes, for example, methyl, ethyl, trimethyl, 1-methylmethyl, 1-ethylmethyl, 1-propylmethyl, 1-methylethylene, 2-methylethyl, 1-ethylethyl, etc., and preferable one is methylene and ethylene. The C₃₋₄ alkyl having a cyclic structure is an alkylene selected from the following formulae.

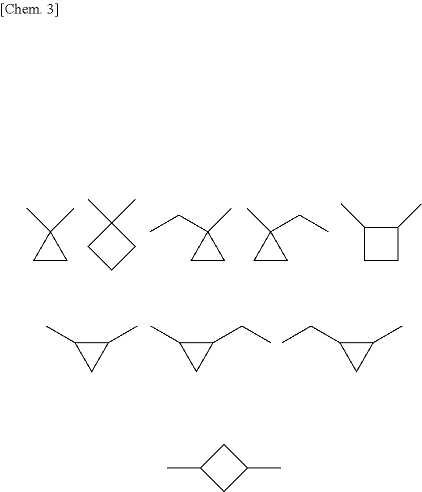

Exemplary C₃₋₁₀ cycloalkyl-C₁₋₄ alkyl groups include cyclopropylmethyl, cyclobutylmethyl, cyclopentylmethyl, cyclohexylmethyl, cyclohepylmethyl, cyclooctylmethyl, cyclohexylethyl, cyclohexylpropyl, cyclohexylbutyl, and adamantylmethyl.

The term “phenyl-C₁₋₄ alkyl” refers to a group wherein “phenyl” is substituted on “C₁₋₄ alkyl” as defined above. Exemplary phenyl-C₁₋₄ alkyl groups include benzyl, phenethyl, phenylpropyl, and phenylbutyl.

The term “heteroaryl-C₁₋₄ alkyl” refers to a group wherein “heteroaryl” as defined above is substituted on “C₁₋₄ alkyl” as defined above. Exemplary heteroaryl-C₁₋₄ alkyl groups include pyridylmethyl, pyrimidylmethyl, imidazolylmethyl, thiazolylmethyl, quinolylmethyl, pyridylethyl, and pyridylpropyl.

The term “heterocyclyl-C₁₋₄ alkyl” refers to a group wherein “heterocyclyl” as defined above is substituted on “C₁₋₄ alkyl” as defined above. Exemplary heterocyclyl-C₁₋₄ alkyl groups include piperidylmethyl, pyrrolidylmethyl, morpholinylmethyl, tetrahydofuranylmethyl, and tetrahydropyranylmethyl.

The term “aminocarbonyl” refers to a group wherein a hydrogen atom of formyl group is replaced with an amino group.

As used herein, the term “partially unsaturated” refers to a ring moiety that includes at least one double or triple bond between atoms that constitute the ring. The term “partially unsaturated” is intended to encompass rings having multiple sites of unsaturation, but is not intended to include aryl or heteroaryl moieties, as herein defined.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are generally well known in the art. For example, S. M. Berge et al. describe pharmaceutically acceptable salts in detail in J. Pharmaceutical Sciences, 1977, 66, 1-19, incorporated herein by reference. Pharmaceutically acceptable salts of the compounds of the present invention include those derived from suitable inorganic and organic acids and bases. Examples of pharmaceutically acceptable nontoxic acid addition salts are salts of an amino group formed with inorganic acids such as hydrochloric acid, hydrobromic acid, phosphoric acid, sulfuric acid and perchloric acid; those with organic acids such as acetic acid, oxalic acid, maleic acid, tartaric acid, citric acid, succinic acid or malonic acid; or those obtained by using other methods used in the art such as ion exchange. The pharmaceutically acceptable salts of the compounds of the present invention also include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

The salts derived from appropriate bases include alkali metal, alkaline earth metal, ammonium and N⁺(C₁₋₄ alkyl)₄ salts. Such alkali or alkaline earth metal salts typically include sodium, lithium, potassium, calcium, magnesium, and the like. The pharmaceutically acceptable salts of the compounds of the present invention also include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are meant to include all isomeric (e.g. enantiomeric, diastereomeric, and geometric (or conformational)) forms of the structures: for example, the R and S configurations for each asymmetric center, Z and E double bond isomers, and Z and E conformational isomers. Therefore, single stereo-chemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atom. For example, compounds having the present structures including the replacement of hydrogen with deuterium or tritium, or the replacement of a carbon with a ¹³C- or ¹⁴C-enriched carbon are within the scope of the present invention. Such compounds are useful, for example, as analytical tools, as probes in biological assays, or as therapeutic agents in accordance with the present invention.

### 3. Description of Exemplary Embodiments

In certain embodiments, the present invention provides inhibitors of PDE1. In one embodiment, the compounds provided by the present invention include the compound of formula I:

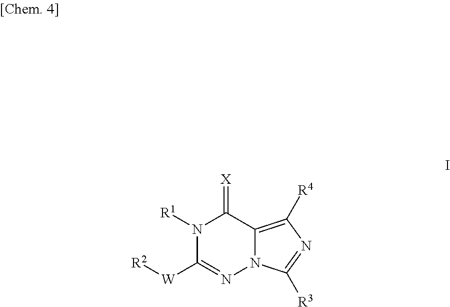

or a pharmaceutically acceptable salt thereof, wherein:
R¹ is selected from
(i) a hydrogen,
(ii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy),
(c) a hydroxyl,
(d) a phenoxy (said group being optionally substituted with the same or different 1 to 4 halogen, C₁₋₆ alkyl, or C₁₋₆ alkoxy), and
(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and
(iii) a C₃₋₁₀ cycloalkyl; a phenyl; a 5-6 membered monocyclic heteroaryl; a 4-8 membered saturated or partially unsaturated monocyclic heterocyclyl; a C₃₋₁₀ cycloalkyl-C₁₋₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;
wherein each of said groups in the aforesaid (iii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl);
X is O or S;
W is a covalent bond, —C≡C—, —CH═CH—, —O—, or —N(R⁵)—;
R⁵ is a hydrogen or a C₁₋₆ alkyl;
R² is selected from
(i) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy),
(c) a hydroxyl,
(d) a phenoxy (said group being optionally substituted with the same or different 1 to 4 halogen, C₁₋₆ alkyl, or C₁₋₆ alkoxy), and
(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and
(ii) a C₃₋₁₀ cycloalkyl; a phenyl; a 5-6 membered heteroaryl; a 4-8 membered saturated or partially unsaturated heterocyclyl; a C₃₋₁₀ cycloalkyl-C₁₋₄ alkyl; a phenyl-C₁₋₄ alkyl; a 5 or 6-membered heteroaryl-C₁₋₄ alkyl; or a 4-8 membered saturated or partially unsaturated heterocyclyl-C₁₋₄ alkyl;
wherein each of said groups in the aforesaid (ii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl);
alternatively, R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, hydroxy, or aminocarbonyl (said amino being optionally substituted with the same or different 1 to 2 C₁₋₆ alkyl)),
(c) a C₁₋₆ alkoxy (said group being optionally substituted with the same or different 1 to 3 halogen, C₁₋₆ alkoxy, or hydroxy),
(d) a phenyl (said group being optionally substituted with the same or different 1 to 4 halogen, cyano, C₁₋₆ alkyl, or C₁₋₆ alkoxy),
(e) a 5-6 membered monocyclic heteroaryl (said group being optionally substituted with the same or different 1 to 4 halogen, cyano, C₁₋₆ alkyl, or C₁₋₆ alkoxy),
(f) a 5-6 membered monocyclic heteroaryloxy (said group being optionally substituted with the same or different 1 to 4 halogen, cyano, C₁₋₆ alkyl, or C₁₋₆ alkoxy),
(g) a C₁₋₆ alkylcarbonyl (said alkyl being optionally substituted with the same or different 1 to 3 halogen, C₁₋₆ alkoxy, or amino (said amino being optionally substituted with the same or different 1 to 2 C₁₋₆ alkyl)), and
(h) an aminocarbonyl (said amino being optionally substituted with the same or different 1 to 2 C₁₋₆ alkyl);
R³ is selected from
(i) a hydrogen,
(ii) a halogen,
(iii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen),
(c) a hydroxy, and
(d) an oxo), or
(iv) a C₃₋₈ cycloalkyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo),
(v) a C₂₋₆ alkenyl (said group being optionally substituted with the same or different 1 to 4 halogen),
(vi) a C₃₋₈ cycloalkenyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo), and
(vii) 4-8 membered saturated or partially unsaturated monocyclic heterocyclyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano);
R⁴ is selected from
(i) a hydrogen,
(ii) a halogen,
(iii) a C₁₋₆ alkyl (said group being optionally substituted with the same or different 1 to 3 halogen), or
(iv) a cyano;
provided that when R¹ is a hydrogen, then W is —O— or —N(R⁵)— and R4 is a hydrogen.
As defined generally above, R¹ is selected from
(i) a hydrogen,
(ii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy),
(c) a hydroxyl,
(d) a phenoxy (said group being optionally substituted with the same or different 1 to 4 halogen, C₁₋₆ alkyl, or C₁₋₆ alkoxy), and
(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and
(iii) a C₃₋₁₀ cycloalkyl; a phenyl; a 5-6 membered monocyclic heteroaryl; a 4-8 membered saturated or partially unsaturated monocyclic heterocyclyl; a C₃₋₁₀ cycloalkyl-C₁₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;
wherein each of said groups in the aforesaid (iii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl). In some embodiments, R¹ is a C₅₋₆ cycloalkyl-C₁₋₄ alkyl (said group being optionally substituted with the same or different 1 to 4 halogen, C₁₋₆ alkyl, or C₁₋₆ alkoxy). In some embodiments, R¹ is a phenyl-C₁₋₄ alkyl (said group being optionally substituted with the same or different 1 to 4 halogen, C₁₋₆ alkyl, or C₁₋₆ alkoxy). In some embodiments, R¹ is a C₁₋₆ alkyl (said group being optionally substituted with the same or different 1 to 3 halogen, C₁₋₆ alkoxy, or hydroxy). In some embodiments, R¹ is a hydrogen.

As defined generally above, W is a covalent bond, —O—, or —N(R⁵)—. In some embodiments, W is a covalent bond. In some embodiments, W is a —O—. In some embodiments, W is —N(R⁵)—.

As defined generally above, R⁵ is a hydrogen or a C₁₋₆ alkyl. In some embodiments, R⁵ is a hydrogen. In some embodiments, R⁵ is a C₁₋₆ alkyl.

As defined generally above, R² is selected from
(i) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy),
(c) a hydroxyl,
(d) a phenoxy (said group being optionally substituted with the same or different 1 to 4 halogen, C₁₋₆ alkyl, or C₁₋₆ alkoxy), and
(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and
(ii) a C₃₋₁₀ cycloalkyl; a phenyl; a 5-6 membered heteroaryl; a 4-8 membered saturated or partially unsaturated heterocyclyl; a C₃₋₁₀ cycloalkyl-C₁₋₄ alkyl; a phenyl-C₁₋₄ alkyl; a 5 or 6-membered heteroaryl-C₁₋₄ alkyl; or a 4-8 membered saturated or partially unsaturated heterocyclyl-C₁₋₄ alkyl;
wherein each of said groups in the aforesaid (ii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl). In some embodiments, R² is a C₃₋₁₀ cycloalkyl (said group being optionally substituted with the same or different 1 to 4 halogen, C₁₋₆ alkyl (said group being optionally substituted with the same or different 1 to 3 halogen, C₁₋₆ alkoxy, or hydroxy), or C₁₋₆ alkoxy (said group being optionally substituted with the same or different 1 to 3 halogen, C₁₋₆ alkoxy, or hydroxy)). In some embodiments, R² is a C₃₋₁₀ cycloalkyl-C₁₋₄ alkyl (said group being optionally substituted with the same or different 1 to 4 halogen, C₁₋₆ alkyl (said group being optionally substituted with the same or different 1 to 3 halogen, C₁₋₆ alkoxy, or hydroxy), or C₁₋₆ alkoxy (said group being optionally substituted with the same or different 1 to 3 halogen, C₁₋₆ alkoxy, or hydroxy)). In some embodiments, R² is a phenyl-C₁₋₄ alkyl (said group being optionally substituted with the same or different 1 to 4 halogen, C₁₋₆ alkyl, or C₁₋₆ alkoxy). In some embodiments, R² is a 5-6 membered monocylic heteroaryl-C₁₋₄ alkyl (said group being optionally substituted with the same or different 1 to 4 halogen, C₁₋₆ alkyl, or C₁₋₆ alkoxy). In some embodiments, R² is a C₁₋₆ alkyl (said group being optionally substituted with the same or different 1 to 3 halogen, C₁₋₆ alkoxy, or hydroxy).

As defined generally above, R³ is selected from
(i) a hydrogen,
(ii) a halogen,
(iii) a C₁₋₆ alkyl (said group being optionally substituted with the same or different 1 to 4 group(s) selected from
(a) a halogen,
(b) a C₁₋₆ alkoxy (said group being optionally substituted with the same or different 1 to 3 halogen),
(c) a hydroxy, and
(d) an oxo), or
(iv) a C₃₋₈ cycloalkyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo),
(v) a C₂₋₆ alkenyl (said group being optionally substituted with the same or different 1 to 4 halogen),
(vi) a C₃₋₈ cycloalkenyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo), and
(vii) 4-8 membered saturated or partially unsaturated monocyclic heterocyclyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano). In some embodiments, R³ is a hydrogen. In some embodiments, R³ is a halogen. In some embodiments, R³ is a C₁₋₆ alkyl (said group being optionally substituted with the same or different 1 to 3 halogen, C₁₋₆ alkoxy, or hydroxy). In some embodiments, R³ is a C₃₋₈ cycloalkyl (said group being optionally substituted with the same or different 1 to 3 halogen, C₁₋₆ alkoxy, or hydroxy). In some embodiments, R³ is a C₁₋₆ alkenyl (said group being optionally substituted with the same or different 1 to 4 halogen). In some embodiments, R³ is a C₃₋₈ cycloalkenyl (said group being optionally substituted with the same or different 1 to 3 halogen, C₁₋₆ alkoxy, or hydroxy). In some embodiments, R³ is a 4-8 membered saturated or partially unsaturated monocyclic heterocyclyl (said group being optionally substituted with the same or different 1 to 3 halogen, C₁₋₆ alkoxy, or hydroxy). In some embodiments, R³ is a isopropyl (said group being optionally substituted with the same or different 1 to 3 halogen, C₁₋₆ alkoxy, or hydroxy). In some embodiments, R³ is a tetrahydropyranyl (said group being optionally substituted with the same or different 1 to 3 halogen, C₁₋₆ alkoxy, or hydroxy).

In some embodiments, R³ is a dihydropyranyl.

As defined generally above, R⁴ is selected from
(i) a hydrogen,
(ii) a halogen,
(iii) a C₁₋₆ alkyl (said group being optionally substituted with the same or different 1 to 3 halogen), or
(iv) a cyano. In some embodiments, R⁴ is a hydrogen. In some embodiments, R⁴ is a halogen. In some embodiments, R⁴ is a C₁₋₆ alkyl (said group being optionally substituted with the same or different 1 to 3 halogen). In some embodiments, R⁴ is a cyano.

As defined generally above R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, hydroxy, or aminocarbonyl (said amino being optionally substituted with the same or different 1 to 2 C₁₋₆ alkyl)),
(c) a C₁₋₆ alkoxy (said group being optionally substituted with the same or different 1 to 3 halogen, C₁₋₆ alkoxy, or hydroxy),
(d) a phenyl (said group being optionally substituted with the same or different 1 to 4 halogen, cyano, C₁₋₆ alkyl, or C₁₋₆ alkoxy),
(e) a 5-6 membered monocyclic heteroaryl (said group being optionally substituted with the same or different 1 to 4 halogen, cyano, C₁₋₆ alkyl, or C₁₋₆ alkoxy),
(f) a 5-6 membered monocyclic heteroaryloxy (said group being optionally substituted with the same or different 1 to 4 halogen, cyano, C₁₋₆ alkyl, or C₁₋₆ alkoxy),
(g) a C₁₋₆ alkylcarbonyl (said alkyl being optionally substituted with the same or different 1 to 3 halogen, C₁₋₆ alkoxy, or amino (said amino being optionally substituted with the same or different 1 to 2 C₁₋₆ alkyl)), and
(h) an aminocarbonyl (said amino being optionally substituted with the same or different 1 to 2 C₁₋₆ alkyl).

In certain embodiments, the present invention provides any compounds selected from those depicted in the Examples disclosed herein, or a pharmaceutically acceptable salt thereof.

### 4. Pharmaceutically Acceptable Compositions, Uses, Formulation and Administration

According to another embodiment, the present invention provides a composition comprising the compound of the present invention or a pharmaceutically acceptable salt, an ester, or salt of an ester thereof; and a pharmaceutically acceptable carrier, adjuvant, or vehicle. In certain embodiments, the amount of the compound in the composition of the present invention is an amount such that is effective to measurably inhibit PDE1 in a biological sample or in a patient. In certain embodiments, the composition of the present invention is formulated for administration to a patient in need of such composition. In some embodiments, the composition of the present invention is formulated for oral administration to a patient in need thereof.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which a composition is formulated. The pharmaceutically acceptable carriers, adjuvants or vehicles that may be used in the compositions of the present invention include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or another derivative of the compound of the present invention that, upon administration to a recipient, is capable of providing, either directly or indirectly, the compound of the present invention or an inhibitorily active metabolite or residue thereof.

As used herein, the term “inhibitorily active metabolite or residue” means a metabolite or residue of the compound of the present invention, which can be an inhibitor of PDE1.

The composition of the present invention may be administered orally, parenterally, by inhalation spray, topically, rectally, nasally, buccally, vaginally or via an implanted reservoir. The term “parenteral” as used herein includes subcutaneous, intraperitoneal, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of the present invention may be aqueous or oleaginous suspension. The suspension may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. The acceptable vehicles and solvents that may be employed in the present invention include, for example, water, Ringer's solution and isotonic sodium chloride solution. In addition, sterile fixed oils are conventionally employed as a solvent or suspending medium in the present invention.

For this purpose, any bland fixed oil may be employed including synthetic mono- or di-glycerides. Fatty acids such as oleic acid and its glyceride derivatives are useful in the preparation of injectables, as are natural pharmaceutically-acceptable oils such as olive oil or castor oil, especially in their polyoxyethylated forms. These oil solutions or suspensions may also contain a long-chain alcohol diluent or dispersant such as carboxymethyl cellulose or similar dispersing agents that are commonly used in the formulation of pharmaceutically acceptable dosage forms including emulsions and suspensions. Other commonly used surfactants such as Tweens, Spans and other emulsifying agents or bioavailability enhancers which are commonly used in the manufacture of pharmaceutically acceptable solid, liquid, or other dosage forms may also be used for the purposes of the formulation.

The pharmaceutically acceptable compositions of the present invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. The diluents useful for oral administration in a capsule form include lactose and dried cornstarch. When aqueous suspensions are required for oral use, the active ingredient is combined with emulsifying and suspending agents. If desired, certain sweetening, flavoring or coloring agents may also be added to the compositions.

Alternatively, the pharmaceutically acceptable composition of the present invention may be administered in the form of suppositories for rectal administration. The composition can be prepared by mixing the active ingredient with a suitable non-irritating excipient that is solid at room temperature but liquid at a rectal temperature and therefore melt in the rectum to release the active ingredient. Such an excipient includes cocoa butter, beeswax and polyethylene glycols.

The pharmaceutically acceptable composition of the present invention may also be administered topically, especially when the target of treatment includes areas or organs readily accessible by topical application, including in the case of the diseases of eyes, skins, or lower intestinal tracts. Suitable topical formulations are readily prepared for each of these areas or organs.

The topical application for lower intestinal tracts can be effected in a rectal suppository formulation (see above) or in a suitable enema formulation. Topically-transdermal patches may also be used for the application.

For topical applications, the pharmaceutically acceptable composition may be also formulated in a suitable ointment containing the active component suspended or dissolved in one or more carrier. The carrier for topical administration of the compound of the present invention includes, but is not limited to, mineral oil, liquid petrolatum, white petrolatum, propylene glycol, polyoxyethylene, polyoxypropylene compound, emulsifying wax and water. Alternatively, the pharmaceutically acceptable composition can be formulated in a suitable lotion or cream containing the active component suspended or dissolved in one or more pharmaceutically acceptable carrier. Such a suitable carrier includes, but is not limited to, mineral oil, sorbitan monostearate, polysorbate 60, cetyl esters wax, cetearyl alcohol, 2-octyldodecanol, benzyl alcohol and water.

For ophthalmic uses, the pharmaceutically acceptable composition may be formulated as micronized suspensions in isotonic pH-adjusted sterile saline, or preferably, as solutions in isotonic pH-adjusted sterile saline, either with or without a preservative such as benzylalkonium chloride. Alternatively, for ophthalmic uses, the pharmaceutically acceptable composition may be formulated in an ointment such as petrolatum.

The pharmaceutically acceptable composition of the present invention may also be administered by nasal aerosol or inhalation. Such a composition is prepared according to techniques well-known in the art of pharmaceutical formulations and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable composition of the present invention is formulated for oral administration. Such a formulation may be administered with or without food. In some embodiments, the pharmaceutically acceptable composition of the present invention is administered without food. In other embodiments, the pharmaceutically acceptable composition of the present invention is administered with food.

The amount of the compound of the present invention that may be combined with carrier materials to produce a composition in a single dosage form will vary depending upon a variety of factors, including a host treated and particular modes of administration. Preferably, the composition may be formulated so that a dosage between 0.01-100 mg/kg body weight/day of the composition can be administered to a patient receiving the composition.

It should also be understood that a specific dosage and treatment regimen for any particular patient will depend upon a variety of factors, including the activity of the specific compound employed, the age, body weight, general health, sex of the patient, diet, time of administration, rate of excretion, drug combination, and the judgment of the treating physician and the severity of the particular disease being treated. The amount of the compound of the present invention in the composition will also depend upon which compound is comprised in the composition.

Uses of Compounds and Pharmaceutically Acceptable Compositions

Phosphodiesterases (PDEs) are enzymes that catalyze the hydrolysis of cyclic phosphate bonds of cyclic guanosine monophosphate (cGMP) and/or cyclic adenosine monophosphate (cAMP). Lugnier, C., Pharmacology & Therapeutics (2006), 109, 366. The PDE superfamily can be grouped into 11 families (PDE1-11) based on their sequences, regulation and substrate specificities. Each family can contain multiple subtypes, each of which is the product of individual genes. In particular, the PDE1 family, consisting of PDE1A, PDE1B and PDE1C, are so-called dual substrate enzymes that hydrolyze both cGMP and cAMP, and are regulated by Ca²⁺ and calmodulin. PDE1A is expressed throughout the brain, especially in the hippocampus and cerebellum, and at lower levels in the striatum, as well as in the peripheral vasculature. PDE1B, by contrast, is expressed primarily in the striatum and cerebellum, and is often found in the regions with high dopaminergic tone and dopamine D1 receptor expression. PDE1C is primarily expressed in the heart, olfactory epithelium, and striatum. Considering these expression patterns, the compound that is selective for PDE1B over PDE1A and/or PDE1C may have fewer effects on the cardiovascular system.

Due to the expression pattern of the PDE1 family, inhibition of PDE1 may be useful in the treatment of disorders involving learning and memory by enhancing neuronal plasticity. The increased levels of intracellular cAMP and cGMP caused by PDE1 inhibition trigger cascades that ultimately lead to the phosphorylation and activation of the transcription factors, cAMP Responsive Element Binding Protein (CREB) and Serum Response Factor (SRF). Josselyn, S. A., Nguyen, P. V., Current Drug Targets—CNS & Neurological Disorders (2005) 4, 481. Activation of CREB and SRF can lead to the expression of plasticity-related genes which mediate the processes that are critical for neuronal plasticity such as remodeling of dendritic spines. PDE1 inhibitors may therefore be useful in the treatment of cognitive symptoms of disorders such as Alzheimer's Disease, Parkinson's Disease, Stroke, Schizophrenia, Down Syndrome, Fetal Alcohol Syndrome and others.

Due to its location in the striatum and its role in modulating levels of secondary messengers such as cyclic nucleotides, PDE1 is also a regulator of locomotor activity. Reed, T. M. J., et al., Journal of Neuroscience (2002) 22, 5189). Due to their ability to increase levels of cyclic nucleotides in the striatum, PDE1 inhibitors are expected to potentiate the effects of D1 agonists by inhibiting the degradation of cAMP and cGMP. This potentiation of dopamine signaling may be useful in the treatment of diseases including, but not limited to Parkinson's Disease, depression and cognitive disorders including Cognitive Impairment Associated with Schizophrenia.

The activity of the compound utilized in the present invention as an inhibitor of PDE1 or an agent for treatment for a neurological or psychiatric disorder, may be assayed in vitro or in vivo. An in vivo assessment of the efficacy of the compounds of the invention may be made using an animal model of a neurological or psychiatric disorder, e.g. a rodent or primate model. Cell-based assays may be performed using, e.g. a cell line isolated from a tissue that expresses PDE1, or a cell line that recombinantly expresses PDE1. Additionally, biochemical or mechanism-based assays, e.g. measuring cAMP or cGMP levels, Northern blot, RT-PCR, etc., may be performed. In vitro assays include assays that determine cell morphology, protein expression, and/or the cytotoxicity, enzyme inhibitory activity, and/or the subsequent functional consequences of treatment of cells with compounds of the invention. Alternate in vitro assays quantify the ability of the inhibitor to bind to protein or nucleic acid molecules within the cell. Inhibitor binding may be measured by radiolabelling the inhibitor prior to binding, isolating the inhibitor/target molecule complex and determining the amount of radiolabel bound. Alternatively, inhibitor binding may be determined by running a competition experiment where new inhibitors are incubated with purified proteins or nucleic acids bound to known radioligands. Detailed conditions for assaying a compound utilized in the present invention as an inhibitor of PDE1 are set forth in the Examples below. The aforementioned assays are exemplary and not intended to limit the scope of the invention. The skilled practitioner can appreciate that modifications can be made to conventional assays to develop equivalent assays that obtain the same result.

As used herein, the terms “treatment,” “treat,” and “treating” denote reversing, alleviating, delaying the onset of, or inhibiting the progress of a disease or disorder, or one or more symptom thereof, as described herein. In some embodiments, treatment may be conducted after one or more symptoms have developed. In other embodiments, treatment may be conducted in the absence of symptoms. For example, treatment may be conducted to a susceptible individual prior to the onset of symptoms (e.g. in light of a history of symptoms and/or in light of genetic or other susceptibility factors). Treatment may also be continued after symptoms have resolved, for example in order to prevent or delay their recurrence.

In some embodiments, the compounds and compositions, according to the method of the present invention, may be administered in any amount and any route of administration effective for treating or lessening the severity of a disease associated with PDE 1.

In some embodiments, the compounds and compositions, according to the method of the present invention, may be administered in any amount and any route of administration effective for treating or lessening the severity of a neurological or psychiatric disorder.

In some embodiments, the neurological or psychiatric disorder is selected from schizophrenia or psychosis including schizophrenia (including paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (including phencyclidine, ketamine and other dissociative anesthetics, amphetamine and other psychostimulants, and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Down syndrome, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and postpartum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, Parkinsonism-ALS dementia complex and basal ganglia calcification), medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias {including drug e.g. L-DOPA induced dyskinesia tremor (such as rest tremor, postural tremor, and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)}; urinary incontinence; neuronal damage including ocular damage, retinopathy or macular degeneration of the eye, tinnitus, hearing impairment and loss, and brain edema; emesis; and sleep disorders including insomnia and narcolepsy.

In some embodiments, the neurological or psychiatric disorder is selected from the group consisting of Alzheimer's Disease, Parkinson's Disease, depression, cognitive impairment, stroke, schizophrenia, Down Syndrome, and Fetal Alcohol Syndrome. In some embodiments, the neurological or psychiatric disorder is Alzheimer's Disease. In some embodiments, the neurological or psychiatric disorder is Parkinson's Disease. In some embodiments, the neurological or psychiatric disorder is depression. In some embodiments, the neurological or psychiatric disorder is cognitive impairment. In some embodiments, the neurological or psychiatric disorder is stroke. In some embodiments, the neurological or psychiatric disorder is schizophrenia including positive, negative and cognitive symptoms. In some embodiments, the neurological or psychiatric disorder is Down Syndrome. In some embodiments, the neurological or psychiatric disorder is Fetal Alcohol Syndrome.

In some embodiments, the neurological or psychiatric disorder involves a deficit in cognition (e.g. a deficiency in one or more cognitive domains as defined by the Diagnostic and Statistical Manual of Mental Disorders, 5th Ed., American Psychiatric Publishing (2013) (“DSM-5”), which includes: complex attention, executive function, learning and memory, language, perceptual-motor, and social cognition). In some embodiments, the neurological or psychiatric disorder is associated with a deficiency in dopamine signaling. In some embodiments, the neurological or psychiatric disorder is associated with basal ganglia dysfunction. In some embodiments, the neurological or psychiatric disorder is associated with dysregulated locomotor activity.

In some embodiments, the neurological or psychiatric disorder is associated with a deficiency in cyclic nucleotide signaling molecules. In some embodiments, the neurological or psychiatric disorder is associated with a deficiency in cAMP and/or cGMP. In some embodiments, the neurological or psychiatric disorder is associated with low activity of cAMP Responsive Element Binding Protein (CREB), Serum Response Factor (SRF), or both.

In some embodiments, the compounds and compositions, according to the method of the present invention, may be administered in any amount and any route of administration effective for treating or lessening the severity of a circulatory or cardiovascular disorder. In some embodiments, the circulatory or cardiovascular disorder is selected from the group consisting of cerebrovascular disease, stroke, congestive heart disease, hypertension, pulmonary hypertension and sexual dysfunction.

In some embodiments, the compounds and compositions, according to the method of the present invention, may be administered in any amount and any route of administration effective for treating or lessening the severity of respiratory and inflammatory diseases. In some embodiments, respiratory and inflammatory diseases are selected from the group consisting of asthma, chronic obstructive pulmonary disease, allergic rhinitis, and autoimmune and inflammatory diseases.

In some embodiments, the present invention provides a method of treating the neurological or psychiatric disorder described herein, comprising administering the compound of the invention in conjunction with one or more pharmaceutical agent to a patient in need thereof. Such suitable pharmaceutical agents that may be used in combination with the compound of the present invention include anti-Parkinson's drugs, anti-Alzheimer's drugs, anti-depressants, anti-psychotics, anti-ischemics, CNS depressants, anti-cholinergics, and nootropics.

Such suitable anti-Parkinson's drugs include, but are not limited to, dopamine replacement therapy (e.g. L-DOPA, carbidopa, COMT inhibitors such as entacapone), dopamine agonists (e.g. D1 agonists, D2 agonists, mixed D1/D2 agonists; bromocriptine, pergolide, cabergoline, ropinirole, pramipexole, or apomorphine in combination with domperidone), histamine H2 antagonists, and monoamine oxidase inhibitors such as selegiline and tranylcypromine.

In some embodiments, the compounds of the invention may be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine receptor agonist may be combined in the form of its pharmaceutically acceptable salt: for example, alentemol hydrobromide, bromocriptine mesylate, fenoldopam mesylate, naxagolide hydrochloride and pergolide mesylate. Lisuride and pramipexole are commonly used in the non-salt form.

Such suitable anti-Alzheimer's drugs include, but are not limited to, beta-secretase inhibitors, gamma-secretase inhibitors, HMG-CoA reductase inhibitors, NSAID's including ibuprofen, vitamin E, and anti-amyloid antibodies. In some embodiments, the anti-Alzheimer's drug is memantine.

Such suitable anti-depressants and anti-anxiety agents include, but are not limited to norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists.

In particular, the suitable anti-depressant and anti-anxiety agents include, but are not limited to, amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone; and pharmaceutically acceptable salts thereof.

The exact amounts of the compounds or compositions of the present invention required will vary from subject to subject, depending on the species, age, and general condition of the subject, the severity of infection, the particular agent used therein, modes of administration, and the like. The compounds of the invention are preferably formulated in a dosage unit form for ease of administration and uniformity of dosage. The expression “dosage unit form” as used herein refers to a physically discrete unit of the agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or concomitant with the specific compound employed, and other factors well known in the medical arts. The term “patient”, as used herein, means an animal, preferably a mammal, and most preferably a human.

The pharmaceutically acceptable composition of the present invention can be administered to humans and other animals orally, rectally, parenterally, intracisternally, intravaginally, intraperitoneally, topically (such as in powders, ointments, or drops), bucally, as an oral or nasal spray, or the like, depending on the severity of the infection being treated. In certain embodiments, the compound of the invention may be administered orally or parenterally at dosage levels of about 0.01 mg/kg to about 50 mg/kg, and preferably from about 1 mg/kg to about 25 mg/kg, of a subject body weight per day, one or more time a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. The acceptable vehicles and solvents that may be employed in the present invention include, for example, water, Ringer's solution, U.S.P. and isotonic sodium chloride solution. In addition, sterile fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, the delayed absorption of the parenterally-administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of releasing the compound can be controlled. Examples of other biodegradable polymers include poly(orthoesters) and poly(anhydrides). The injectable depot formulations are also prepared by entrapping the compound in liposomes or microemulsions that are compatible with body tissues.

The compositions for rectal or vaginal administration are preferably suppositories which can be prepared by mixing the compounds of the present invention with suitable non-irritating excipients or carriers such as cocoa butter, polyethylene glycol or a suppository wax which are solid at the ambient temperature but liquid at the body temperature and therefore melt in the rectum or vaginal cavity to release the active compound.

Solid dosage forms for oral administration include capsules, tablets, pills, powders, and granules. In such solid dosage forms, the active compound is mixed with at least one inert pharmaceutically-acceptable excipient or carrier such as sodium citrate or dicalcium phosphate and/or a) fillers or extenders such as starches, lactose, sucrose, glucose, mannitol, and silicic acid, b) binders such as, for example, carboxymethyl-cellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition wherein the active ingredient(s) are only, or preferentially, released in a certain part of the intestinal tract, optionally, in a delayed manner. Examples of embedding compositions that can be used herein include polymeric substances and waxes.

The active compounds can also be in micro-encapsulated form with one or more excipient as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the active compound may be admixed with at least one inert diluent such as sucrose, lactose or starch. Such dosage forms may also comprise, as is normal practice, additional substances other than inert diluents, e.g. tableting lubricants and other tableting aids such as magnesium stearate and microcrystalline cellulose. In the case of capsules, tablets and pills, the dosage forms may also comprise buffering agents. They may optionally contain opacifying agents and can also be of a composition wherein the active ingredient(s) are only, or preferentially, released in a certain part of the intestinal tract, optionally, in a delayed manner.

Dosage forms for topical or transdermal administration of the compound of the present invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations such as ear drops and eye drops are also contemplated as being within the scope of the present invention. Additionally, the present invention encompasses the use of transdermal patches, which have the added advantage of providing controlled delivery of the compound to the body. Such dosage forms can be made by dissolving or dispensing the compound in a proper medium. Absorption enhancers can also be used to increase the fluidity of the compound across the skin. The flow rate can be controlled by either providing a rate controlling membrane or dispersing the compound in a polymer matrix or gel.

One embodiment of the present invention provides a method of inhibiting PDE1 in a biological sample comprising the step of contacting said biological sample with the compound of the present invention, or a composition comprising said compound. In some embodiments, the PDE1 is PDE1A. In some embodiments, the PDE1 is PDE1B. In some embodiments, the PDE1 is PDE1C. Another embodiment of the present invention provides a method of inhibiting PDE1B selectively over PDE1A and/or PDE1C. Still another embodiment of the present invention provides a method of inhibiting PDE1B selectively over PDE1A. Still another embodiment of the present invention provides a method of inhibiting PDE1B selectively over PDE1C. Still another embodiment of the present invention provides a method of inhibiting PDE1B selectively over PDE1A and PDE1C. In some embodiments, the selectivity for PDE1B over PDE1A and/or PDE1C is up to and including five-fold. In some embodiments, the selectivity for PDE1B over PDE1A and/or PDE1C is up to and including ten-fold. In some embodiments, the selectivity for PDE1B over PDE1A and/or PDE1C is up to and including twenty-fold. In some embodiments, the selectivity for PDE1B over PDE1C is up to and including fifty-fold. In some embodiments, the selectivity for PDE1B over PDE1C is up to and including one hundred-fold. In some embodiments, the selectivity for PDE1B over PDE1C is up to and including two hundred-fold. The selectivity for one PDE1 isoform over another one refers to the inverse ratio of IC₅₀ values against each respective isoform as determined using the HTRF PDE1 inhibition assay or the SPA assay described in the Examples. For example, the selectivity of the compound of the present invention for PDE1B over PDE1C refers to the ratio IC₅₀ (PDE1C)/IC₅₀ (PDE1B), wherein IC₅₀ (PDE1C) is the IC₅₀ value of the compound against PDE1C as determined using the described HTRF PDE1 inhibition assay or the SPA assay, and IC₅₀ (PDE1B) is the IC₅₀ value of the compound against PDE1B as determined using the described HTRF PDE1 inhibition assay or the SPA assay.

Still another embodiment of the present invention provides a method of modulating cyclic nucleotide levels in a biological sample comprising the step of contacting said biological sample with the compound of the present invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of enzymes in a biological sample is useful for a variety of purposes that are known to one of skill in the art. Examples of such purposes include, but are not limited to biological assays, gene expression studies, and biological target identification.

Another embodiment of the present invention relates to a method of inhibiting PDE1 in a patient comprising the step of administering to said patient the compound of the present invention, or a composition comprising said compound. In some embodiments, the PDE1 is PDE1B. Still another embodiment of the present invention provides a method of inhibiting PDE1B in a patient selectively over PDE1A and/or PDE1C. Still another embodiment of the present invention provides a method of inhibiting PDE1B in a patient selectively over PDE1A. Still another embodiment of the present invention provides a method of inhibiting PDE1B in a patient selectively over PDE1C. Still another embodiment of the present invention provides a method of inhibiting PDE1B in a patient selectively over PDE1A and PDE1C. In some embodiments, the selectivity for PDE1B over PDE1A and/or PDE1C is up to and including five-fold. In some embodiments, the selectivity for PDE1B over PDE1A and/or PDE1C is up to and including ten-fold. In some embodiments, the selectivity for PDE1B over PDE1A and/or PDE1C is up to and including twenty-fold. In some embodiments, the selectivity for PDE1B over PDE1C is up to and including fifty-fold. In some embodiments, the selectivity for PDE1B over PDE1C is up to and including one hundred-fold. In some embodiments, the selectivity for PDE1B over PDE1C is up to and including two hundred-fold. The selectivity for one PDE1 isoform over another one refers to the inverse ratio of IC₅₀ values against each respective isoform as determined using the HTRF PDE1 inhibition assay or the SPA assay described in the Examples. For example, the selectivity of the compound of the present invention for PDE1B over PDE1C refers to the ratio IC₅₀ (PDE1C)/IC₅₀ (PDE1B), wherein IC₅₀ (PDE1C) is the IC₅₀ value of the compound against PDE1C as determined using the described HTRF PDE1 inhibition assay or the SPA assay, and IC₅₀ (PDE1B) is the IC₅₀ value of the compound against PDE1B as determined using the described HTRF PDE1 inhibition assay or the SPA assay.

Depending upon the particular condition or disease to be treated, additional therapeutic agents, which are normally administered to treat the condition or disease, may be administered in combination with the compounds and compositions of the present invention. As used herein, additional therapeutic agents that are normally administered to treat a particular disease or condition are known as “appropriate for the disease or condition being treated” in the art.

In certain embodiments, a combination of 2 or more therapeutic agents may be administered together with the compounds of the invention. In certain embodiments, a combination of 3 or more therapeutic agents may be administered with the compounds of the invention.

Other examples of agents with which the inhibitors of the present invention may be combined include, without limitation: vitamins and nutritional supplements, antiemetics (e.g. 5-HT3 receptor antagonists, dopamine antagonists, NK1 receptor antagonists, histamine receptor antagonists, cannabinoids, benzodiazepines, or anti-cholinergics), agents for treating Multiple Sclerosis (MS) such as beta interferon (e.g. Avonex (Registered Trademark) and Rebif (Registered Trademark)), Copaxone (Registered Trademark), and mitoxantrone; treatments for asthma such as albuterol and Singulair (Registered Trademark); anti-inflammatory agents such as corticosteroids, TNF blockers, IL-1 RA, azathioprine, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspoglutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In certain embodiments, the compounds of the present invention or a pharmaceutically acceptable composition comprising the same may be administered in combination with antisense agents, a monoclonal or polyclonal antibody or an siRNA therapeutic.

Those additional agents may be administered separately from a composition comprising the compound of the present invention as part of a multiple dosage regimen. Alternatively, those agents may be part of a single dosage form, mixed together with the compound of the present invention in a single composition. If administered as part of a multiple dosage regime, two active agents may be administered simultaneously, sequentially or within a period of time from one another, normally within five hours from one another.

As used herein, the terms “combination,” “combined,” and related terms refer to the simultaneous or sequential administration of therapeutic agents in accordance with the present invention. For example, the compound of the present invention may be administered with another therapeutic agent simultaneously or sequentially in separate unit dosage forms or together in a single unit dosage form. Accordingly, the present invention provides a single unit dosage form comprising the compound of formula I, an additional therapeutic agent, and a pharmaceutically acceptable carrier, adjuvant, or vehicle.

The amounts of both the compound of the present invention and an additional therapeutic agent (in the compositions which comprise an additional therapeutic agent as described above) that may be combined with carrier materials to produce a single dosage form may vary depending upon a host treated and the particular mode of administration. Preferably, the compositions of the present invention should be formulated so that a dosage of between 0.01-100 mg/kg body weight/day of the compound of the present invention can be administered.

In those compositions which comprise an additional therapeutic agent, that additional therapeutic agent and the compound of the present invention may act synergistically. Therefore, the amount of the additional therapeutic agent in such compositions will be less than that required in a monotherapy utilizing only the therapeutic agent. In such compositions, a dosage of between 0.01-100 μg/kg body weight/day of the additional therapeutic agent can be administered.

The amount of the additional therapeutic agent present in the compositions of the present invention may be no more than the amount that would normally be administered in a composition comprising the therapeutic agent as the only active agent. Preferably, the amount of the additional therapeutic agent in the compositions of the present invention may range from about 50% to 100% of the amount normally present in a composition comprising the therapeutic agent as the only active agent.

Another embodiment of the present invention provides a medicament comprising at least one compound of formula I or a pharmaceutically acceptable salt thereof and a pharmaceutically acceptable carrier.

Still another embodiment of the present invention provides the use of the compound of formula I in the manufacture of a medicament for the treatment of a neurological or psychiatric disorder.

Still another embodiment of the present invention provides the use of the compound of formula I in the manufacture of a medicament for the treatment of a circulatory or cardiovascular disorder.

Still another embodiment of the present invention provides the use of the compound of formula I in the manufacture of a medicament for the treatment of respiratory and inflammatory diseases.

The present invention also includes the following illustrative embodiments:
Item 1. A compound of formula I:

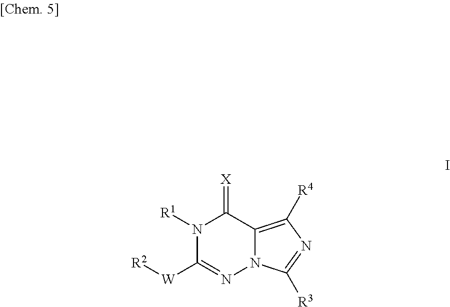

or a pharmaceutically acceptable salt thereof, wherein:
R¹ is selected from
(i) a hydrogen,
(ii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy),
(c) a hydroxyl,
(d) a phenoxy (said group being optionally substituted with the same or different 1 to 4 halogen, C₁₋₆ alkyl, or C₁₋₆ alkoxy), and
(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and
(iii) a C₃₋₁₀ cycloalkyl; a phenyl; a 5-6 membered monocyclic heteroaryl; a 4-8 membered saturated or partially unsaturated monocyclic heterocyclyl; a C₃₋₁₀ cycloalkyl-C₁₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;
wherein each of said groups in the aforesaid (iii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl);
X is O or S;
W is a covalent bond, —C≡C—, —CH═CH—, —O—, or —N(R⁵)—;
R⁵ is a hydrogen or a C₁₋₆ alkyl;
R² is selected from
(i) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy),
(c) a hydroxyl,
(d) a phenoxy (said group being optionally substituted with the same or different 1 to 4 halogen, C₁₋₆ alkyl, or C₁₋₆ alkoxy), and
(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and
(ii) a C₃₋₁₀ cycloalkyl; a phenyl; a 5-6 membered heteroaryl; a 4-8 membered saturated or partially unsaturated heterocyclyl; a C₃₋₁₀ cycloalkyl-C₁₋₄ alkyl; a phenyl-C₁₋₄ alkyl; a 5 or 6-membered heteroaryl-C₁₋₄ alkyl; or a 4-8 membered saturated or partially unsaturated heterocyclyl-C₁₋₄ alkyl;
wherein each of said groups in the aforesaid (ii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl);
alternatively, R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, hydroxy, or aminocarbonyl (said amino being optionally substituted with the same or different 1 to 2 C₁₋₆ alkyl)),
(c) a C₁₋₆ alkoxy (said group being optionally substituted with the same or different 1 to 3 halogen, C₁₋₆ alkoxy, or hydroxy),
(d) a phenyl (said group being optionally substituted with the same or different 1 to 4 halogen, cyano, C₁₋₆ alkyl, or C₁₋₆ alkoxy),
(e) a 5-6 membered monocyclic heteroaryl (said group being optionally substituted with the same or different 1 to 4 halogen, cyano, C₁₋₆ alkyl, or C₁₋₆ alkoxy),
(f) a 5-6 membered monocyclic heteroaryloxy (said group being optionally substituted with the same or different 1 to 4 halogen, cyano, C₁₋₆ alkyl, or C₁₋₆ alkoxy),
(g) a C₁₋₆ alkylcarbonyl (said alkyl being optionally substituted with the same or different 1 to 3 halogen, C₁₋₆ alkoxy, or amino (said amino being optionally substituted with the same or different 1 to 2 C₁₋₆ alkyl)), and
(h) an aminocarbonyl (said amino being optionally substituted with the same or different 1 to 2 C₁₋₆ alkyl);
R³ is selected from
(i) a hydrogen,
(ii) a halogen,
(iii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen),
(c) a hydroxy, and
(d) an oxo), or
(iv) a C₃₋₈ cycloalkyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo),
(v) a C₂₋₆ alkenyl (said group being optionally substituted with the same or different 1 to 4 halogen),
(vi) a C₃₋₈ cycloalkenyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo), and
(vii) 4-8 membered saturated or partially unsaturated monocyclic heterocyclyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano);
R⁴ is selected from
(i) a hydrogen,
(ii) a halogen,
(iii) a C₁₋₆ alkyl (said group being optionally substituted with the same or different 1 to 3 halogen), or
(iv) a cyano; provided that when R¹ is a hydrogen, then W is —O— or —N(R⁵)— and R⁴ is a hydrogen.
Item 2. The compound of Item 1, or a pharmaceutically acceptable salt thereof, wherein R¹ is selected from
(i) a hydrogen,
(ii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy), and
(c) a hydroxyl), and
(iii) a C₃₋₁₀ cycloalkyl; a C₃₋₁₀ cycloalkyl-C₁₋₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;
wherein each of said groups in the aforesaid (iii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy), and
(g) a 5-6 membered monocyclic heteroaryl (said group being optionally substituted with the same or different 1 to 4 halogen, cyano, C₁₋₆ alkyl, or C₁₋₆ alkoxy).
Item 3. The compound of Item 1 or 2, or a pharmaceutically acceptable salt thereof, wherein R¹ is selected from
(i) a hydrogen,
(ii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy), and
(c) a hydroxyl),
(iii) a C₃₋₈ cycloalkyl (said group being optionally substituted with the same or different 1 to 4 halogen, C₁₋₆ alkyl, or C₁₋₆ alkoxy), and
(iv) C₃₋₈ cycloalkyl-C₁₋₄ alkyl (said group being optionally substituted with the same or different 1 to 4 halogen, C₁₋₆ alkyl, or C₁₋₆ alkoxy);
W is —O—, or —N(R⁵)—;
R⁵ is a hydrogen, or a C₁₋₆ alkyl; and
R² is a C₃₋₁₀ cycloalkyl-C₁₋₄ alkyl; a phenyl-C₁₋₄ alkyl; a 5 or 6-membered monocyclic heteroaryl-C₁₋₄ alkyl; a 4-8 membered saturated or partially unsaturated monocyclic heterocyclyl-C₁₋₄ alkyl; a C₃₋₁₀ cycloalkyl; or a 4-8 membered saturated or partially unsaturated monocyclic heterocyclyl;
wherein each of said groups in R² is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl);
alternatively, R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, hydroxyl, or aminocarbonyl (said amino being optionally substituted with the same or different 1 to 2 C₁₋₆ alkyl)),
(c) a C₁₋₆ alkoxy (said group being optionally substituted with the same or different 1 to 3 halogen, C₁₋₆ alkoxy, or hydroxy),
(d) a phenyl (said group being optionally substituted with the same or different 1 to 4 halogen, cyano, C₁₋₆ alkyl, or C₁₋₆ alkoxy),
(e) a 5-6 membered monocyclic heteroaryl (said group being optionally substituted with the same or different 1 to 4 halogen, cyano, C₁₋₆ alkyl, or C₁₋₆ alkoxy),
(f) a 5-6 membered monocyclic heteroaryloxy (said group being optionally substituted with the same or different 1 to 4 halogen, cyano, C₁₋₆ alkyl, or C₁₋₆ alkoxy),
(g) a C₁₋₆ alkylcarbonyl (said alkyl being optionally substituted with the same or different 1 to 3 halogen, C₁₋₆ alkoxy, or amino (said amino being optionally substituted with the same or different 1 to 2 C₁₋₆ alkyl)), and
(h) an aminocarbonyl (said amino being optionally substituted with the same or different 1 to 2 C₁₋₆ alkyl).
Item 4. The compound of any one of Items 1 to 3, or a pharmaceutically acceptable salt thereof, wherein R¹ is a C₁₋₆ alkyl (said group being optionally substituted with the same or different 1 to 3 halogen, or C₁₋₆ alkoxy); and
R² is a C₃₋₁₀ cycloalkyl-C₁₋₄ alkyl; a phenyl-C₁₋₄ alkyl; a 5 or 6-membered monocyclic heteroaryl-C₁₋₄ alkyl; or a C₃₋₁₀ cycloalkyl;
wherein each of said groups in R² is optionally substituted with the same or different 1 to 4 group(s) selected from
(a) a halogen,
(b) a C₁₋₆ alkyl (said group being optionally substituted with the same or different 1 to 3 halogen, hydroxy or C₁₋₆ alkoxy),
(c) a C₁₋₆ alkoxy (said group being optionally substituted with the same or different 1 to 3 halogen, hydroxy or C₁₋₆ alkoxy),
(d) a cyano,
(e) a phenyl (said group being optionally substituted with the same or different 1 to 4 halogen, cyano, C₁₋₆ alkyl, or C₁₋₆ alkoxy), and
(f) a 5-6 membered monocyclic heteroaryl (said group being optionally substituted with the same or different 1 to 4 halogen, cyano, C₁₋₆ alkyl, or C₁₋₆ alkoxy).
Item 5. The compound of Item 4, or a pharmaceutically acceptable salt thereof, wherein R² is a C₃₋₈ cycloalkyl-C₁₋₄ alkyl or a C₃₋₈ cycloalkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, hydroxy or C₁₋₆ alkoxy), and
(c) a C₁₋₆ alkoxy (said group being optionally substituted with the same or different 1 to 3 halogen, hydroxy or C₁₋₆ alkoxy).
Item 6. The compound of Item 1, or a pharmaceutically acceptable salt thereof, wherein R¹ is a C₃₋₁₀ cycloalkyl; a C₃₋₁₀ cycloalkyl-C₁₋₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy), and
(g) a 5-6 membered monocyclic heteroaryl (said group being optionally substituted with the same or different 1 to 4 halogen, cyano, C₁₋₆ alkyl, or C₁₋₆ alkoxy);
W is —O— or —N(R⁵)—;
R⁵ is a hydrogen or a C₁₋₆ alkyl; and
R² is a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy), and
(c) a hydroxy);
alternatively, R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a phenyl (said group being optionally substituted with the same or different 1 to 4 halogen, cyano, C₁₋₆ alkyl, or C₁₋₆ alkoxy), and
(e) a 5-6 membered monocyclic heteroaryl (said group being optionally substituted with the same or different 1 to 4 halogen, cyano, C₁₋₆ alkyl, or C₁₋₆ alkoxy).
Item 7. The compound of Item 6, or a pharmaceutically acceptable salt thereof, wherein R¹ is a C₃₋₈ cycloalkyl-C₁₋₄ alkyl; or a phenyl-C₁₋₄ alkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy), and
(c) a C₁₋₆ alkoxy (said group being optionally substituted with the same or different 1 to 3 halogen or hydroxy).
Item 8. The compound of any one of Items 1 to 7, or a pharmaceutically acceptable salt thereof, wherein X is O.
Item 9. The compound of any one of Items 1 to 8, or a pharmaceutically acceptable salt thereof, wherein W is —O.
Item 10. The compound of any one of Items 1 to 9, or a pharmaceutically acceptable salt thereof, wherein R³ is selected from
(i) a C₁₋₆ alkyl (said group being optionally substituted with the same or different 1 to 3 halogen, hydroxy or C₁₋₆ alkoxy),
(ii) a C₃₋₈ cycloalkyl (said group being optionally substituted with the same or different 1 to 4 halogen, C₁₋₆ alkyl, hydroxy or C₁₋₆ alkoxy), and
(iii) a 4-8 membered saturated or partially unsaturated monocyclic heterocyclyl (said group being optionally substituted with the same or different 1 to 4 halogen, hydroxy, C₁₋₆ alkyl, or C₁₋₆ alkoxy).
Item 11. The compound of Item 10, or a pharmaceutically acceptable salt thereof, wherein R³ is a C₁₋₆ alkyl (said group being optionally substituted with the same or different 1 to 3 halogen, hydroxy or C₁₋₆ alkoxy), a tetrahydropyranyl (said group being optionally substituted with the same or different 1 to 4 halogen, hydroxy, or C₁₋₆ alkoxy), or a dihydropyranyl.
Item 12. The compound of any one of Items 1 to 11, or a pharmaceutically acceptable salt thereof, wherein R⁴ is selected from
(i) a hydrogen,
(ii) a halogen, or
(iii) a C₁₋₆ alkyl (said group being optionally substituted with the same or different 1 to 3 halogen).
Item 13. The compound of any one of Items 1 to 12, or a pharmaceutically acceptable salt thereof, wherein R⁴ is a hydrogen.
Item 14. The compound of any one of Items 1 to 5, or a pharmaceutically acceptable salt thereof, wherein R¹ is a C₁₋₆ alkyl (said group being optionally substituted with the same or different 1 to 3 halogen, or C₁₋₆ alkoxy);
R² is a C₃₋₈ cycloalkyl-C₁₋₄ alkyl or a C₃₋₈ cycloalkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, hydroxy or C₁₋₆ alkoxy), and
(c) a C₁₋₆ alkoxy (said group being optionally substituted with the same or different 1 to 3 halogen, hydroxy or C₁₋₆ alkoxy);
X is O; and W is —O—.
Item 15. The compound of Item 1, having the structure of formula Ia:

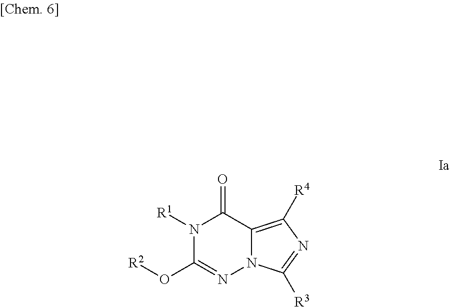

or a pharmaceutically acceptable salt thereof, wherein:
R¹ is a C₁₋₆ alkyl (said group being optionally substituted with the same or different 1 to 3 halogen or C₁₋₆ alkoxy);
R² is a C₃₋₈ cycloalkyl-C₁₋₄ alkyl or a C₃₋₈ cycloalkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, hydroxy or C₁₋₆ alkoxy), and
(c) a C₁₋₆ alkoxy (said group being optionally substituted with the same or different 1 to 3 halogen, hydroxy or C₁₋₆ alkoxy);
R³ is selected from
(i) a C₁₋₆ alkyl (said group being optionally substituted with the same or different 1 to 3 halogen, hydroxy or C₁₋₆ alkoxy),
(ii) a C₃₋₈ cycloalkyl (said group being optionally substituted with the same or different 1 to 4 halogen, C₁₋₆ alkyl, hydroxy or C₁₋₆ alkoxy), and
(iii) a 4-8 membered saturated or partially unsaturated monocyclic heterocyclyl (said group being optionally substituted with the same or different 1 to 4 halogen, hydroxy, C₁₋₆ alkyl, or C₁₋₆alkoxy); and
R⁴ is selected from
(i) a hydrogen,
(ii) a halogen, or
(iii) a C₁₋₆ alkyl (said group being optionally substituted with the same or different 1 to 3 halogen).
Item 16. The compound of any one of Items 1 to 15, or a pharmaceutically acceptable salt thereof, wherein R³ is a tetrahydropyranyl (said group being optionally substituted with hydroxy), or a dihydropyranyl; and R⁴ is a hydrogen.
Item 17. The compound of Item 1, selected from the group consisting of:
2-[(4,4-difluorocyclohexyl)methoxy]-3-ethyl-7-(tetrahydro-2H-pyran-4-yl)imidazo[5,1-][1,2,4]triazin-4(3H)-one (Example 1);
3-(4-chlorobenzyl)-2-methoxy-7-(tetrahydro-2H-pyran-4-yl)imidazo[5,1-f][1,2,4]triazin-4(3H)-one (Example 109);
3-[(4,4-difluorocyclohexyl)methyl]-2-methoxy-7-(tetrahydro-2H-pyran-4-yl)imidazo[5,1-][1,2,4]triazin-4(3H)-one (Example 115);
5-chloro-2-[(4,4-difluorocyclohexyl)methoxy]-3-ethyl-7-(tetrahydro-2H-pyran-4-yl)-imidazo[5,1-f][1,2,4]triazin-4(3H)-one (Example 119);
2-{[(5-chloropyridin-2-yl)methyl]amino}-3-methyl-7-(propan-2-yl)imidazo[5,1-f][1,2,4]triazin-4(3H)-one (Example 149);
3-methyl-7-(tetrahydro-2H-pyran-4-yl)-2-{[trans-4-(trifluoromethyl)cyclohexyl]methoxy}-imidazo[5,1-][1,2,4]triazin-4(3H)-one (Example 245);
2-[(trans-4-ethoxycyclohexyl)methoxy]-3-methyl-7-(tetrahydro-2H-pyran-4-yl)imidazo[5,1-][1,2,4]triazin-4(3H)-one (Example 246);
3-methyl-7-(tetrahydro-2H-pyran-4-yl)-2-{[cis-4-(trifluoromethyl)cyclohexyl]methoxy }-imidazo[5,1-f][1,2,4]triazin-4(3H)-one (Example 248);
2-(cyclohexyloxy)-3-methyl-7-(tetrahydro-2H-pyran-4-yl)imidazo[5,1-f][1,2,4]triazin-4(3H)-one (Example 255);
2-{[trans-4-(ethoxymethyl)cyclohexyl]oxy}-3-methyl-7-(tetrahydro-2H-pyran-4-yl)-imidazo-[5,1-][1,2,4]triazin-4(3H)-one (Example 256);
2-{[trans-4-(ethoxymethyl)cyclohexyl]methoxy}-3-methyl-7-(tetrahydro-2H-pyran-4-yl)-imidazo[5,1-][1,2,4]triazin-4(3H)-one (Example 288);
3-(¹³C,²H₃)methyl-7-(tetrahydro-2H-pyran-4-yl)-2-{[trans-4-(trifluoromethyl)cyclohexyl]-methoxy}imidazo[5,1-f][1,2,4]triazin-4(3H)-one (Example 294);
2-[(4,4-difluorocyclohexyl)oxy]-3-ethyl-7-(tetrahydro-2H-pyran-4-yl)imidazo[5,1-f][1,2,4]triazin-4(3H)-one (Example 304);
3-methyl-7-(tetrahydro-2H-pyran-4-yl)-2-{[trans-4-(trifluoromethyl)cyclohexyl]oxy}-imidazo[5,1-f][1,2,4]triazin-4(3H)-one (Example 305);
3-ethyl-2-{[trans-4-(methoxymethyl)cyclohexyl]oxy }-7-(tetrahydro-2H-pyran-4-yl)-imidazo[5,1-][1,2,4]triazin-4(3H)-one (Example 317);
7-(4-hydroxytetrahydro-2H-pyran-4-yl)-3-methyl-2-{[trans-4-(trifluoromethyl)cyclohexyl]-methoxy}imidazo[5,1-f][1,2,4]triazin-4(3H)-one (Example 326); and
7-(3,6-dihydro-2H-pyran-4-yl)-3-methyl-2-{[trans-4-(trifluoromethyl)cyclohexyl]methoxy}-imidazo[5,1-f][1,2,4]triazin-4(3H)-one (Example 328); or a pharmaceutically acceptable salt thereof.
Item 18. A composition comprising the compound according to any one of Items 1 to 17 and a pharmaceutically acceptable carrier, adjuvant, or vehicle.
Item 19. A method of inhibiting PDE1 in a patient in need thereof, comprising administering to said patient the composition according to Item 18.
Item 20. A method of inhibiting PDE1 in a biological sample, comprising contacting the biological sample with the compound according to any one of Items 1 to 17.
Item 21. A method for treating a neurological or psychiatric disorder in a patient in need thereof, comprising administering to said patient the composition according to Item 18.
Item 22. The method according to Item 21, wherein the neurological or psychiatric disorder is Alzheimer's Disease, Parkinson's Disease, depression, cognitive impairment, stroke, schizophrenia, Down Syndrome, or Fetal Alcohol Syndrome.
Item 23. The method according to Item 21, wherein the neurological or psychiatric disorder involves a deficiency in one or more cognitive domain as defined by DSM-5.

General Preparation

The compounds of the present invention may be prepared for example according to the processes illustrated in the following Preparation Methods 1 to 9. These processes may be optionally modified in view of common general knowledge in the field of organic synthesis. Each compound that is used as a starting material may be used in the form of its salt if needed.

In the case where any functional groups other than a reaction site are changed under the reaction condition as specified or are not preferable in implementing the process as specified, the groups may be optionally protected even where there is no explicit designation of use of a protective group and then deprotected after completion of a reaction or a series of reactions to give a desired compound in the following processes. Such a protective group includes a common protective group described in literatures such as T. W. Greene and P. G. M. Wuts, “Protective Groups in Organic Synthesis”, 3rd Ed., John Wiley and Sons, Inc., New York (1999). Such a protective group may be introduced and deprotected according to a conventional method used in the field of organic synthetic chemistry such as the method described in T. W. Greene and P. G. M. Wuts, “Protective Groups in Organic Synthesis”, 3rd Ed., John Wiley and Sons, Inc., New York (1999).

Examples of the base used herein include an organic base such as diisopropylethylamine, triethylamine, pyridine, lithium diisopropylamide, n-butyl lithium, hexamethyldisilazane lithium, hexamethyldisilazane sodium, and hexamethyldisilazane potassium; an inorganic base such as sodium hydride, potassium hydride, potassium fluoride, cesium fluoride, lithium hydroxide, sodium hydroxide, potassium hydroxide, sodium carbonate, potassium carbonate, cesium carbonate, sodium phosphate, sodium hydrogen carbonate, potassium hydrogen carbonate, potassium phosphate, sodium dihydrogen phosphate, disodium hydrogenphosphate, potassium dihydrogen phosphate, and dipotassium hydrogenphosphate; and a metal alkoxide such as sodium methoxide, sodium tert-butoxide, and potassium tert-butoxide.

Examples of the inert solvent used herein include a halogenated hydrocarbon such as chloroform and dichloromethane; an aromatic hydrocarbon such as benzene and toluene; an alcohol solvent such as methanol, ethanol, 2-propanol, and n-butanol; an ether solvent such as diethyl ether, tetrahydrofuran, 1,2-dimethoxyethane, and 1,4-dioxane; an aprotic polar solvent such as acetonitrile, acetone, methyl ethyl ketone, dimethylformamide, dimethylacetamide, N-methyl-2-pyrrolidinone, and dimethyl-sulfoxide; a basic solvent such as pyridine; water; and a mixed solvent thereof.

Examples of the transition metal used herein include tris(dibenzylideneacetone)dipalladium (0), tetrakis(triphenylphosphine)palladium (0), bis(tri-tert-butylphosphine)palladium (0), palladium (II) chloride, palladium (II) acetate, bis(acetonitrile)palladium (II) chloride, bis(triphenylphosphine)palladium (II) chloride, dichlorobis(tri-O-tolylphosphine)palladium (II), and [1,1′-bis(diphenylphosphino)ferrocene]dichloropalladium (II). One of the transition metals may be used alone or in combination with a copper catalyst such as copper (I) iodide.

Examples of the ligand used herein include triphenylphosphine, tri-O-tolylphosphine, tri-tert-butylphosphine, tricyclohexylphosphine, 1,1′-bis(diphenylphosphino)ferrocene, 2,2-bis(diphenylphosphino)-1,1′-binaphthyl, 2-dicylohexylphosphino-2′,6′-dimethoxybiphenyl, 2-dicyclohexylphosphino-2′,4′,6′-triisopropylbiphenyl, and 2-dicyclohexylphosphino-2′,6′-diisopropoxybiphenyl.

Examples of the halogenating agent used herein include phosphorus oxychloride, phosphorus oxybromide, oxalyl chloride, N-chlorosuccinimide, and N-bromosuccinimide.

Examples of the sulfonylating agent used herein include p-toluenesulfonyl chloride, and methylsulfonyl chloride.

Preparation Method 1

Compounds of Formulae (1-5) and (1-6) among the compounds of Formula (1) may be prepared for example in the following manner.

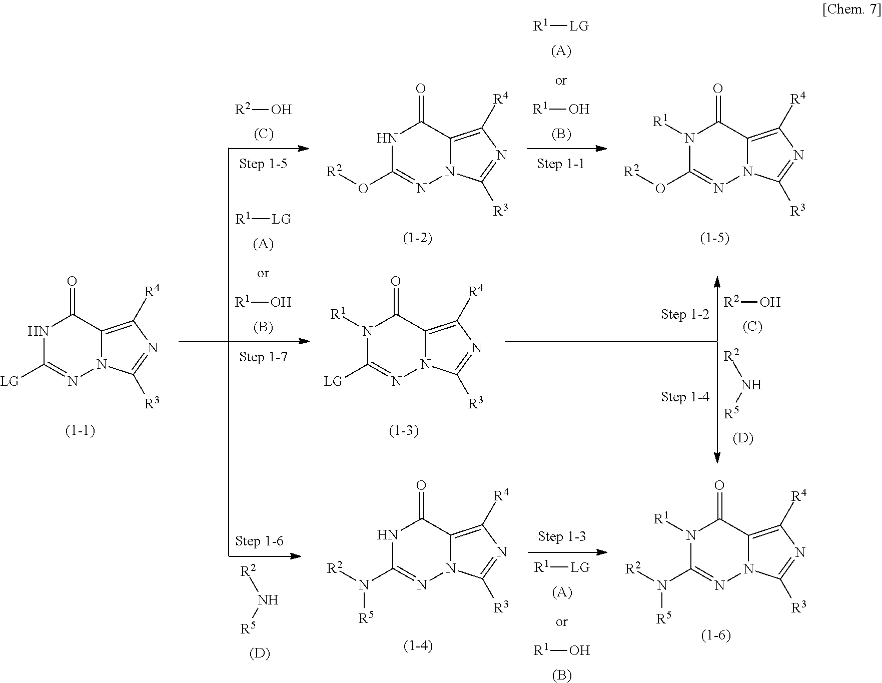

In the scheme, R¹, R², R³, R⁴, and R⁵ are as defined herein, and LG denotes a leaving group such as iodine atom, bromine atom, chlorine atom, and a substituted sulfonyloxy group (e.g. methanesulfonyloxy group, p-toluenesulfonyloxy group).

Step 1-1: Preparation of Compound (1-5)

Compound (1-5) may be prepared by substitution reaction of Compound (1-2) with Compound (A) in the presence of a base in an appropriate inert solvent. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Compound (1-5) may be also prepared by Mitsunobu reaction of Compound (1-2) with alcohol (B) in an appropriate inert solvent. The reaction may proceed specifically in the presence of Mitsunobu agent such as a combination of triphenylphosphine and diethyl azodicarboxylate, and a combination of triphenylphosphine and diisopropyl azodicarboxylate, or a cyanomethylene phosphorane agent. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Step 1-2: Preparation of Compound (1-5)

Compound (1-5) may be also prepared by substitution reaction of Compound (1-3) with alcohol (C) in the presence of a base in an appropriate inert solvent. The reaction may proceed in the presence of an additive if necessary. The reaction temperature is generally in the range between about −20° C. and about 100° C. The reaction time varies depending on conditions such as a reaction temperature, a base, a starting material, and a solvent that are used herein, and is generally in the range between 10 minutes and 96 hours. Examples of the additive used herein include a crown ether such as 12-crown-4, 15-crown-5, and 18-crown-6; and a salt such as sodium iodide and potassium iodide.

Step 1-3: Preparation of Compound (1-6)

Compound (1-6) may be prepared by substitution reaction of Compound (1-4) with Compound (A) or Mitsunobu reaction with alcohol (B) under a similar reaction condition to Step 1-1.

Step 1-4: Preparation of Compound (1-6)

Compound (1-6) may be also prepared by substitution reaction of Compound (1-3) with amine (D) in an appropriate inert solvent. The reaction may proceed in the presence of a base, an additive, etc. if necessary. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours. Examples of the additive used herein include sodium iodide and potassium iodide.

Compound (1-6) may be also prepared by coupling Compound (1-3) with amine (D) in the presence of a transition metal catalyst and a base in an appropriate inert solvent. The reaction may proceed in the presence of a ligand if necessary. The reaction temperature is generally in the range between about −20° C. and about 200° C. The reaction time varies depending on conditions such as a reaction temperature, a base, a starting material, and a solvent that are used herein, and is generally in the range between 10 minutes and 48 hours.

Step 1-5: Preparation of Compound (1-2)

Compound (1-2) may be prepared by substitution reaction of Compound (1-1) with alcohol (C) under a similar reaction condition to Step 1-2.

Step 1-6: Preparation of Compound (1-4)

Compound (1-4) may be prepared by substitution reaction of Compound (1-1) with amine (D) under a similar reaction condition to Step 1-4.

Step 1-7: Preparation of Compound (1-3)

Compound (1-3) may be prepared by substitution reaction of Compound (1-1) with Compound (A) or Mitsunobu reaction with alcohol (B) under a similar reaction condition to Step 1-1.

Preparation Method 2

Compounds of Formulae (2-1) and (2-2) among the compounds of Formula (1) may be prepared for example in the following manner.

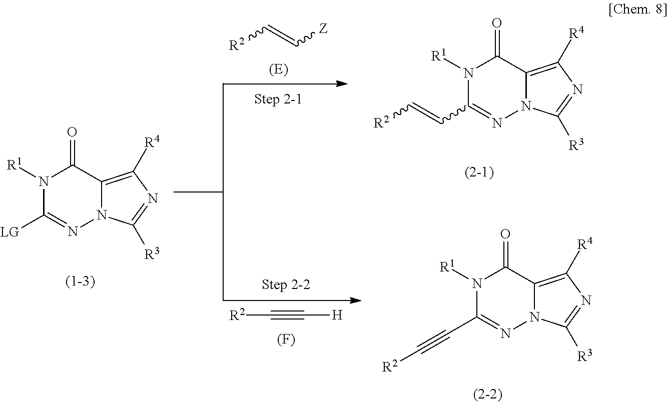

In the scheme, R¹, R², R³, R⁴, and LG are as defined herein, and Z denotes a boronic acid group (—B(OH)₂), a boronic acid ester group (e.g. pinacol boronate ester), an organic boryl group (e.g. —B(Et)₃), an organic tin group (e.g. —Sn(n-Bu)₃), zinc halide (e.g. ZnCl, ZnBr), magnesium halide (e.g. MgCl, MgBr).

Step 2-1: Preparation of Compound (2-1)

Compound (2-1) may be prepared by coupling Compound (1-3) with Compound (E) in the presence of a transition metal catalyst in an appropriate inert solvent. The reaction may proceed in the presence of a ligand, a base, etc. if necessary. The reaction temperature is generally in the range between about −20° C. and about 200° C. The reaction time varies depending on conditions such as a reaction temperature, a base, a starting material, and a solvent that are used herein, and is generally in the range between 10 minutes and 48 hours.

Step 2-2: Preparation of Compound (2-2)

Compound (2-2) may be prepared by coupling Compound (1-3) with acetylene (F) in the presence of a transition metal catalyst in an appropriate inert solvent. The reaction may proceed in the presence of a ligand, a base, etc. if necessary. The reaction temperature is generally in the range between about −20° C. and about 200° C. The reaction time varies depending on conditions such as a reaction temperature, a base, a starting material, and a solvent that are used herein, and is generally in the range between 10 minutes and 48 hours.

Preparation Method 3

Compound of Formula (3-1) among the compounds of Formula (1) may be prepared for example in the following manner.

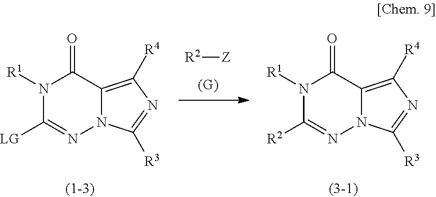

In the scheme, R¹, R², R³, R⁴, LG and Z are as defined herein. Compound (3-1) may be prepared by coupling Compound (1-3) with Compound (G) under a similar reaction condition to Step 2-1.

Preparation Method 4

Compound of Formula (1-5) among the compounds of Formula (1) may be prepared for example in the following manner.

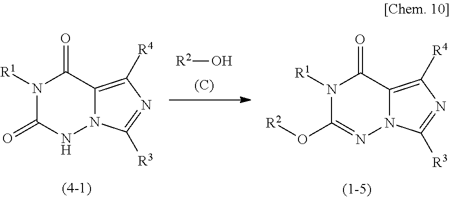

In the scheme, R¹, R², R³, and R⁴ are as defined herein.

Compound (1-5) may be prepared by Mitsunobu reaction of Compound (4-1) with alcohol (C) under a similar reaction condition to Step 1-1.

Preparation Method 5

Compounds of Formulae (5-2) and (5-3) among the compounds of Formula (1) may be prepared for example in the following manner.

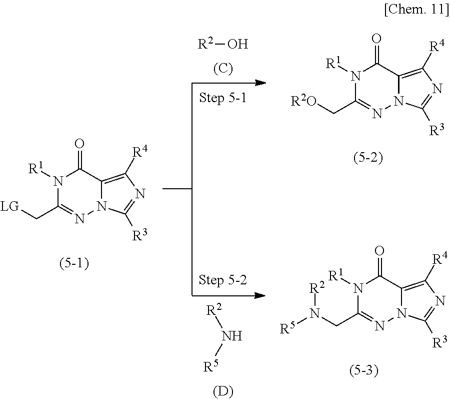

In the scheme, R¹, R², R³, R⁴, and R⁵ are as defined herein, and LG denotes a leaving group such as iodine atom, bromine atom, chlorine atom, and a substituted sulfonyloxy group (e.g. methanesulfonyl group, p-toluenesulfonyl group).

Step 5-1: Preparation of Compound (5-2)

Compound (5-2) may be prepared by substitution reaction of Compound (5-1) with alcohol (C) under a similar reaction condition to Step 1-2.

Step 5-2: Preparation of Compound (5-3)

Compound (5-3) may be prepared by substitution reaction of Compound (5-1) with amine (D) under a similar reaction condition to Step 1-4.

Preparation Method 6

Compound of Formula (1-1) may be prepared for example in the following manner.

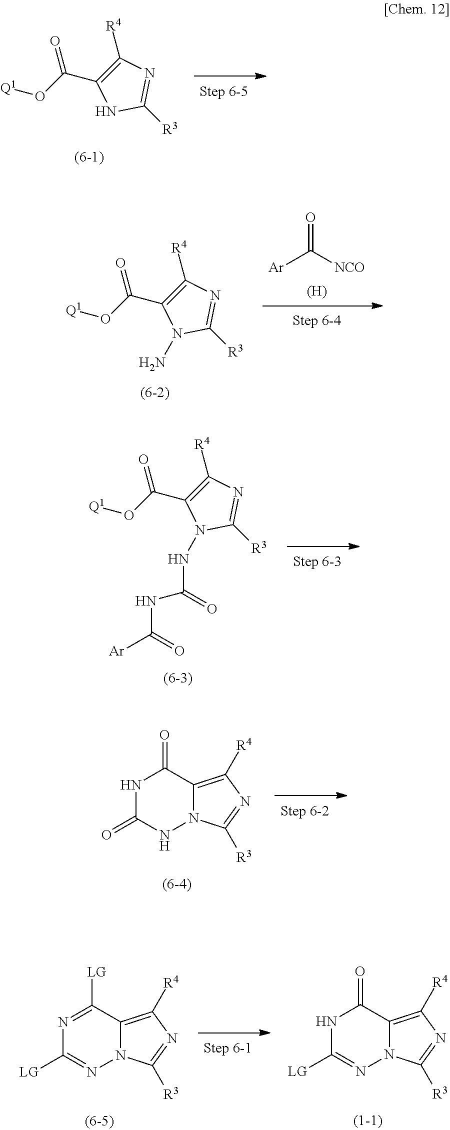

In the scheme, R³, R⁴, and LG are as defined herein, Q¹ denotes C₁₋₆ alkyl, and Ar denotes optionally substituted phenyl.

Step 6-1: Preparation of Compound (1-1)

Compound (1-1) may be prepared by reacting Compound (6-5) with a base in an appropriate inert solvent. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Step 6-2: Preparation of Compound (6-5)

Compound (6-5) may be prepared by reacting Compound (6-4) with a halogenating agent or a sulfonylating agent. The reaction may proceed in the presence of a base, an inert solvent, etc. if necessary. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Step 6-3: Preparation of Compound (6-4)

Compound (6-4) may be prepared by reacting Compound (6-3) in the presence of a base in an appropriate inert solvent. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Step 6-4: Preparation of Compound (6-3)

Compound (6-3) may be prepared by reacting Compound (6-2) with isocyanate (H) in an appropriate inert solvent. The reaction may proceed in the presence of a base if necessary. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Step 6-5: Preparation of Compound (6-2)

Compound (6-2) may be prepared by reacting Compound (6-1) with an aminating agent in an appropriate inert solvent. The reaction may proceed in the presence of a base if necessary. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours. Examples of the aminating agent used herein include 2-[(aminooxy)sulfonyl]-1,3,5-trimethylbenzene and O-(diphenylphosphoryl)-hydroxylamine.

Preparation Method 7

Compounds of Formulae (1-3), (4-1), and (5-1) may be prepared for example in the following manner.

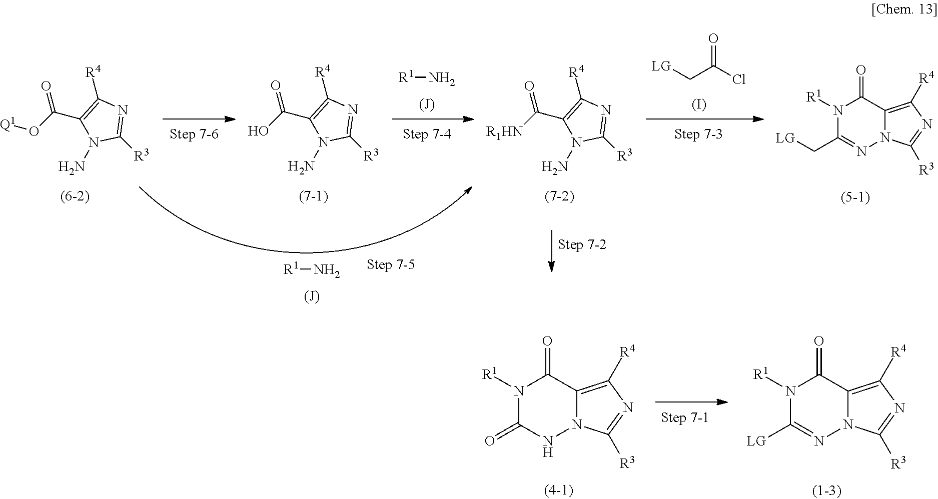

In the scheme, R¹, R³, R⁴, LG, and Q¹ are as defined herein.

Step 7-1: Preparation of Compound (1-3)

Compound (1-3) may be prepared by halogenating or sulfonylating Compound (4-1) under a similar reaction condition to Step 6-2.

Step 7-2: Preparation of Compound (4-1)

Compound (4-1) may be prepared by reacting Compound (7-2) with a carbonylating agent in an appropriate inert solvent. The reaction may proceed in the presence of a base if necessary. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours. Examples of the carbonylating agent used herein include carbonyldiimidazole, triphosgene, and di(N-succinimidyl)carbonate.

Step 7-3: Preparation of Compound (5-1)

Compound (5-1) may be prepared by reacting Compound (7-2) with Compound (I). The reaction may proceed in the presence of a base and an inert solvent if necessary. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Step 7-4: Preparation of Compound (7-2)

Compound (7-2) may be prepared by reacting Compound (7-1) with amine (J) in the presence of a condensing agent, and if necessary a base, in an inert solvent, or by reacting amine (J) with an acid halide or an acid anhydride derived from Compound (7-1) in the presence of a base in an inert solvent. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Examples of the condensing agent used herein include dicyclohexylcarbodiimide (DCC), diisopropylcarbodiimide (DIPC), 1-ethyl-3-(3-dimethylaminopropyl)-carbodiimide (WSC), benzotriazol-1-yl-tris(dimethylamino)phosphonium hexafluorophosphate (BOP), diphenylphosphonyl diamide (DPPA), N,N-carbonyldiimidazole (CDI), and 0-(7-aza-benzotriazol-1-yl)-N,N,N′,N′-tetramethyluronium hexafluorophosphate (HATU). The reaction may proceed with an additional additive such as N-hydroxysuccinimide (HOSu), 1-hydroxybenzotriazole (HOBt), and 3-hydroxy-4-oxo-3,4-dihydro-1,2,3-benzotriazine (HOOBt) if necessary.

Step 7-5: Preparation of Compound (7-2)

Compound (7-2) may be also prepared by reacting Compound (6-2) with amine (J) in an appropriate inert solvent. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Step 7-6: Preparation of Compound (7-1)

Compound (7-1) may be prepared by hydrolyzing Compound (6-2) under a similar reaction condition to Step 6-1.

Preparation Method 8

Compounds of Formulae (8-2) and (8-3) among the compounds of Formula (1) may be prepared for example in the following manner.

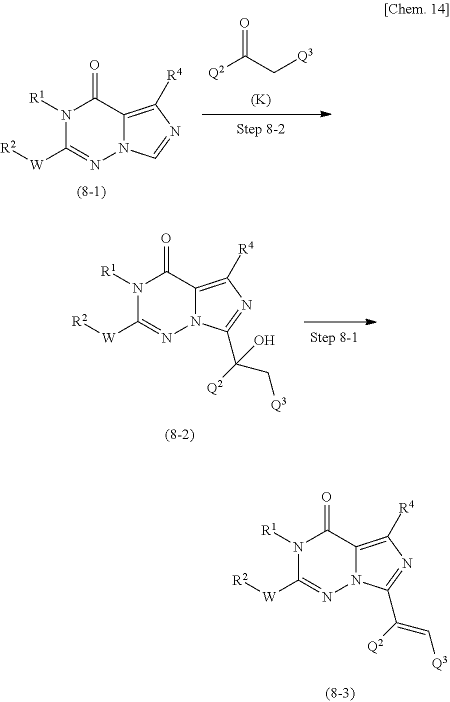

In the scheme, R¹, R², R⁴, and W are as defined herein, and Q² and Q³ independently denote hydrogen atom or optionally substituted C₁₋₆ alkyl, or Q² and Q³ may combine together with the carbon atoms which they bind to form optionally substituted saturated carbon ring, unsaturated carbon ring, saturated heterocycle or unsaturated heterocycle.

Step 8-1: Preparation of Compound (8-3)

Compound (8-3) may be prepared by reacting Compound (8-2) with a halogenating agent or a sulfonylating agent in the presence of a base in an appropriate inert solvent. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Step 8-2: Preparation of Compound (8-2)

Compound (8-2) may be prepared by reacting Compound (8-1) with Compound (K) in the presence of a base in an appropriate inert solvent. The reaction temperature is generally in the range between about −100° C. and a boiling point of the solvent that is used in this step. The reaction time varies depending on conditions such as a reaction temperature, a base, a starting material, and a solvent that are used herein, and is generally in the range between 10 minutes and 48 hours.

Preparation Method 9

Compounds of Formulae (9-1) and (9-2) among the compounds of Formula (1) may be prepared for example in the following manner.

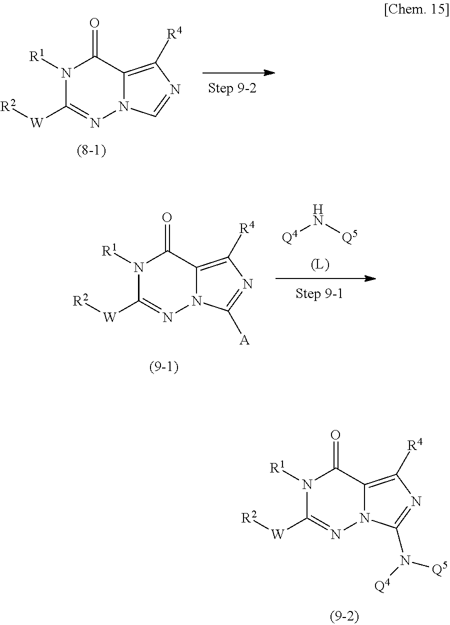

In the scheme, R¹, R², R⁴, and W are as defined herein, A denotes halogen atom, and Q⁴ and Q⁵ independently denote hydrogen atom or optionally substituted C₁₋₆ alkyl, or Q⁴ and Q⁵ may combine together with the nitrogen atom which they bind to form optionally substituted saturated heterocycle or partially unsaturated heterocycle.

Step 9-1: Preparation of Compound (9-2)

Compound (9-2) may be prepared by substitution or coupling reaction of Compound (6-2) with amine (L) under a similar reaction condition to Step 1-4.

Step 9-2: Preparation of Compound (9-1)

Compound (9-1) may be prepared by reacting Compound (8-1) with a halogenating agent in an appropriate inert solvent. The reaction may proceed in the presence of a base. The reaction temperature is generally in the range between about −20° C. and a boiling point of the solvent that is used in this step. The reaction time varies depending on conditions such as a reaction temperature, a base, a starting material, and a solvent that are used herein, and is generally in the range between 10 minutes and 48 hours.

Each intermediate and desired compound in each Preparation Method may be isolated and purified according to a conventional purification method used in the field of organic synthetic chemistry such as neutralization, filtration, extraction, washing, drying, concentration, recrystallization, and various types of chromatography. Each intermediate may be used in a next step without purification.

An optically active compound in the present invention may be prepared by starting from a suitable optically-active material or intermediate or by optically resolving a final racemic product. The optical resolution includes a physical separation using an optically active column and a chemical separation such as fractionated crystallization. Diastereomers of the compound of the present invention may be prepared for example by fractionated crystallization.

A pharmaceutically acceptable salt of the compound of Formula (1) may be prepared by mixing the compound of Formula (1) with a pharmaceutically acceptable acid in a solvent such as water, methanol, ethanol, and acetone.

### EXAMPLES

As depicted in the Examples below, in certain exemplary embodiments, compounds are prepared according to the following procedures. It will be appreciated that, although the general methods depict the synthesis of certain compounds of the present invention, the following methods, and other methods known to one of ordinary skill in the art, can be applied to all compounds and subclasses and species of each of the compounds as described herein.

In the examples below, unless otherwise indicated, all temperatures are set forth in degrees Celsius and all parts and percentages are by weight. Reagents were purchased from commercial suppliers, such as Sigma-Aldrich Chemical Company, and were used without further purification unless otherwise indicated. Reagents were prepared by the following standard literature procedures known to those skilled in the art. Solvents were purchased from Aldrich in Sure-Seal bottles and used as received. All solvents requiring purification or drying were treated using standard methods known to those skilled in the art, unless otherwise indicated.

The reactions set forth below were done generally at the ambient temperature, unless otherwise indicated. The reaction flasks were fitted with rubber septa for introduction of substrates and reagents via syringe. Analytical thin layer chromatography (TLC) was performed using glass-backed silica gel pre-coated plates (Merck Art 5719) and eluted with appropriate solvent ratios (v/v). Reactions were assayed by TLC or LCMS, and terminated as judged by the consumption of starting material. Visualization of the TLC plates was done with UV light (254 wavelength) or with an appropriate TLC visualizing solvent, such as basic aqueous KMnO₄ solution activated with heat. Flash column chromatography (See, e.g. Still et al., J. Org. Chem., 43: 2923 (1978)) was performed using silica gel 60 (Merck Art 9385) or various MPLC systems. Reactions under microwave irradiation conditions were carried out in a Biotage initiator microwave system.

The compound structures in the examples below were confirmed by one or more of the following methods: proton magnetic resonance spectroscopy, mass spectroscopy, and melting point. Proton magnetic resonance CH NMR) spectra were determined using a JEOL or Bruker NMR spectrometer operating at 300 or 400 MHz field strength. Chemical shifts are reported in the form of delta (δ) values given in parts per million (ppm) relative to an internal standard, such as tetramethylsilane (TMS). Alternatively, ¹H NMR spectra were referenced to signals from residual protons in deuterated solvents as follows: CDCl₃=7.25 ppm; DMSO-d₆=2.49 ppm; C₆D₆=7.16 ppm; CD₃OD=3.30 ppm. Peak multiplicities are designated as follows: s, singlet; d, doublet; dd, doublet of doublets; t, triplet; dt, doublet of triplets; q, quartet; quint, quintet; seat, sextet; sept, septet; br, broadened; and m, multiplet. Coupling constants are given in Hertz (Hz). Mass spectra (MS) data were obtained using Agilent Technologies 1200 Series/Agilent Technologies 6110 Quadrupole LC/MS, Waters ACQUITY UPLC or Shimadzu LCMS-2020. Waters supercritical fluid system (SFC) was used to separate chiral compounds with the following methods.

Method A:
Column: AD-H (4.6×250 mm, 5 μm)
Co-Solvent: MeOH (0.5% DEA)
Column Temperature: 40° C.
CO₂ Flow Rate: 2.55 mL/min
Co-Solvent Flow Rate: 0.45 mL/min

Method B:
Column: OZ-H (4.6×250 mm, 5 μm)
Co-Solvent: MeOH (0.1% DEA)
Column Temperature: 40° C.
CO₂ Flow Rate: 1.95 mL/min
Co-Solvent Flow Rate: 1.05 mL/min

As used herein, and unless otherwise specified, “Me” means methyl, “Et” means ethyl, “Pr” means propyl, “Bu” means butyl, “Ms” means mesyl, “Ac” means acetyl, “BINAP” means 2,2-bis(diphenylphosphino)-1,1′-binaphthyl, “Boc” means tert-butoxycarbonyl, “CDI” means carbonyldiimidazole, “DCM” means dichloromethane, “DEAD” means diethyl azodicarboxylate, “DIAD” means diisopropyl azodicarboxylate, “DIEA” means diisopropylethylamine, “DME” means dimethoxyethane, “DMF” means dimethylformamide, “DMSO” means dimethyl sulfoxide, “dppf” means 1,1′-bis(diphenylphosphino)ferrocene, “EtOAc” means ethyl acetate, “EtOH” means ethanol, “HATU” means O-(7-azabenzotriazol-1-yl)-N,N,N′,N′-tetramethyluronium hexafluorophosphate, “IPA” means Isopropanol, “KHMDS” means potassium bis(trimethylsilyl)amide, “LDA” means lithium diisopropylamide, “LiHMDS” means lithium bis(trimethylsilyl)amide, “MeCN” means acetonitrile, “MeOH” means methanol, “NBS” means N-bromosuccinimide, “NCS” means N-chlorosuccinimide, “NIS” means N-iodosuccinimide, “NMP” means N-methylpyrrolidone, “PE” means petroleum ether, “RT” or “r.t.” means room temperature, “Ruphos” means 2-dicyclohexylphosphino-2′,6′-diisopropoxy-1,1′-biphenyl, “TEA” means triethylamine, “TFA” means trifluoroacetic acid, “THF” means tetrahydrofuran, “X-phos” means (2′,4′,6′-triisopropylbiphenyl-2-yl)phosphine, “h” or “hr” means hour(s), “min” means minute(s), “cat.” means catalytic, and “aq” means aqueous.

### Reference Example 1

### 2-Chloro-7-(tetrahydro-2H-pyran-4-yl)imidazo[5,1-f][1,2,4]triazin-4(3H)-one

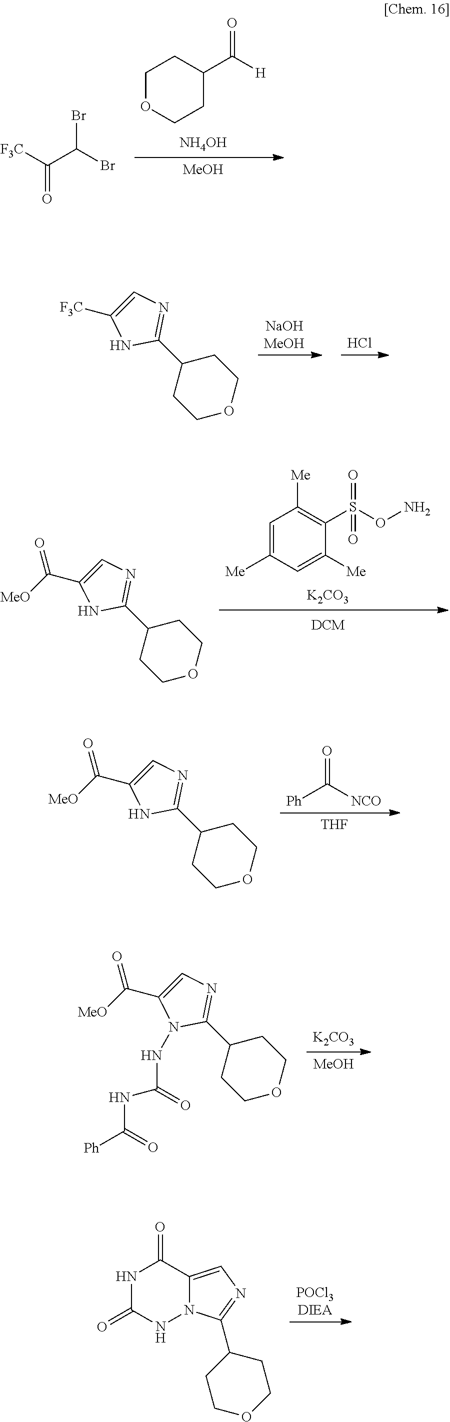

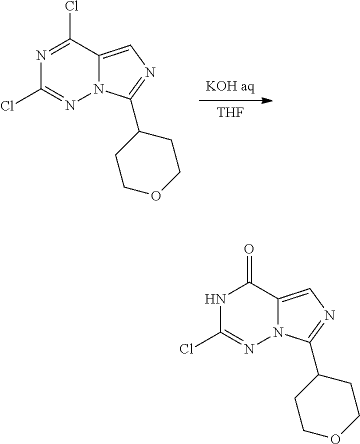 [SMILES: Clc1nc(Cl)c2cnc(C3CCOCC3)n2n1 ; "KOH aq" ; "THF" ; O=c1[nH]c(Cl)nn2c(C3CCOCC3)ncc12]

To a solution of 2,4-dichloro-7-(tetrahydro-2H-pyran-4-yl)imidazo[5,1-f][1,2,4]triazine (1.2 g) in tetrahydrofuran (20 mL) was added 2N KOH (20 mL). The mixture was stirred for 2 h at 50° C., and then was neutralized with 1N HCl. The mixture was filtered to give the titled compound (0.78 g, yield 70%).

LC-MS (m/z)=255 [M+H]⁺. ¹H-NMR (400 MHz, DMSO-d₆): δ 1.79-1.88 (m, 4H), 3.34-3.38 (m, 1H), 3.46-3.53 (m, 2H), 3.91-3.95 (m, 2H), 7.76 (s, 1H), 13.01 (br, 1H).

### 2-(Tetrahydro-2H-pyran-4-yl)-5-(trifluoromethyl)-1H-imidazole

A mixture of sodium acetate trihydrate (27.2 g, 200 mmol) and 3,3-dibromo-1,1,1-trifluoropropan-2-one (26.98 g, 100 mmol) in water (75 ml) was heated under reflux for 1 h. The mixture was then cooled to r.t. and was slowly added to a solution of tetrahydro-2H-pyran-4-carbaldehyde (90 mmol, 10.27 g) and concentrated ammonium hydroxide solution (50 mL) in MeOH (150 mL). The mixture was stirred at r.t. for 18 h and was then evaporated under reduced pressure. The aqueous residue was extracted with EtOAc (150 mL×3) and the combined organic solution was dried over magnesium sulfate and concentrated in vacuo to give an oil. The oil was then triturated in water with a trace of MeOH to afford the titled compound as a crystalline solid (19.8 g, yield 90%). LC-MS (m/z)=221 [M+H]⁺.

### Methyl 2-(tetrahydro-2H-pyran-4-yl)-1H-imidazole-5-carboxylate

To a solution of 2-(tetrahydro-2H-pyran-4-yl)-5-(trifluoromethyl)-1H-imidazole (85 mmol) in MeOH (200 mL) was added NaOH solution (2.7 M, 50 mL) and the mixture was stirred at 95° C. overnight. Then conc. HCl (25 mL) was added. The mixture was stirred at that temperature for 4 h. EtOAc (250 mL) was added to the reaction vessel and the resulting biphasic mixture was transferred to a separatory funnel. The layers were separated and the water phase was extracted with EtOAc (150 mL×3). The combined organics were dried over anhydrous Na₂SO₄, filtered and concentrated in vacuum to afford the title compound as a solid (16.5 g, yield 80%).

LC-MS (m/z)=210 [M+H]⁺.

### Methyl 1-amino-2-(tetrahydro-2H-pyran-4-yl)-1H-imidazole-5-carboxylate

To a solution of methyl 2-(tetrahydro-2H-pyran-4-yl)-1H-imidazole-5-carboxylate (70 g, 0.34 mol) in DCM (250 mL) was added 2-[(aminooxy)sulfonyl]-1,3,5-trimethylbenzene (110 g, 0.51 mol) and K₂CO₃ (94 g, 0.64 mol). The reaction mixture was cooled to 0° C. and stirred at that temperature for 15 h. Water (50 mL) was added to the reaction vessel and the resulting biphasic mixture was transferred to a separatory funnel. The layers were separated. The combined organics were dried over anhydrous Na₂SO₄, filtered and concentrated in vacuum. The resulting solid was purified by flash column chromatography to provide the title compound as a white solid (25 g, yield 35%). LC-MS (m/z)=225 [M+H]⁺.

### Methyl 1-[(benzoylcarbamoyl)amino]-2-(tetrahydro-2H-pyran-4-yl)-1H-imidazole-5-carboxylate

To a solution of methyl 1-amino-2-(tetrahydro-2H-pyran-4-yl)-1H-imidazole-5-carboxylate (5 g, 22.2 mmol) in THF (75 mL) was added benzoyl isocyanate (3.59 g, 24.42 mmol). The reaction mixture was heated to and stirred at that temperature for 12 h. The combined organics was concentrated in vacuo to give the title compound (5 g, yield 80%).

LC-MS (m/z)=373 [M+H]⁺. ¹H-NMR (400 MHz, DMSO-d₆): δ 1.67-1.88 (m, 4H), 3.04-3.12 (m, 1H), 3.40-3.46 (m, 2H), 3.71 (s, 3H), 3.89-3.94 (m, 2H), 7.56-7.60 (m, 2H), 7.67-7.71 (m, 2H), 8.06-8.08 (m, 2H), 11.19 (s, 1H), 11.34 (s, 1H).

### 7-(Tetrahydro-2H-pyran-4-yl)imidazo[5,1-f][1,2,4]triazine-2,4(1H,3H)-dione

To a solution of methyl 1-[(benzoylcarbamoyl)amino]-2-(tetrahydro-2H-pyran-4-yl)-1H-imidazole-5-carboxyl ate (5 g, 13.43 mmol) in methanol (45 mL) was added potassium carbonate (2.23 g, 16.11 mmol). The combined organics concentrated in vacuo. Water (20 mL) was added to the reaction. The mixture was neutralized with 1 N HCl, filtered and washed with MeOH to give the title compound (1.8 g, yield 56%).

LC-MS (m/z)=237 [M+H]⁺. ¹H-NMR (400 MHz, DMSO-d₆): δ 1.75-1.85 (m, 4H), 3.32-3.35 (m, 1H), 3.38-3.49 (m, 2H), 3.92-3.95 (m, 2H), 7.50 (br, 1H), 7.74 (s, 1H), 11.15 (s, 1H).

### 2,4-Dichloro-7-(tetrahydro-2H-pyran-4-yl)imidazo[5,1-f][1,2,4]triazine

The mixture of 7-(tetrahydro-2H-pyran-4-yl)imidazo[5,1-f][1,2,4]triazine-2,4(1H,3H)-dione (1.8 g) in phosphoryl trichloride (20 mL) and N,N-Diisopropylethylamine (1.48 g) was stirred for 3 h at 120° C. The pH was adjusted to 7-8 and a white precipitate formed. After filtration, the title compound was collected as a yellow solid (1.2 g, yield 60%). LC-MS (m/z)=273 [M+H]⁺. ¹H-NMR (400 MHz, DMSO-d₆): δ 1.24-1.31 (m, 2H), 1.82-1.87 (m, 2H), 3.40-3.54 (m, 1H), 3.47-3.54 (m, 2H), 3.92-3.96 (m, 2H), 7.88 (s, 1H).

The compounds of Reference Examples 2 to 4 were synthesized in a similar manner to Reference Example 1.

| | |
|---|---|
| | [Chem. 17] |
| 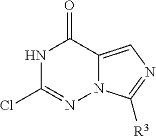 | |

| No. | R³— | ¹H-NMR |
|---|---|---|
| 2 | 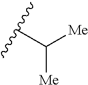 [SMILES: *C(C)C] | (400 MHz, DMSO-d₆): δ 1.26-1.27 (m, 6H), 3.35 (s, 1H), 7.72 (s, 1H), 12.99 (br, 1H). |
| 3 | 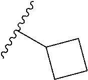 [SMILES: *C1CCC1] | (400 MHz, DMSO-d₆): δ 1.81-1.89 (m, 1H), 1.93-2.04 (m, 1H), 2.20-2.38 (m, 4H), 3.16-3.85 (m, 1H), 7.15 (s, 1H). |
| 4 | 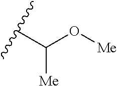 [SMILES: *C(C)OC] | (400 MHz, DMSO-d₆): δ 1.51 (d, J = 5.2 Hz, 3H), 3.18 (s, 3H), 4.84 (q, J = 4.0 Hz, 1H), 7.81 (s, 1H), 13.09 (br, 1H). |

### Reference Example 5

### 2-Chloro-3-ethyl-7-(tetrahydro-2H-pyran-4-yl)imidazo[5,1-f][1,2,4]triazin-4(3H)-one

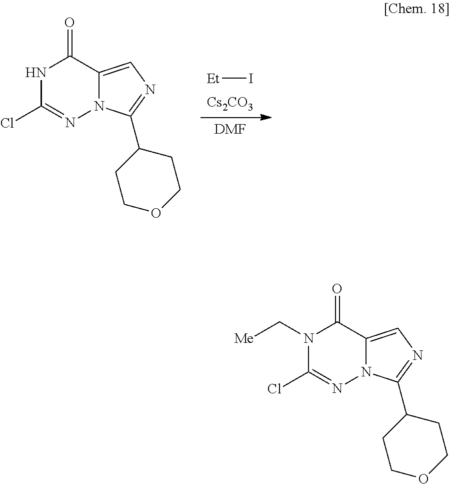

2-Chloro-7-(tetrahydro-2H-pyran-4-yl)imidazo[5,1-f][1,2,4]triazin-4(3H)-one (10.0 g, 39.3 mmol) and cesium carbonate (20.8 g, 63.8 mmol) were dissolved in DMF (50 mL) and heated to 60° C. Ethyl iodide (5.3 mL, 51.5 mmol) was dropwised and stirred at 60° C. for 2 hours. The reaction mixture was filtered through a filter paper and the filter cake was washed chloroform and concentrated. Water, saturated NH₄Cl aq. and EtOAc were added and organic layer was separated. The aqueous layer was extracted with EtOAc (200 mL×5) and chloroform (150 mL×2). The organic layers were combined and dried with sodium sulfate, and the solvent was removed. The resin was washed with isopropyl ether to give the titled compound (9.45 g, yield 85%) as a yellow solid.

¹H-NMR (400 MHz, CDCl₃): δ 1.36 (t, J=7.1 Hz, 3H), 1.88-1.93 (m, 2H), 2.03-2.13 (m, 2H), 3.41 (tt, J=11.7, 3.9 Hz, 1H), 3.59 (td, J=11.7, 2.2 Hz, 2H), 4.09 (dq, J=11.7, 2.2 Hz, 2H), 4.23 (q, J=7.2 Hz, 2H), 7.83 (s, 1H).

### Reference Example 6

### 2-Chloro-7-(1-methoxyethyl)-3-methylimidazo[5,1-f][1,2,4]triazin-4(3H)-one

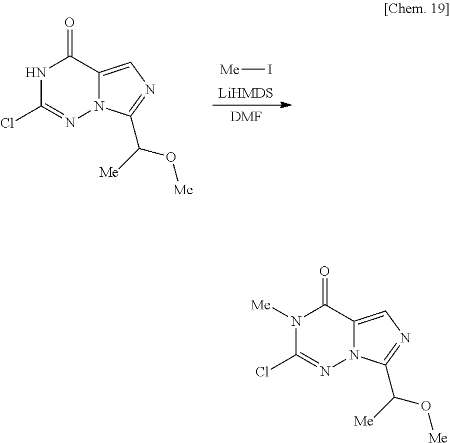

A solution of 2-chloro-7-(1-methoxyethyl)imidazo[5,1-f][1,2,4]triazin-4(3H)-one (533 mg, 2.38 mmol) in anhydrous THF (30 mL) was added 1 M LiHMDS (3.5 ml, 3.53 mmol) at 0° C. under nitrogen atmosphere. Mel (1.0 g, 7.14 mmol) was added into upper solution at 0° C. for 10 min. The reaction was heated to 50° C. for 2 h. The reaction was cooled to r.t., then quenched by adding saturated NH₄Cl (100 mL). The crude product was extracted from water by EtOAc (30 mL×4), and then purified through silica gel column to give the titled compound (280 mg, yield 50%).

LC-MS (m/z)=243 [M+H]⁺. ¹H-NMR (400 MHz, DMSO-d₆): δ 1.66 (d, J=6.4 Hz, 3H), 3.34 (s, 3H), 3.65 (s, 3H), 4.94 (q, J=5.0 Hz, 1H), 7.91 (s, 1H).

### Reference Example 7

### 2-Chloro-3-[(4,4-difluorocyclohexyl)methyl]-7-(tetrahydro-2H-pyran-4-yl)imidazo[5,1-f][1,2,4]triazin-4(3H)-one

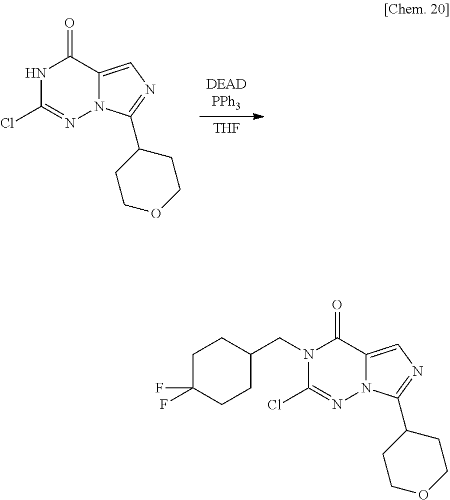 [SMILES: O=c1[nH]c(Cl)nn2c(C3CCOCC3)ncc12 ; "DEADPPh3" ; "THF" ; O=c1c2cnc(C3CCOCC3)n2nc(Cl)n1CC1CCC(F)(F)CC1]

To a mixture of 2-Chloro-7-(tetrahydro-2H-pyran-4-yl)imidazo[5,1-f][1,2,4]triazin-4(3H)-one (1.02 g, 4 mmol), triphenylphosphine (1.36 g, 5.2 mmol) and 4,4-difluorocyclohexylmethanol (783 mg, 5.2 mmol) in DCM (10 mL) was added DEAD (1.05 g, 5.2 mmol) at room temperature. After stirring for 1 h, the mixture was concentrated in vacuo and purified by silica gel column chromatography (hexane/EtOAc) to give the titled compound (671 mg, yield 43%).

LC-MS (m/z)=387 [M+H]⁺.

The compounds of Reference Examples 8 to 20 were synthesized in a similar manner to Reference Example 5, 6 or 7.

| | |
|---|---|
| | [Chem. 21] |
| 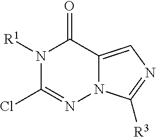 | |

| No. | R¹— | R³— | ¹H-NMR or LC-MS |
|---|---|---|---|
| 8 | Me— | 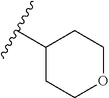 [SMILES: *C1CCOCC1] | ¹H-NMR (400 MHz, CDCl₃): δ 1.90-1.93 (m, 2H), 2.03-2.13 (m, 2H), 3.42 (tt, J = 11.6, 4.0 Hz, 1H), 3.57-3.65 (m, 5H), 4.08-4.11 (m, 2H), 7.84 (s, 1H). |
| 9 | ⁿPr— | 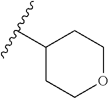 [SMILES: *C1CCOCC1] | ¹H-NMR (400 MHz, CDCl₃): δ 1.00-1.04 (t, J = 14.8 Hz, 3H), 1.75-1.81 (q, J = 7.8 Hz, 2H), 1.90-1.94 (m, 2H), 2.07-2.14 (m, 2H), 3.42-3.45 (m, 1H), 3.57-3.64 (m, 2H), 4.09-4.14 (m, 4H), 7.84 (s, 1H). |
| 10 | 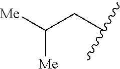 [SMILES: *CC(C)C] | 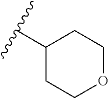 [SMILES: *C1CCOCC1] | ¹H-NMR (400 MHz, CDCl₃): δ 0.99 (d, J = Hz, 6H), 1.91-1.95 (m, 2H), 2.08- 2.24 (m, 3H), 3.39-3.47 (m, 1H), 3.58- 3.64 (m, 2H), 4.01 (d, J = 7.6 Hz, 2H), 4.09-4.12 (m, 2H), 7.84 (s, 1H). |
| 11 | 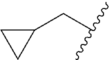 [SMILES: *CC1CC1] | 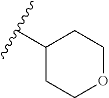 [SMILES: *C1CCOCC1] | ¹H-NMR (400 MHz, CDCl₃): δ 0.47-0.60 (m, 4H), 1.22-1.30 (m, 1H), 1.88-1.92 (m, 2H), 2.02-2.12 (m, 2H), 3.41 (tt, J = 11.6, 4.0 Hz, 1H), 3.58 (td, J = 11.7, 2.2 Hz, 2H), 4.06-4.09 (m, 4H), 7.82 (s, 1H). |
| 12 | 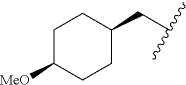 [SMILES: *C[C@H]1CC[C@@H](OC)CC1] | 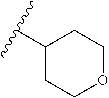 [SMILES: *C1CCOCC1] | ¹H-NMR (400 MHz, CDCl₃): δ 1.32-1.52 (m, 6H), 1.81-2.02 (m, 5H), 2.02-2.17 (m, 2H), 3.31 (s, 3H), 3.36-3.51 (m, 2H), 3.55-3.69 (m, 2H), 3.96-4.19 (m, 4H), 7.83 (s, 1H). |
| 13 | 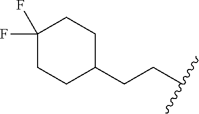 [SMILES: *CCC1CCC(F)(F)CC1] | 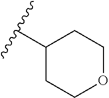 [SMILES: *C1CCOCC1] | LC-MS (m/z) = 401 [M + H]⁺. |
| 14 | 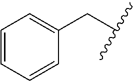 [SMILES: *Cc1ccccc1] | 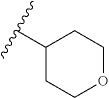 [SMILES: *C1CCOCC1] | ¹H-NMR (400 MHz, CDCl₃): δ 1.89-1.92 (m, 2H), 2.03-2.14 (m, 2H), 3.41 (tt, J = 11.6, 3.9 Hz, 1H), 3.59 (td, J = 11.8, 2.1 Hz, 2H), 4.07-4.11 (m, 2H), 5.37 (s, 2H), 7.31-7.36 (m, 5H), 7.88 (s, 1H). |
| 15 | 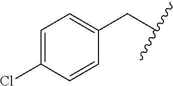 [SMILES: *Cc1ccc(Cl)cc1] | 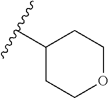 [SMILES: *C1CCOCC1] | ¹H-NMR (400 MHz, CDCl₃): δ 1.88-1.92 (m, 2H), 2.03-2.13 (m, 2H), 3.41 (tt, J = 11.7, 3.9 Hz, 1H), 3.59 (td, J = 11.7, 2.1 Hz, 2H), 4.07-4.11 (m, 2H), 5.33 (s, 2H), 7.30-7.35 (m, 4H), 7.89 (s, 1H). |
| 16 | Me— | 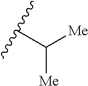 [SMILES: *C(C)C] | ¹H-NMR (400 MHz, CDCl₃): δ 1.32 (m, 6H), 3.40-3.44 (m, 1H), 3.55 (s, 3 H), 7.76 (s, 1H). |
| 17 | Et— | 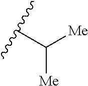 [SMILES: *C(C)C] | ¹H-NMR (400 MHz, CDCl₃): δ 1.34-1.40 (m, 9H), 3.46 (m, 1H), 4.24 (m, 2H), 7.82 (s, 1H). |
| 18 | Me— | 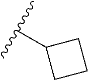 [SMILES: *C1CCC1] | ¹H-NMR (400 MHz, DMSO-d₆): δ 2.01- 2.18 (m, 2H), 2.41-2.46 (m, 2H), 2.48- 2.56 (m, 2H), 3.62 (s, 3H), 3.97-4.01 (m, 1H), 7.86 (s, 1H). |
| 19 | Et— | 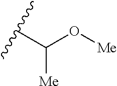 [SMILES: *C(C)OC] | ¹H-NMR (400 MHz, DMSO-d₆): δ 1.37- 1.40 (m, 3H), 1.67 (d, J = 6.4 Hz, 3H), 3.35 (s, 3H), 4.23-4.28 (m, 2H), 4.92- 4.97 (m, 1H), 7.90 (s, 1H). |
| 20 | 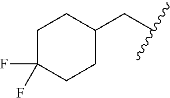 [SMILES: *CC1CCC(F)(F)CC1] | 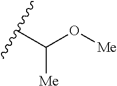 [SMILES: *C(C)OC] | LC-MS (m/z) = 361 [M + H]⁺. |

### Reference Example 21

### 2-Chloro-3-cyclopropyl-7-(tetrahydro-2H-pyran-4-yl)imidazo[5,1-f][1,2,4]triazin-4(3H)-one

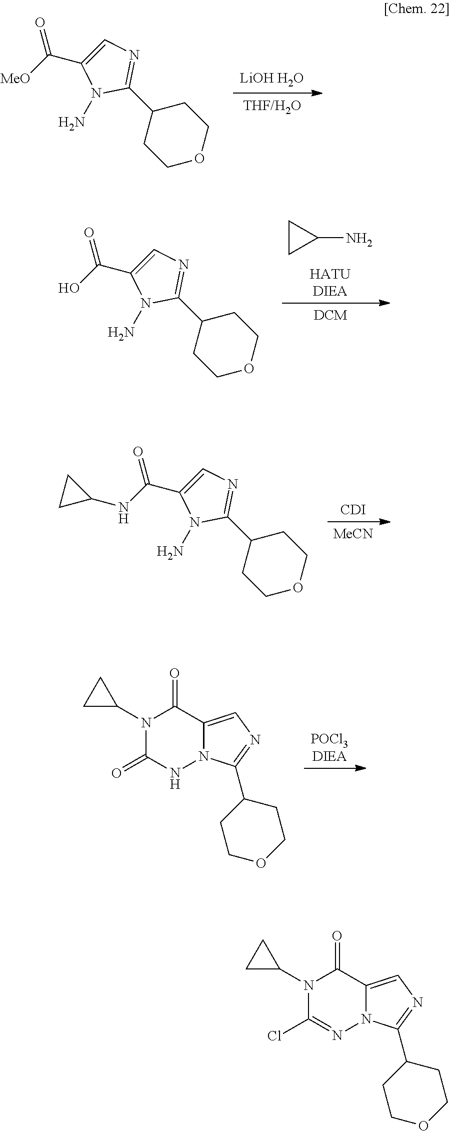

A solution of 3-cyclopropyl-7-(tetrahydro-2H-pyran-4-yl)imidazo[5,1-f][1,2,4]triazine-2,4(1H,3H)-dione (200 mg, 0.724 mmol) in phosphoryl trichloride (0.8 mL) and DIEA (0.16 mL, 0.941 mmol) was stirred for 22 h at 100° C. Upon completion, the reaction mixture was poured into ice water. After neutralization with NaOH aq., the aqueous phase was extracted with EtOAc. The combined organic phases were dried over Na₂SO₄, and concentrated in vacuo. The obtained compound was used to the next reaction without further purification (157 mg, yield 74%).

LC-MS (m/z)=295 [M+H]⁺.

### 1-Amino-2-(tetrahydro-2H-pyran-4-yl)-1H-imidazole-5-carboxylic acid

A solution of methyl 1-amino-2-(tetrahydro-2H-pyran-4-yl)-1H-imidazole-5-carboxylate (1 g, 4.44 mmol) and lithium hydroxide monohydrate (279 mg, 6.66 mmol) in THF (5 mL) and H₂O (1.5 mL) was stirred for 1 h at room temperature. Upon completion, HCl aq was added to adjust the pH to 6-7 at 0° C. and a white precipitate formed. After filtration, the titled compound was collected (540 mg, yield 57%) as a white solid. LC-MS (m/z)=212 [M+H]⁺.

### 1-Amino-N-cyclopropyl-2-(tetrahydro-2H-pyran-4-yl)-1H-imidazole-5-carboxamide

To a mixture of 1-amino-2-(tetrahydro-2H-pyran-4-yl)-1H-imidazole-5-carboxylic acid (422 mg, 2.0 mmol), cycropropylamine (0.21 mL, 3.0 mmol) and DIEA (1.6 mL, 9.0 mmol) in DCM (6 mL) was added HATU (1.14 g, 3.0 mmol) at room temperature. After stirring for 1 h, the mixture was purified by silica gel column chromatography (chloroform/MeOH) to give the titled compound (382 mg, yield 76%). LC-MS (m/z)=251 [M+H]⁺.

### 3-Cyclopropyl-7-(tetrahydro-2H-pyran-4-yl)imidazo[5,1-f][1,2,4]triazine-2,4(1H,3H)-dione

A solution of 1-amino-N-cyclopropyl-2-(tetrahydro-2H-pyran-4-yl)-1H-imidazole-5-carboxamide (310 mg, 1.24 mmol) and CDI (302 mg, 1.86 mmol) in MeCN (6 mL) was stirred for 2 h at 70° C. After the mixture was diluted with EtOAc at 0° C., the resulting precipitation was filtered to give the titled compound (197 mg, yield 57%). LC-MS (m/z)=277 [M+H]⁺.

### Reference Example 22

### 2-Methoxy-7-(tetrahydro-2H-pyran-4-yl)imidazo[5,1-f][1,2,4]triazin-4(3H)-one

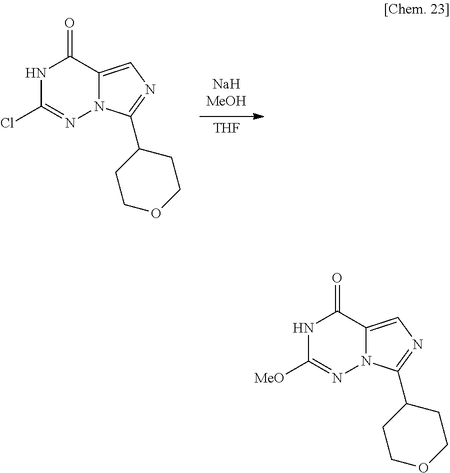

To a mixture of MeOH (41 ml, 1.02 mmol) in THF (4 mL) was added NaH (103 mg, 2.36 mmol) at room temperature and the mixture was stirred for 10 min. And then 2-chloro-7-(tetrahydro-2H-pyran-4-yl)imidazo[5,1-f][1,2,4]triazin-4(3H)-one (200 mg, 0.787 mmol) was added to the mixture and stirred at 60° C. for overnight. The reaction was quenched with Na₂SO₄ 10H₂O, filtered, and concentrated in vacuo. The obtained compound was used to the next reaction without further purification. LC-MS (m/z)=251 [M+H]⁺.

### Reference Example 23

### 2-(Pyrrolidin-1-yl)-7-(tetrahydro-2H-pyran-4-yl)imidazo[5,1-f][1,2,4]triazin-4(3H)-one

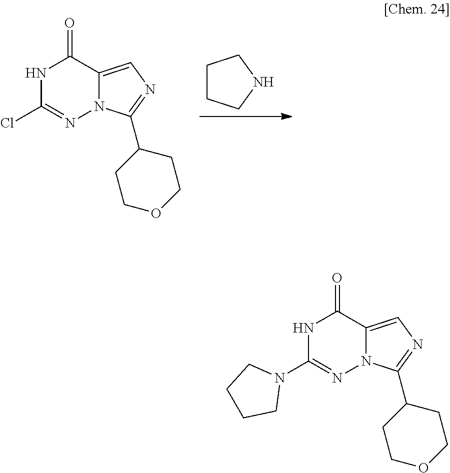 [SMILES: O=c1[nH]c(Cl)nn2c(C3CCOCC3)ncc12 ; C1CCNC1 ; O=c1[nH]c(N2CCCC2)nn2c(C3CCOCC3)ncc12]

A solution of 2-chloro-7-(tetrahydro-2H-pyran-4-yl)imidazo[5,1-f][1,2,4]triazin-4(3H)-one (1.0 g, 3.93 mmol) and pyrrolidine (2.0 mL) was heated at 100° C. for 6 h under nitrogen atmosphere. Upon completion, the resulting mixture was diluted with sat. ammonium chloride aq., and extracted with chloroform. The combined organic extracts were dried over sodium sulfate, filtered, and concentrated in vacuo. The residue was purified by silica gel column chromatography (chloroform/MeOH) to give the titled compound (1.13 g, yield 99%) as white solids.

LC-MS (m/z)=290 [M+H]⁺. ¹H-NMR (400 MHz, CDCl₃): δ 1.93-1.96 (m, 2H), 2.05-2.16 (m, 6H), 3.33-3.40 (m, 1H), 3.51-3.62 (m, 6H), 4.07-4.11 (m, 2H), 7.75 (s, 1H), 9.10 (br s, 1H).

### Reference Example 24

### 2-(Chloromethyl)-7-cyclopentyl-3-methylimidazo[5,1-f][1,2,4]triazin-4(3H)-one

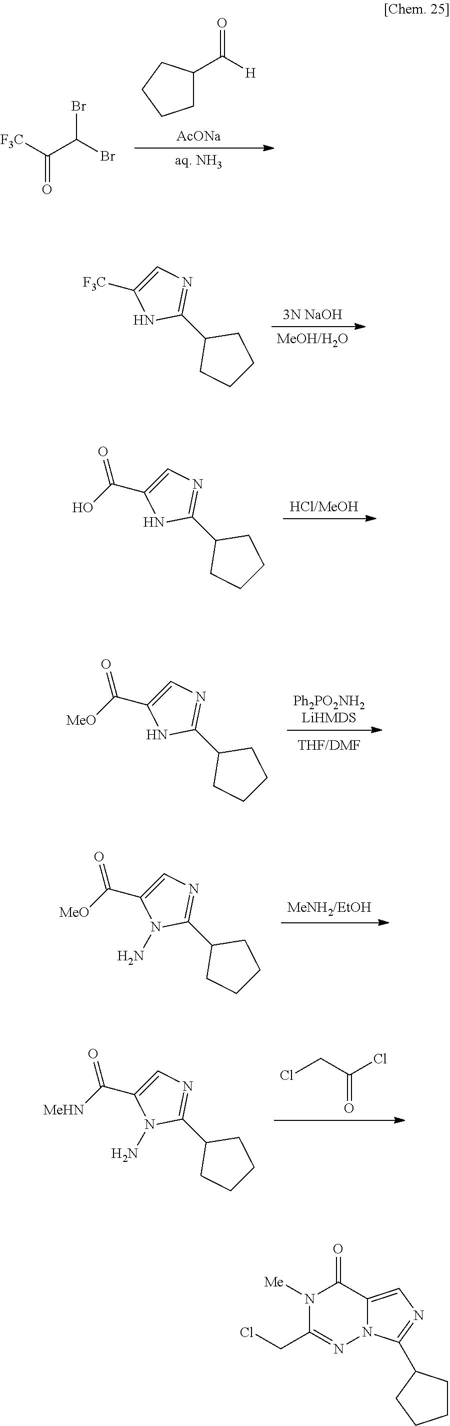

A solution of 1-amino-2-cyclopentyl-N-methyl-1H-imidazole-5-carboxamide (1.70 g, 8.2 mmol) in 2-chloroacetyl chloride (3 mL) was stirred under nitrogen at 80° C. for 3 h. After cooling to r.t., the reaction mixture was quenched with NaHCO₃ aq. (10 mL) and extracted with DCM (10 ml×3). The combined organic phases were dried and concentrated in vacuo. The residue was purified by silica gel chromatography (elution with 5% MeOH in DCM) to give the titled compound (1.3 g, yield 59%) as brown oil.

¹H-NMR (400 MHz, CD₃OD): δ 1.75-1.94 (m, 6H), 2.10-2.18 (m, 2H), 3.64-3.70 (m, 4H), 4.77 (s, 2H), 7.74 (s, 1H).

### 2-Cyclopentyl-5-(trifluoromethyl)-1H-imidazole

A mixture of 3,3-dibromo-1,1,1-trifluoropropan-2-one (8.1 g, 30 mmol) and sodium acetate (8.1 g, 60 mmol) in water (54 mL) was heated to reflux for 30 mins, and then the mixture was cooled to room temperature. Cyclopentanecarbaldehyde (2.65 g, 27 mmol) and Ammonium Hydroxide (33 mL) in methanol (135 mL) was added. The mixture was stirred at room temperature overnight. Upon the completion, methanol was removed and the aqueous was extracted with ethyl acetate (50 mL×2). The combined organic phase was dried and concentrated. The crude (5.5 g) was used without further purification for next step.

LC-MS (m/z)=205 [M+H]⁺.

### 2-Cyclopentyl-1H-imidazole-5-carboxylic acid

A mixture of 2-cyclopentyl-5-(trifluoromethyl)-1H-imidazole (5.5 g, 27 mmol) and sodium hydroxide (3 g, 73 mmol) in water/methanol (40 mL/60 mL) was stirred at room temperature overnight. Upon the completion, methanol was removed and the pH of aqueous solution was adjusted to pH=2 with 1N hydrochloric acid. The solvent was removed under reduce pressure and the residue was dissolved with ethanol (15 mL), filtered and the filtrate was concentrated. The crude (4.5 g) was used without further purification for next step.

### Methyl 2-cyclopentyl-1H-imidazole-5-carboxylate

The hydrochloric gas was bubbled into the solution of 2-cyclopentyl-1H-imidazole-5-carboxylic acid (4.5 g) in methanol (50 mL) with stirring for 2 hours, and then the mixture was heated for reflux overnight. Upon the completion, the mixture was washed with sodium bicarbonate solution (30mL×3), dried, and concentrated. The crude was purified by silica gel (eluted with petroleum ether:ethyl acetate=50:1 to petroleum ether: ethyl acetate=10:1) to give the titled compound (3 g, yield 62%) as a white solid. LC-MS (m/z)=195 [M+H]⁺.

### Methyl 1-amino-2-cyclopentyl-1H-imidazole-5-carboxylate

To a mixture of methyl 2-cyclopentyl-1H-imidazole-5-carboxylate (3.10 g, 16.0 mmol) in anhydrous DMF (30 mL) was added LiHMDS (1N in THF, 21 mL, 21 mmol) at −10° C. After stirring for 10 min, O-(diphenylphosphoryl)-hydroxylamine (4.5 g, 19 mmol) was added at 0° C., and another 20 ml of anhydrous DMF was added. The reaction was warmed to room temperature and stirred for 16 hours. Upon completion, the mixture was quenched with water (100 mL), and extracted with ethyl acetate (100 mL×3). The combined organic phases were dried and concentrated to give a crude product. Purification by silica gel chromatography (elution with 2%MeOH in DCM) gave the titled compound (2.30 g, yield 71%) as yellow oil. LC-MS (m/z)=210 [M+H]⁺.

### 1-Amino-2-cyclopentyl-N-methyl-1H-imidazole-5-carboxamide

A solution of methyl 1-amino-2-cyclopentyl-1H-imidazole-5-carboxylate (2.30 g, 11.0 mmol) in 20 mL of CH₃NH₂/EtOH in a sealed tube was stirred at 80° C. for 16 h. After cooling to room temperature, most of the solvent was removed in vacuo to give the crude, which was used for next step without further purification.

### Reference Example 25

### 2-(Chloromethyl)-3-methyl-7-(propan-2-yl)imidazo[5,1-f][1,2,4]triazin-4(3H)-one

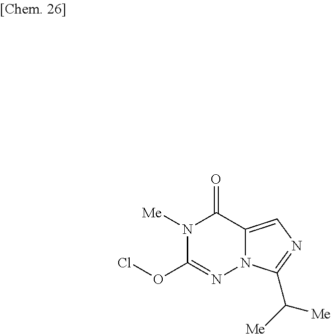 [SMILES: CC(C)c1ncc2c(=O)n(C)c(OCl)nn12]

The titled compound was synthesized in a similar manner to Reference Example 24.

¹H NMR (400 MHz, CDCl₃): δ 1.39 (d, J=6.8 Hz, 6H), 3.50 (sept, J=6.8 Hz, 1H), 3.62 (s, 3H), 4.56 (s, 2H), 7.81 (s, 1H).

### Reference Example 26

### 3-(5-Methylpyridin-2-yl)propan-1-ol

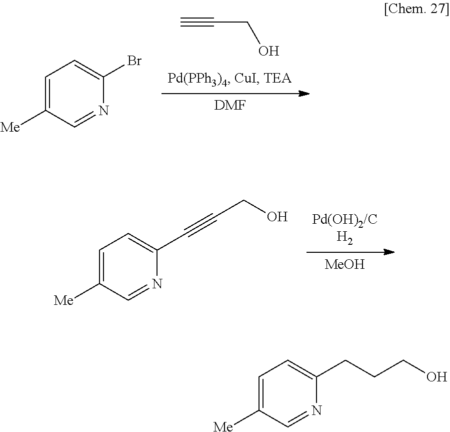

To a solution of 3-(5-methylpyridin-2-yl)prop-2-yn-1-ol in MeOH (7.0 mL) was added Pd(OH)₂/C (500 mg). The mixture was stirred for 4 h at room temperature under H₂ atmosphere. Upon completion, the reaction mixture was filtered through a Celite filter, and the filtrate was concentrated to dryness. The residue was purified by amino silica gel column chromatography (hexane/EtOAc) to give the titled compound (183 mg, yield 42% for 2 steps) as a colorless oil.

LC-MS (m/z)=152 [M+H]⁺. ¹H-NMR (400 MHz, CDCl₃): δ 1.85-1.91 (m, 2H), 2.21 (s, 3H), 2.80-2.87 (m, 2H), 3.58-3.63 (m, 2H), 6.99-7.01 (m, 1H), 7.33-7.36 (m, 1H), 8.22 (s, 1H).

### 3-(5-Methylpyridin-2-yl)prop-2-yn-1-ol

To a solution of 2-bromo-5-methylpyridine (500 mg, 2.91 mmol) and prop-2-yn-1-ol (223 μl, 3.78 mmol) in DMF (0.3 mL) and TEA (1.62 ml) was added Pd(PPh₃)₄ (336 mg, 0.029 mmol) and CuI (111 mg, 0.582 mmol). The mixture was heated overnight at 70° C. under nitrogen atmosphere. Upon completion, the reaction mixture was cooled and partitioned between ethyl acetate and brine. The aqueous layer was extracted with EtOAc, and the combined organic phases were dried with sodium sulfate and concentrated to dryness. The residue was used to the next reaction without further purification.

### Reference Example 27

### 3-(5-Fluoropyridin-2-yl)propan-1-ol

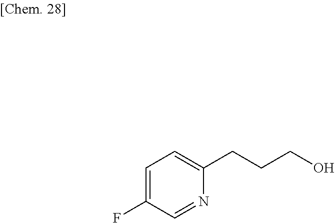 [SMILES: OCCCc1ccc(F)cn1]

The titled compound was synthesized in a similar manner to Reference Example 27.

¹H-NMR (300 MHz, CDCl₃): δ 1.86-1.95 (m, 2H), 2.87 (t, J=7.0 Hz, 2H), 3.62 (t, J=6.2 Hz, 2H), 7.10-7.14 (m, 1H), 7.24-7.31 (m, 1H), 8.28-8.29 (m, 1H).

### Reference Example 28

### 5-Fluoro-2-[(3S)-pyrrolidin-3-yloxy]pyridine

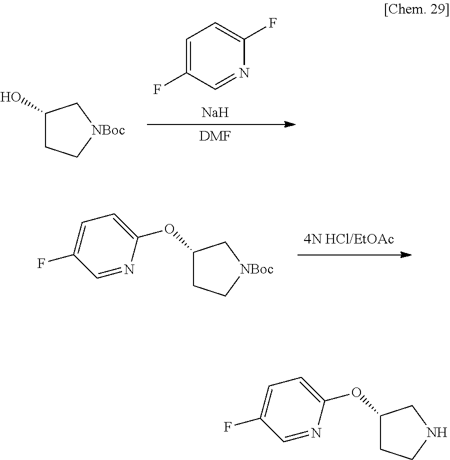

tert-Butyl (3S)-3-[(5-fluoropyridin-2-yl)oxy]pyrrolidine-1-carboxylate was dissolved in MeOH (3.0 mL) then 4 N-HCl/AcOEt (6.0 mL) was added at 0° C. The reaction mixture was stirred at room temperature for 1 h and concentrated to obtain the desired product as a colorless solid (560 mg, yield 89%).

¹H-NMR (300 MHz, CD₃OD): δ 2.23-2.35 (m, 2H), 3.44 (t, J=7.7 Hz, 2H), 3.52 (brs, 2H), 5.57-5.62 (m, 1H), 6.81-6.92 (m, 1H), 7.51-7.61 (m, 1H), 8.01 (dd, J=6.5, 3.0 Hz, 1H).

### tert-Butyl (3S)-3-[(5-fluoropyridin-2-yl)oxy]pyrrolidine-1-carboxylate

tert-Butyl (3S)-3-hydroxypyrrolidine-1-carboxylate (500 mg, 2.67 mmol) and 2,5-difluoropyridine (360 μL, 4.00 mmol) were dissolved in DMF (3.0 mL). After addition of NaH (160 mg, 4.00 mmol, 60% in paraffin) the reaction mixture was stirred at 50° C. for 23 hr. The mixture was cooled to room temperature and quenched with H₂O. The aqueous phase was extracted with toluene. The combined organic phases were dried over MgSO₄, concentrated and purified by silica gel column chromatography (hexane/EtOAc) to give the desired product as a colorless oil (695 mg, yield 92%)

¹H-NMR (300 MHz, CDCl₃): δ 1.46 (s, 9H), 2.14 (s, 2H), 3.41-3.69 (m, 4H), 5.46 (s, 1H), 6.69 (dd, J=9.0, 3.3 Hz, 1H), 7.35 (d, J=7.7 Hz, 1H), 7.96 (s, 1H).

### Reference Example 29

### 5-Fluoro-2-[(3R)-pyrrolidin-3-yloxy]pyridine

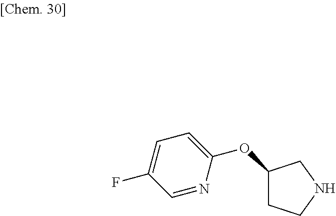 [SMILES: Fc1ccc(O[C@@H]2CCNC2)nc1]

The titled compound was synthesized in a similar manner to Reference Example 29.

¹H-NMR (300 MHz, CD₃OD): δ 2.23-2.35 (m, 2H), 3.44 (t, J=7.7 Hz, 2H), 3.52 (brs, 2H), 5.57-5.62 (m, 1H), 6.81-6.92 (m, 1H), 7.51-7.61 (m, 1H), 8.01 (dd, J=6.5, 3.0 Hz, 1H).

### Reference Example 30

### 2-Chloro-3-methyl-7-(tetrahydro-2H-pyran-4-yl)imidazo[5,1-f][1,2,4]triazin-4(3H)-one

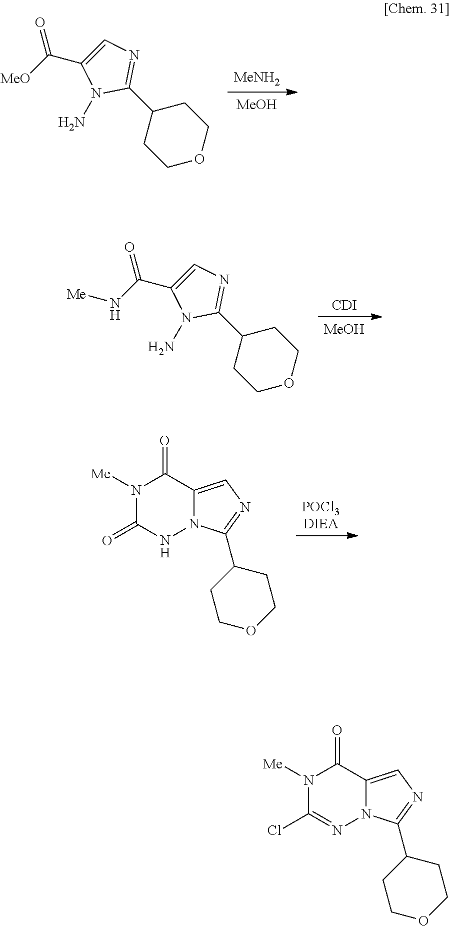

A mixture of 3-methyl-7-(tetrahydro-2H-pyran-4-yl)imidazo[5,1-f][1,2,4]triazine-2,4(1H,3H)-dione (2.68 g, 10.73 mmol), phosphoryl trichloride (10 mL, 107 mmol)) and DIEA (2.81 mL, 16.09 mmol) was stirred for 15 h at 100° C. Upon completion, the reaction mixture was poured into ice water. After neutralization with NaOH aq., the resulting precipitation was filtered to give crude product (2.56 g). The crude was purified with silica gel column chromatography (hexane/EtOAc) to give the titled compound (2.17 g, 8.08 mmol, yield 75%).

LC-MS (m/z)=270 [M+H]⁺. ¹H-NMR (400 MHz, CDCl₃): δ 1.86-1.95 (m, 2H), 2.01-2.14 (m, 2H), 3.36-3.47 (m, 1H), 3.55-3.66 (m, 5H), 4.05-4.13 (m, 2H), 7.84 (s, 1H).

### 1-Amino-N-methyl-2-(tetrahydro-2H-pyran-4-yl)-1H-imidazole-5-carboxamide

A mixture of methyl 1-amino-2-(tetrahydro-2H-pyran-4-yl)-1H-imidazole-5-carboxylate (10 g, 44.4 mmol) and 40% methylamine MeOH solution (115 mL, 444 mmol) was stirred for 4 h at 60° C. After evaporation, EtOAc (50 mL) and diisopropyl ether (50 mL) was added to the residue. The obtained suspension was stirred for 1 h at 0° C. The titled compound was collected by filtration (9.3 g, 41.5 mmol, yield 93%). LC-MS (m/z)=225 [M+H]⁺.

### 3-Methyl-7-(tetrahydro-2H-pyran-4-yl)imidazo[5,1-f][1,2,4]triazine-2,4(1H,3H)-dione

A solution of 1-Amino-N-methyl-2-(tetrahydro-2H-pyran-4-yl)-1H-imidazole-5-carboxamide (5 g, 22.3 mmol) and CDI (5.42 g, 33.4 mmol) in MeCN (50 mL) was stirred for 3 h at 60° C. Upon completion, the suspension was stirred for 30 min at 0° C. The precipitation was filtered to give crude product (6.95 g) including imidazole. 2-propanol (70 mL) was added to the crude and the obtained suspension was stirred for 2.5 h at 90° C. After the suspension was cooled to 0° C., The titled compound was collected by filtration (5.11 g, 20.42 mmol, yield 91%).

LC-MS (m/z)=251 [M+H]⁺.

### Reference Example 31

### 2-(Chloromethyl)-3-ethyl-7-(tetrahydro-2H-pyran-4-yl)imidazo[5,1-f][1,2,4]triazin-4(3H)-one

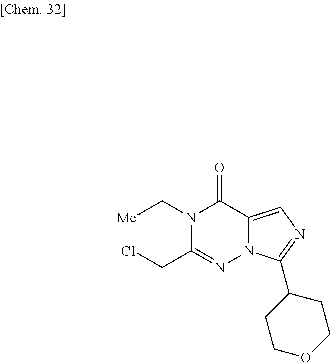 [SMILES: CCn1c(CCl)nn2c(C3CCOCC3)ncc2c1=O]

The titled compound was synthesized in a similar manner to Reference Example 24.

¹H-NMR (400 MHz, CDCl₃): δ 1.39 (t, J=7.1 Hz, 3H), 1.87-1.97 (m, 2H), 2.04-2.17 (m, 2H), 3.38-3.49 (m, 1H), 3.55-3.65 (m, 2H), 4.06-4.14 (m, 2H), 4.18 (q, J=7.1 Hz, 2H), 4.53 (s, 2H), 7.83 (s, 1H).

The compounds of Reference Examples 32 to 33 were synthesized in a similar manner to Reference Example 30.

| | |
|---|---|
| | [Chem. 33] |
| 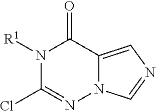 | |

| No. | R¹— | ¹H NMR or LC-MS |
|---|---|---|
| 32 | Me— | ¹H NMR (400 MHz, CDCl₃): δ 3.66 (s, 3H), 7.92 |
| | | (s, 1 H), 8.08 (s, 1 H). |
| 33 | Et— | ¹H NMR (400 MHz, DMSO-d₆): δ 1.23 (t, J = 7.2 |
| | | Hz, 3H), 4.08 (q, J = 7.6 Hz, 2H), 7.84 (s, 1H), |
| | | 8.50 is, 1H). |

The compounds of Reference Examples 34 to 37 were synthesized in a similar manner to Reference Example 5, 6 or 7.

| | |
|---|---|
| | [Chem. 34] |
| 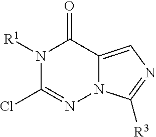 | |

| No. | R¹— | R³— | ¹H NMR or LC-MS |
|---|---|---|---|
| 34 | 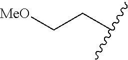 [SMILES: *CCOC] | 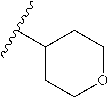 [SMILES: *C1CCOCC1] | ¹H NMR (400 MHz, CDCl₃): (400 MHz, CDCl₃): δ 1.90-1.93 (m, 2H), 2.10-2.21 (m, 2H), 3.36 (s, 3H), 3.60 (t, J = 11.2 Hz, 3H), 3.68 (t, J = 5.2 Hz, 2H), 4.15 (dd, J = 11.6, 3.6 Hz, 2H), 4.43 (t, J = 5.6 Hz, 2H), 8.07 (s, 1H). |
| 35 | 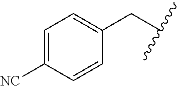 [SMILES: *Cc1ccc(C#N)cc1] | 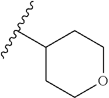 [SMILES: *C1CCOCC1] | LC-MS (m/z) = 370 [M + H]⁺. |
| 36 | 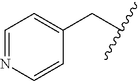 [SMILES: *Cc1ccncc1] | 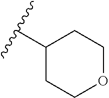 [SMILES: *C1CCOCC1] | LC-MS (m/z) = 346 [M + H]⁺. |
| 37 | 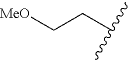 [SMILES: *CCOC] | 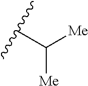 [SMILES: *C(C)C] | LC-MS (m/z) = 271 [M + H]⁺. |

### Example 1

### 2-[(4,4-Difluorocyclohexyl)methoxyl-3-ethyl-7-(tetrahydro-2H-pyran-4-yl)imidazo[5,1-f][1,2,4]triazin-4(3H)-one

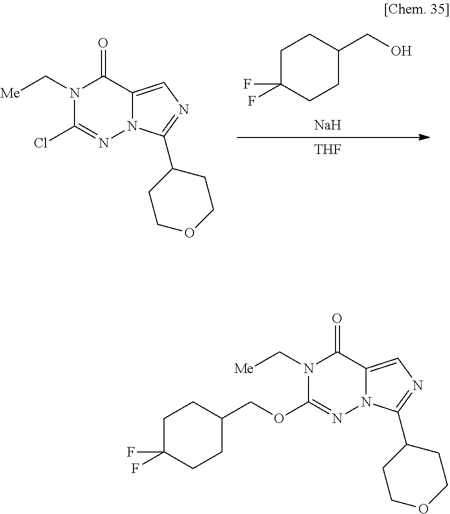

To a suspension of sodium hydride (55% oil suspension, 5.8 mg, 0.133 mmol) in THF (1.0 mL), (4,4-difluorocyclohexyl)methanol (15.3 mg, 0.102 mmol) was added dropwise. The reaction mixture was stirred at 0° C. for 30 min. After 2-chloro-3-ethyl-7-(tetrahydro-2H-pyran-4-yl)imidazo[5,1-f][1,2,4]triazin-4(3H)-one (29 mg, 0.102 mmol) was added thereto, the mixture was stirred for 3.5 h at room temperature. Upon completion, the reaction mixture was diluted with H₂O and saturated NaCl aq. The aqueous layer was extracted with EtOAc and the combined organic layer was concentrated in vacuo. The residue was purified by silica gel column chromatography (hexane/EtOAc) to give the titled compound (24 mg, yield 60%) as a white solid.

LC-MS (m/z)=397 [M+H]⁺. ¹H-NMR (400 MHz, CDCl₃): δ 1.27 (t, J=7.1 Hz, 3H), 1.47-1.57 (m, 2H), 1.71-1.98 (m, 7H), 2.05-2.23 (m, 4H), 3.34 (tt, J=11.6, 4.0 Hz, 1H), 3.58 (td, J=11.6, 2.0 Hz, 2H), 4.01-4.12 (m, 4H), 4.25 (d, J=6.0 Hz, 2H), 7.77 (s, 1H).

### Example 2

### 3-Methyl-2-[(5-methylpyridin-2-yl)methoxy]-7-(propan-2-yl)imidazo[5,1-f][1,2,4]triazin-4(3H)-one

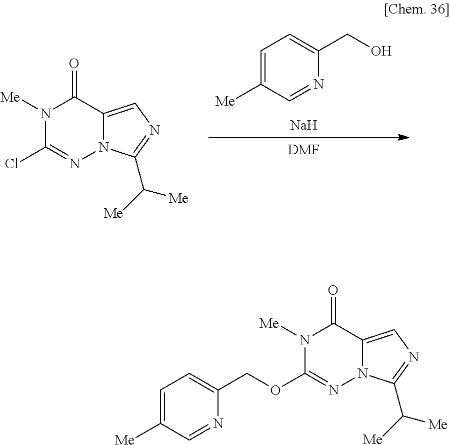

A solution of (5-methylpyridin-2-yl)methanol (192 mg, 1.77 mmol) in anhydrous DMF (10 mL) was added 60% NaH (71 mg, 1.77 mmol) at 0 oC for 0.5 h, and then 2-chloro-7-isopropyl-3-methylimidazo[5,1-f][1,2,4]triazin-4(3H)-one (100 mg, 0.44 mmol) was added at 0° C. for another 0.5 h. The mixture was stirred at 25° C. for 1 h. The reaction was quenched with water, extracted with EtOAc, concentrated and then purified through flash-column to give the titled compound (40 mg, yield 33%).

LC-MS (m/z)=314.0 [M+H]⁺. ¹H-NMR (400 MHz, CD₃OD): δ 1.19-1.21 (m, 6H), 2.28 (s, 3H), 3.31-3.34 (m, 1H), 3.35 (s, 3H), 5.41 (s, 2H), 7.44-7.46 (m, 1H), 7.56 (s, 1H), 7.63-7.65 (m, 1H), 8.32 (s, 1H).

### Example 3

### 3-Methyl-7-(propan-2-yl)-2-[2-(pyridin-4-yl)ethoxy]imidazo[5,1-f][1,2,4]triazin-4(3H)-one

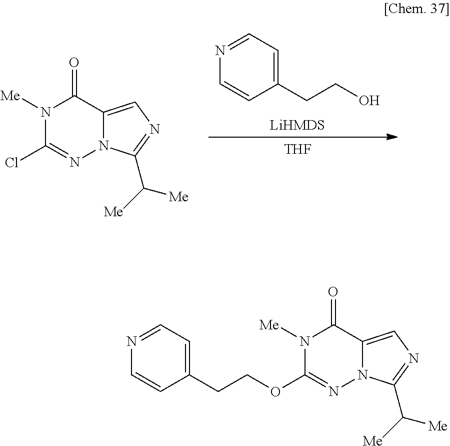

To a mixture of 2-(pyridin-4-yl)ethanol (2.46 g, 20 mmol) in THF (16 mL) at −78° C. was added LiHMDS (22 mL, 22 mmol) dropwise. The mixture was stirred for 20 min at −78° C., and then 2-chloro-3-methyl-7-(propan-2-yl)imidazo[5,1-f][1,2,4]triazin-4(3H)-one (452 mg, 2 mmol) was added thereto. The mixture was warmed to r.t., and stirred overnight. The mixture was cooled to 0° C., quenched with water, extracted with EtOAc, concentrated and then purified by a preparative HPLC to afford the titled compound (12 mg, yield 2%) as a white solid.

LC-MS (m/z)=314 [M+H]⁺. ¹H-NMR (400 MHz, CDCl₃): δ 1.39 (d, J=7.2 Hz, 6H), 3.16-3.19 (m, 2H), 3.33 (s, 3H), 3.40-3.44 (m, 1H), 4.63 (t, J=6.8 Hz, 2H), 7.22-7.24 (m, 2H), 7.77 (s, 1H), 8.59-8.60 (m, 2H).

The compounds of Examples 4 to 114 were synthesized in a similar manner to Example 1, 2 or 3.

| | | | | [Chem. 38] |
|---|---|---|---|---|
| 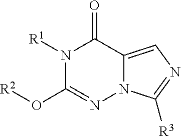 | | | | |
| No. | R¹— | R²— | R³— | ¹H-NMR |
| 4 | Me— | 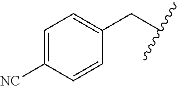 [SMILES: *Cc1ccc(C#N)cc1] | 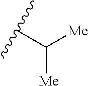 [SMILES: *C(C)C] | (400 MHz, CD₃OD): δ 1.33-1.36 (m, 6H), 3.45 (s, 3H), 3.45-3.51 (m, 1H), 5.55-5.59 (m, 2H), 7.67 (s, 1H), 7.71-7.74 (m, 2H), 7.79-7.81 (m, 2H). |
| 5 | Me— | 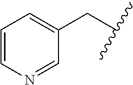 [SMILES: *Cc1cccnc1] | 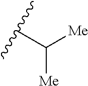 [SMILES: *C(C)C] | (400 MHz, CD₃OD): δ 1.35-1.37 (m, 6H), 3.1 (s, 3H), 3.49-3.59 (m, 1H), 5.53-5.56 (m, 2H), 7.51-7.54 (m, 1H), 7.64-7.67 (m, 1H), 8.05- 8.07 (m, 1H), 8.56-8.57 (m, 1H). 8.75-8.76(m, 1H). |
| 6 | Me— | 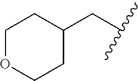 [SMILES: *CC1CCOCC1] | 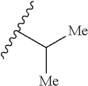 [SMILES: *C(C)C] | (400 MHz, DMSO-d₆): δ 1.40 (d, J = 7.2 Hz, 6H), 1.51-1.58 (m, 2H), 1.66-1.76 (m, 2H), 2.14-2.17 (m, 1H), 3.40-3.50 (m, 6H), 4.04-4.08 (m, 2H), 4.24 (d, J = 6.0 Hz, 2H), 7.78 (m, 1H). |
| 7 | Me— | 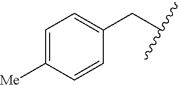 [SMILES: *Cc1ccc(C)cc1] | 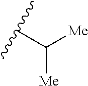 [SMILES: *C(C)C] | (400 MHz, CDCl₃): δ 1.41 (d, J = 8.0 Hz, 6H), 2.39 (s, 3H), 3.40 (s, 3H), 3.46-3.49 (m, 1H) 5.37 (s, 2H), 7.23-7.25 (m, 2H), 7.36-7.38 (m, 2H), 7.77 (s, 1H). |
| 8 | Me— | 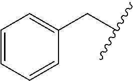 [SMILES: *Cc1ccccc1] | 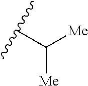 [SMILES: *C(C)C] | (400 MHz, CDCl₃): δ 1.41 (d, J = 7.2 Hz, 6H), 3.43 (s, 3H), 3.45-3.49 (m, 1H), 5.41 (s, 2H), 7.41-7.49 (m, 5H), 7.78 (s, 1H). |
| 9 | Me— | 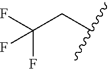 [SMILES: *CC(F)(F)F] | 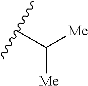 [SMILES: *C(C)C] | (400 MHz, CD₃OD): δ 1.25-1.27 (m, 6H), 3.32 (s, 3H), 3.38-3.43 (m, 1H), 4.84-4.90 (m, 2H), 7.59 (s, 1H). |
| 10 | Me— | 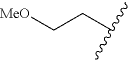 [SMILES: *CCOC] | 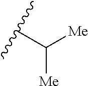 [SMILES: *C(C)C] | (400 MHz, DMSO-d₆): δ 1.34 (d, J = 7.2 Hz, 6H), 3.41-3.46 (m, 7H), 3.79-3.81 (m, 2H), 4.53-4.55 (m, 2H), 7.75-7.78 (m, 1H). |
| 11 | Me— | 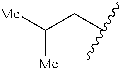 [SMILES: *CC(C)C] | 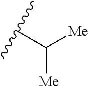 [SMILES: *C(C)C] | (400 MHz, DMSO-d₆): δ 1.07 (d, J = 6.4 Hz, 6H), 1.40 (d, J = 7.2 Hz, 6H), 2.15-2.21 (m, 1H), 3.40-3.47 (m, 4H), 4.16 (d, J = 6.4 Hz, 2H), 7.77 (m, 1H). |
| 12 | Me— | 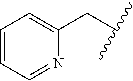 [SMILES: *Cc1ccccn1] | 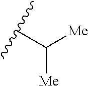 [SMILES: *C(C)C] | (400 MHz, CDCl₃): δ 1.35 (d, J = 6.8 Hz, 6H), 3.39-3.42 (m, 1H), 3.50 (s, 3H), 5.54 (s, 2H), 7.26-7.28 (m, 1H), 7.47-7.49 (m, 1H), 7.77- 7.80 (m, 2H), 8.65-8.67 (m, 1H). |
| 13 | Me— | 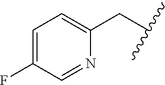 [SMILES: *Cc1ccc(F)cn1] | 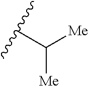 [SMILES: *C(C)C] | (400 MHz, CDCl₃): δ 1.37 (d, J = 6.0 Hz, 6H), 3.40-3.43 (m, 1H), 3.49 (s, 3H), 5.52 (s, 2H), 7.50-7.52 (m, 2H), 7.80 (s, 1H), 8.52-8.53 (m, 1H). |
| 14 | Me— | 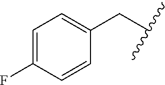 [SMILES: *Cc1ccc(F)cc1] | 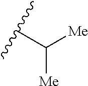 [SMILES: *C(C)C] | (400 MHz, CDCl₃): δ 1.41 (d, J = 6.0 Hz, 6H), 3.42 (s, 3H), 3.46-3.48 (m, 1H), 5.39 (s, 2H), 7.11-7.15 (m, 2H), 7.46-7.49 (m, 2H), 7.79 (s, 1H). |
| 15 | Me— | 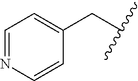 [SMILES: *Cc1ccncc1] | 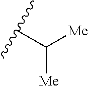 [SMILES: *C(C)C] | (400 MHz, CD₃OD): δ 1.30-1.32 (m, 6H), 3.41-3.47 (m, 1H), 3.50 (s, 3H), 5.57 (s, 2H), 7.58-7.60 (m, 2H), 7.68 (s, 1H), 8.58-8.60 (m, 2H). |
| 16 | Et— | 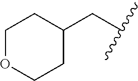 [SMILES: *CC1CCOCC1] | 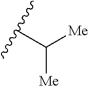 [SMILES: *C(C)C] | (400 MHz, CD₃OD): δ 1.15-1.18 (m, 3H), 1.26-1.28 (m, 6H), 1.37- 1.46 (m, 2H), 1.67 (d, J = 13.2 Hz, 2H), 2.05 (br, 1H), 3.35-3.41 (m, 3H), 3.88-3.98 (m, 4H), 4.20-4.22 (m, 2H), 7.56 (s, 1H). |
| 17 | Et— | 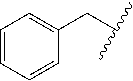 [SMILES: *Cc1ccccc1] | 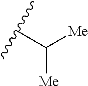 [SMILES: *C(C)C] | (400 MHz, CDCl₃): δ 1.26 (t, J = 7.2 Hz, 3H), 1.40 (d, J = 6.8 Hz, 6H), 3.46 (sept, J = 6.8 Hz, 1H), 4.06 (q, J = 7.2 Hz, 2H), 5.42 (s, 2H), 7.37-7.48 (m, 5H), 7.76 (s, 1H). |
| 18 | Et— | 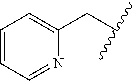 [SMILES: *Cc1ccccn1] | 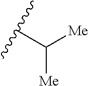 [SMILES: *C(C)C] | (400 MHz, CDCl₃): δ 1.30-1.35 (m, 9H), 3.39 (sept, J = 6.8 Hz, 1H), 4.13 (q, J = 7.2 Hz, 2H), 5.54 (s, 2H), 7.29-7.32 (m, 1H), 7.45 (d, J = 8.0 Hz, 1H), 7.75-7.80 (m, 2H), 8.64 (d, J = 4.8 Hz, 1H). |
| 19 | Et— | 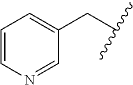 [SMILES: *Cc1cccnc1] | 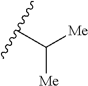 [SMILES: *C(C)C] | (400 MHz, CDCl₃): δ 1.26 (t, J = 7.2 Hz, 3H), 1.40 (d, J = 7.2 Hz, 6H), 3.45 (sept, J = 7.2 Hz, 1H), 4.05 (q, J = 7.2 Hz, 2H), 5.45 (s, 2H), 7.37-7.40 (m, 1H), 7.77 (s, 1H), 7.81 (d, J = 7.6 Hz, 1H), 8.66 (d, J = 4.4 Hz, 1H), 8.76 (s, 1H). |
| 20 | Et— | 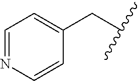 [SMILES: *Cc1ccncc1] | 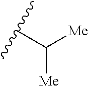 [SMILES: *C(C)C] | (400 MHz, CDCl₃): δ 1.30-1.37 (m, 9H), 3.39 (sept, J = 6.8 Hz, 1H), 4.11 (q, J = 7.2 Hz, 2H), 5.44 (s, 2H), 7.36 (d, J = 5.2 Hz, 2H), 7.78 (s, 1H), 8.69 (d, J = 6.0 Hz, 2H). |
| 21 | Et— | 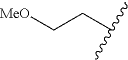 [SMILES: *CCOC] | 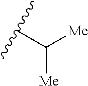 [SMILES: *C(C)C] | (400 MHz, CDCl₃): δ 1.28 (t, J = 7.2 Hz, 3H), 1.39 (d, J = 6.8 Hz, 6H), 3.36-3.46 (m, 4H), 3.78 (t, J = 4.8 Hz, 2H), 4.06 (q, J = 7.2 Hz, 2H), 4.54 (t, J = 4.8 Hz, 2H), 7.76 (s, 1H). |
| 22 | Me— | 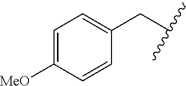 [SMILES: *Cc1ccc(OC)cc1] | 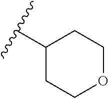 [SMILES: *C1CCOCC1] | (300 MHz, CDCl₃): δ 1.86-1.91 (m, 2H), 2.01-2.15 (m, 2H), 3.31-3.41 (m, 4H), 3.54-3.62 (m, 2H), 3.81 (s, 3H), 4.06-4.12 (m, 2H), 5.31 (s, 2H), 6.91 (d, J = 8.8 Hz, 2H), 7.37 (d, J = 8.8 Hz, 2H), 7.74 (s, 1H). |
| 23 | Me— | 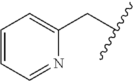 [SMILES: *Cc1ccccn1] | 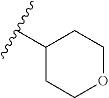 [SMILES: *C1CCOCC1] | (400 MHz, CDCl₃): δ 1.77-1.80 (m, 2H), 1.99 (ddd, J = 25.1, 11.8, 4.3 Hz, 2H), 3.23-3.30 (m, 1H), 3.47 (s, 3H), 3.50-3.56 (m, 2H), 4.01-4.05 (m, 2H), 5.50 (s, 2H), 7.27-7.30 (m, 1H), 7.42-7.44 (m, 1H), 7.73-7.77 m, 2H), 8.61-8.63 (m, 1H). |
| 24 | Me— | 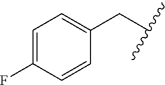 [SMILES: *Cc1ccc(F)cc1] | 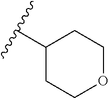 [SMILES: *C1CCOCC1] | (400 MHz, CDCl₃): δ 1.80-1.84 (m, 2H), 1.97-2.09 (m, 2H), 3.25-3.34 (m, 4H), 3.50-3.56 (m, 2H), 4.02- 4.07 (m, 2H), 5.30 (s, 2H), 7.01- 7.07 (m, 2H), 7.36-7.40 (m, 2H), 7.71 (s, 1H). |
| 25 | Me— | 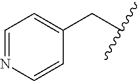 [SMILES: *Cc1ccncc1] | 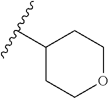 [SMILES: *C1CCOCC1] | (400 MHz, CDCl₃): δ 1.77-1.81 (m, 2H), 1.98-2.08 (m, 2H), 3.27 (tt, J = 11.6, 3.9 Hz, 1H), 3.47 (s, 3H), 3.54 (td, J = 11.7, 2.2 Hz, 2H), 4.04-4.08 (m, 2H), 5.40 (s, 2H), 7.33-7.34 (m, 2H), 7.76 (s, 1H), 8.65-8.67 (m, 2H). |
| 26 | Me— | 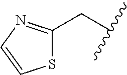 [SMILES: *Cc1nccs1] | 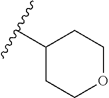 [SMILES: *C1CCOCC1] | (400 MHz, CDCl₃): δ 1.84-1.88 (m, 2H), 1.99-2.09 (m, 2H), 3.30-3.38 (m, 1H), 3.42 (s, 3H), 3.55 (td, J = 11.7, 2.2 Hz, 2H), 4.03-4.08 (m, 2H), 5.69 (s, 2H), 7.43 (d, J = 3.2 Hz, 1H), 7.76 (s, 1H), 7.83 (d, J = 3.2 Hz, 1H). |
| 27 | Me— | 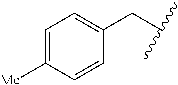 [SMILES: *Cc1ccc(C)cc1] | 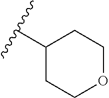 [SMILES: *C1CCOCC1] | (400 MHz, CDCl₃): δ 1.85-1.89 (m, 2H), 2.02-2.12 (m, 2H), 2.36 (s, 3H), 3.31-3.38 (m, 4H), 3.58 (td, J = 11.7, 2.2 Hz, 2H), 4.06-4.11 (m, 2H), 5.34 (s, 2H), 7.20 (d, J = 7.8 Hz, 2H), 7.13 (d, J = 8.0 Hz, 2H), 7.74 (s, 1H). |
| 28 | Me— | 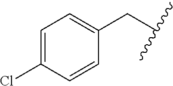 [SMILES: *Cc1ccc(Cl)cc1] | 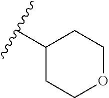 [SMILES: *C1CCOCC1] | (400 MHz, CDCl₃): δ 1.82-1.86 (m, 2H), 2.01-2.11 (m, 2H), 3.28-3.35 (m, 1H), 3.39 (s, 3H), 3.56 (td, J = 11.7, 2.2 Hz, 2H), 4.05-4.10 (m, 2H), 5.34 (s, 2H), 7.37 (s, 4H), 7.74 (s, 1H). |
| 29 | Me— | 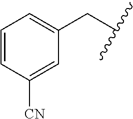 [SMILES: *Cc1cccc(C#N)c1] | 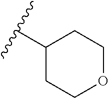 [SMILES: *C1CCOCC1] | (400 MHz, CDCl₃): δ 1.81-1.85 (m, 2H), 2.02-2.12 (m, 2H), 3.27-3.35 (m, 1H), 3.42 (s, 3H), 3.54-3.59 (m, 2H), 4.06-4.12 (m, 2H), 5.41 (s, 2H), 7.52-7.56 (m, 1H), 7.67-7.70 (m, 2H), 7.75-7.76 (m, 2H). |
| 30 | Me— | 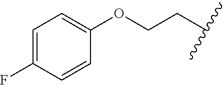 [SMILES: *CCOc1ccc(F)cc1] | 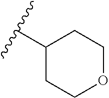 [SMILES: *C1CCOCC1] | (400 MHz, CDCl₃): δ 1.86-1.90 (m, 2H), 2.03-2.14 (m, 2H), 3.28-3.40 (m, 4H), 3.55 (td, J = 11.7, 2.2 Hz, 2H), 4.04-4.09 (m, 2H), 4.30-4.33 (m, 2H), 4.68-4.70 (m, 2H), 6.84- 6.87 (m, 2H), 6.95-7.01 (m, 2H), 7.75 (s, 1H). |
| 31 | Me— | 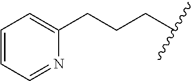 [SMILES: *CCCc1ccccn1] | 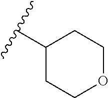 [SMILES: *C1CCOCC1] | (400 MHz, CDCl₃): δ 1.85-1.90 (m, 2H), 2.01-2.11 (m, 2H), 2.28-2.35 (m, 2H), 2.97 (t, J = 7.6 Hz, 2H), 3.26-3.33 (m, 4H), 3.54 (td, J = 11.7, 2.2 Hz, 2H), 4.03-4.08 (m, 2H), 4.44 (t, J = 6.2 Hz, 2H), 7.11- 7.17 (m, 2H), 7.60 (td, J = 7.7, 1.8 Hz, 1H), 7.74 (s, 1H), 8.52-8.54 (m, 1H). |
| 32 | Me— | 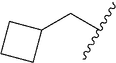 [SMILES: *CC1CCC1] | 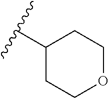 [SMILES: *C1CCOCC1] | (400 MHz, CDCl₃): δ 1.90-2.21 (m, 10H), 2.82-2.85 (m, 1H), 3.33-3.41 (m, 4H), 3.56-3.63 (m, 2H), 4.09- 4.12 (m, 2H), 4.33 (d, J = 6.4 Hz, 2H), 7.78 (s, 1H). |
| 33 | Me— | 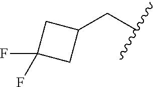 [SMILES: *CC1CC(F)(F)C1] | 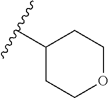 [SMILES: *C1CCOCC1] | (400 MHz, CDCl₃): δ 1.89-1.92 (m, 2H), 2.06-2.16 (m, 2H), 2.52-2.57 (m, 2H), 2.71-2.85 (m, 3H), 3.31- 3.42 (m, 4H), 3.56-3.62 (m, 2H), 4.08-4.13 (m, 2H), 4.44-4.46 (m, 2H), 7.78 (s, 1H). |
| 34 | Me— | 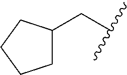 [SMILES: *CC1CCCC1] | 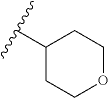 [SMILES: *C1CCOCC1] | (400 MHz, CDCl₃): δ 1.35-1.43 (m, 2H), 1.61-1.72 (m, 4H), 1.84-1.94 (m, 4H), 2.05-2.15 (m, 2H), 2.38- 2.46 (m, 1H), 3.32-3.39 (m, 1H), 3.41 (s, 3H), 3.56-3.63 (m, 2H), 4.08-4.11 (m, 2H), 4.25 (d, J = 6.4 Hz, 2H), 7.73 (s, 1H). |
| 35 | Me— | 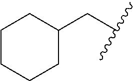 [SMILES: *CC1CCCCC1] | 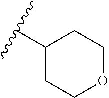 [SMILES: *C1CCOCC1] | (400 MHz, CDCl₃): δ 1.06-1.37 (m, 5H), 1.73-1.94 (m, 8H), 2.01-2.15 (m, 2H), 3.32-3.39 (m, 1H), 3.41 (s, 3H), 3.56-3.63 (m, 2H), 4.08-4.11 (m, 2H), 4.17 (d, J = 5.6 Hz, 2H), 7.77 (s, 1H). |
| 36 | Me— | 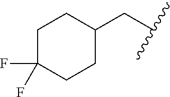 [SMILES: *CC1CCC(F)(F)CC1] | 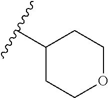 [SMILES: *C1CCOCC1] | (400 MHz, CDCl₃): δ 1.43-1.53 (m, 2H), 1.67-1.93 (m, 7H), 2.02-2.20 (m, 4H), 3.27-3.35 (m, 1H), 3.38 (s, 3H), 3.55 (td, J = 11.7, 2.2 Hz, 2H), 4.04-4.08 (m, 2H), 4.22 (d, J = 6.1 Hz, 2H), 7.75 (s, 1H). |
| 37 | Me— | 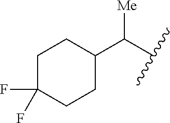 [SMILES: *C(C)C1CCC(F)(F)CC1] | 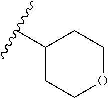 [SMILES: *C1CCOCC1] | (400 MHz, CDCl₃): δ 1.42 (d, J = 6.3 Hz, 3H), 1.45-1.56 (m, 2H), 1.64-1.95 (m, 7H), 2.02-2.17 (m, 4H), 3.26-3.38 (m, 4H), 3.53-3.59 (m, 2H), 4.06-4.09 (m, 2H), 4.96- 5.02 (m, 1H), 7.76 (s, 1H). |
| 38 | Me— | 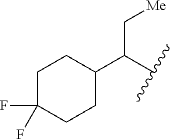 [SMILES: *C(CC)C1CCC(F)(F)CC1] | 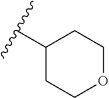 [SMILES: *C1CCOCC1] | (400 MHz, CDCl₃): δ 0.98 (t, J = 7.4 Hz, 3H), 1.45-1.53 (m, 2H), 1.63-1.90 (m, 9H), 2.04-2.18 (m, 4H), 3.25-3.32 (m, 1H), 3.38 (s, 3H), 3.52-3.59 (m, 2H), 4.05-4.11 m, 2H), 4.90-4.94 (m, 1H), 7.76 (s, 1H). |
| 39 | Me— | 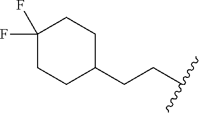 [SMILES: *CCC1CCC(F)(F)CC1] | 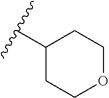 [SMILES: *C1CCOCC1] | (400 MHz, DMSO-d₆): δ 1.19-1.29 (m, 3H), 1.62-1.64 (m, 1H), 1.64- 1.88 (m, 10H), 1.99-2.01 (m, 2H), 3.27 (s, 3H), 3.45-3.51 (m, 2H), 3.92-3.97 (m, 2H), 4.40-4.43 (m, 2H), 7.66 (s, 1H). |
| 40 | Me— | 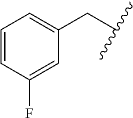 [SMILES: *Cc1cccc(F)c1] | 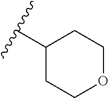 [SMILES: *C1CCOCC1] | (400 MHz, CDCl₃): δ 1.81-1.86 (m, 2H), 2.01-2.11 (m, 2H), 3.28-3.36 (m, 1H), 3.42 (s, 3H), 3.57 (td, J = 11.8, 2.1 Hz, 2H), 4.06-4.10 (m, 2H), 5.37 (s, 2H), 7.04-7.09 (m, 1H), 7.13-7.17 (m, 1H), 7.20-7.22 (m, 1H), 7.35-7.40 (m, 1H), 7.76 (s, 1H). |
| 41 | Me— | 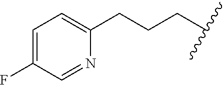 [SMILES: *CCCc1ccc(F)cn1] | 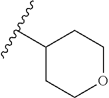 [SMILES: *C1CCOCC1] | (300 MHz, CDCl₃): δ 1.83-1.89 (m, 2H), 1.98-2.12 (m, 2H), 2.23-2.32 (m, 2H), 2.95 (t, J = 7.7 Hz, 2H), 3.24-3.34 (m, 4H), 3.53 (td, J = 11.4, 2.2 Hz, 2H) 4.01-4.07 (m, 2H), 4.41 (t, J = 6.2 Hz, 2H), 7.15 (dd, J = 8.8, 4.4 Hz, 1H), 7.32 (td, J = 8.4, 2.9 Hz, 1H), 7.73 (s, 1H), 8.37 (d, J = 2.9 Hz, 1H). |
| 42 | Me— | 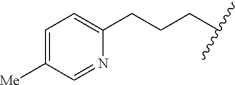 [SMILES: *CCCc1ccc(C)cn1] | 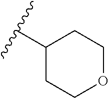 [SMILES: *C1CCOCC1] | (300 MHz, CDCl₃): δ 1.84-1.88 (m, 2H), 1.98-2.11 (m, 2H), 2.23-2.32 (m, 5H), 2.91 (t, J = 7.7 Hz, 2H), 3.24-3.32 (m, 4H), 3.49-3.57 (m, 2H), 4.02-4.06 (m, 2H), 4.41 (t, J = 6.2 Hz, 2H), 7.04 (d, J = 8.1 Hz, 1H), 7.39 (d, J = 8.1 Hz, 1H), 7.72- 7.73 (m, 1H), 8.33 (s, 1H). |
| 43 | Me— | 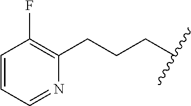 [SMILES: *CCCc1ncccc1F] | 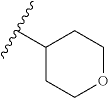 [SMILES: *C1CCOCC1] | (400 MHz, CDCl₃): δ 1.86-1.90 (m, 2H), 2.05-2.09 (m, 2H), 2.28-2.37 (m, 2H), 3.03-3.07 (m, 2H), 3.28- 3.32 (m, 1H), 3.35 (s, 3H), 3.55 (td, J = 11.7, 2.2 Hz, 2H), 4.05-4.08 (m, 2H), 4.45 (t, J = 6.3 Hz, 2H), 7.16- 7.20 (m, 1H), 7.32-7.37 (m, 1H), 7.75 (s, 1H), 8.34 (td, J = 3.0, 1.6 Hz, 1H). |
| 44 | Me— | 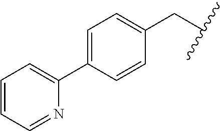 [SMILES: *Cc1ccc(-c2ccccn2)cc1] | 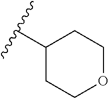 [SMILES: *C1CCOCC1] | (400 MHz, CDCl₃): δ 1.85-1.90 (m, 2H), 2.02-2.13 (m, 2H), 3.31-3.40 (m, 1H), 3.43 (s, 3H), 3.59 (td, J = 11.7, 2.2 Hz, 2H), 4.05-4.12 (m, 2H), 5.44 (s, 2H), 7.26 (m, 1H), 7.55 (d, Jr 8.5 Hz, 2H), 7.71-7.79 (m, 31), 8.03 (dt, J = 8.5, 1.9 Hz, 2H), 8.69-8.70 (m, 1H). |
| 45 | Me— | 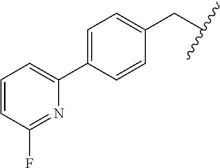 [SMILES: *Cc1ccc(-c2cccc(F)n2)cc1] | 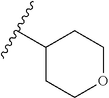 [SMILES: *C1CCOCC1] | (400 MHz, CDCl₃): δ 1.85-1.89 (m, 2H), 2.06-2.10 (m, 2H), 3.33-3.36 (m, 1H), 3.43 (s, 3H), 3.59 (td, J = 11.7, 2.0 Hz, 2H), 4.07-4.10 (m, 2H), 5.44 (s, 2H), 7.48 (td, J = 8.4, 2.9 Hz, 1H), 7.54 (d, J = 8.0 Hz, 2H), 7.72 (dd, J = 8.8, 4.4 Hz, 1H), 7.76 (s, 1H), 7.98 (d, J = 8.3 Hz, 2H), 8.54 (d, J = 2.9 Hz, 1H). |
| 46 | Me— | 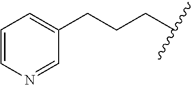 [SMILES: *CCCc1cccnc1] | 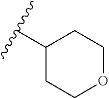 [SMILES: *C1CCOCC1] | (400 MHz, CDCl₃): δ 1.85-1.89 (m, 2H), 2.06-2.10 (m, 2H), 2.16-2.23 (m, 2H), 2.82 (t, J = 7.6 Hz, 2H), 3.28-3.31 (m, 1H), 3.36 (s, 3H), 3.55 (td, J = 11.6, 2.3 Hz, 2H), 4.05-4.08 (m, 2H), 4.40 (t, J = 6.2 Hz, 2H), 7.23-7.27 (m, 1H), 7.52- 7.54 (m, 1H), 7.76 (s, 1H), 8.47- 8.49 (m, 2H). |
| 47 | Me— | 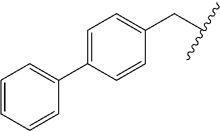 [SMILES: *Cc1ccc(-c2ccccc2)cc1] | 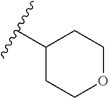 [SMILES: *C1CCOCC1] | (400 MHz, CDCl₃): δ 1.86-1.89 (m, 2H), 2.07-2.11 (m, 2H), 3.35-3.38 (m, 1H), 3.43 (s, 3H), 3.59 (td, J = 11.7, 2.2 Hz, 2H), 4.08-4.10 (m, 2H), 5.43 (s, 2H), 7.35-7.37 (m, 1H), 7.43-7.45 (m, 2H), 7.51-7.52 (m, 2H), 7.56-7.59 (m, 2H), 7.61- 7.64 (m, 2H), 7.76 (s, 1H). |
| 48 | Me— | 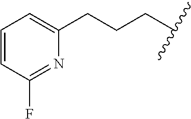 [SMILES: *CCCc1cccc(F)n1] | 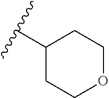 [SMILES: *C1CCOCC1] | (400 MHz, CDCl₃): δ 1.85-1.89 (m, 2H), 2.05-2.12 (m, 2H), 2.30 (dt, J = 15.0, 6.3 Hz, 2H), 2.96 (t, J = 7.6 Hz, 2H), 3.29-3.34 (m, 1H), 3.36 (d, J = 5.1 Hz, 3H), 3.55 (td, J = 11.7, 2.2 Hz, 2H), 4.05-4.08 (m, 2H), 4.43 (t, J = 6.2 Hz, 2H), 7.16 (dd, J = 8.7, 4.3 Hz, 1H), 7.33 (td, J = 8.4, 2.9 Hz, 1H), 7.76 (s, 1H), 8.39 (d, J = 2.9 Hz, 1H). |
| 49 | Me— | 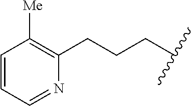 [SMILES: *CCCc1ncccc1C] | 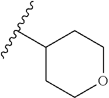 [SMILES: *C1CCOCC1] | (300 MHz, CDCl₃): δ 1.90 (d, J = 10.3 Hz, 2H), 2.09 (ddd, J = 24.9, 11.7, 4.4 Hz, 2H), 2.34 (dt, J = 17.9, 5.1 Hz, 5H), 2.98 (t, J = 7.3 Hz, 2H), 3.32 (ddd, J = 17.2, 9.9, 5.5 Hz, 4H), 3.58 (td, J = 11.4, 2.4 Hz, 2H), 4.09 (t, J = 5.9 Hz, 2H) 4.50 (t, J = 6.6 Hz, 2H), 7.08 (dd, J = 7.3, 5.1 Hz, 1H), 7.45 (d, J = 6.6 Hz, 1H), 7.77 (s, 1H), 8.38 (d, J = 4.4 Hz, 1H). |
| 50 | Me— | 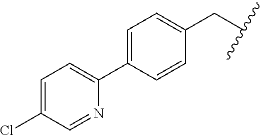 [SMILES: *Cc1ccc(-c2ccc(Cl)cn2)cc1] | 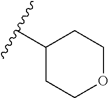 [SMILES: *C1CCOCC1] | (400 MHz, CDCl₃): δ 1.80-1.92 (m, 2H), 2.01-2.18 (m, 2H), 3.38-3.40 (m, 1H), 3.43 (s, 3H), 3.56-3.60 (m, 2H), 4.07-4.11 (m, 2H), 5.45 (s, 2H), 7.55 (d, J = 8.4 Hz, 2H), 7.71 (ddd, J = 18.8, 8.5, 1.6 Hz, 2H), 7.78 (s, 1H), 8.01 (d, J = 8.4 Hz, 2H), 8.63 (dd, J = 2.4, 0.7 Hz, 1H). |
| 51 | Me— | 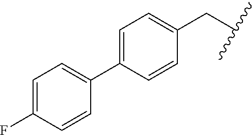 [SMILES: *Cc1ccc(-c2ccc(F)cc2)cc1] | 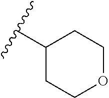 [SMILES: *C1CCOCC1] | (400 MHz, CDCl₃): δ 1.87-1.92 (m, 2H), 2.05-2.16 (m, 2H), 3.38 (tt, J = 11.7, 3.9 Hz, 1H), 3.45 (s, 3H), 3.58-3.64 (m, 2H), 4.09-4.13 (m, 2H), 5.44 (s, 2H), 7.12-7.18 (m, 2H), 7.51-7.61 (m, 6H), 7.78 (s, 1H). |
| 52 | Me— | 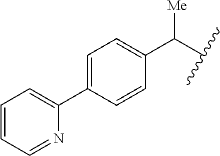 [SMILES: *C(C)c1ccc(-c2ccccn2)cc1] | 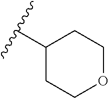 [SMILES: *C1CCOCC1] | (400 MHz, CDCl₃): δ 1.58-1.63 (m, 1H), 1.77 (d, J = 6.6 Hz, 3H), 1.88 (tt, J = 18.0, 5.2 Hz, 2H), 2.03-2.10 (m, 1H), 3.21-3.24 (m, 1H), 3.47 (s, 3H), 3.49-3.60 (m, 2H), 3.97-4.11 (m, 2H), 5.98 (q, J = 6.7 Hz, 1H), 7.24-7.26 (m, 1H), 7.50 (d, J = 8.3 Hz, 2H), 7.69-7.78 (m, 3H), 8.00 (d, J = 8.3 Hz, 2H), 8.68 (d, J = 4.6 Hz, 1H). |
| 53 | Me— | 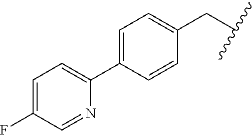 [SMILES: *Cc1ccc(-c2ccc(F)cn2)cc1] | 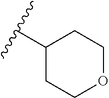 [SMILES: *C1CCOCC1] | (300 MHz, CDCl₃): δ 1.89 (t, J = 6.2 Hz, 2H), 2.03-2.13 (m, 2H), 3.33-3.41 (m, 1H), 3.45 (s, 3H), 3.61 (td, J = 11.6, 2.0 Hz, 2H), 4.11 (dt, J = 9.5, 2.2 Hz, 2H), 5.46 (s, 2H), 7.52 (dq, J = 18.5 4.9 Hz, 3H), 7.72-7.78 (m, 2H), 8.00 (d, J = 8.1 Hz, 2H), 8.56 (d, J = 2.9 Hz, 1H). |
| 54 | Et— | 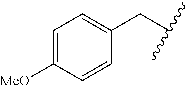 [SMILES: *Cc1ccc(OC)cc1] | 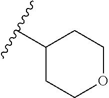 [SMILES: *C1CCOCC1] | (400 MHz, CDCl₃): δ 1.24 (t, J = 7.2 Hz, 3H), 1.90-1.93 (m, 2H), 2.09-2.13 (m, 2H); 138 (tt, J = 11.5, 3.9 Hz, 1H), 3.60-3.63 (m, 2H), 3.84 (s, 3H), 4.04 (q, J = 7.1 Hz, 2H), 4.12-4.13 (m, 2H), 5.35 (s, 2H), 6.94 (d, J = 8.8 Hz, 2H), 7.40 (d, J = 8.5 Hz, 2H), 7.76 (s, 1H). |
| 55 | Et— | 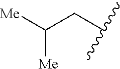 [SMILES: *CC(C)C] | 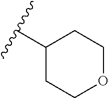 [SMILES: *C1CCOCC1] | (400 MHz, CDCl₃): δ 1.08 (d, J = 6.8 Hz, 6H), 1.28 (t, J = 7.1 Hz, 3H), 1.90-1.94 (m, 2H), 2.05-2.23 (m, 3H), 3.34-3.37 (m, 1H), 3.58- 3.61 (m, 2H), 4.06-4.14 (m, 6H), 7.77 (s, 1H). |
| 56 | Et— | 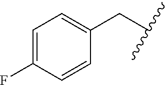 [SMILES: *Cc1ccc(F)cc1] | 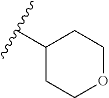 [SMILES: *C1CCOCC1] | (400 MHz, CDCl₃): δ 1.26 (t, J = 7.1 Hz, 3H), 1.87-1.95 (m, 2H), 2.05-2.16 (m, 2H), 3.35-3.37 (m, 1H), 3.60 (td, J = 11.6, 2.3 Hz, 2H), 4.06-4.10 (m, 4H), 5.38 (s, 2H), 7.11-7.13 (m, 2H), 7.44-7.47 (m, 2H), 7.77 (s, 1H). |
| 57 | Et— | 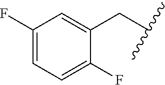 [SMILES: *Cc1cc(F)ccc1F] | 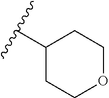 [SMILES: *C1CCOCC1] | (400 MHz, CDCl₃): δ 1.27 (t, J = 7.1 Hz, 3H), 1.85-1.89 (m, 2H), 2.03-2.13 (m, 2H), 3.31-3.40 (m, 1H), 3.57-3.65 (m, 2H), 4.03-4.14 (m, 4H), 5.45 (s, 2H), 7.05-7.20 (m, 3H), 7.77 (s, 1H). |
| 58 | Et— | 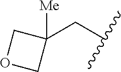 [SMILES: *CC1(C)COC1] | 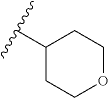 [SMILES: *C1CCOCC1] | (400 MHz, CDCl₃): δ 1.29 (t, J = 7.2 Hz, 3H), 1.48 (s, 3H), 1.90-1.94 (m, 2H), 2.06-2.16 (m, 2H), 3.33- 3.41 (m 1H), 3.60 (tt, J = 12.8, 2.8 Hz, 2H), 4.04-4.12 (m, 41), 4.48 (s, 2H), 4.54 (d, J = 6.1 Hz, 2H), 4.63 (d, J = 6.1 Hz, 2H), 7.79 (s, 1H). |
| 59 | Et— | 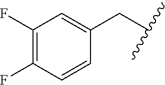 [SMILES: *Cc1ccc(F)c(F)c1] | 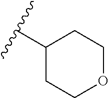 [SMILES: *C1CCOCC1] | (400 MHz, CDCl₃): δ 1.20 (t, J = 7.1 Hz, 3H), 1.77-1.82 (m, 2H), 1.97-2.09 (m, 2H), 3.23-3.31 (m, 1H), 3.52 (td, J = 11.6, 2.1 Hz, 2H), 3.94-4.07 (m, 4H), 5.29 (s, 2H), 7.08-7.25 (m, 3H), 7.70 (s, 1H). |
| 60 | Et— | 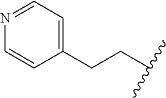 [SMILES: *CCc1ccncc1] | 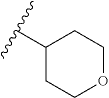 [SMILES: *C1CCOCC1] | (400 MHz, CDCl₃): δ 1.13 (t, J = 7.1 Hz, 3H), 1.90-1.94 (m, 2H), 2.11-2.17 (m, 2H), 3.19 (t, J = 6.3 Hz, 2H), 3.35-3.38 (m, 1H), 3.58- 3.61 (m, 2H), 3.95 (q, J = 7.1 Hz, 2H), 4.10-4.13 (m, 2H), 4.66 (t, J = 6.3 Hz, 2H), 7.25 (d, J = 5.1 Hz, 2H), 7.78 (s, 1H), 8.61 (d, J = 5.1 Hz, 2H). |
| 61 | Et— | 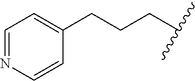 [SMILES: *CCCc1ccncc1] | 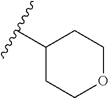 [SMILES: *C1CCOCC1] | (400 MHz, CDCl₃): δ 1.25 (t, J = 7.1 Hz, 3H), 1.82-1.89 (m, 2H), 2.01-2.11 (m, 2H), 2.15-2.22 (m, 2H), 2.78-2.82 (m, 2H), 3.28 (tt, J = 11.6, 3.9 Hz, 1H), 3.53 (td, J = 11.6, 2.1 Hz, 2H), 3.96-4.07 (m, 4H), 4.38 (t, J = 6.1 Hz, 2H), 7.12 (d, J = 6.1 Hz, 2H), 7.73 (s, 1H), 8.51 (d, J = 6.1 Hz, 2H). |
| 62 | Et— | 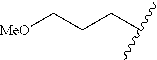 [SMILES: *CCCOC] | 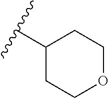 [SMILES: *C1CCOCC1] | (400 MHz, CDCl₃): δ 1.24 (t, J = 7.1 Hz, 3H), 1.86-1.90 (m, 2H), 2.02-2.12 (m, 4H), 3.28-3.37 (m, 4H), 3.51-3.58 (m, 4H), 3.98-4.08 (m, 4H), 4.45 (t, J = 6.1 Hz, 2H), 7.73 (s, 1H). |
| 63 | Et— | 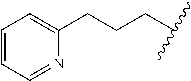 [SMILES: *CCCc1ccccn1] | 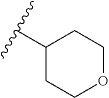 [SMILES: *C1CCOCC1] | (400 MHz, CDCl₃): δ 1.23 (t, J = 7.1 Hz, 3H), 1.84-1.88 (m, 2H), 1.99-2.10 (m, 2H), 2.27-2.34 (m, 2H), 2.95 (t, J = 7.6 Hz, 2H), 3.28 (tt, J = 11.5, 3.9 Hz, 1H), 3.53 (td, J = 11.6, 2.2 Hz, 2H), 3.94-4.05 (m, 4H), 4.43 (t, J = 6.2 Hz, 2H), 7.10- 7.16 (m, 2H), 7.59 (td, J = 7.7, 1.9 Hz, 1H), 7.72 (s, 1H), 8.51-8.53 (m, 1H). |
| 64 | Et— | 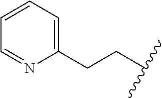 [SMILES: *CCc1ccccn1] | 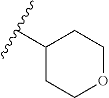 [SMILES: *C1CCOCC1] | (400 MHz, CDCl₃): δ 1.01 (t, J = 7.1 Hz, 3H), 1.88-1.92 (m, 2H), 2.02-2.13 (m, 2H), 3.28-3.37 (m, 3H), 3.56 (td, J = 11.7, 2.2 Hz, 2H), 3.87 (q, J = 7.1 Hz, 2H), 4.05-4.09 (m, 2H), 4.78 (t, J = 6.3 Hz, 2H), 7.16-7.23 (m, 2H), 7.62-7.66 (m, 1H), 7.72 (s, 1H), 8.55-8.57 (m, 1H). |
| 65 | Et— | 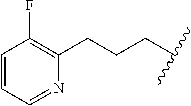 [SMILES: *CCCc1ncccc1F] | 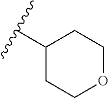 [SMILES: *C1CCOCC1] | (400 MHz, CDCl₃): δ 1.25 (t, J = 7.1 Hz, 3H), 1.86-1.90 (m, 2H), 2.05-2.09 (m, 2H), 2.32 (dt, J = 14.7, 6.5 Hz, 2H), 3.04 (td, J = 7.6, 2.0 Hz, 2H), 3.31 (tt, J = 11.5, 3.9 Hz, 1H), 3.55 (td, J = 11.6, 2.3 Hz, 2H), 4.00 (q, J = 7.1 Hz, 2H), 4.04- 4.08 (m, 2H), 4.45 (t, J = 6.3 Hz, 2H), 7.16-7.20 (m, 1H), 7.32-7.37 (m, 1H), 7.75 (s, 1H), 8.35 (td, J = 3.0, 1.5 Hz, 1H). |
| 66 | Et— | 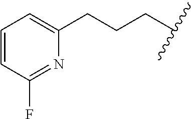 [SMILES: *CCCc1cccc(F)n1] | 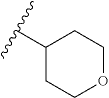 [SMILES: *C1CCOCC1] | (400 MHz, CDCl₃): δ 1.20-1.29 (m, 3H), 1.82-1.93 (m, 2H), 2.00-2.15 (m, 2H), 2.24-2.34 (m, 2H), 2.96 (t, J = 7.6 Hz, 2H), 3.30 (dt, J = 11.5, 3.9 Hz, 1H), 3.55 (td, J = 11.7, 2.2 Hz, 2H) 4.01 (q J = 7.1 Hz, 2H), 4.06 (ddd, J = 11.5, 4.1, 2.5 Hz, 2H), 4.40-4.46 (m, 2H), 7.15 (dd, J = 8.5, 4.4 Hz, 1H), 7.33 (td, J = 8.4, 3.0 Hz, 1H), 7.75 (s, 1H), 8.39 (d, J = 2.9 Hz, 1H). |
| 67 | Et— | 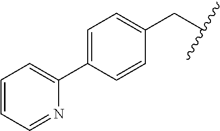 [SMILES: *Cc1ccc(-c2ccccn2)cc1] | 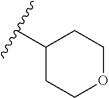 [SMILES: *C1CCOCC1] | (400 MHz, CDCl₃): δ 1.26 (t, J = 7.1 Hz, 3H), 1.85-1.89 (m, 2H), 2.06-2.10 (m, 2H), 3.35 (tt, J = 11.5, 3.9 Hz, 1H), 3.59 (td, J = 11.7, 2.2 Hz, 2H), 4.05-4.09 (m, 4H), 5.45 (s, 2H), 7.26-7.27 (m, 1H), 7.55 (d, J = 8.5 Hz, 2H), 7.73- 7.78 (m, 3H), 8.03-8.05 (m, 2H), 8.69-8.70 (m, 1H). |
| 68 | Et— | 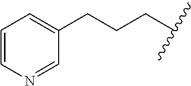 [SMILES: *CCCc1cccnc1] | 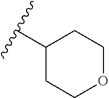 [SMILES: *C1CCOCC1] | (400 MHz, CDCl₃): δ 1.27 (t, J = 7.1 Hz, 3H), 1.85-1.89 (m, 2H), 2.02-2.13 (m, 2H), 2.19 (dt, J = 7.6, 3.2 Hz, 2H), 2.83 (t, J = 7.7 Hz, 2H), 3.30 (tt, J = 11.5, 3.9 Hz, 1H), 3.54 (td, J = 11.6, 2.3 Hz, 2H), 4.01 (q, J = 7.2 Hz, 2H), 4.05-4.08 (m, 2H), 4.41 (t, J = -6.2 Hz, 2H), 7.25- 7.28 (m, 1H), 7.54-7.56 (m, 1H). 7.76 (s, 1H), 8.48-8.50 (m, 2H). |
| 69 | Et— | 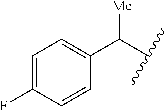 [SMILES: *C(C)c1ccc(F)cc1] | 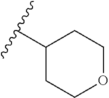 [SMILES: *C1CCOCC1] | (400 MHz, CDCl₃): δ 1.29 (t, J = 7.1 Hz, 3H), 1.62-1.69 (m, 1H), 1.73 (d, J = 6.5 Hz, 3H), 1.82-1.88 (m, 1H), 1.91-2.02 (m, 1H), 2.03- 2.14 (m, 1H), 3.23 (tt, J = 11.7, 3.9 Hz, 1H), 3.51-3.61 (m, 2H), 4.04- 4.14 (m, 4H), 5.94 (q, J = 6.5 Hz, 1H), 7.04-7.10 (m, 2H), 7.37-7.42 (m, 2H), 7.72 (s, 1H). |
| 70 | Et— | 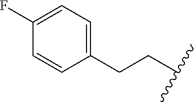 [SMILES: *CCc1ccc(F)cc1] | 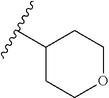 [SMILES: *C1CCOCC1] | (400 MHz, CDCl₃): δ 1.13 (t, J = 7.1 Hz, 3H), 1.89-1.92 (m, 2H), 2.06-2.17 (m, 2H), 3.13 (t, J = 6.4 Hz, 2H), 3.34 (tt, J = 11.5, 3.8 Hz, 1H), 3.55-3.61 (m, 2H), 3.96 (q, J = 7.1 Hz, 2H), 4.08-4.12 (m, 2H), 4.56 (t, J = 6.4 Hz, 2H), 7.01-7.07 (m, 2H), 7.22-7.26 (m, 2H), 7.76 (s, 1H). |
| 71 | Et— | 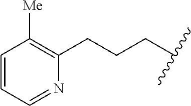 [SMILES: *CCCc1ncccc1C] | 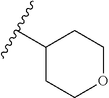 [SMILES: *C1CCOCC1] | (300 MHz, CDCl₃): δ 1.27 (t, J = 7.0 Hz 3H), 1.91 (d, J = 11.0 Hz, 2H), 2.10 (ddd, J = 24.6, 11.7, 3.7 Hz, 2H), 2.29-2.39 (m, 5H), 2.97 (t, J = 7.7 Hz, 2H), 3.30-3.37 (m, 1H), 3.58 (dd, J = 11.7, 9.5 Hz, 2H), 4.00-4.11 (m, 4H), 4.51 (t, J = 6.6 Hz, 2H), 7.09 (dd, J = 7.7, 4.8 Hz, 1H), 7.45 (d, J = 8.1 Hz, 1H), 7.77 (s, 1H), 8.39 (d, J = 4.4 Hz, 1H). |
| 72 | Et— | 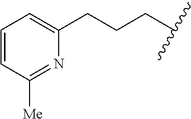 [SMILES: *CCCc1cccc(C)n1] | 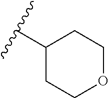 [SMILES: *C1CCOCC1] | (300 MHz, CDCl₃): δ 1.26 (t, J = 7.3 Hz 3H), 1.90 (t, J = 6.6 Hz, 2H), 2.09 (ddd, J = 24.9, 11.7, 4.4 Hz, 2H), 2.27-2.36 (m, 2H), 2.52 (d, J = 5.1 Hz, 3H), 2.94 (t, J = 7.3 Hz, 2H), 3.32 (tt, J = 11.4, 4.0 Hz, 1H), 3.53-3.61 (m, 2H), 4.04 (ddd, J = 23.8, 10.6, 4.4 Hz, 4H), 4.46 (t, J = 6.2 Hz, 2H), 6.99 (dd, J = 11.7, 7.3 Hz, 2H), 7.51 (t, J = 7.3 Hz, 1H), 7.76 (s, 1H). |
| 73 | Et— | 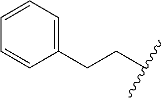 [SMILES: *CCc1ccccc1] | 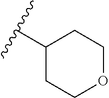 [SMILES: *C1CCOCC1] | (300 MHz, CDCl₃): δ 1.13 (t, J = 7.0 Hz, 3H), 1.91 (t, J = 6.6 Hz, 2H), 2.11 (tt, J = 16.1, 5.7 Hz, 2H), 3.16 (t, J = 6.611z, 2H), 3.35 (tt, J = 11.4, 3.9 Hz, 1H), 3.59 (td, J = 11.7, 2.2 Hz, 2H), 3.96 (q, J = 7.1 Hz, 2H), 4.10 (dt, J = 9.3, 2.2 Hz, 2H), 4.59 (t, J = 7.0 Hz, 2H), 7.28 (dd, J = 6.6, 2.9 Hz, 3H), 7.36 (dd, J = 10.6, 4.0 Hz, 2H), 7.77 (s, 1H). |
| 74 | Et— | 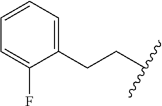 [SMILES: *CCc1ccccc1F] | 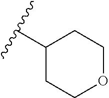 [SMILES: *C1CCOCC1] | (300 MHz, CDCl₃): δ 1.13 (t, J = 7.0 Hz, 3H), 1.91 (t, J = 6.6 Hz, 2H), 2.10 (ddd, J = 24.6, 12.1, 4.0 Hz, 2H), 3.21 (t, J = 6.6 Hz, 2H), 3.35 (tt, J = 11.7, 3.9 Hz, 1H), 3.60 (td, J = 11.4, 2.2 Hz, 2H), 3.96 (q, J = 6.8 Hz, 2H), 4.10 (t, J = 5.9 Hz, 2H), 4.60 (t, J = 6.2 Hz, 2H), 7.05- 7.15 (m, 2H), 7.25-7.30 (m, 2H), 7.76 (s, 1H). |
| 75 | Et— | 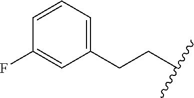 [SMILES: *CCc1cccc(F)c1] | 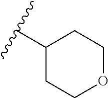 [SMILES: *C1CCOCC1] | (300 MHz, CDCl₃): δ 1.13 (t, J = 7.0 Hz, 3H), 1.91 (t, J = 6.2 Hz, 2H), 2.12 (cldd, J = 24.9, 11.7, 4.4 Hz, 2H), 3.16 (t, J = 6.2 Hz, 2H), 3.35 (tt, J = 11.7, 3.9 Hz, 1H), 3.59 (td, J = 11.7, 2.2 Hz, 2H), 3.96 (q, J = 7.1 Hz, 2H), 4.11 (dd, J = 8.4, 3.3 Hz, 2H), 4.59 (t, J = 6.2 Hz, 2H), 7.01 (dt, J = 21.0, 6.6 Hz, 3H), 7.32 (q, J = 7.6 Hz, 1H), 7.76 (s, 1H). |
| 76 | Et— | 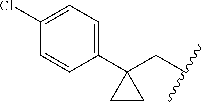 [SMILES: *CC1(c2ccc(Cl)cc2)CC1] | 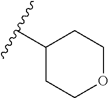 [SMILES: *C1CCOCC1] | (300 MHz, CDCl₃): δ 1.07 (s, 4H), 1.15 (t, J = 7.3 Hz, 3H), 1.87 (d, J = 12.5 Hz, 2H), 2.08 (ddd, J = 24.6, 12.1, 4.0 Hz, 2H), 3.29 (dq, J = 15.2 3.9 Hz, 1H), 3.58 (td, J = 11.7, 2.2 Hz, 2H), 3.98 (q, J = 7.1 Hz, 2H), 4.10 (d, J = 11.7 Hz, 2H), 4.36 (s, 2H), 7.31(t, J = 2.6 Hz, 4H), 7.75 (s, 1H). |
| 77 | Et— | 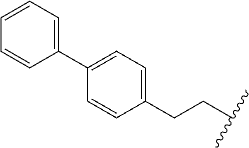 [SMILES: *CCc1ccc(-c2ccccc2)cc1] | 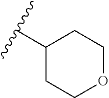 [SMILES: *C1CCOCC1] | (300 MHz, CDCl₃): δ 1.16 (t, J = 7.3 Hz, 3H), 1.92 (d, J = 11.0 Hz, 2H), 2.12 (ddd, J = 24.8, 11.9, 3.9 Hz, 2H), 3.21 (t, J = 6.6 Hz, 2H), 3.31-3.40 (m, 1H), 3.60 (td, J = 11.6, 2.0 Hz, 2H) 3.99 (q, J = 7.1 Hz, 2H), 4.11 (d, J = 10.3 Hz, 2H), 4.63 (t, J = 6.6 Hz, 2H), 7.36 (dd, J = 7.7, 2.6 Hz, 3H), 7.45 (t, J = 7.3 Hz, 2H), 7.59 (d, J = 8.1 Hz, 4H), 7.76 (s, 1H). |
| 78 | Et— | 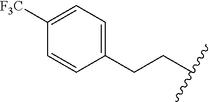 [SMILES: *CCc1ccc(C(F)(F)F)cc1] | 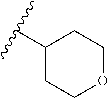 [SMILES: *C1CCOCC1] | (300 MHz, CDCl₃): δ 1.12 (t, J = 7.0 Hz, 3H), 1.91 (t, J = 6.6 Hz, 2H), 2.12 (ddd, J = 25.1, 11.6, 4.2 Hz, 2H), 3.23 (t, J = 6.2 Hz, 2H), 3.34 (tt, J = 11.4, 3.9 Hz, 1H), 3.58 (td, J = 11.4, 2.2 Hz, 2H), 3.95 (q, J = 7.1 Hz, 2H), 4.11 (dd, J = 8.4, 3.3 Hz, 2H), 4.62 (t, J = 6.6 Hz, 2H), 7.41 (d, J = 8.1 Hz, 2H), 7.62 (d, J = 8.8 Hz, 2H), 7.76 (s, 1H). |
| 79 | Et— | 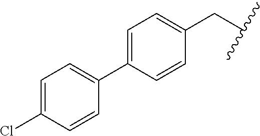 [SMILES: *Cc1ccc(-c2ccc(Cl)cc2)cc1] | 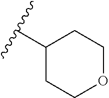 [SMILES: *C1CCOCC1] | (400 MHz, CDCl₃): δ 1.29 (t, J = 7.1 Hz, 3H), 1.86-1.91 (m, 2H), 2.05-2.16 (m, 2H), 3.36 (tt, J = 11.7, 3.8 Hz, 1H), 3.60 (td, J = 11.7, 2.2 Hz, 2H), 4.06-4.13 (m, 4H), 5.45 (s, 2H), 7.41-7.44 (m, 2H), 7.51-7.54 (m, 4H), 7.60-7.62 (m, 2H), 7.77 (s, 1H). |
| 80 | Et— | 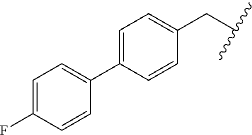 [SMILES: *Cc1ccc(-c2ccc(F)cc2)cc1] | 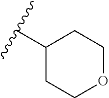 [SMILES: *C1CCOCC1] | (400 MHz, CDCl₃): δ 1.29 (t, J = 7.1 Hz, 3H), 1.86-1.91 (m, 2H), 2.05-2.16 (m, 2H), 3.37 (tt, J = 11.6. 3.9 Hz, 1H), 3.61 (td, J = 11.6, 2.1 Hz, 2H), 4.06-4.13 (m, 4H), 5.45 (s, 2H), 7.12-7.18 (m, 2H), 7.51-7.60 (m, 6H), 7.77 (s, 1H). |
| 81 | Et— | 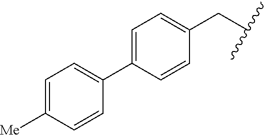 [SMILES: *Cc1ccc(-c2ccc(C)cc2)cc1] | 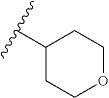 [SMILES: *C1CCOCC1] | (400 MHz, CDCl₃): δ 1.29 (t, J = 7.1 Hz, 3H), 1.86-1.92 (m, 2H), 2.05-2.15 (m, 2H), 2.40 (s, 3H), 3.37 (tt, J = 11.7, 4.0 Hz, 1H), 3.61 (td, J = 11.7, 2.1 Hz, 2H), 4.06-4.13 (m, 4H), 5.45 (s, 2H), 7.28 (br s, 2H), 7.48-7.52 (m, 4H), 7.62-7.64 (m, 2H), 7.77 (s, 1H). |
| 82 | Et— | 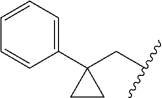 [SMILES: *CC1(c2ccccc2)CC1] | 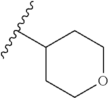 [SMILES: *C1CCOCC1] | (400 MHz, CDCl₃): δ 1.04-1.08 (m, 4H), 1.12 (t, J = 7.2 Hz, 3H), 1.83- 1.87 (m, 2H), 2.10 (ddd, J = 25.1. 11.6, 4.0 Hz, 2H), 3.34 (t, J = 11.2 Hz, 1H), 3.56 (td, J = 11.7, 2.0 Hz, 2H), 3.96 (q, J = 7.1 Hz, 2H), 4.06- 4.09 (m, 2H), 4.37 (s, 2H), 7.21- 7.25 (m, 1H), 7.31 (t, J = 7.4 Hz, 2H), 7.37-7.38 (m, 2H), 7.75 (s, 1H). |
| 83 | Et— | 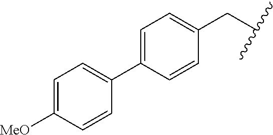 [SMILES: *Cc1ccc(-c2ccc(OC)cc2)cc1] | 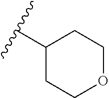 [SMILES: *C1CCOCC1] | (400 MHz, CDCl₃): δ 1.28 (t, J = 7.1 Hz, 3H), 1.87-1.91 (m, 2H). 2.05-2.15 (m, 2H), 3.37 (tt, J = 11.6, 3.9 Hz, 1H), 3.61 (td, J = 11.6, 2.0 Hz, 2H), 3.86 (s, 3H), 4.04-4.13 (m, 4H), 5.44 (s, 2H), 6.97-7.01 (m, 2H), 7.49-7.55 (m, 4H), 7.58-7.61 (m, 2H), 7.77 (s, 1H). |
| 84 | Et— | 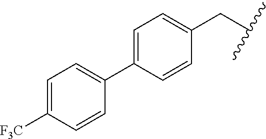 [SMILES: *Cc1ccc(-c2ccc(C(F)(F)F)cc2)cc1] | 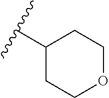 [SMILES: *C1CCOCC1] | (400 MHz, CDCl₃): δ 1.30 (t, J = 7.1 Hz, 3H), 1.87-1.90 (m, 2H), 2.06-2.16 (m, 2H), 3.37 (tt, J = 11.6, 3.8 Hz, 1H), 3.60 (td, J = 11.6, 2.0 Hz, 2H), 4.07-4.13 (m, 4H), 5.47 (s, 2H), 7.56 (d, J = 7.8 Hz, 2H), 7.65-7.67 (m, 2H), 7.68- 7.73 (m, 4H), 7.78 (s, 1H). |
| 85 | Et— | 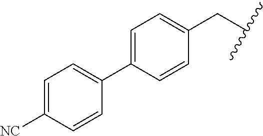 [SMILES: *Cc1ccc(-c2ccc(C#N)cc2)cc1] | 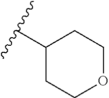 [SMILES: *C1CCOCC1] | (400 MHz, CDCl₃): δ 1.29 (t, J = 7.1 Hz, 3H), 1.86-1.90 (m, 2H), 2.05-2.16 (m, 2H), 3.36 (tt, J = 11.7, 3.7 Hz, 1H), 3.60 (td, J = 11.7, 1.7 Hz, 2H), 4.06-4.13 (m, 4H), 5.47 (s, 2H), 7.57 (d, J = 8.3 Hz, 2H), 7.65 (d, J = 8.3 Hz, 2H), 7.69 (d, J = 8.3 Hz, 2H), 7.75 (d, J = 8.3 Hz, 2H), 7.77 (s, 1H). |
| 86 | Et— | 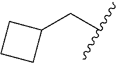 [SMILES: *CC1CCC1] | 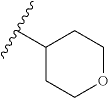 [SMILES: *C1CCOCC1] | (400 MHz, CDCl₃): δ 1.25-1.29 (m, 3H), 1.90-2.19 (m, 10H), 2.83-2.85 (m, 1H), 3.35-3.37 (m, 1H), 3.57- 3.63 (m, 2H), 4.02-4.12 (m, 4H), 4.34 (d, J = 6.4 Hz, 2H), 7.77 (s, 1H). |
| 87 | Et— | 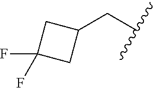 [SMILES: *CC1CC(F)(F)C1] | 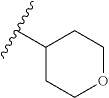 [SMILES: *C1CCOCC1] | (400 MHz, CDCl₃): δ 1.27 (t, J = 7.2 Hz, 3H), 1.88-1.92 (m, 2H), 2.05-2.16 (m, 2H), 2.48-2.88 (m, 5H), 3.30-3.37 (m, 1H), 3.56-3.62 (m, 2H), 4.02-4.12 (m, 4H), 4.45 (d, J = 6.0 Hz, 2H), 7.79 (s, 1H). |
| 88 | Et— | 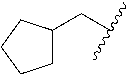 [SMILES: *CC1CCCC1] | 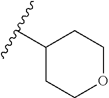 [SMILES: *C1CCOCC1] | (400 MHz, CDCl₃): δ 1.27 (t, J = 7.2 Hz, 3H), 1.37-1.43 (m, 2H), 1.64-1.70 (m, 4H), 1.85-1.94 (m, 4H), 2.05-2.16 (m, 2H), 2.39-2.46 (m, 1H), 3.31-3.39 (m, 1H), 3.56- 3.63 (m, 2H), 4.02-4.11 (m, 4H), 4.26 (d, J = 7.2 Hz, 2H), 7.77 (s, 1H). |
| 89 | Et— | 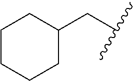 [SMILES: *CC1CCCCC1] | 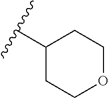 [SMILES: *C1CCOCC1] | (400 MHz, CDCl₃): δ 1.07-1.17 (m, 2H), 1.26-1.34 (m, 6H), 1.73-1.94 (m, 8H), 2.05-2.16 (m, 2H), 3.31- 3.38 (m, 1H), 3.56-3.62 (m, 2H), 4.02-4.11 (m, 4H), 4.17 (d, J = 5.6 Hz, 2H), 7.77 (s, 1H). |
| 90 | Et— | 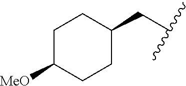 [SMILES: *C[C@H]1CC[C@@H](OC)CC1] | 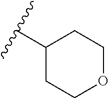 [SMILES: *C1CCOCC1] | (400 MHz, CDCl₃): δ 1.27 (t, J = 7.1 Hz, 3H), 1.43-1.66 (m, 6H), 1.86-2.02 (m, 5H), 2.04-2.16 (m, 2H), 3.29-3.39 (m, 4H), 3.47-3.52 (m, 1H), 3.55-3.64 (m, 2H), 4.01- 4.13 (m, 4H), 4.20 (d, J = 6.3 Hz, 2H), 7.77 (s, 1H). |
| 91 | Et— | 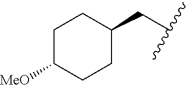 [SMILES: *C[C@H]1CC[C@H](OC)CC1] | 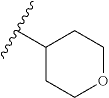 [SMILES: *C1CCOCC1] | (400 MHz, CDCl₃): δ 1.11-1.35 (m, 7H), 1.80-1.99 (m, 5H), 2.04-2.24 (m, 4H), 3.08-3.20 (m, 1H), 3.29- 3.41 (m, 1H), 3.38 (s, 3H), 3.54- 3.65 (m, 2H), 3.99-4.15 (m, 4H), 4.19 (d J = 6.1 Hz, 2H), 7.77 (s, 1H). |
| 92 | Et— | 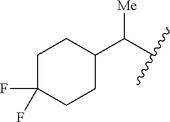 [SMILES: *C(C)C1CCC(F)(F)CC1] | 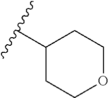 [SMILES: *C1CCOCC1] | (400 MHz, CDCl₃): δ 1.24 (t, J = 7.1 Hz, 1H), 1.41-1.95 (m, 12H), 2.02-2.18 (m, 4H), 3.25-3.33 (m, 1H), 3.53-3.59 (m, 2H), 3.92-4.09 (m, 4H), 5.00-5.06 (m, 1H), 7.75 (s, 1H). |
| 93 | Et— | 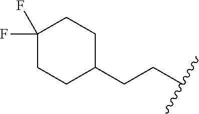 [SMILES: *CCC1CCC(F)(F)CC1] | 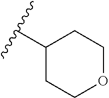 [SMILES: *C1CCOCC1] | (400 MHz, DMSO-d₆): δ 1.14-1.16 (m, 4H), 1.23-1.26 (m, 3H), 1.32- 1.33 (m, 1H), 1.75-1.83 (m, 9H), 1.84-1.88 (m, 2H), 3.47-3.49 (m, 2H), 3.90-3.96 (m, 4H), 3.42-4.45 (m, 2H), 7.66 (s, 1H). |
| 94 | ⁿPr— | 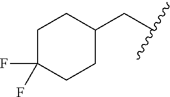 [SMILES: *CC1CCC(F)(F)CC1] | 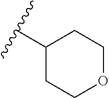 [SMILES: *C1CCOCC1] | (400 MHz, CDCl₃): δ 0.98 (t, J = 7.4 Hz, 3H), 1.52-1.56 (m, 2H), 1.69 (q, J = 7.4 Hz, 2H), 1.73-1.85 (m, 2H), 1.89-1.97 (m, 5H), 2.01- 2.24 (m, 4H), 3.30-3.38 (m, 1H), 3.56-3.62 (m, 2H), 3.92-3.96 (m, 2H), 4.08-4.12 (m, 2H), 4.25 (d, J = 6.0 Hz, 2H), 7.78 (s, 1H). |
| 95 | 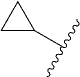 [SMILES: *C1CC1] | 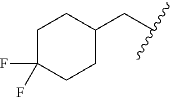 [SMILES: *CC1CCC(F)(F)CC1] | 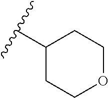 [SMILES: *C1CCOCC1] | (400 MHz, CDCl₃): δ 0.83-0.91 (m, 2H), 1.15-1.23 (m, 2H), 1.50-1.67 (m, 2H), 1.69-2.04 (m, 7H), 2.06- 2.27 (m, 4H), 2.66-2.74 (m, 1H), 3.33-3.44 (m, 1H), 3.57 (td, J = 11.7, 2.2 Hz, 2H), 4.05-4.12 (m, 2H), 4.24 (d, J = 5.4 Hz, 2H), 7.78 (s, 1H). |
| 96 | 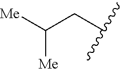 [SMILES: *CC(C)C] | 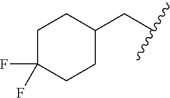 [SMILES: *CC1CCC(F)(F)CC1] | 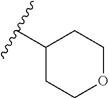 [SMILES: *C1CCOCC1] | (400 MHz, CDCl₃): δ 0.96 (d, J = 6.8 Hz, 6H), 1.51-1.57 (m, 2H), 1.73-1.86 (m, 2H), 1.92 (d, J = 13.2 Hz, 6H), 2.06-2.15 (m, 2H), 2.17- 2.23 (m, 2H), 3.33-3.38 (m, 1H), 3.56-3.63 (m, 2H), 3.81 (d, J = 6.0 Hz, 2H), 4.10-4.13 (m, 2H), 4.25 (d, J = 6.0 Hz, 2H), 7.79 (s, 1H). |
| 97 | 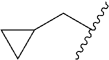 [SMILES: *CC1CC1] | 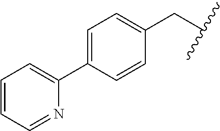 [SMILES: *Cc1ccc(-c2ccccn2)cc1] | 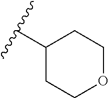 [SMILES: *C1CCOCC1] | (300 MHz, CDCl₃): δ 0.40 (m, 2H), 0.48-0.54 (m, 2H), 1.16-1.27 (m, 1H), 1.90 (d, J = 11.0 Hz, 2H), 2.11 (ddd, J = 24.6, 12.1, 3.7 Hz, 2H), 3.38 (tt, J = 11.4, 3.9 Hz, 1H), 3.61 (td, J = 11.4. 2.2 Hz, 2H), 3.90 (d, J = 7.3 Hz, 2H), 4.10 (dd, J = 13.6, 4.0 Hz, 2H), 5.48 (s, 2H), 7.25-7.29 (m, 2H), 7.57 (d, J = 8.1 Hz, 2H), 7.78 (ddd, J = 11.9, 7.5 3.5 Hz, 2H), 8.06 (d, J = 8.1 Hz 2H), 8.71 (dd, J = 3.7, 2.2 Hz, 1H). |
| 98 | 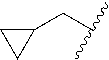 [SMILES: *CC1CC1] | 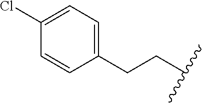 [SMILES: *CCc1ccc(Cl)cc1] | 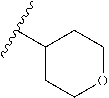 [SMILES: *C1CCOCC1] | (300 MHz, CDCl₃): δ 0.32 (q, J = 5.1 Hz, 2H), 0.44 (dd, J = 13.6, 4.8 Hz, 2H), 0.88-1.00 (m, 1H), 1.91 (d, J = 11.0 Hz, 2H), 2.13 (ddd, J = 24.9, 12.1, 3.7 Hz, 2H), 3.14 (t, J = 6.6 Hz, 2H), 3.35 (tt, J = 11.4, 3.9 Hz, 1H), 3.58 (td, J = 11.7, 2.2 Hz, 2H), 3.77 (d, J = 7.3 Hz, 2H), 4.10 (t, J = 7.3 Hz, 2H), 4.58 (t, J = 6.6 Hz, 2H), 7.22 (d, J = 8.8 Hz, 2H), 7.33 (t, J = 4.4 Hz, 2H), 7.76 (s, 1H). |
| 99 | 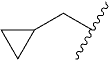 [SMILES: *CC1CC1] | 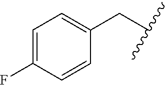 [SMILES: *Cc1ccc(F)cc1] | 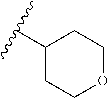 [SMILES: *C1CCOCC1] | (300 MHz, CDCl₃): δ 0.35-0.53 (m, 4H), 1.14-1.19 (m, 1H), 1.90 (t, J = 6.6 Hz, 2H), 2.12 (tt, J = 16.1, 5.9 Hz, 2H), 3.36 (td, J = 8.6, 4.9 Hz, 1H), 3.60 (td, J = 11.6, 2.4 Hz, 2H), 3.87 (d, J = 6.6 Hz, 2H), 4.11 (dq, J = 11.6, 3.2 Hz, 2H), 5.38 (s, 2H), 7.12 (q, J = 5.6 Hz, 2H), 7.45 (td, J = 5.9, 2.7 Hz, 2H), 7.78 (s, 1H). |
| 100 | 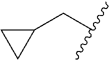 [SMILES: *CC1CC1] | 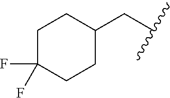 [SMILES: *CC1CCC(F)(F)CC1] | 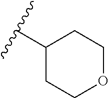 [SMILES: *C1CCOCC1] | (400 MHz, CDCl₃): δ 0.42-0.46 (m, 2H), 0.51-0.57 (m, 2H), 1.15- 1.20 (m, 1H), 1.52-1.59 (m, 2H), 1.73-1.88 (m, 2H), 1.89-1.97 (m, 5H), 2.08-2.16 (m, 2H), 2.18-2.23 (m, 2H), 3.35-3.38 (m, 1H), 3.57- 3.63 (m, 2H), 3.88 (d, J = 7.2 Hz, 2H), 4.10-4.13 (m, 2H), 4.28 (d, J = 6.0 Hz, 2H), 7.80 (s, 1H). |
| 101 | 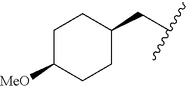 [SMILES: *C[C@H]1CC[C@@H](OC)CC1] | Me— | 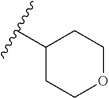 [SMILES: *C1CCOCC1] | (400 MHz, CDCl₃): δ 1.34-1.46 (m, 6H), 1.75-1.86 (m, 1H), 1.86-1.98 (m, 4H), 2.04-2.20 (m, 2H), 3.31 (s, 3H), 3.32-3.40 (m, 1H), 3.40-3.44 (m, 1H), 3.54-3.63 (m, 2H), 3.85 (d, J = 7.3 Hz, 2H), 4.04 (s, 3H), 4.07-4.14 (m, 2H), 7.77 (s, 1H). |
| 102 | 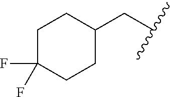 [SMILES: *CC1CCC(F)(F)CC1] | Et— | 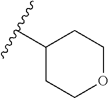 [SMILES: *C1CCOCC1] | (400 MHz, CDCl₃): δ 1.35-1.52 (m, 5H), 1.55-1.80 (m, 4H), 1.81-1.96 (m, 3H), 2.04-2.18 (m, 4H), 3.34 (tt, J = 11.6, 3.9 Hz, 1H), 3.58 (td, J = 11.6, 2.3 Hz, 2H), 3.89 (d, J = 7.1 Hz, 2H), 4.06-4.13 (m, 2H), 4.46 (q, J = 7.1 Hz, 2H), 7.78 (s, 1H). |
| 103 | 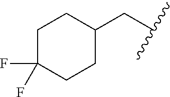 [SMILES: *CC1CCC(F)(F)CC1] | 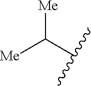 [SMILES: *C(C)C] | 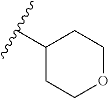 [SMILES: *C1CCOCC1] | (400 MHz, CDCl₃): δ 1.35-1.45 (m, 2H), 1.46 (d, J = 6.1 Hz, 6H), 1.58- 1.70 (m, 1H), 1.70-1.79 (m, 3H), 1.80-1.96 (m, 3H), 2.04-2.18 (m, 4H), 3.27-3.39 (m, 1H), 3.52-3.63 (m, 2H), 3.88 (d, J = 7.1 Hz, 2H), 4.06-4.14 (m, 2H), 5.19-5.30 (m, 1H), 7.78 (s, 1H). |
| 104 | 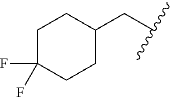 [SMILES: *CC1CCC(F)(F)CC1] | 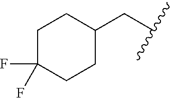 [SMILES: *CC1CCC(F)(F)CC1] | 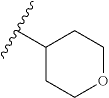 [SMILES: *C1CCOCC1] | (400 MHz, CDCl₃): δ 1.40-1.90 (m, 16H), 2.04-2.19 (m, 6H), 3.28-3.36 (m, 1H), 3.53-3.59 (m, 2H), 3.86- 3.88 (m, 2H), 4.06-4.10 (m, 2H), 4.24-4.26 (m, 2H), 7.77 (s, 1H). |
| 105 | 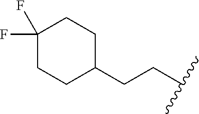 [SMILES: *CCC1CCC(F)(F)CC1] | Me— | 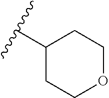 [SMILES: *C1CCOCC1] | (400 MHz, DMSO-d₆): δ 1.15-1.22 (m, 2H), 1.41-1.54 (m, 3H), 1.79- 2.02 (m, 11H), 2.36-2.46 (m, 2H), 3.89-3.99 (m, 4H), 4.01 (s, 3H), 7.67 (s, 1H). |
| 106 | 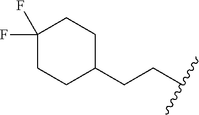 [SMILES: *CCC1CCC(F)(F)CC1] | Et— | 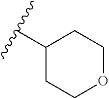 [SMILES: *C1CCOCC1] | (400 MHz, DMSO-d₆): δ 1.16-1.19 (m, 3H), 1.38-1.41 (m, 4H), 1.50- 1.53 (m, 2H), 1.83-1.86 (m, 8H), 1.97-2.00 (m, 2H), 3.45-3.52 (m, 2H), 3.88-3.97 (m, 4H), 4.40-4.45 (m, 2H), 7.67 (s, 1H). |
| 107 | 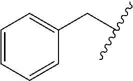 [SMILES: *Cc1ccccc1] | Me— | 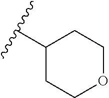 [SMILES: *C1CCOCC1] | (300 MHz, CDCl₃): δ 1.91 (t, J = 6.6 Hz 2H), 2.11 (tt, J = 16.1, 5.9 Hz, 2H), 3.35 (tt, J = 11.4, 3.9 Hz, 1H), 3.57 (td, J = 11.7, 2.2 Hz, 2H), 4.07 (dt, J = 11.2, 4.8 Hz, 5H), 5.15 (s, 2H), 7.34 (dt, J = 12.8, 4.6 Hz, 5H), 7.81 (s, 1H). |
| 108 | 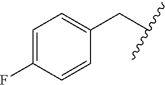 [SMILES: *Cc1ccc(F)cc1] | Me— | 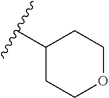 [SMILES: *C1CCOCC1] | (300 MHz, CDCl₃): δ 1.86-1.91 (m, 2H), 2.01-2.15 (m, 2H), 3.28-3.38 (m, 1H), 3.55 (td, J = 11.4, 2.2 Hz, 2H), 4.03-4.10 (m, 5H), 5.09 (s, 2H), 6.95-7.02 (m, 2H), 7.33-7.38 (m, 2H), 7.79 (s, 1H). |
| 109 | 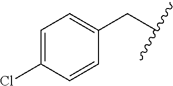 [SMILES: *Cc1ccc(Cl)cc1] | Me— | 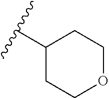 [SMILES: *C1CCOCC1] | (300 MHz, CDCl₃): δ 1.86-1.91 (m, 2H), 2.01-2.15 (m, 2H), 3.28-3.38 (m, 1H), 3.55 (td, J = 11.7, 2.2 Hz, 2H), 4.02-4.09 (m, 5H), 5.09 (s, 2H), 7.25-7.32 (m, 4H), 7.79 (s, 1H). |
| 110 | 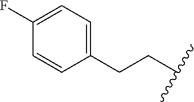 [SMILES: *CCc1ccc(F)cc1] | Me— | 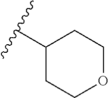 [SMILES: *C1CCOCC1] | (300 MHz, CDCl₃): δ 1.87-1.92 (m, 2H), 2.02-2.16 (m, 2H), 2.87-2.92 (m, 2H), 3.29-3.38 (m, 1H), 3.56 (td, J = 11.4, 2.2 Hz, 2H), 3.93 (s, 3H), 4.06-4.16 (m, 4H), 6.94-7.00 (m, 2H), 7.14-7.18 (m, 2H), 7.77 (s, 1H). |
| 111 | Me— | 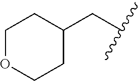 [SMILES: *CC1CCOCC1] | 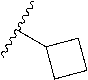 [SMILES: *C1CCC1] | (400 MHz, CDCl₃): δ 1.47-1.58 (m, 2H), 1.72-1.75 (m, 2H), 1.99-2.18 (m, 3H), 2.36-2.44 (m, 2H), 2.50- 2.60 (m, 2H), 3.40 (s, 3H), 3.43- 3.50 (m, 2H), 3.90-3.98 (m, 1H), 4.04-4.08 (m, 2H), 4.24 (d, J = 6.4 Hz, 2H), 7.80 (s, 1H). |
| 112 | Me— | 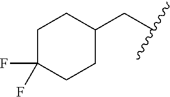 [SMILES: *CC1CCC(F)(F)CC1] | 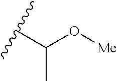 [SMILES: *C(C)OC] | (400 MHz, CDCl₃): δ 1.45-1.56 (m, 2H), 1.67 (d, J = 6.8 Hz, 3H), 1.72- 1.87 (m, 2H), 1.93-1.96 (m, 3H), 2.17-2.21 (m, 2H), 3.32 (s, 3H), 3.43 (s, 3H), 4.28 (d, J = 6.4 Hz, 2H), 4.91 (q, J = 5.1 Hz, 1H), 7.85 (s, 1H). |
| 113 | Et— | 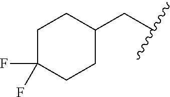 [SMILES: *CC1CCC(F)(F)CC1] | 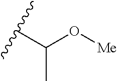 [SMILES: *C(C)OC] | (400 MHz, CDCl₃): δ 1.34-1.47 (m, 2H), 1.58-1.65 (m, 6H), 1.72-1.81 (m, 2H), 1.94-1.97 (m, 3H), 2.09- 2.13 (m, 2H), 3.38 (s, 3H), 4.24- 4.26 (m, 2H), 4.43-4.49 (m, 2H), 5.48 (q, J = 5.4 Hz, 1H), 7.69 (s, 1H). |
| 114 | 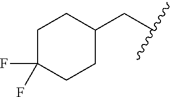 [SMILES: *CC1CCC(F)(F)CC1] | Me— | 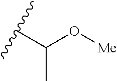 [SMILES: *C(C)OC] | (400 MHz, CDCl₃): δ 1.41-1.48 (m, 2H), 1.64-1.66 (m, 1H), 1.69 (d, J = 6.4 Hz, 3H), 1.73-1.76 (m, 3H), 1.86-1.89 (m, 1H), 2.09-2.16 2H), 3.36 (s, 3H), 3.91 (d, J = 7.2 Hz, 2H), 4.09 (s, 3H), 4.92 (q, J = 5.0 Hz, 1H), 7.85 (s, 1H). |

### Example 115

### 3-[(4,4-Difluorocyclohexyl)methyl]-2-methoxy-7-(tetrahydro-2H-pyran-4-yl)imidazo[5,1-f][1,2,4]triazin-4(3H)-one

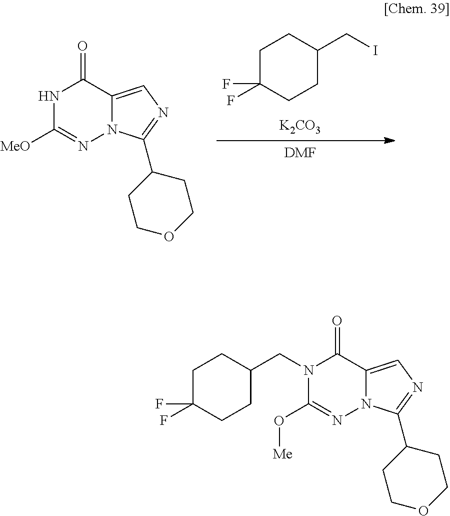

2-Methoxy-7-(tetrahydro-2H-pyran-4-yl)imidazo[5,1-f][1,2,4]triazin-4(3H)-one (50.0 mg, 0.2 mmol) and potassium carbonate (44 mg, 0.32 mmol) were dissolved in DMF (50 mL). 1,1-difluoro-4-(iodomethyl)cyclohexane (78 mg, 0.3 mmol) was added dropwise and stirred at room temperature for overnight. Upon completion, the reaction mixture was partitioned between ethyl acetate and water. The aqueous layer was extracted with EtOAc, and the combined organic phases were dried with sodium sulfate and concentrated to dryness. The residue was purified by amino silica gel column chromatography (hexane/EtOAc) to give the titled compound (17.3 mg, yield 23%) as a white solid.

LC-MS (m/z)=383 [M+H]⁺. ¹H-NMR (300 MHz, CDCl₃): δ 1.33-1.46 (m, 2H), 1.59-1.93 (m, 7H), 2.03-2.17 (m, 4H), 3.30-3.40 (m, 1H), 3.56 (td, J=11.6, 2.0 Hz, 2H), 3.87 (d, J=7.3 Hz, 2H), 4.04-4.11 (m, 5H), 7.76 (s, 1H).

### Example 116

### 5-Fluoro-2-[(4-fluorobenzyl)oxy]-3-methyl-7-(tetrahydro-2H-pyran-4-yl)imidazo[5,1-f][1,2,4]triazin-4(3H)-one

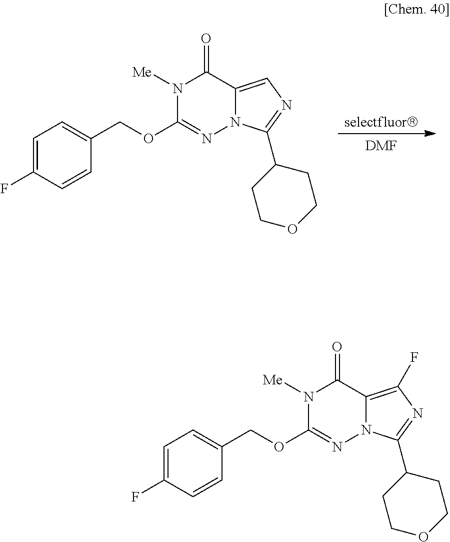

To a solution of 2-[(4-fluorobenzyl)oxy]-3-methyl-7-(tetrahydro-2H-pyran-4-yl)imidazo[5,1-f][1,2,4]triazin-4(3H)-one (0.1 g, 0.28 mmol) in DMF (1.0 mL) was added Selectfluor (Registered Trademark) (0.165 g, 0.419 mmol). The mixture was heated for 80 minutes at 70° C. under nitrogen atmosphere. Upon completion, the reaction mixture was cooled and partitioned between ethyl acetate and brine. The aqueous layer was extracted with EtOAc, and the combined organic phases were dried with sodium sulfate and concentrated to dryness. The residue was purified by amino silica gel column chromatography (hexane/EtOAc) to give the titled compound (28.0 mg, yield 13%) as a white solid.

LC-MS (m/z)=377 [M+H]⁺. ¹H-NMR (300 MHz, CDCl₃): δ 1.79-1.83 (m, 2H), 1.94-2.08 (m, 2H), 3.24-3.39 (m, 4H), 3.54 (td, J=11.6, 2.0 Hz, 2H), 4.04-4.08 (m, 2H), 5.32 (s, 2H), 7.06-7.11 (m, 2H), 7.39-7.44 (m, 2H).

### Example 117

### 5-Chloro-2-[(4-fluorobenzyl)oxy]-3-methyl-7-(tetrahydro-2H-pyran-4-yl)imidazo[5,1-f][1,2,4]triazin-4(3H)-one

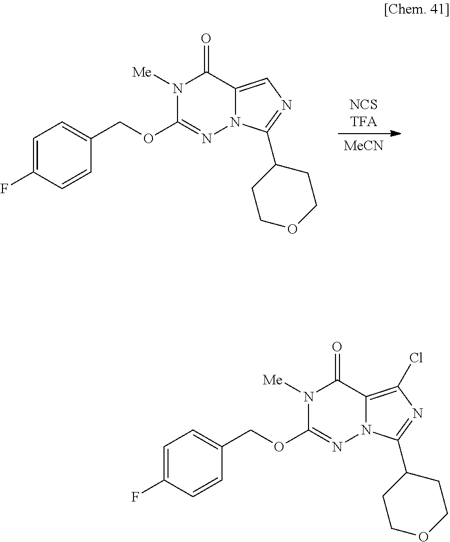 [SMILES: "MeCN" ; Cn1c(OCc2ccc(F)cc2)nn2c(C3CCOCC3)ncc2c1=O ; Cn1c(OCc2ccc(F)cc2)nn2c(C3CCOCC3)nc(Cl)c2c1=O ; "NCSTFA"]

To a solution of 2-[(4-fluorobenzyl)oxy]-3-methyl-7-(tetrahydro-2H-pyran-4-yl)imidazo[5,1-f][1,2,4]triazin-4(3H)-one (0.1 g, 0.28 mmol) in MeCN (1.0 mL) was added N-chlorosuccinimide (55.7 mg, 0.419 mmol) and TFA (103 μl, 1.40 mmol). The mixture was heated for 4 hours at 80° C. under nitrogen atmosphere. Upon completion, the reaction mixture was quenched with sat. NaHCO₃. The aqueous layer was extracted with EtOAc, and the combined organic phases were dried with sodium sulfate and concentrated to dryness. The residue was purified by silica gel column chromatography (hexane/EtOAc) to give the titled compound (28 mg, yield 26%) as a white solid.

LC-MS (m/z)=393 [M+H]⁺. ¹H-NMR (300 MHz, CDCl₃): δ 1.79-1.86 (m, 2H), 1.99-2.13 (m, 2H), 3.25-3.37 (m, 4H), 3.50-3.59 (m, 2H), 4.05-4.09 (m, 2H), 5.33 (s, 2H), 7.06-7.13 (m, 2H), 7.39-7.44 (m, 2H).

The compounds of Examples 118 and 119 were synthesized in a similar manner to Example 117.

[Chem. 42]

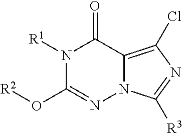

| No. | R¹— | R²— | R³— | ¹H-NMR |
|---|---|---|---|---|
| 118 | Et— | 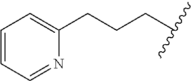 [SMILES: *CCCc1ccccn1] | 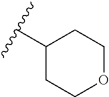 [SMILES: *C1CCOCC1] | (300 MHz, CDCl₃): δ 1.23 (t, J = 7.3 Hz, 3H), 1.80-1.85 (m, 2H), 1.96-2.10 (m, 2H), 2.26-2.35 (m, 2H), 2.95 (t, J = 7.3 Hz, 2H), 3.20-3.31 (m, 1H), 3.51 (td, J = 11.7, 2.2 Hz, 2H), 3.92-4.06 (m, 4H), 4.42 (t, J = 6.6 Hz, 2H), 7.11-7.16 (m, 2H), 7.57-7.63 (m, 1H), 8.51-8.53 (m, 1H). |
| 119 | Et— | 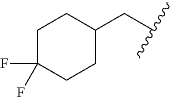 [SMILES: *CC1CCC(F)(F)CC1] | 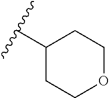 [SMILES: *C1CCOCC1] | (400 MHz, CDCl₃): δ 1.27 (t, J = 7.1 Hz, 3H), 1.50-1.55 (m, 2H), 1.71-1.94 (m, 7H), 2.04-2.14 (m, 2H), 2.16- 2.25 (m, 2H), 3.27-3.34 (m, 1H), 3.56 (td, J = 11.7, 2.1 Hz, 2H), 4.02 (q, J = 7.1 Hz, 2H), 4.08 (dq, J = 11.7, 2.1 Hz, 2H), 4.23 (d, J = 6.0 Hz, 2H). |

### Example 120

### 2-[(4-Fluorobenzyl)oxy]-3,5-dimethyl-7-(tetrahydro-2H-pyran-4-yl)imidazo[5,1-f][1,2,4]triazin-4(3H)-one

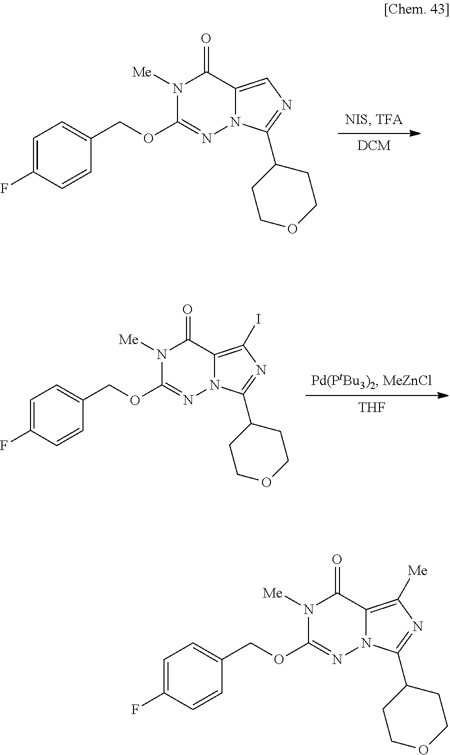

To a solution of 2-[(4-fluorobenzyl)oxy]-5-iodo-3-methyl-7-(tetrahydro-2H-pyran-4-yl)imidazo[5,1-f][1,2,4]triazin-4(3H)-one (30 mg, 0.062 mmol) in THF (0.5 mL) were added Pd(PtBu₃)₂ (6.3 mg, 0.012 mmol) and MeZnCl (2M in THF, 0.248 ml, 0.496 mmol). The mixture was stirred for 4 h at room temperature under nitrogen atmosphere. Upon completion, the reaction mixture was quenched with H₂O. The aqueous layer was extracted with EtOAc, and the combined organic phases were dried with sodium sulfate and concentrated to dryness. The residue was purified by amino silica gel column chromatography (hexane/EtOAc) to give the titled compound (20 mg, yield 87%) as a white solid.

LC-MS (m/z)=373 [M+H]⁺. ¹H-NMR (300 MHz, CDCl₃): δ 1.79-1.85 (m, 2H), 2.01-2.16 (m, 2H), 2.56 (s, 3H), 3.23-3.35 (m, 4H), 3.55 (td, J=11.7, 2.2 Hz, 2H), 4.05-4.11 (m, 2H), 5.32 (s, 2H), 7.05-7.12 (m, 2H), 7.40-7.44 (m, 2H).

### 2-[(4-Fluorobenzyl)oxy]-5-iodo-3-methyl-7-(tetrahydro-2H-pyran-4-yl)imidazo[5,1-f][1,2,4]triazin-4(3H)-one:

To a solution of 2-[(4-fluorobenzyl)oxy]-3-methyl-7-(tetrahydro-2H-pyran-4-yl)imidazo[5,1-f][1,2,4]triazin-4(3H)-one (0.1 g, 0.28 mmol) in MeCN (1.0 mL) were added N-iodosuccinimide (126 mg, 0.558 mmol) and TFA (103 μl, 1.40 mmol). The mixture was stirred for 4 h at room temperature under nitrogen atmosphere. Upon completion, the reaction mixture was quenched with sat. NaHCO₃. The aqueous layer was extracted with EtOAc, and the combined organic phases were dried with sodium sulfate and concentrated to dryness. The residue was purified by silica gel column chromatography (hexane/EtOAc) to give the titled compound (129 mg, yield 96%) as a white solid.

LC-MS (m/z)=485 [M+H]⁺. ¹H-NMR (300 MHz, CDCl₃): δ 1.78-1.84 (m, 2H), 2.02-2.16 (m, 2H), 3.25-3.36 (m, 4H), 3.54 (td, J=11.7, 2.2 Hz, 2H), 4.05-4.11 (m, 2H), 5.33 (s, 2H), 7.06-7.13 (m, 2H), 7.38-7.44 (m, 2H).

The compounds of Examples 121 and 122 were synthesized in a similar manner to Example 120.

[Chem. 44]

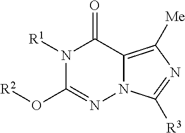

| No. | R¹— | R²— | R³— | ¹H-NMR |
|---|---|---|---|---|
| 121 | Et— | 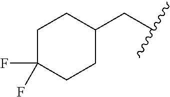 [SMILES: *CC1CCC(F)(F)CC1] | 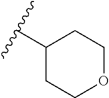 [SMILES: *C1CCOCC1] | (400 MHz, CDCl₃): δ 1.26 (t, J = 7.1 Hz, 3H), 1.46-1.57 (m, 2H), 1.71-1.95 (m, 7H), 2.06- 2.23 (m, 4H), 2.59 (s, 3H), 3.25-3.33 (m, 1H), 3.56 (td, J = 11.7, 2.0 Hz, 2H), 4.00 (q, J = 7.1 Hz, 2H), 4.09 (dq, J = 11.7, 2.0 Hz, 2H), 4.22 (d, J = 5.9 Hz, 2H). |
| 122 | 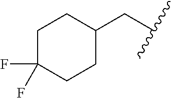 [SMILES: *CC1CCC(F)(F)CC1] | Me— | 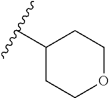 [SMILES: *C1CCOCC1] | (400 MHz, CDCl₃): δ 1.36-1.46 (m, 2H), 1.61-1.76 (m, 4H), 1.82-1.90 (m, 3H), 2.07- 2.17 (m, 4H), 2.59 (s, 3H), 3.32 (tt, J = 11.8, 3.9 Hz, 1H), 3.56 (td, J = 11.8, 2.2 Hz, 2H), 3.85 (d, J = 7.3 Hz, 2H), 4.03 (s, 3H), 4.09 (dq, J = 11.8, 2.2 Hz, 2H). |

### Example 123

### 2-[(4,4-Difluorocyclohexyl)methoxy]-3-ethyl-7-(tetrahydro-2H-pyran-4-yl)imidazo[5,1-f][1,2,4]triazin-4(3H)-thione

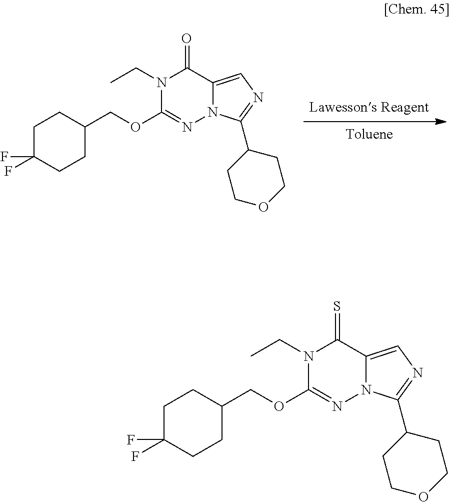

To a solution of 2-[(4,4-difluorocyclohexyl)methoxy]-3-ethyl-7-(tetrahydro-2H-pyran-4-yl)imidazo[5,1-f][1,2,4]triazin-4(3H)-one (68 mg, 0.18 mmol) in toluene (4 mL) was added Lawesson's reagent (149 mg, 0.37 mmol) at room temperature. The mixture was heated for 4 hr at 100° C. under nitrogen atmosphere. Upon completion, the reaction mixture was concentrated. The residue was purified by silica gel column chromatography (CHCl₃/MeOH) to give the titled compound (60 mg, yield 81%) as a pale yellow oil.

LC-MS (m/z)=413 [M+H]⁺. ¹H-NMR (400 MHz, CDCl₃): δ 7.92 (s, 1H), 4.55 (q, J=7.0 Hz, 2H), 4.26 (d, J=5.9 Hz, 2H), 4.09-4.06 (m, 2H), 3.58-3.54 (m, 2H), 3.39-3.34 (m, 1H), 2.20-1.68 (m, 10H), 1.57-1.46 (m, 2H), 1.31 (t, J=7.0 Hz, 3H).

### Example 124

### 2-[(2R)-2-phenylpyrrolidin-1-yl]-7-(tetrahydro-2H-pyran-4-yl)imidazo[5,1-f][1,2,4]triazin-4(3H)-one

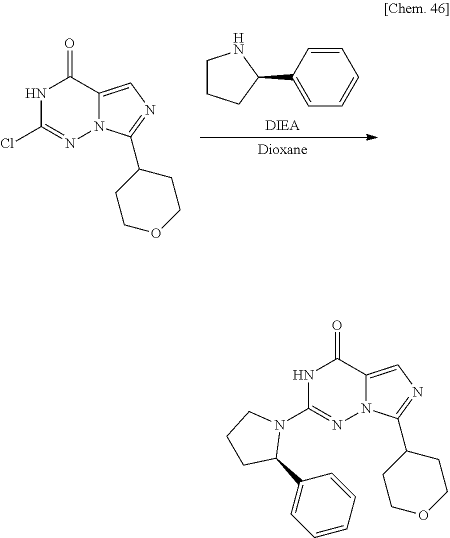

A solution of 2-chloro-7-(tetrahydro-2H-pyran-4-yl)imidazo[5,1-f][1,2,4]triazin-4(3H)-one (61 mg, 0.24 mmol), (2R)-2-phenylpyrrolidine (71 mg, 0.48 mmol) and DIEA (155 mg, 1.2 mmol) in anhydrous dioxane (3 mL) was refluxed for 16 h. The mixture was concentrated to dryness. The residue was purified by preparative HPLC (MeCN and H₂O with 0.1% NH₃.H₂O as mobile phase) to give the titled compound (36 mg, yield 41%).

¹H NMR (400 MHz, CD₃OD): δ 1.16-1.27 (m, 1H), 1.40-1.55 (m, 1H), 1.67-1.78 (m, 1H), 1.82-1.98 (m, 2H), 1.98-2.20 (m, 2H), 2.39-2.55 (m, 1H), 3.00-3.15 (m, 1H), 3.26-3.40 (m, 1H), 3.44-3.55 (m, 1H), 3.65-3.72 (m, 1H), 3.72-3.82 (m, 1H), 3.85-3.93 (m, 1H), 3.93-4.03 (m, 1H), 5.00-5.10 (m, 1H), 7.15-7.23 (m, 1H), 7.23-7.35 (m, 4H), 7.53 (s, 1H).

### Example 125

### 2-[(2R)-2-(4-chlorophenyl)pyrrolidin-1-yl]-7-(propan-2-yl)imidazo[5,1-f][1,2,4]triazin-4(3H)-one

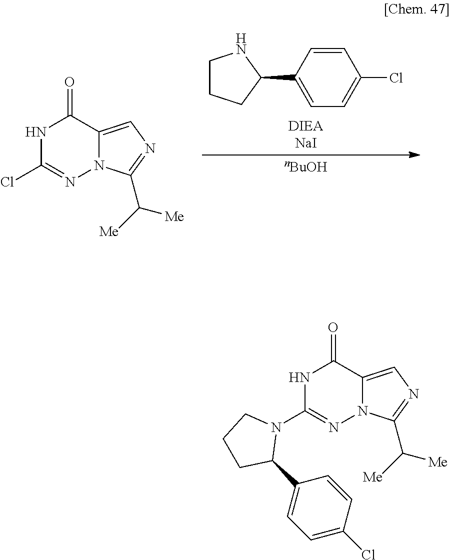

A solution of (2R)-2-(4-chlorophenyl)pyrrolidine (55 mg, 0.3 mmol), 2-chloro-7-(propan-2-yl)imidazo[5,1-f][1,2,4]triazin-4(3H)-one (64 mg, 0.3 mmol), NaI (45 mg, 0.3 mmol), and DIPEA (78 mg, 0.6 mmol) in n-BuOH (2 mL) was stirred at 160° C. for 12 h under microwave irradiation. Upon completion, the mixture was quenched by water and extracted with EtOAc. The combined organic layers were washed with brine, dried over Na₂SO₄ and concentrated in vacuo. The residue was purified by reversed phase chromatography (0.01% NH₃ in Water and MeCN) to give the titled compound as a white solid (30 mg, yield 45%).

¹H NMR (400 MHz, CD₃OD): δ 0.99-1.03 (m, 3H), 1.22-1.24 (m, 3H), 1.86-1.93 (m, 1H), 2.00-2.08 (m, 2H), 2.40-2.47 (m, 1H), 3.14-3.23 (m, 1H), 3.67-3.73 (m, 1H), 3.86-3.91 (m, 1H), 4.92-5.11 (m, 1H), 7.25-7.31 (m, 4H), 7.47 (s, 1H).

The compounds of Examples 126 to 134 were synthesized in a similar manner to Example 124 or 125.

[Chem. 48]

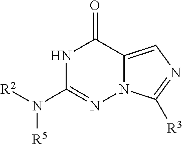

| No. | R²R⁵N— | R³— | ¹H-NMR |
|---|---|---|---|
| 126 | 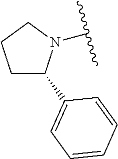 [SMILES: *N1CCC[C@H]1c1ccccc1] | 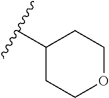 [SMILES: *C1CCOCC1] | (400 MHz, CD₃OD): δ 1.16-1.27 (m, 1H), 1.40-1.55 (m, 1H), 1.67-1.78 (m, 1H), 1.82- 1.98 (m, 2H), 1.98-2.20 (m, 2H), 2.39-2.55 (m, 1H), 3.00-3.15 (m, 1H), 3.26-3.40 (m, 1H), 3.44-3.55 (m, 1H), 3.65-3.72 (m, 1H), 3.72- 3.82 (m, 1H), 3.85-3.93 (m, 1H), 3.93-4.03 (m, 1H), 5.00-5.10 (m, 1H), 7.15-7.23 (m, 1H), 7.23-7.35 (m, 4H), 7.53 (s, 1H). |
| 127 | 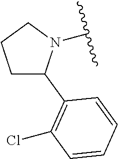 [SMILES: *N1CCCC1c1ccccc1Cl] | 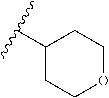 [SMILES: *C1CCOCC1] | (400 MHz, CD₃OD): δ 1.08-1.12 (m, 1H), 1.29-1.46 (m, 1H), 1.73-1.76 (m, 1H), 1.80- 1.89 (m, 2H), 2.08-2.14 (m, 2H), 2.51-2.56 (m, 1H), 2.99-3.03 (m, 1H), 3.32-3.37 (m, 1H), 3.45-3.50 (m, 1H), 3.69-3.75 (m, 2H), 3.91- 3.96 (m, 2H), 5.45 (dd, J = 8.0, 5.2 Hz, 1H), 7.19-7.25 (m, 3H), 7.40-7.42 (m, 1H), 7.54 (s, 1H). |
| 128 | 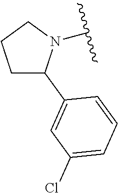 [SMILES: *N1CCCC1c1cccc(Cl)c1] | 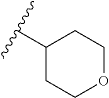 [SMILES: *C1CCOCC1] | (400 MHz, CD₃OD): δ 1.25-1.26 (m, 1H), 1.48-1.59 (m, 1H), 1.74-1.78 (m, 1H), 1.86- 1.91 (m, 2H), 2.03-2.16 (m, 2H), 2.44-2.51 (m, 1H), 3.01-3.09 (m, 1H), 3.34-3.41 (m, 1H), 3.48-3.55 (m, 1H), 3.69-3.72 (m, 1H), 3.79- 3.83 (m, 1H), 3.88-3.93 (m, 1H), 3.98-4.02 (m, 1H), 5.00-5.04 (m, 1H), 7.20-7.22 (m, 2H), 7.28-7.32 (m, 2H), 7.56 (s, 1H). |
| 129 | 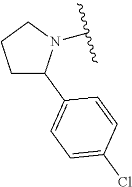 [SMILES: *N1CCCC1c1ccc(Cl)cc1] | 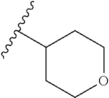 [SMILES: *C1CCOCC1] | (400 MHz, CD₃OD): δ 1.18 (br d, J = 11.6 Hz, 1H), 1.46-1.56 (m, 1H), 1.72 (br d, J = 11.6 Hz, 1H), 1.84-1.93 (m, 2H), 2.03-2.16 (m, 2H), 2.48-2.55 (m, 1H), 3.00-3.07 (m, 1H), 3.33- 3.36 (m, 1H), 3.45-3.52 (m, 1H), 3.65-3.71 (m, 1H), 3.78-3.82 (m, 1H), 3.86-3.92 (m, 1H), 3.96-4.00 (m, 1H), 5.01-5.04 (m, 1H), 7.27 (d, J = 8.8 Hz, 2H), 7.32 (d, J = 8.8 Hz, 2H), 7.55 (s, 1H). |
| 130 | 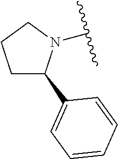 [SMILES: *N1CCC[C@@H]1c1ccccc1] | 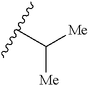 [SMILES: *C(C)C] | (400 MHz, CDCl₃): δ 1.26 (d, J = 7.0 Hz, 3H), 1.36 (d, J = 7.0 Hz, 3H), 1.95-2.20 (m, 3H), 2.42-2.56 (m, 1H), 3.27-3.43 (m, 1H), 3.66- 3.77 (m, 1H), 3.80-3.90 (m, 1H), 4.90-5.00 (m, 1H), 7.20-7.43 (m, 5H), 7.72 (s, 1H), 7.88 (br, 1H). |
| 131 | 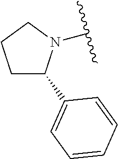 [SMILES: *N1CCC[C@H]1c1ccccc1] | 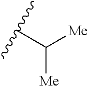 [SMILES: *C(C)C] | (400 MHz, CDCl₃): δ 1.24 (d, J = 6.8 Hz, 3H), 1.35 (d, J = 6.8 Hz, 3H), 1.95-2.17 (m, 3H), 2.40-2.55 (m, 1H), 3.25-3.40 (m, 1H), 3.65- 3.77 (m, 1H), 3.80-3.93 (m, 1H), 4.95-5.05 (m, 1H), 7.20-7.44 (m, 5H), 7.72 (s, 1H), 8.36 (br, 1H). |
| 132 | 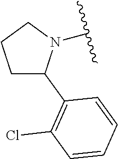 [SMILES: *N1CCCC1c1ccccc1Cl] | 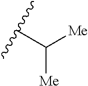 [SMILES: *C(C)C] | (400 MHz, CDCl₃): δ 1.18 (d, J = 7.0 Hz, 3H), 1.32 (d, J = 7.0 Hz, 3H), 1.97-2.16 (m, 3H), 2.45-2.60 (m, 1H), 3.25-3.40 (m, 1H), 3.70- 3.80 (m, 1H), 3.90-4.00 (m, 1H), 5.31-5.41 (m, 1H), 7.14-7.28 (m, 3H), 7.37-7.47 (m, 1H), 7.81 (s, 1H). |
| 133 | 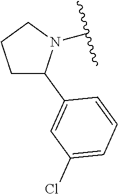 [SMILES: *N1CCCC1c1cccc(Cl)c1] | 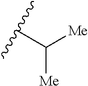 [SMILES: *C(C)C] | (400 MHz, CDCl₃): δ 1.17 (d, J = 7.2 Hz, 3H), 1.34 (d, J = 7.2 Hz, 3H), 1.95-2.20 (m, 3H), 2.40-2.55 (m, 1H), 3.20-3.34 (m, 1H), 3.65- 3.75 (m, 1H), 3.82-3.98 (m, 1H), 4.96-5.07 (m, 1H), 7.10-7.20 (m, 1H), 7.20-7.31 (m, 3H), 7.73 (s, 1H), 9.25 (br, 1H). |
| 134 | 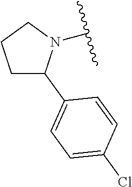 [SMILES: *N1CCCC1c1ccc(Cl)cc1] | 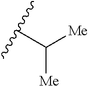 [SMILES: *C(C)C] | (400 MHz, CDCl₃): δ 1.20 (d, J = 6.8 Hz, 3H), 1.34 (d, J = 6.8 Hz, 3H), 1.90-2.16 (m, 3H), 2.40-2.54 (m, 1H), 3.20-3.35 (m, 1H), 3.64- 3.75 (m, 1H), 3.85-3.95 (m, 1H), 5.00-5.10 (m, 1H), 7.19 (d, J = 8.6 Hz, 2H), 7.29 (d, J = 8.6 Hz, 2H), 7.72 (s, 1H), 9.17 (br, 1H). |

### Example 135

### 2-{[(5-Methoxypyridin-2-yl)methyl]amino}-3-methyl-7-(propan-2-yl)imidazo[5,1-f][1,2,4]triazin-4(3H)-one

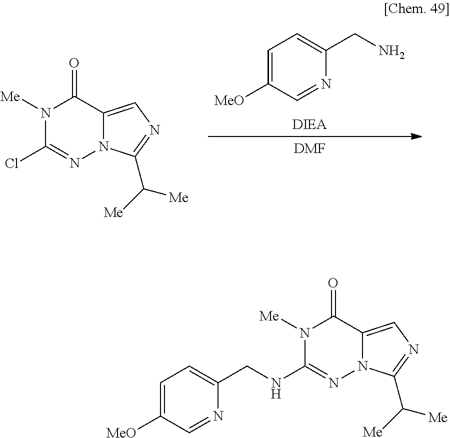

To a solution of 1-(5-methoxypyridin-2-yl)methanamine (138 mg, 1 mmol) and 2-chloro-3-methyl-7-(propan-2-yl)imidazo[5,1-f][1,2,4]triazin-4(3H)-one (90 mg, 0.4 mmol) in DMF (1.5 mL) was added DIEA (104 mg, 0.8 mmol). The reaction mixture was stirred at 50° C. for 10 h and purified by preparative HPLC (MeCN and H₂O with 0.01% NH₃.H₂O as mobile phase) to give the titled compound (40 mg, yield 30%).

LC-MS (m/z)=329 [M+H]⁺. ¹H-NMR (400 MHz, DMSO-d₆): δ 1.40 (d, J=7.2 Hz, 6H), 3.46-3.52 (m, 1H), 3.53 (s, 3H), 3.89 (s, 3H), 4.55 (d, J=4.0 Hz, 2H), 6.07 (s, 1H), 7.27-7.31 (m, 2H), 7.75 (s, 1H), 8.26 (d, J=2.4 Hz, 1H).

### Example 136

### 2-[(2-Fluoro-4-methylbenzyl)amino]-3-methyl-7-(tetrahydro-2H-pyran-4-yl)imidazo[5,1-f][1,2,4]triazin-4(3H)-one

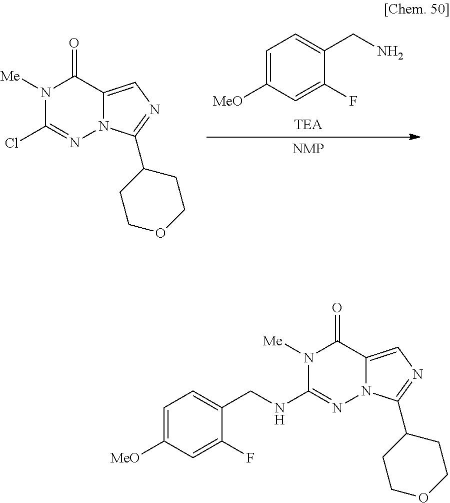

To a solution of 2-chloro-3-methyl-7-(tetrahydro-2H-pyran-4-yl)imidazo[5,1-f][1,2,4]triazin-4(3H)-one (40 mg, 0.149 mmol) and TEA (23 mg, 0.224 mmol) in NMP (1 mL) was added 2-fluoro-4-methylbenzylamine (25 mg, 0.179 mmol). The mixture was heated overnight at 100° C. Upon completion, the reaction mixture was cooled and partitioned between ethyl acetate and brine. The combined organic extracts were dried over sodium sulfate which was then filtrated. After concentration under reduced pressure, the residue was purified by silica gel column chromatography (chloroform/MeOH) to give the titled compound as a pale yellow solid (28 mg, yield 50%).

LC-MS (m/z)=372 [M+H]⁺. ¹H-NMR (400 MHz, CDCl₃): δ 1.82-1.91 (m, 2H), 2.04-2.18 (m, 2H), 2.35 (s, 3H), 3.35-3.42 (m, 1H), 3.43 (s, 3H), 3.55-3.65 (m, 2H), 4.06-4.15 (m, 2H), 4.54 (d, J=5.6 Hz, 2H), 4.63-4.75 (m, 1H), 6.89-6.96 (m, 2H), 7.27-7.33 (m, 1H), 7.75 (s, 1H).

### Example 137

### 3-Methyl-2-{[(6-methylpyridin-2-yl)methyl]amino}-7-(propan-2-yl)imidazo[5,1-f][1,2,4]triazin-4(3H)-one

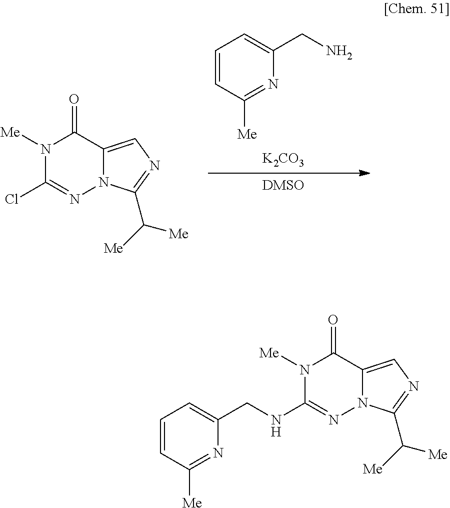

To a solution of 2-chloro-3-methyl-7-(propan-2-yl)imidazo[5,1-f][1,2,4]triazin-4(3H)-one (100 mg, 0.44 mmol) in DMSO (5 mL) were added 1-(6-methylpyridin-2-yl)methanamine (54 mg, 0.44 mmol) and K₂CO₃ (122 mg, 0.88 mmol). The mixture was stirred at 100° C. overnight and purified by column chromatography (EtOAc/petroleum Ether) to give the titled compound as a white solid (8 mg, yield 6%).

LC-MS (m/z)=313 [M+H]⁺. ¹H-NMR (400 MHz, CDCl₃): δ 1.39 (d, J=6.4 Hz, 6H), 2.58 (s, 3H), 3.44-3.51 (m, 1H), 3.54 (s, 3H), 4.56 (d, J=4.4 Hz, 2H), 6.33 (s, 1H), 7.10-7.16 (m, 2H), 7.59-7.63 (m, 1H), 7.74 (s, 1H).

### Example 138

### 3-Methyl-7-(propan-2-yl)-2-(1,2,3,4-tetrahydronaphthalen-1-ylamino)imidazo[5,1-f][1,2,4]triazin-4(3H)-one

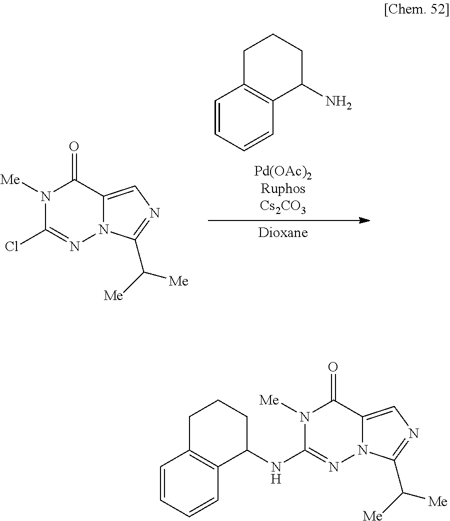 [SMILES: CC(C)c1ncc2c(=O)n(C)c(Cl)nn12 ; NC1CCCc2ccccc21 ; "Dioxane" ; "Pd(OAc)2RuphosCs2CO3" ; CC(C)c1ncc2c(=O)n(C)c(NC3CCCc4ccccc43)nn12]

A mixture of 2-chloro-3-methyl-7-(propan-2-yl)imidazo[5,1-f][1,2,4]triazin-4(3H)-one (150 mg, 0.66 mmol), 1,2,3,4-tetrahydronaphthalen-1-amine (146 mg, 1.0 mmol), Cs₂CO₃ (324 mg, 1.0 mmol), Ruphos (10 mg, 0.021 mmol), and Pd(OAc)₂ (10 mg, 0.045 mmol) in dioxane (3 mL) was stirred at 110 oC for 16 h. The reaction mixture was filtered and the filtrate was purified by preparative HPLC (MeCN and H₂O with 0.05% NH₃.H₂O as mobile phase) to afford the titled compound (18 mg, yield 8%).

¹H-NMR (400 MHz, CDCl₃): δ 1.35-1.47 (m, 6H), 1.84-1.95 (m, 2H), 2.05-2.20 (m, 2H), 2.75-2.95 (m, 2H), 3.37 (s, 3H), 3.41-3.54 (m, 1H), 4.25-4.40 (m, 1H), 5.10-5.23 (m, 1H), 7.14-7.30 (m, 3H), 7.35-7.43 (m, 1H), 7.77 (s, 1H).

The compounds of Examples 139 to 205 were synthesized in a similar manner to Example 135, 136, 137 or 138.

[Chem. 53]

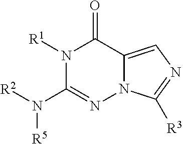

| No. | R¹— | R²R⁵N— | R³— | ¹H-NMR |
|---|---|---|---|---|
| 139 | Me— | 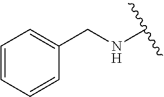 [SMILES: *NCc1ccccc1] | 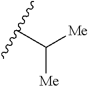 [SMILES: *C(C)C] | (400 MHz, CDCl₃): δ 1.37 (d, J = 7.2 Hz, 6H), 3.42-3.49 (m, 4H), 4.40-4.45 (m, 1H), 4.53-4.54 (m, 2H), 7.34-7.45 (m, 5H), 7.74 (s, 1H). |
| 140 | Me— | 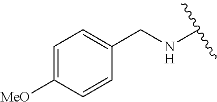 [SMILES: *NCc1ccc(OC)cc1] | 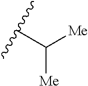 [SMILES: *C(C)C] | (400 MHz, CDCl₃): δ 1.38 (d, J = 6.8 Hz, 6H), 3.39-3.50 (m, 4H), 3.82 (s, 3H), 4.45 (s, 3H), 6.91 (d, J = 8.4 Hz, 2H), 7.34 (d, J = 8.4 Hz, 2H), 7.73 (s, 1H). |
| 141 | Me— | 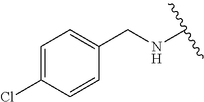 [SMILES: *NCc1ccc(Cl)cc1] | 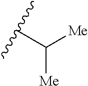 [SMILES: *C(C)C] | (400 MHz, CDCl₃): δ 1.35 (d, J = 6.8 Hz, 6H), 3.34-3.43 (m, 4H), 4.45-4.63 (m, 3H), 7.30-7.37 (m, 4H), 7.73 (s, 1H). |
| 142 | Me— | 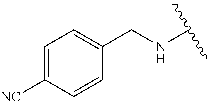 [SMILES: *NCc1ccc(C#N)cc1] | 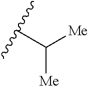 [SMILES: *C(C)C] | (400 MHz, CDCl₃): δ 1.20 (d, J = 7.2 Hz, 6H), 3.25-3.39 (m, 1H), 3.48 (s, 3H), 4.62 (d, J = 5.6 Hz, 2H), 4.80-4.90 (m, 1H), 7.52 (d, J = 8.4 Hz, 2H), 7.66 (d, J = 8.4 Hz, 2H), 7.74 (s, 1H). |
| 143 | Me— | 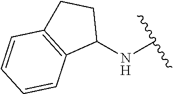 [SMILES: *NC1CCc2ccccc21] | 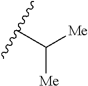 [SMILES: *C(C)C] | (400 MHz, CDCl₃): δ 1.39-1.42 (m, 6H), 1.96-2.05 (m, 1H), 2.73-2.83 (m, 1H), 2.92-3.16 (m, 2H), 3.40 (s, 3H), 3.41-3.52 (m, 1H), 4.32-4.40 (m, 1H), 5.43-5.48 (m, 1H), 7.26- 7.33 (m, 3H), 7.43 (d, J = 7.2 Hz, 1H), 7.76 (s, 1H). |
| 144 | Me— | 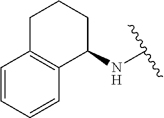 [SMILES: *N[C@@H]1CCCc2ccccc21] | 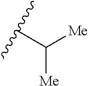 [SMILES: *C(C)C] | (400 MHz, CDCl₃): δ 1.39-1.42 (m, 6H), 1.86-1.92 (m, 2H), 2.10-2.14 (m, 2H), 2.80-2.92 (m, 2H), 3.97 (s, 3H), 3.42-3.49 (m, 1H), 4.31 (d, J = 7.2 Hz, 1H), 5.18 (dd, J = 12.0, 5.2 Hz, 1H), 7.17-7.26 (m, 3H), 7.39 (d, J = 10.0 Hz, 1H), 7.76 (s, 1H). |
| 145 | Me— | 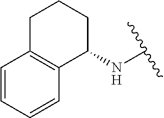 [SMILES: *N[C@H]1CCCc2ccccc21] | 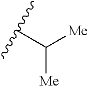 [SMILES: *C(C)C] | (400 MHz, CDCl₃): δ 1.39-1.42 (m, 6H), 1.86-1.92 (m, 2H), 2.10-2.14 (m, 2H), 2.80-2.92 (m, 2H), 3.97 (s, 3H), 3.42-3.49 (m, 1H), 4.31 (d, J = 7.2 Hz, 1H), 5.18 (dd, J = 12.0, 5.2 Hz, 1H), 7.17-7.26 (m, 3H), 7.39 (d, J = 10.0 Hz, 1H), 7.76 (s, 1H). |
| 146 | Me— | 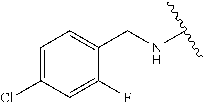 [SMILES: *NCc1ccc(Cl)cc1F] | 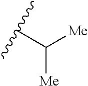 [SMILES: *C(C)C] | (400 MHz, DMSO-d₆): δ 1.14 (d, J = 6.8 Hz, 6H), 3.21 (sept, J = 6.8 Hz, 1H), 3.37 (s, 3H), 4.45 (d, J = 5.2 Hz, 2H), 7.23-7.25 (m, 1H), 7.40-7.43 (m, 1H), 7.50-7.54 (m, 3H). |
| 147 | Me— | 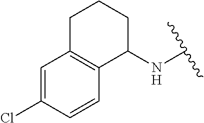 [SMILES: *NC1CCCc2cc(Cl)ccc21] | 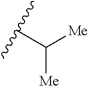 [SMILES: *C(C)C] | (400 MHz, CDCl₃): δ 1.37 (d, J = 6.8 Hz, 3H), 1.39 (d, J = 6.8 Hz, 3H), 1.92- 1.96 (m, 2H), 2.06-2.12 (m, 1H), 2.16- 2.21 (m, 1H), 2.82-2.97 (m, 2H), 3.42 (sept, J = 6.8 Hz, 1H), 3.40 (s, 3H), 4.30 (d, J = 7.6 Hz, 1H), 5.23 (dd, J = 12.8, 6.4 Hz, 1H), 7.47-7.49 (m, 2H), 7.53 (d, J = 8.4 Hz, 1H), 7.78 (s, 1H). |
| 148 | Me— | 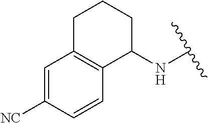 [SMILES: *NC1CCCc2cc(C#N)ccc21] | 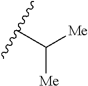 [SMILES: *C(C)C] | (400 MHz, CDCl₃): δ 1.39 (d, J = 6.8 Hz, 3H), 1.41 (d, J = 6.8 Hz, 3H), 1.85- 1.90 (m, 2H), 2.08-2.13 (m, 2H), 2.75-2.90 (m, 2H), 3.37 (s, 3H), 3.44 (sept, J = 6.8 Hz, 1H), 4.24 (d, J = 7.2 Hz, 1H), 5.14 (dd, J = 12.0, 5.2 Hz, 1H), 7.17-7.20 (m, 2H), 7.33 (d, J = 8.0 Hz, 1H), 7.77 (s, 1H). |
| 149 | Me— | 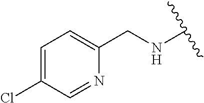 [SMILES: *NCc1ccc(Cl)cn1] | 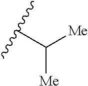 [SMILES: *C(C)C] | (400 MHz, CDCl₃): δ 1.38 (d, J = 7.1 Hz, 6H), 3.39-3.49 (m, 1H), 3.53 (s, 3H), 4.60 (d, J = 4.4 Hz, 2H), 5.91- 6.00 (m, 1H), 7.33 (d, J = 8.3 Hz, 1H), 7.72 (dd, J = 8.4, 2.3 Hz, 1H), 7.75 (s, 1H), 8.55 (d, J = 2.4 Hz, 1H). |
| 150 | Me— | 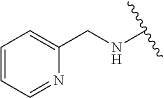 [SMILES: *NCc1ccccn1] | 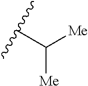 [SMILES: *C(C)C] | (400 MHz, CDCl₃): δ 1.40 (d, J = 7.2 Hz, 6H), 3.45-3.52 (m, 1H), 3.56 (s, 3H), 4.63 (d, J = 4.0 Hz, 2H), 6.28 (s, 1H), 7.26-7.29 (m, 1H), 7.37 (d, J = 8.0 Hz, 1H), 7.72-7.77 (m, 2H), 8.59 (d, J = 5.2 Hz, 1H). |
| 151 | Me— | 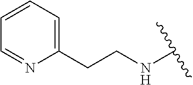 [SMILES: *NCCc1ccccn1] | 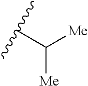 [SMILES: *C(C)C] | (400 MHz, CDCl₃): δ 1.38 (s, 3H), 1.39 (s, 3H), 3.14 (t, J = 5.9 Hz, 2H), 3.41-3.48 (m, 1H), 3.45 (s, 3H), 3.72- 3.68 (m, 2H), 6.75-6.78 (m, 1H), 7.20- 7.24 (m, 2H), 7.66-7.70 (m, 1H), 7.72 (s, 1H), 8.53 (d, J = 4.5 Hz, 1H). |
| 152 | Me— | 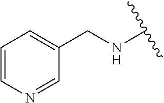 [SMILES: *NCc1cccnc1] | 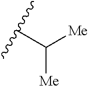 [SMILES: *C(C)C] | (400 MHz, CDCl₃): δ 1.33 (s, 3H), 1.35 (s, 3H), 3.45 (s, 3H), 3.42-3.53 (m, 1H), 4.57 (d, J = 5.2 Hz, 2H), 4.70 (t, J = 4.8 Hz, 1H), 7.31-7.34 (m, 1H), 7.74 (s, 1H), 7.76-7.77 (m, 1H), 8.57-8.58 (m, 1H), 8.69 (d, J = 1.8 Hz, 1H). |
| 153 | Me— | 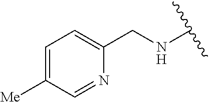 [SMILES: *NCc1ccc(C)cn1] | 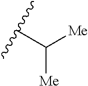 [SMILES: *C(C)C] | (400 MHz, DMSO-d₆): δ 1.36- 1.41 (m, 6H), 2.37 (s, 3H), 3.45- 3.52 (m, 1H), 3.55 (s, 3H), 4.57 (d, J = 4.0 Hz, 2H), 6.21 (s, 1H), 7.25 (s, 1H), 7.54 (dd, J = 8.0, 1.6 Hz, 1H), 7.75 (s, 1H), 8.40 (s, 1H). |
| 154 | Me— | 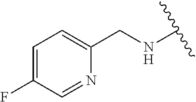 [SMILES: *NCc1ccc(F)cn1] | 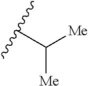 [SMILES: *C(C)C] | (400 MHz, DMSO-d₆): δ 1.39 (d, J = 7.2 Hz, 6H), 3.43-3.50 (m, 1H), 3.54 (s, 3H), 4.61 (d, J = 4.4 Hz, 2H), 6.01 (s, 1H), 7.37-7.40 (m, 1H), 7.46-7.51 (m, 1H), 7.76 (s, 1H), 8.45 (d, J = 2.4 Hz, 1H). |
| 155 | Me— | 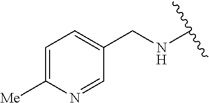 [SMILES: *NCc1ccc(C)nc1] | 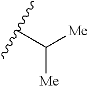 [SMILES: *C(C)C] | (400 MHz, CDCl₃): δ 1.34 (s, 3H), 1.36 (s, 3H), 2.55 (s, 3H), 3.43 (s, 3H), 3.38-3.44 (m, 1H), 4.51 (d, J = 5.3 Hz, 2H), 5.00 (br, 1H), 7.16 (d, J = 7.9 Hz, 1H), 7.66 (dd, J = 7.8, 2.2 Hz, 1H), 7.71 (s, 1H), 8.53 (s, 1H). |
| 156 | Me— | 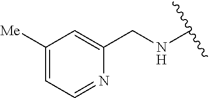 [SMILES: *NCc1cc(C)ccn1] | 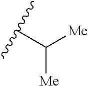 [SMILES: *C(C)C] | (400 MHz, CDCl₃): δ 1.39 (d, J = 7.2 Hz, 6H), 2.39 (s, 3H), 3.44- 3.51 (m, 1H), 3.54 (s, 3H), 4.56 (d, J = 4.0 Hz, 2H), 6.25 (s, 1H), 7.07-7.09 (m, 1H), 7.18 (s, 1H), 7.74 (s, 1H), 8.41-8.43 (m, 1H). |
| 157 | Me— | 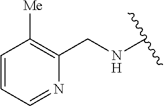 [SMILES: *NCc1ncccc1C] | 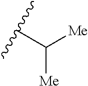 [SMILES: *C(C)C] | (400 MHz, CDCl₃): δ 1.42 (d, J = 7.2 Hz, 6H), 2.39 (s, 3H), 3.49-3.54 (m, 1H), 3.59 (s, 3H), 4.51 (d, J = 3.2 Hz, 2H), 6.92 (s, 1H), 7.19-7.22 (m, 1H), 7.54-7.56 (m, 1H), 7.76 (s, 1H), 8.42 (d, J = 4.8 Hz, 1H). |
| 158 | Me— | 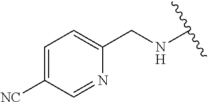 [SMILES: *NCc1ccc(C#N)cn1] | 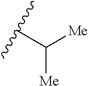 [SMILES: *C(C)C] | (400 MHz, CDCl₃): δ 1.34 (d, J = 6.8 Hz, 6H), 3.36-3.43 (m, 1H), 3.55 (s, 3H), 4.72 (d, J = 4.8 Hz, 2H), 5.98-6.01 (m, 1H), 7.53 (d, J = 8.0 Hz, 1H), 7.74 (s, 1H), 8.00-8.02 (m, 1H), 8.87 (s, 1H). |
| 159 | Me— | 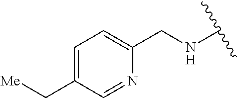 [SMILES: *NCc1ccc(CC)cn1] | 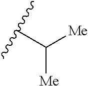 [SMILES: *C(C)C] | (400 MHz, CDCl₃): δ 1.28 (t, J = 7.6 Hz, 3H), 1.40 (d, J = 6.8 Hz, 6H), 2.69 (q, J = 7.6 Hz, 2H), 3.45-3.52 (m, 1H), 3.55 (s, 3H), 4.58 (d, J = 4.0 Hz, 2H), 6.22 (s, 1H), 7.29 (s, 1H), 7.56-7.58 (m, 1H), 7.75 (s, 1H), 8.43 (s, 1H). |
| 160 | Me— | 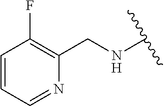 [SMILES: *NCc1ncccc1F] | 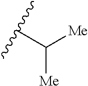 [SMILES: *C(C)C] | (400 MHz, CDCl₃): δ 1.39 (d, J = 7.2 Hz, 6H), 3.46-3.53 (m, 1H), 3.56 (s, 3H), 4.68 (d, J = 3.6 Hz, 2H), 6.19 (s, 1H), 7.30-7.34 (m, 1H), 7.46-7.50 (m, 1H), 7.75 (s, 1H), 8.41 (d, J = 4.8 Hz, 1H). |
| 161 | Me— | 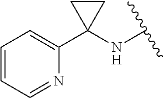 [SMILES: *NC1(c2ccccn2)CC1] | 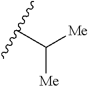 [SMILES: *C(C)C] | (400 MHz, CDCl₃): δ 1.17 (d, J = 6.8 Hz, 6H), 1.38-1.41 (m, 2H), 1.59-1.64 (m, 2H), 3.13-3.20 (m, 1H), 3.49 (s, 3H), 5.45 (s, 1H), 7.08-7.11 (m, 1H), 7.26-7.27 (m, 1H), 7.58-7.62 (m, 1H), 7.69 (s, 1H), 8.48 (d, J = 4.0 Hz, 1H). |
| 162 | Me— | 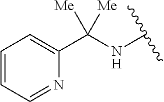 [SMILES: *NC(C)(C)c1ccccn1] | 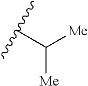 [SMILES: *C(C)C] | (400 MHz, CDCl₃): δ 1.34 (d, J = 7.2 Hz, 6H), 1.89 (s, 6H), 3.34-3.41 (m, 1H), 3.57 (s, 3H), 5.33-5.36 (m, 1H), 7.43 (s, 1H), 7.49-7.51 (m, 1H), 7.72 (s, 1H), 7.77-7.81 (m, 1H), 8.54 (d, J = 5.2 Hz, 1H). |
| 163 | Me— | 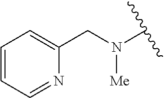 [SMILES: *N(C)Cc1ccccn1] | 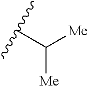 [SMILES: *C(C)C] | (400 MHz, CDCl₃): δ 1.33 (s, 3H), 1.35 (s, 3H), 2.85 (s, 3H), 3.38-3.45 (m, 1H), 3.57 (s, 3H), 4.42 (s, 2H), 7.22-7.25 (m, 1H), 7.37 (d, J = 7.8 Hz, 1H), 7.69-7.73 (m, 1H), 7.76 (s, 1H), 8.60 (d, J = 4.3 Hz, 1H). |
| 164 | Me— | 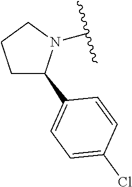 [SMILES: *N1CCC[C@@H]1c1ccc(Cl)cc1] | 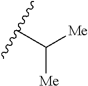 [SMILES: *C(C)C] | (400 MHz, CDCl₃): δ 1.16 (d, J = 7.2 Hz, 3H), 1.37 (d, J = 7.2 Hz, 3H), 1.88-1.95 (m, 1H), 2.01-2.08 (m, 1H), 2.15-2.21 (m, 1H), 2.38-2.44 (m, 1H), 3.27-3.34 (m, 2H), 3.49 (s, 3H), 3.82-3.88 (m, 1H), 5.00- 5.04 (m, 1H), 7.26-7.32 (m, 4H), 7.69 (s, 1H). |
| 165 | Me— | 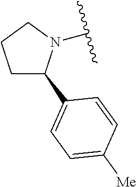 [SMILES: *N1CCC[C@@H]1c1ccc(C)cc1] | 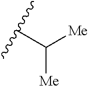 [SMILES: *C(C)C] | (400 MHz, CD₃OD): δ 1.13 (d, J = 7.1 Hz, 3H), 1.33 (d, J = 7.0 Hz, 3H), 1.87-1.98 (m, 1H), 2.00-2.10 (m, 1H), 2.14-2.20 (m, 1H), 2.27 (s, 3H), 2.37-2.43 (m, 1H), 3.36- 3.46 (m, 2H), 3.51 (s, 3H), 3.91- 3.98 (m, 1H), 5.01-5.06 (m, 1H), 7.10 (d, J = 8.2 Hz, 2H), 7.30 (d, J = 8.0 Hz, 2H), 7.56 (s, 1H). |
| 166 | Me— | 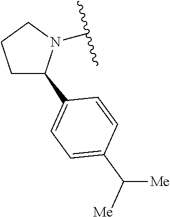 [SMILES: *N1CCC[C@@H]1c1ccc(C(C)C)cc1] | 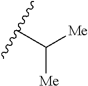 [SMILES: *C(C)C] | (400 MHz, CDCl₃): δ 1.15 (d, J = 6.8 Hz, 3H), 1.20 (d, J = 1.6 Hz, 3H), 1.22 (d, J = 0.8 Hz, 3 H), 1.38 (d, J = 7.2 Hz, 3H), 1.91-2.01 (m, 2H), 2.15-2.19 (m, 1H), 2.37-2.43 (m, 1H), 2.83-2.88 (m, 1H), 3.29-3.39 (m, 2H), 3.50 (s, 3H), 3.82-3.88 (m, 1H), 5.00-5.04 (m, 1H), 7.15 (d, J = 8.0 Hz, 2H), 7.28 (d, J = 8.0 Hz, 2H), 7.68 (s, 1H). |
| 167 | Me— | 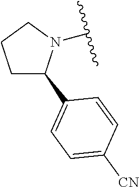 [SMILES: *N1CCC[C@@H]1c1ccc(C#N)cc1] | 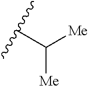 [SMILES: *C(C)C] | (400 MHz, CDCl₃): δ 1.10 (d, J = 7.1 Hz, 3H), 1.35 (d, J = 6.9 Hz, 3H), 1.84-1.95 (m, 1H), 2.05-2.25 (m, 2H), 2.42-2.48 (m, 1H), 3.20- 3.28 (m, 1H), 3.32-3.37 (m, 1H), 3.52 (s, 3H), 3.85-3.93 (m, 1H), 5.08-5.12 (m, 1H), 7.48 (d, J = 8.3 Hz, 2H), 7.61 (d, J = 8.3 Hz, 2H), 7.69 (s, 1H). |
| 168 | Me— | 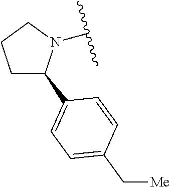 [SMILES: *N1CCC[C@@H]1c1ccc(CC)cc1] | 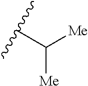 [SMILES: *C(C)C] | (400 MHz, CDCl₃): δ 1.16-1.21 (m, 6H), 1.38 (d, J = 7.2 Hz, 3H), 1.92-1.96 (m, 2H), 2.03-2.07 (m, 1H), 2.14-2.20 (m, 1H), 2.37-2.43 (m, 2H), 3.28-3.39 (m, 2H), 3.48 (s, 3H), 3.81-3.88 (m, 1H), 4.99- 5.04 (m, 1H), 7.12 (d, J = 8.0 Hz, 2H), 7.27 (d, J = 8.0 Hz, 2H), 7.67 (s, 1H). |
| 169 | Et— | 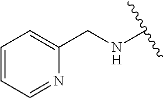 [SMILES: *NCc1ccccn1] | 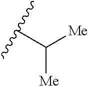 [SMILES: *C(C)C] | (400 MHz, CDCl₃): δ 1.39-1.45 (m, 9H), 3.46-3.50 (m, 1H), 4.12-4.18 (m, 2H), 4.63 (d, J = 3.6 Hz, 2H), 6.34 (s, 1H), 7.26-7.29 (m, 1H), 7.36 (d, J = 7.6 Hz, 1H), 7.72-7.76 (m, 2H), 8.59 (d, J = 4.8 Hz, 1H). |
| 170 | Et— | 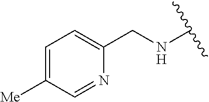 [SMILES: *NCc1ccc(C)cn1] | 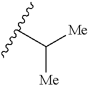 [SMILES: *C(C)C] | (400 MHz, CDCl₃): δ 1.39-1.43 (m, 9H), 2.36 (s, 3H), 3.43-3.51 (m, 1H), 4.12 (q, J = 6.4 Hz, 2H), 4.56 (d, J = 4.4 Hz, 2H), 6.27 (s, 1H), 7.24-7.26 (m, 1H), 7.52-7.55 (m, 1H), 7.73 (s, 1H), 8.40 (s, 1H). |
| 171 | Et— | 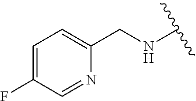 [SMILES: *NCc1ccc(F)cn1] | 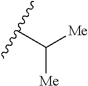 [SMILES: *C(C)C] | (400 MHz, CDCl₃): δ 1.37 (s, 3H), 1.39 (s, 3H), 1.41 (t, J = 7.4 Hz, 3H), 3.39-3.50 (m, 1H), 4.12 (q, J = 7.2 Hz, 2H), 4.61 (d, J = 4.2 Hz, 2H), 6.07 (s, 1H), 7.36-7.39 (m, 1H), 7.44-7.49 (m, 1H), 7.73 (s, 1H), 8.44 (d, J = 2.7 Hz, 1H). |
| 172 | Et— | 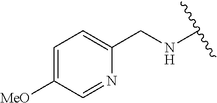 [SMILES: *NCc1ccc(OC)cn1] | 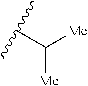 [SMILES: *C(C)C] | (400 MHz, CDCl₃): δ 1.40-1.43 (m, 9H), 3.44-3.51 (m, 1H), 3.89 (s, 3H), 4.10-4.15 (m, 2H), 4.56 (d, J = 4.4 Hz, 2H), 6.12 (s, 1H), 7.25-7.31 (m, 2H), 7.74 (s, 1H), 8.27 (d, J = 2.4 Hz, 1H). |
| 173 | Et— | 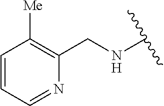 [SMILES: *NCc1ncccc1C] | 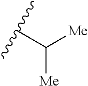 [SMILES: *C(C)C] | (400 MHz, CDCl₃): δ 1.33-1.39 (m, 9H), 2.63 (s, 3H), 3.35-3.47 (m, 3H), 5.29 (s, 2H), 7.18-7.22 (m, 1H), 7.55-7.57 (m, 1H), 7.72 (s, 1H), 8.09 (s, 1H), 8.33 (d, J = 5.2 Hz, 1H). |
| 174 | Et— | 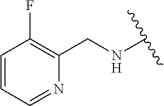 [SMILES: *NCc1ncccc1F] | 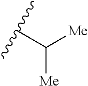 [SMILES: *C(C)C] | (400 MHz, CDCl₃): δ 1.34 (t, J = 7.6 Hz, 3H), 1.38 (d, J = 6.8 Hz, 6H), 3.36-3.48 (m, 3H), 5.37 (s, 2H), 7.01 (s, 1H), 7.33-7.37 (m, 1H), 7.49-7.53 (m, 1H), 7.72 (s, 1H), 8.34 (d, J = 4.8 Hz, 1H). |
| 175 | Et— | 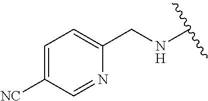 [SMILES: *NCc1ccc(C#N)cn1] | 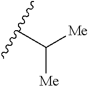 [SMILES: *C(C)C] | (400 MHz, CD₃OD): δ 1.00 (d, J = 8.0 Hz, 6H), 1.26 (t, J = 6.0 Hz, 3H), 1.79 (s, 1 H), 3.05 (q, J = 7.0 Hz, 1H), 4.04 (q, J = 7.4 Hz, 2H), 4.60 (s, 2H), 7.45 (s, 1H), 7.54 (d, J = 8.0 Hz, 1H), 8.02 (d, J = 8.0 Hz, 1H), 8.77 (s, 1H). |
| 176 | Et— | 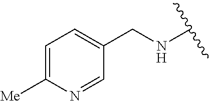 [SMILES: *NCc1ccc(C)nc1] | 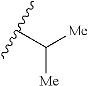 [SMILES: *C(C)C] | (400 MHz, CD₃OD): δ 1.12-1.15 (m, 6H), 1.17-1.20 (m, 3H), 2.39 (s, 3H), 3.23-3.28 (m, 1H), 3.94- 4.00 (m, 2H), 4.44 (s, 2H), 7.16- 7.18 (m, 1H), 7.45 (s, 1H), 7.68- 7.70 (m, 1H), 8.36-8.37 (m, 1H). |
| 177 | Et— | 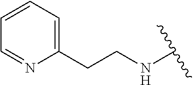 [SMILES: *NCCc1ccccn1] | 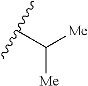 [SMILES: *C(C)C] | (400 MHz, CDCl₃): δ 1.33 (t, J = 7.2 Hz, 3H), 1.88 (d, J = 6.8 Hz, 6H), 3.15 (t, J = 6.0 Hz, 2H), 3.44 (sept, J = 6.8 Hz, 1H), 3.68-3.71 (m, 2H), 4.06 (q, J = 7.2 Hz, 2H), 6.87 (s, 1H), 7.20- 7.25 (m, 2H), 7.66-7.70 (m, 2H), 8.51 (d, J = 4.4 Hz, 1H). |
| 178 | Et— | 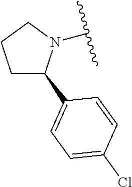 [SMILES: *N1CCC[C@@H]1c1ccc(Cl)cc1] | 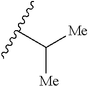 [SMILES: *C(C)C] | (400 MHz, CDCl₃): δ 1.21-1.26 (m, 3H) 1.30-1.34 (m, 3H), 1.38- 1.40 (m, 3H), 1.88-1.93 (m, 1H), 2.02-2.19 (m, 2H), 2.36-2.40 (m, 1H), 3.31-3.36 (m, 2H), 3.79-3.93 (m, 2H), 4.29-4.34 (m, 1H), 5.00- 5.05 (m, 1H), 7.26-7.28 (m, 2H), 7.30-7.33 (m, 2H), 7.68 (s, 1H). |
| 179 | ⁿPr— | 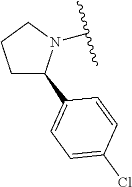 [SMILES: *N1CCC[C@@H]1c1ccc(Cl)cc1] | 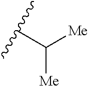 [SMILES: *C(C)C] | (400 MHz, CD₃OD): δ 0.87-0.89 (m, 3H), 1.14-1.16 (m, 3H), 1.33- 1.35 (m, 3H), 1.65-1.70 (m, 1H), 1.73-1.88 (m, 1H), 1.88-1.99 (m, 1H), 1.99-2.15 (m, 1H), 2.15-2.21 (m, 1H), 2.36-2.42 (m, 1H), 3.37- 3.48 (m, 2H), 3.80-3.93 (m, 2H), 4.23-4.30 (m, 1H), 5.05-5.09 (m, 1H), 7.29-7.31 (m, 2H), 7.42-7.44 (m, 2H), 7.58 (s, 1H). |
| 180 | Me— | 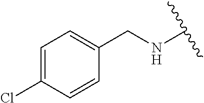 [SMILES: *NCc1ccc(Cl)cc1] | 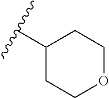 [SMILES: *C1CCOCC1] | 1H-NMR (400 MHz, CDCl₃): δ 1.75-1.79 (m, 2H), 1.94-2.04 (m, 2H), 3.20-3.27 (m, 1H), 3.42 (s, 3H), 3.47- 3.53 (m, 2H), 4.01-4.05 (m, 2H), 4.47 (d, J = 5.1 Hz, 2H), 4.71-4.73 (m, 1H), 7.31 (s, 4H), 7.70 (s, 1H). |
| 181 | Me— | 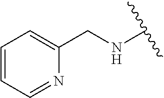 [SMILES: *NCc1ccccn1] | 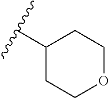 [SMILES: *C1CCOCC1] | (400 MHz, CDCl₃): δ 1.84-1.89 (m, 2H), 2.01-2.12 (m, 2H), 3.30-3.38 (m, 1H), 3.52-3.60 (m, 5H), 4.04-4.09 (m, 2H), 4.58 (d, J = 4.1 Hz, 2H), 6.29- 6.31 (m, 1H), 7.22-7.25 (m, 1H), 7.34-7.36 (m, 1H), 7.68-7.73 (m, 2H), 8.54-8.56 (m, 1H). |
| 182 | Me— | 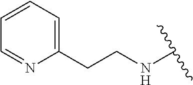 [SMILES: *NCCc1ccccn1] | 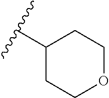 [SMILES: *C1CCOCC1] | (400 MHz, CDCl₃): δ 1.89-1.97 (m, 2H), 2.06-2.21 (m, 2H), 3.14-3.22 (m, 2H), 3.33-3.43 (m, 1H), 3.47 (s, 3H), 3.53-3.63 (m, 2H), 3.66-3.74 (m, 2H), 4.05-4.14 (m, 2H), 6.87-6.96 (m, 1H), 7.21-7.31 (m, 2H), 7.68- 7.77 (m, 2H), 8.51-8.56 (m, 1H). |
| 183 | Me— | 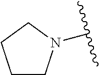 [SMILES: *N1CCCC1] | 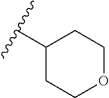 [SMILES: *C1CCOCC1] | (400 MHz, CDCl₃): δ 1.91-2.00 (m, 6H), 2.06-2.16 (m, 2H), 3.31- 3.43 (m, 5H), 3.46 (s, 3H), 3.58 (td, J = 11.6, 2.3 Hz, 2H), 4.09 (dd, J = 8.3, 3.4 Hz, 2H), 7.75 (s, 1H). |
| 184 | Me— | 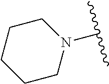 [SMILES: *N1CCCCC1] | 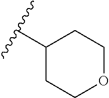 [SMILES: *C1CCOCC1] | (400 MHz, CDCl₃): δ 1.64-1.77 (m, 6H), 1.93 (d, J = 13.2 Hz, 2H), 2.06-2.16 (m, 2H), 3.06-3.09 (m, 4H), 3.33-3.41 (m, 1H), 3.46 (s, 3H), 3.59 (dt, J = 2.4, 11.6 Hz, 2H), 4.07-4.12 (m, 2H), 7.75 (s, 1H). |
| 185 | Me— | 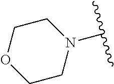 [SMILES: *N1CCOCC1] | 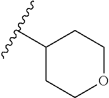 [SMILES: *C1CCOCC1] | (400 MHz, CDCl₃): δ 1.93 (d, J = 12.7 Hz, 2H), 2.06-2.16 (m, 2H), 3.16 (t, J = 4.6 Hz, 4H), 3.34-3.42 (m, 1H), 3.50 (s, 3H), 3.56-3.63 (m, 2H), 3.87 (t, J = 4.5 Hz, 4H), 4.08-4.12 (m, 2H), 7.78 (s, 1H). |
| 186 | Me— | 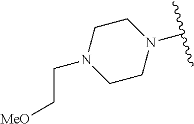 [SMILES: *N1CCN(CCOC)CC1] | 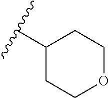 [SMILES: *C1CCOCC1] | (400 MHz, CDCl₃): δ 1.93 (d, J = 12.7 Hz, 2H), 2.05-2.15 (m, 2H), 2.66-2.69 (m, 6H), 3.19 (t, J = 4.8 Hz, 4H), 3.32-3.39 (m, 4H), 3.47 (s, 3H), 3.54-3.61 (m, 4H), 4.07- 4.11 (m, 2H), 7.76 (s, 1H). |
| 187 | Me— | 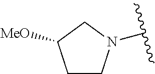 [SMILES: *N1CC[C@H](OC)C1] | 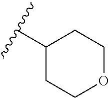 [SMILES: *C1CCOCC1] | (400 MHz, CDCl₃): δ 1.91-1.94 (m, 2H), 2.04-2.17 (m, 4H), 3.32- 3.40 (m, 6H), 3.46 (s, 3H), 3.55- 3.70 (m, 4H), 4.07-4.10 (m, 3H), 7.75 (s, 1H). |
| 188 | Me— | 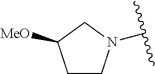 [SMILES: *N1CC[C@@H](OC)C1] | 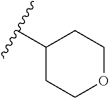 [SMILES: *C1CCOCC1] | (400 MHz, CDCl₃): δ 1.91-1.94 (m, 2H), 2.04-2.17 (m, 4H), 3.34- 3.38 (m, 6H), 3.46 (s, 3H), 3.55- 3.70 (m, 4H), 4.06-4.13 (m, 3H), 7.75 (s, 1H). |
| 189 | Me— | 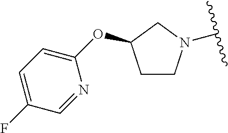 [SMILES: *N1CC[C@@H](Oc2ccc(F)cn2)C1] | 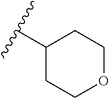 [SMILES: *C1CCOCC1] | (400 MHz, CDCl₃): δ 1.86-1.92 (m, 2H), 2.00-2.14 (m, 2H), 2.21- 2.34 (m, 2H), 3.27-3.35 (m, 1H), 3.39-3.57 (m, 7H), 3.71-3.78 (m, 1H), 3.83-3.87 (m, 1H), 4.02-4.08 (m, 2H), 5.53-5.56 (m, 1H), 6.67- 6.70 (m, 1H), 7.30-7.36 (m, 1H), 7.72 (s, 1H), 7.95-7.96 (m, 1H). |
| 190 | Me— | 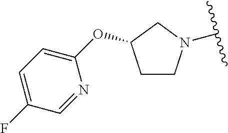 [SMILES: *N1CC[C@H](Oc2ccc(F)cn2)C1] | 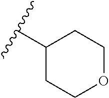 [SMILES: *C1CCOCC1] | (300 MHz, CDCl₃): δ 1.84-1.92 (m, 2H), 2.01-2.15 (m, 2H), 2.22- 2.31 (m, 2H), 3.26-3.58 (m, 8H), 3.70-3.87 (m, 2H), 4.03-4.08 (m, 2H), 5.52-5.56 (m, 1H), 6.66-6.71 (m, 1H), 7.29-7.36 (m, 1H), 7.71 (s, 1H), 7.95 (d, J = 2.9 Hz, 1H). |
| 191 | Me— | 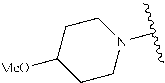 [SMILES: *N1CCC(OC)CC1] | 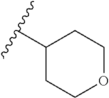 [SMILES: *C1CCOCC1] | (400 MHz, CDCl₃): δ 1.69-1.73 (m, 2H), 1.85-1.88 (m, 2H), 1.92- 2.04 (m, 4H), 2.94-3.00 (m, 2H), 3.35 (s, 3H), 3.41 (s, 3H), 3.26- 3.46 (m, 4H), 3.52-3.58 (m, 2H), 3.99-4.02 (m, 2H), 7.61 (s, 1H). |
| 192 | Me— | 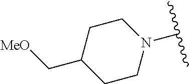 [SMILES: *N1CCC(COC)CC1] | 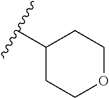 [SMILES: *C1CCOCC1] | (400 MHz, CDCl₃): δ 1.40-1.44 (m, 2H), 1.77-1.87 (m, 5H), 1.91-1.98 (m, 2H), 2.77-2.83 (m, 2H), 3.26-3.28 (m, 2H), 3.30 (s, 3H), 3.42 (s, 3H), 3.38-3.47 (m, 3H), 3.51-3.57 (m, 2H), 3.98-4.01 (m, 2H), 7.60 (s, 1H). |
| 193 | Me— | 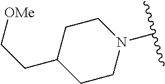 [SMILES: *N1CCC(CCOC)CC1] | 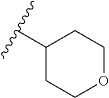 [SMILES: *C1CCOCC1] | (400 MHz, DMSO-d₆): δ 1.24-1.32 (m, 2H), 1.48-1.50 (m, 2H), 1.55-1.59 (m, 1H), 1.74-1.77 (m, 2H), 1.82-1.87 (m, 4H), 2.50 (s, 1H), 2.70-2.76 (m, 2H), 3.23 (s, 3H), 3.28-3.32 (m, 2H), 3.36-3.44 (m, 5H), 3.44-3.50 (m, 2H), 3.92-3.94 (m, 2H), 7.62 (s, 1H). |
| 194 | Me— | 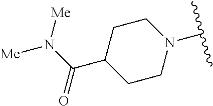 [SMILES: *N1CCC(C(=O)N(C)C)CC1] | 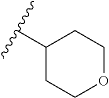 [SMILES: *C1CCOCC1] | (400 MHz, CDCl₃): δ 1.86-2.18 (m, 8H), 2.72-2.80 (m, 1H), 2.85- 2.92 (m, 2H), 3.00 (s, 3H), 3.12 (s, 3H), 3.33-3.41 (m, 1H), 3.46- 3.48 (m, 5H), 3.55-3.61 (m, 2H), 4.09-4.12 (m, 2H), 7.76 (s, 1H). |
| 195 | Me— | 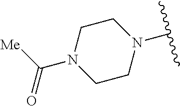 [SMILES: *N1CCN(C(C)=O)CC1] | 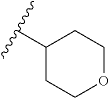 [SMILES: *C1CCOCC1] | (400 MHz, CDCl₃): δ 1.89-1.93 (m, 2H), 2.05-2.12 (m, 2H), 2.18 (s, 3H), 3.13 (t, J = 5.0 Hz, 2H), 3.20 (t, J = 4.8 Hz, 2H), 3.33-3.37 (m, 1H), 3.51 (s, 3H), 3.55-3.61 (m, 2H), 3.68 (t, J = 4.8 Hz, 2H), 3.75-3.86 (m, 2H), 4.08-4.11 (m, 2H), 7.78 (s, 1H). |
| 196 | Me— | 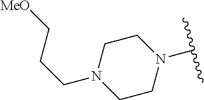 [SMILES: *N1CCN(CCCOC)CC1] | 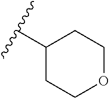 [SMILES: *C1CCOCC1] | (400 MHz, CDCl₃): δ 1.77-1.84 (m, 2H), 1.92-1.95 (m, 2H), 2.06-2.16 (m, 2H), 2.50-2.54 (m, 3H), 2.63 (s, 3H), 3.16-3.19 (m, 4H), 3.33-3.39 (m, 4H), 3.45-3.50 (m, 5H), 3.56-3.62 (m, 2H), 4.09-4.11 (m, 2H), 7.77 (s, 1H). |
| 197 | Me— | 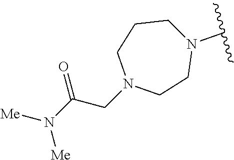 [SMILES: *N1CCCN(CC(=O)N(C)C)CC1] | 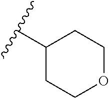 [SMILES: *C1CCOCC1] | (400 MHz, CDCl₃): δ 1.90-1.94 (m, 2H), 2.04-2.14 (m, 4H), 2.97 (s, 3H), 2.97-3.00 (m, 3H), 3.06 (s, 3H), 3.32-3.34 (m, 4H), 3.46 (s, 3H), 3.50 (s, 4H), 3.56-3.62 (m, 2H), 4.07-4.12 (m, 2H), 7.77 (s, 1H). |
| 198 | Et— | 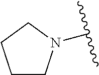 [SMILES: *N1CCCC1] | 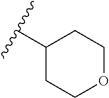 [SMILES: *C1CCOCC1] | (400 MHz, CDCl₃): δ 1.36 (t, J = 7.1 Hz, 3H), 1.92-2.00 (m, 6H), 2.06-2.16 (m, 2H), 3.31-3.42 (m, 5H), 3.58 (td, J = 11.6, 2.1 Hz, 2H), 4.03-4.11 (m, 4H), 7.74 (s, 1H). |
| 199 | ⁿPr— | 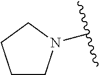 [SMILES: *N1CCCC1] | 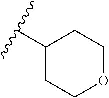 [SMILES: *C1CCOCC1] | (400 MHz, CDCl₃): δ 0.93 (t, J = 7.4 Hz, 3H), 1.71-1.80 (m, 2H), 1.92-2.00 (m, 6H), 2.06-2.16 (m, 2H), 3.30-3.41 (m, 5H), 3.58 (td, J = 11.6, 2.2 Hz, 2H), 3.92-3.96 (m, 2H), 4.07-4.11 (m, 2H), 7.73 (s, 1H). |
| 200 | 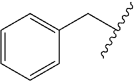 [SMILES: *Cc1ccccc1] | 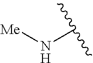 [SMILES: *NC] | 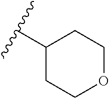 [SMILES: *C1CCOCC1] | (300 MHz, CDCl₃): δ 1.95 (d, J = 11.0 Hz, 2H), 2.12 (tt, J = 18.3, 5.9 Hz, 2H), 2.82 (d, J = 5.1 Hz, 3H), 3.38 (tt, J = 11.4, 3.8 Hz, 1H), 3.58 (td, J = 11.4, 2.2 Hz, 2H), 4.04-4.13 (m, 2H), 5.20 (s, 2H), 7.27 (d, J = 5.9 Hz, 5H), 7.82 (s, 1H). |
| 201 | 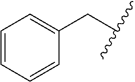 [SMILES: *Cc1ccccc1] | 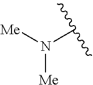 [SMILES: *N(C)C] | 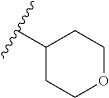 [SMILES: *C1CCOCC1] | (300 MHz, CDCl₃): δ 1.94 (d, J = 13.2 Hz, 2H), 2.11 (ddd, J = 24.6, 11.7, 3.7 Hz, 2H), 2.76 (s, 6H), 3.38 (tt, J = 11.4, 3.9 Hz, 1H), 3.58 (td, J = 11.4, 2.2 Hz, 2H), 4.10 (dd, J = 8.4, 3.3 Hz, 2H), 5.21 (s, 2H), 7.27 (dt, J = 13.2, 4.8 Hz, 5H), 7.76 (s, 1H). |
| 202 | 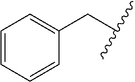 [SMILES: *Cc1ccccc1] | 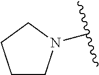 [SMILES: *N1CCCC1] | 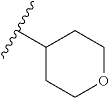 [SMILES: *C1CCOCC1] | (400 MHz, CDCl₃): δ 1.88-1.96 (m, 6H), 2.07-2.17 (m, 2H), 3.27-3.39 (m, 5H), 3.58 (td, J = 11.6, 2.1 Hz, 2H), 4.07-4.12 (m, 2H), 5.22 (s, 2H), 7.24- 7.30 (m, 5H), 7.77 (s, 1H). |
| 203 | 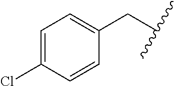 [SMILES: *Cc1ccc(Cl)cc1] | 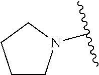 [SMILES: *N1CCCC1] | 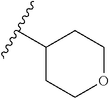 [SMILES: *C1CCOCC1] | (400 MHz, CDCl₃): δ 1.89-1.95 (m, 6H), 2.05-2.17 (m, 2H), 3.26-3.40 (m, 5H), 3.55-3.61 (m, 2H), 4.08-4.11 (m, 2H), 5.17 (s, 2H), 7.16 (d, J = 8.5 Hz, 2H), 7.28 (d, J = 8.5 Hz, 2H), 7.77 (s, 1H). |
| 204 | Me— | 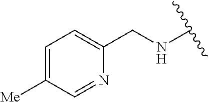 [SMILES: *NCc1ccc(C)cn1] | 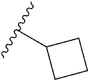 [SMILES: *C1CCC1] | (400 MHz, CDCl₃): δ 2.00-2.05 (m, 1H), 2.09-2.16 (m, 1H), 2.37-2.44 (m, 5H), 2.51-2.61 (m, 2H), 3.53 (s, 3H), 3.98-4.02 (m, 1H), 4.57 (d, J = 4.4 Hz, 2H), 6.19 (s, 1H), 7.25 (s, 1H), 7.54- 7.56 (m, 1H), 7.77 (s, 1H), 8.40 (s, 1H). |
| 205 | Me— | 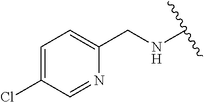 [SMILES: *NCc1ccc(Cl)cn1] | 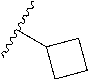 [SMILES: *C1CCC1] | (400 MHz, CDCl₃): δ 2.00-2.13 (m, 1H), 2.09-2.13 (m, 1H), 2.36-2.42 (m, 2H), 2.53-2.58 (m, 2H), 3.54 (s, 3H), 3.95-4.00 (m, 1H), 4.61-4.62 (m, 2H), 5.97 (s, 1H), 7.35 (d, J = 8.4 Hz, 1H), 7.72-7.75 (m, 1H), 7.78 (s, 1H), 8.55 (d, J = 2.4 Hz, 1H). |

### Example 206

### 3-(Cyclopropylmethyl)-2-(pyrrolidin-1-yl)-7-(tetrahydro-2H-pyran-4-yl)imidazo[5,1-f][1,2,4]triazin-4(3H)-one

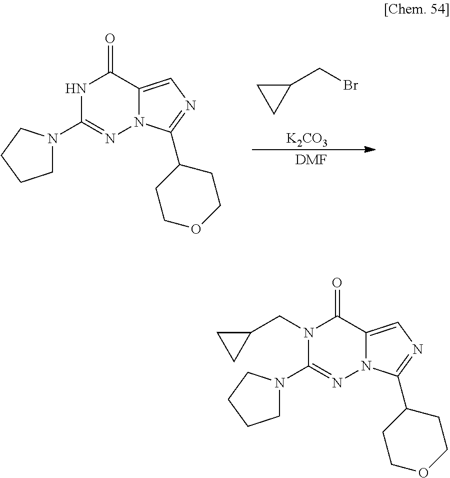

A solution of 2-(pyrrolidin-1-yl)-7-(tetrahydro-2H-pyran-4-yl)imidazo[5,1-f][1,2,4]triazin-4(3H)-one (50 mg, 0.173 mmol), potassium carbonate (48 mg, 0.346 mmol), and (bromomethyl)cyclopropane (25 μL, 0.259 mmol) in DMF (350 μL) was stirred at r.t. for 14 h. The resulting mixture was purified by silica gel column chromatography (chloroform/MeOH) to give the titled compound (35 mg, yield 59%) as white solids.

¹H-NMR (400 MHz, CDCl₃): δ 0.38-0.42 (m, 2H), 0.62-0.67 (m, 2H), 1.31-1.38 (m, 1H), 1.95-2.18 (m, 8H), 3.39-3.64 (m, 7H), 4.07-4.11 (m, 2H), 4.32 (d, J=7.6 Hz, 2H), 7.54 (s, 1H).

The compounds of Examples 207 to 215 were synthesized in a similar manner to Example 206.

[Chem. 55]

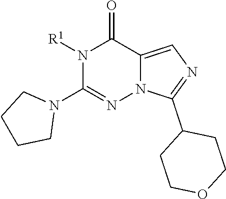

| No. | R¹— | ¹H-NMR |
|---|---|---|
| 207 | 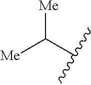 [SMILES: *C(C)C] | (400 MHz, CDCl₃): δ 1.42 (d, J = 6.4 Hz, 6H), 1.96- 2.18 (m, 8H), 3.38-3.46 (m, 1H), 3.53-3.63 (m, 6H), 4.07-4.11 (m, 2H), 5.49-5.56 (m, 1H), 7.48 (s, 1H). |
| 208 | 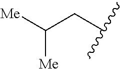 [SMILES: *CC(C)C] | (400 MHz, CDCl₃): δ 1.03 (d, J = 6.8 Hz, 6H), 1.96- 1.99 (m, 6H), 2.08-2.20 (m, 3H), 3.39-3.47 (m, 1H), 3.53-3.64 (m, 6H), 4.08-4.11 (m, 2H), 4.26 (d, J = 6.6 Hz, 2H), 7.50 (s, 1H). |
| 209 | 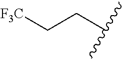 [SMILES: *CCC(F)(F)F] | (400 MHz, CDCl₃): δ 1.97-2.01 (m, 6H), 2.07-2.17 (m, 2H), 2.65-2.76 (m, 2H), 3.39-3.47 (m, 1H), 3.53- 3.63 (m, 6H), 4.07-4.11 (m, 2H), 4.73 (t, J = 6.7 Hz, 2H), 7.51 (s, 1H). |
| 210 | 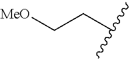 [SMILES: *CCOC] | (400 MHz, CDCl₃): δ 1.96-1.99 (m, 6H), 2.07-2.16 (m, 2H), 3.40-3.45 (m, 4H), 3.53-3.63 (m, 6H), 3.78- 3.81 (m, 2H), 4.08-4.11 (m, 2H), 4.64-4.66 (m, 2H), 7.55 (s, 1H). |
| 211 | 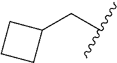 [SMILES: *CC1CCC1] | (400 MHz, CDCl₃): δ 1.85-1.99 (m, 10H), 2.07-2.14 (m, 4H), 2.79-2.86 (m, 1H), 3.38-3.63 (m, 7H), 4.07- 4.11 (m, 2H), 4.46 (d, J = 6.8 Hz, 2H), 7.49 (s, 1H). |
| 212 | 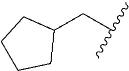 [SMILES: *CC1CCCC1] | (400 MHz, CDCl₃): δ 1.35-1.39 (m, 2H), 1.59-1.69 (m, 4H), 1.80-1.87 (m, 2H), 1.96-1.99 (m, 6H), 2.08- 2.18 (m, 2H), 2.39-2.46 (m, 1H), 3.39-3.63 (m, 7H), 4.08-4.10 (m, 2H), 4.37 (d, J = 7.1 Hz, 2H), 7.49 (s, 1H). |
| 213 | 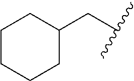 [SMILES: *CC1CCCCC1] | (400 MHz, CDCl₃): δ 1.01-1.11 (m, 2H), 1.18-1.31 (m, 4H), 1.69-1.86 (m, 6H), 1.96-1.99 (m, 5H), 2.08- 2.18 (m, 2H), 3.39-3.46 (m, 1H), 3.53-3.63 (m, 6H), 4.08-4.10 (m, 2H), 4.29 (d, J = 6.1 Hz, 2H), 7.49 (s, 1H). |
| 214 | 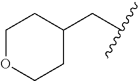 [SMILES: *CC1CCOCC1] | (400 MHz, CDCl₃): δ 1.45-1.54 (m, 2H), 1.72-1.76 (m, 2H), 1.96-2.15 (m, 8H), 3.40-3.65 (m, 10H), 4.00-4.11 (m, 4H), 4.35 (d, J = 6.6 Hz, 2H), 7.49 (s, 1H). |
| 215 | 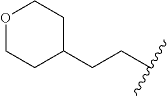 [SMILES: *CCC1CCOCC1] | (400 MHz, CDCl₃): δ 1.31-1.44 (m, 2H), 1.61-1.83 (m, 4H), 1.96-2.20 (m, 8H), 3.37-3.65 (m, 10H), 3.95-4.11 (m, 4H), 4.56 (t, J = 6.2 Hz, 2H), 7.50 (s, 1H). |

### Examples 216 and 217

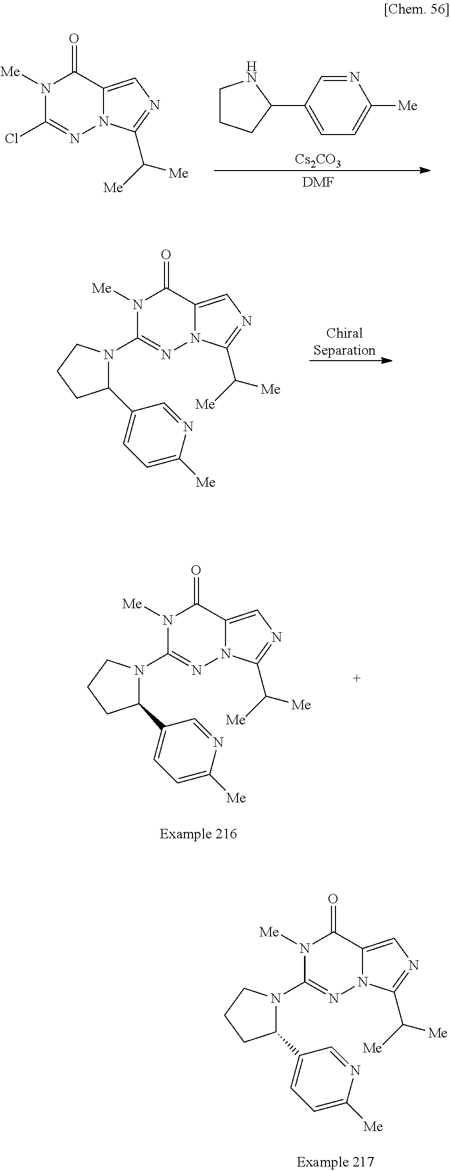

### 3-Methyl-2-[(2R)-2-(6-methylpyridin-3-yl)pyrrolidin-1-yl]-7-(propan-2-yl)imidazo[5,1-f][1,2,4]triazin-4(3H)-one

The chiral separation of 3-methyl-2-[2-(6-methylpyridin-3-yl)pyrrolidin-1-yl]-7-(propan-2-yl)imidazo[5,1-f][1,2,4]triazin-4(3H)-one (150 mg) gave the titled compound. Retention Time: 4.03 min./Method A.

LC-MS (m/z)=353 [M+H]⁺. ¹H-NMR (400 MHz, CDCl₃): δ 1.11-1.12 (m, 3H), 1.32-1.34 (m, 3H), 1.93-2.01 (m, 1H), 2.03-2.19 (m, 1H), 2.18-2.24 (m, 1H), 2.41-2.46 (m, 1H), 2.47 (s, 3H), 3.34-3.41 (m, 1H), 3.45-3.49 (m, 1H), 3.52 (s, 3H), 3.94-4.01 (m, 1H), 5.06-5.11 (m, 1H), 7.27-7.25 (m, 1H), 7.57(s, 1H), 7.83-7.85 (m, 1H), 8.49 (s, 1H).

### 3-Methyl-2-[(2S)-2-(6-methylpyridin-3-yl)pyrrolidin-1-yl]-7-(propan-2-yl)imidazo[5,1-f][1,2,4]triazin-4(3H)-one

The chiral separation of 3-methyl-2-[2-(6-methylpyridin-3-yl)pyrrolidin-1-yl]-7-(propan-2-yl)imidazo[5,1-f][1,2,4]triazin-4(3H)-one (150 mg) gave the titled compound. Retention Time: 4.79 min./Method A.

LC-MS (m/z)=353 [M+H]⁺. ¹H-NMR (400 MHz, CDCl₃): δ 1.11-1.12 (m, 3H), 1.32-1.34 (m, 3H), 1.93-2.01 (m, 1H), 2.03-2.19 (m, 1H), 2.18-2.24 (m, 1H), 2.41-2.46 (m, 1H), 2.47 (s, 3H), 3.34-3.41 (m, 1H), 3.45-3.49 (m, 1H), 3.52 (s, 3H), 3.94-4.01 (m, 1H), 5.06-5.11 (m, 1H), 7.27-7.25 (m, 1H), 7.57(s, 1H), 7.83-7.85 (m, 1H), 8.49 (s, 1H).

### 3-Methyl-2-[2-(6-methylpyridin-3-yl)pyrrolidin-1-yl]-7-(propan-2-yl)imidazo[5,1-f][1,2,4]triazin-4(3H)-one:

A mixture of 2-chloro-3-methyl-7-(propan-2-yl)imidazo[5,1-f][1,2,4]triazin-4(3H)-one (200 mg, 0.89 mmol), 2-methyl-5-(pyrrolidin-2-yl)pyridine (290 mg, 1.73 mmol) and Cs₂CO₃ (582 mg, 1.79 mmol) in anhydrous DMF (20 mL) was heated to 50° C. for 1 h. The reaction was quenched by adding 20 mL saturated NH₄Cl and 20 mL EtOAc. The product was purified through silica gel column to give the titled compound (150 mg, yield 48%).

### Example 218

### 3-Methyl-2-[(2R)-2-(5-methylpyridin-2-yl)pyrrolidin-1-yl]-7-(propan-2-yl)imidazo[5,1-f][1,2,4]triazin-4(3H)-one

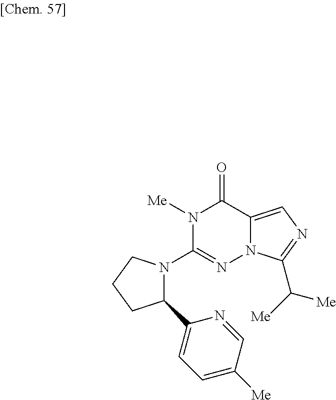 [SMILES: Cc1ccc([C@H]2CCCN2c2nn3c(C(C)C)ncc3c(=O)n2C)nc1]

The titled compound was synthesized in a similar manner to Examples 216 and 217.

Retention Time: 3.76 min./Method B.

LC-MS (m/z)=353 [M+H]⁺. ¹H-NMR (400 MHz, CDCl₃): δ 1.01-1.03 (m, 3H), 1.27-1.28 (m, 3H), 1.92-1.99 (m, 1H), 2.05-2.11 (m, 1H), 2.17-2.21 (m, 1H), 2.30 (s, 3H), 2.43-2.49 (m, 1H), 3.31-3.35 (m, 1H), 3.49-3.51 (m, 1H), 3.52 (s, 3H), 3.95-4.01 (m, 1H), 5.15-5.19 (m, 1H), 7.44-7.46 (m, 1H), 7.55 (s, 1H), 7.60-7.62 (m, 1H), 8.32 (s, 1H).

### Example 219

### 3-Methyl-2-[(2S)-2-(5-methylpyridin-2-yl)pyrrolidin-1-yl]-7-(propan-2-yl)imidazo[5,1-f][1,2,4]triazin-4(3H)-one

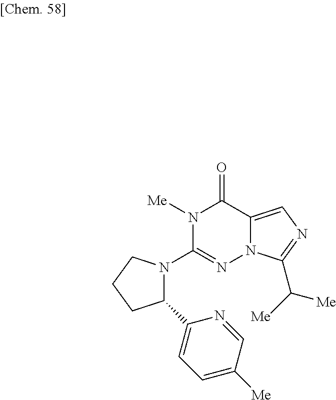 [SMILES: Cc1ccc([C@@H]2CCCN2c2nn3c(C(C)C)ncc3c(=O)n2C)nc1]

The titled compound was synthesized in a similar manner to Examples 216 and 217.

Retention Time: 4.51 min./Method B.

LC-MS (m/z)=353 [M+H]⁺. ¹H-NMR (400 MHz, CDCl₃): δ 1.01-1.03 (m, 3H), 1.27-1.28 (m, 3H), 1.92-1.99 (m, 1H), 2.05-2.11 (m, 1H), 2.17-2.21 (m, 1H), 2.30 (s, 3H), 2.43-2.49 (m, 1H), 3.31-3.35 (m, 1H), 3.49-3.51 (m, 1H), 3.52 (s, 3H), 3.95-4.01 (m, 1H), 5.15-5.19 (m, 1H), 7.44-7.46 (m, 1H), 7.55 (s, 1H), 7.60-7.62 (m, 1H), 8.32 (s, 1H).

### Example 220

### 2-[4-(2-Methoxyethyl)-1,4-diazepan-1-yl]-3-methyl-7-(tetrahydro-2H-pyran-4-yl)imidazo[5,1-f][1,2,4]triazin-4(3H)-one

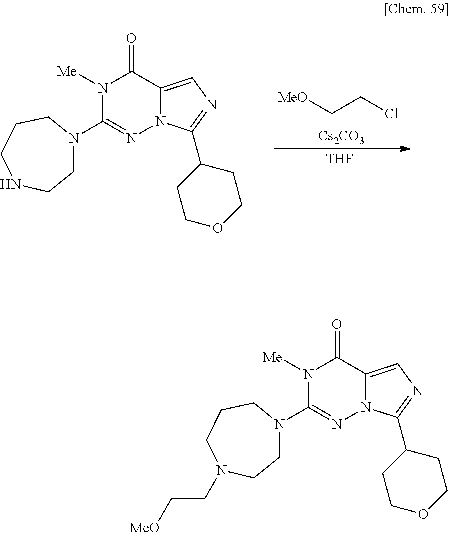 [SMILES: Cn1c(N2CCCNCC2)nn2c(C3CCOCC3)ncc2c1=O ; "Cs2CO3" ; COCCCl ; "THF" ; COCCN1CCCN(c2nn3c(C4CCOCC4)ncc3c(=O)n2C)CC1]

To a solution of 2-(1,4-diazepan-1-yl)-3-methyl-7-(tetrahydro-2H-pyran-4-yl)imidazo[5,1-f][1,2,4]triazin-4(3H)-one (40 mg, 0.12 mmol) in THF (2 mL) were added 2-chloroethyl methyl ether (23 mg, 0.24 mmol) and Cs₂CO₃ (117 mg, 0.36 mmol). The mixture was stirred at 50° C. overnight. The product was purified by reversed phase (0.01% NH₃ in water and MeCN) to give the titled compound (8 mg, yield 17%).

LC-MS (m/z)=391.2 [M+H]⁺. ¹H-NMR (400 MHz, CDCl₃): δ 1.91-2.15 (m, 6H), 2.78 (t, J=5.2 Hz, 2H), 2.85 (t, J=5.6 Hz, 2H), 2.90-2.91 (m, 2H), 3.31-3.52 (m, 1H), 3.38 (s, 3H), 3.39-3.42 (m, 2H), 3.45 (s, 5H), 3.52-3.61 (m, 4H), 4.09 (d, J=11.6 Hz, 2H), 7.75 (s, 1H).

### Example 221

### 2-[4-(Methoxyacetyl)-1,4-diazepan-1-yl]-3-methyl-7-(tetrahydro-2H-pyran-4-yl)imidazo[5,1-f][1,2,4]triazin-4(3H)-one

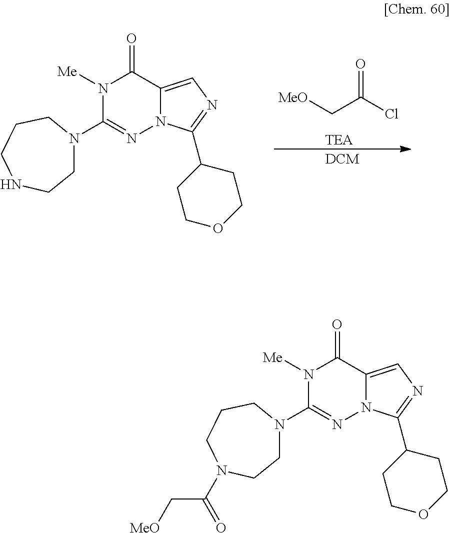

A solution of 2-(1,4-diazepan-1-yl)-3-methyl-7-(tetrahydro-2H-pyran-4-yl)imidazo[5,1-f][1,2,4]triazin-4(3H)-one (20 mg, 0.06 mmol), methoxyacetyl chloride (26 mg, 0.24 mmol), and TEA (0.2 mL) in DCM (1 mL) was stirred at room temperature for 2 h. And then the solution was washed with water and extracted with EtOAc, and the combined organic layers were washed with water and brine, dried with Na₂SO₄, and concentrated to give a residue which was then purified by reversed phase (0.01% NH₃ in Water and MeCN) to give the titled compound as a white solid (15 mg, yield 70%).

LC-MS (m/z)=405.2 [M+H]⁺. ¹H-NMR (400 MHz, CD₃OD): δ 1.89-2.14 (m, 6H), 3.36 (s, 1H), 3.39-3.42 (m, 4H), 3.48-3.50 (m, 5H), 3.58-3.68 (m, 4H), 3.72-3.77 (m, 2H), 3.83-3.86 (m, 1H), 4.04-4.07 (m, 2H), 4.15 (s, 1H), 4.23 (s, 1H), 7.66-7.67 (m, 1H).

### Example 222

### 2-{4-[(Dimethylamino)acetyl]-1,4-diazepan-1-yl}-3-methyl-7-(tetrahydro-2H-pyran-4-yl)imidazo[5,1-f][1,2,4]triazin-4(3H)-one

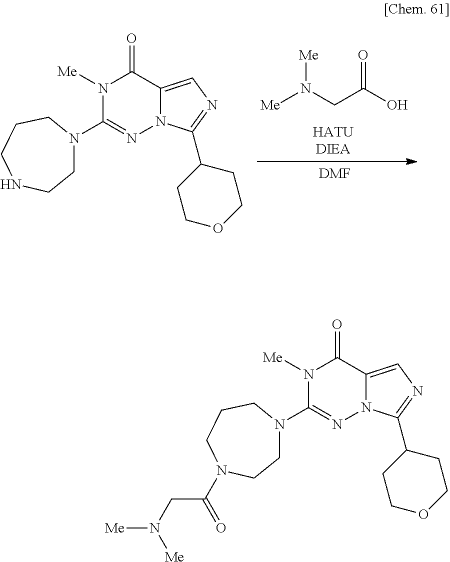

To a solution of N,N-dimethylglycine (10 mg, 0.099 mmol) and 2-(1,4-diazepan-1-yl)-3-methyl-7-(tetrahydro-2H-pyran-4-yl)imidazo[5,1-f][1,2,4]triazin-4(3H)-one (22 mg, 0.066 mmol) in DMF (3 mL) were added HATU (38 mg, 0.099 mmol) and DIEA (21 mg, 0.166 mmol). The reaction mixture was stirred at room temperature overnight and purified by preparative HPLC (MeCN and H₂O with 0.01% NH₃.H₂O as mobile phase) to give the titled compound as a white solid (10 mg, yield 36%).

LC-MS (m/z)=418.2 [M+H]⁺; ¹H-NMR (400 MHz, CDCl₃): δ 1.87-1.92 (m, 2H), 2.04-2.12 (m, 4H), 2.29-2.33 (m, 6H), 3.16-3.18 (m, 2H), 3.30-3.41 (m, 4H), 3.46-3.51 (m, 4H), 3.55-3.61 (m, 2H), 3.71-3.78 (m, 2H), 3.82-3.87 (m, 2H), 4.08-4.10 (m, 2H), 7.61-7.77 (s, 1H).

### Example 223

### 2-[(4-Chlorophenoxy)methyl]-3-methyl-7-(propan-2-yl)imidazo[5,1-f][1,2,4]triazin-4(3H)-one

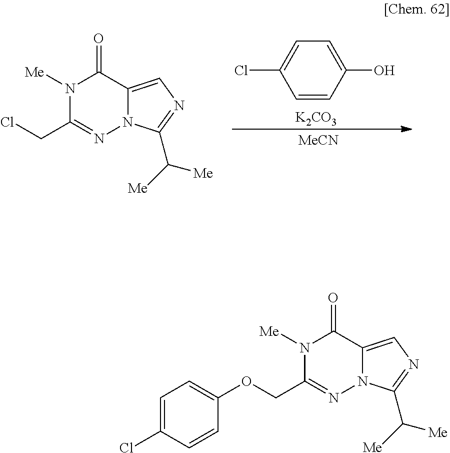

To the mixture of 2-(chloromethyl)-3-methyl-7-(propan-2-yl)imidazo[5,1-f][1,2,4]triazin-4(3H)-one (111 mg, 0.46 mmol) and K₂CO₃ (127 mg, 0.92 mmol) in MeCN (4 mL) was added 4-chlorophenol (71 mg, 0.55 mmol) at r.t. The mixture was stirred at 25° C. for 12 h. The reaction mixture was filtered and purified by preparative HPLC (MeCN and H₂O with 0.05% NH₃.H₂O as mobile phase) to give the titled compound (55 mg, yield 37%).

¹H-NMR (400 MHz, CDCl₃): δ 1.42 (d, J=7.2 Hz, 6H), 3.51-3.54 (m, 1H), 3.62 (s, 3H), 5.07 (s, 2H), 6.99-7.01 (m, 2H), 7.28-7.33 (m, 2H), 7.84 (s, 1H).

### Example 224

### 2-[(4-Chlorophenoxy)methyl]-7-cyclopentyl-3-methylimidazo[5,1-f][1,2,4]triazin-4(3H-one

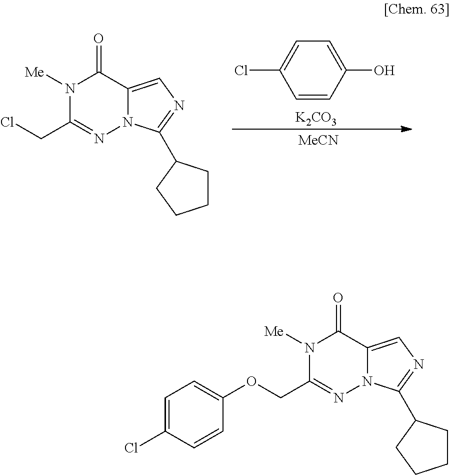

To the mixture of 2-(chloromethyl)-7-cyclopentyl-3-methylimidazo[5,1-f][1,2,4]triazin-4(3H)-one (100 mg, 0.37 mmol) and K₂CO₃ (102 mg, 0.74 mmol) in MeCN (4 mL) was added 4-chlorophenol (100 mg, 0.44 mmol) at r.t. And then the mixture was heated to 80° C. for 12 h. The reaction mixture was filtered and purified by preparative HPLC (MeCN and H₂O with 0.05% NH₃.H₂O as mobile phase) to give the titled compound (25 mg, yield 19%).

¹H-NMR (400 MHz, CDCl₃): δ 1.72-1.75 (m, 2H), 1.89-1.97 (m, 4H), 2.12-2.19 (m, 2H), 3.55-3.63 (m, 4H), 5.06 (s, 2H), 6.98-7.02 (m, 2H), 7.29-7.32 (m, 2H), 7.83 (s, 1H).

The compounds of Examples 225 to 228 were synthesized in a similar manner to Example 223 or 224.

[Chem. 64]

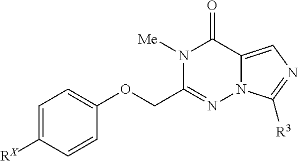

| No. | R^{X}— | R³— | ¹H-NMR |
|---|---|---|---|
| 225 | Me— | 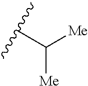 [SMILES: *C(C)C] | (400 MHz, CDCl₃): δ 1.42 (d, J = 6.8 Hz, 6H), 2.33 (s, 3H), 3.45-3.55 (m, 1H), 3.63 (s, 3H), 5.06 (s, 2H), 6.92- 6.97 (m, 2H), 7.12-7.17 (m, 2H), 7.82 (s, 1H). |
| 226 | F— | 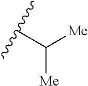 [SMILES: *C(C)C] | (400 MHz, CDCl₃): δ 1.42 (d, J = 7.2 Hz, 6H), 3.49-3.56 (m, 1H), 3.63 (s, 3H), 5.06 (s, 2H), 6.99-7.29 (m, 4H), 7.83 (s, 1H). |
| 227 | Me— | 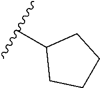 [SMILES: *C1CCCC1] | (400 MHz, CDCl₃): δ 1.72-1.75 (m, 2H), 1.89-1.97 (m, 4H), 2.12-2.14 (m, 2H), 2.33 (s, 3H), 3.58-3.62 (m, 4H), 5.05 (s, 2H), 6.95 (d, J = 8.4 Hz, 2H), 7.15 (d, J = 8.0 Hz, 2H), 7.82 (s, 1H). |
| 228 | F— | 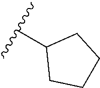 [SMILES: *C1CCCC1] | (400 MHz, CDCl₃): δ 1.74-1.75 (m, 2H), 1.88-1.96 (m, 4H), 2.12-2.14 (m, 2H), 3.55-3.62 (m, 4H), 5.05 (s, 2H), 6.98-7.07 (m, 4H), 7.82 (s, 1H). |

### Example 229

### 2-[(6-Chloro-3,4-dihydroquinolin-1(2H)-yl)methyl]-3-methyl-7-(propan-2-yl)imidazo[5,1-f][1,2,4]triazin-4(3H)-one

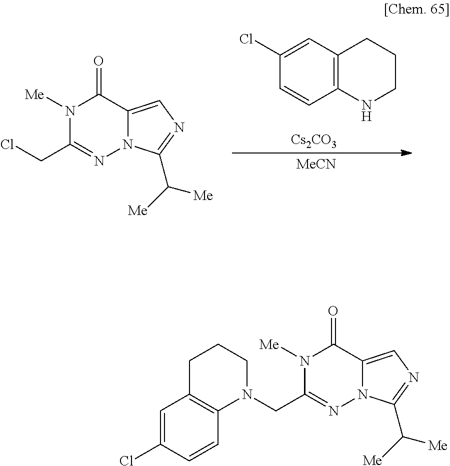 [SMILES: "MeCN" ; CC(C)c1ncc2c(=O)n(C)c(CCl)nn12 ; "Cs2CO3" ; Clc1ccc2c(c1)CCCN2 ; CC(C)c1ncc2c(=O)n(C)c(CN3CCCc4cc(Cl)ccc43)nn12]

A mixture of 2-(chloromethyl)-3-methyl-7-(propan-2-yl)imidazo[5,1-f][1,2,4]triazin-4(3H)-one (96 mg, 0.4 mmol), Cs₂CO₃ (260 mg, 0.8 mmol) and 6-chloro-1,2,3,4-tetrahydroquinoline (134 mg, 0.8 mmol) in MeCN (3 mL) was heated at 80° C. over 12 h. The precipitate was filtered and the filtrate was purified by preparative HPLC (MeCN and H₂O with 0.05% NH₃.H₂O as mobile phase) to give the titled compound (40 mg, yield 27%).

¹H-NMR (400 MHz, CDCl₃): δ 1.30 (d, J=6.8 Hz, 6H), 2.03-2.06 (m, 2H), 2.80-2.84 (m, 2H), 3.37-3.41 (m, 3H), 3.54 (s, 3H), 4.49 (s, 2H), 6.50-6.52 (m, 1H), 6.98-7.03 (m, 2H), 7.85 (s, 1H).

### Example 230

### 3-Methyl-2-({methyl[4-(trifluoromethyl)phenyl]amino}methyl)-7-(propan-2-yl)imidazo[5,1-f][1,2,4]triazin-4(3H)-one

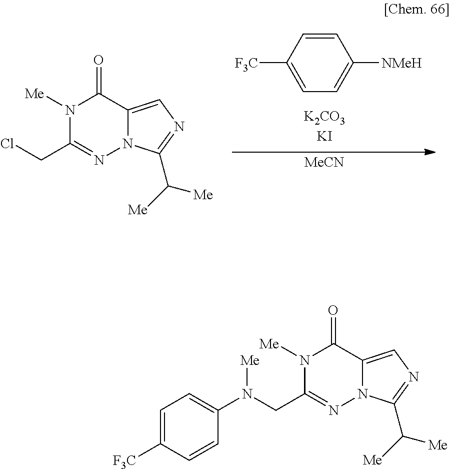

A mixture of 2-(chloromethyl)-3-methyl-7-(propan-2-yl)imidazo[5,1-f][1,2,4]triazin-4(3H)-one (100 mg, 0.42 mmol), N-methyl-4-(trifluoromethyl)aniline (88 mg, 0.50 mmol), K₂CO₃ (116 mg, 0.84 mmol) and KI (14 mg, 0.084 mmol) in MeCN (3 mL) was stirred at 50° C. for 16 h. The reaction mixture was filtered and purified by preparative HPLC (MeCN and H₂O with 0.05% NH₃.H₂O as mobile phase) to give the titled compound (15 mg, yield 10%).

¹H-NMR (400 MHz, CD₃CN): δ 1.08 (d, J=7.2 Hz, 6H), 3.11 (sept, J=7.2 Hz, 1H), 3.18 (s, 3H), 3.44 (s, 3H), 4.73 (s, 2H), 6.93 (d, J=8.4 Hz, 2H), 7.49 (d, J=8.4 Hz, 2H), 7.60 (s, 1H).

### Example 231

### 7-Cyclopentyl-3-methyl-2-[(1-oxo-1,3-dihydro-2H-isoindol-2-yl)methyl]imidazo[5,1-f][1,2,4]triazin-4(3H)-one

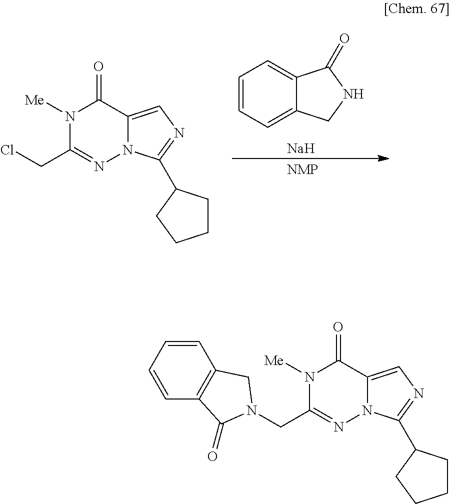

To a solution of 2,3-dihydro-1H-isoindol-1-one (60 mg) in anhydrous NMP (2 mL) was added NaH (60% in mineral oil, 30 mg) at 0° C. under N₂ atmosphere and the mixture was stirred at r.t. for 20 min. After cooling to 0° C., 2-(chloromethyl)-7-cyclopentyl-3-methylimidazo[5,1-f][1,2,4]triazin-4(3H)-one (100 mg) in NMP (1 mL) was added thereto and stirred for 30 min at 0° C. The reaction mixture was quenched with water (3 mL) and extracted with EtOAc (5 mL×3). The combined organic phases were washed with water (10 mL×2) and concentrated to give a crude product, which was purified by preparative HPLC (MeCN and H₂O with 0.05% NH₃.H₂O as mobile phase) to afford the the titled compound (23 mg, yield 17%).

¹H-NMR (400 MHz, CDCl₃): δ 1.58-1.72 (m, 4H), 1.88-1.98 (m, 4H), 3.37 (quint, J=8.4 Hz, 1H), 3.54 (s, 3H), 4.52 (s, 2H), 4.87 (s, 2H), 7.49-7.54 (m, 2H), 7.60-7.61 (m, 1H), 7.81 (s, 1H), 7.92 (d, J=7.6 Hz, 1H).

The compounds of Examples 232 to 235 were synthesized in a similar manner to Example 229, 230 or 231.

[Chem. 68]

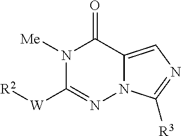

| No. | R²—W— | R³— | ¹H-NMR |
|---|---|---|---|
| 232 | 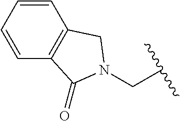 [SMILES: *CN1Cc2ccccc2C1=O] | 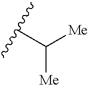 [SMILES: *C(C)C] | (400 MHz, CDCl₃): δ 1.33 (d, J = 6.8 Hz, 6H), 3.35-3.39 (m, 1H), 3.56 (s, 3H), 4.54 (s, 2H), 4.91 (s, 2H), 7.50-7.57 (m, 2H), 7.62-7.64 (m, 1H), 7.82 (s, 1H), 7.94 (d, J = 7.2 Hz, 1H). |
| 233 | 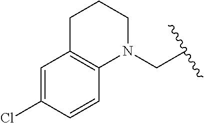 [SMILES: *CN1CCCc2cc(Cl)ccc21] | 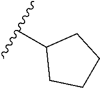 [SMILES: *C1CCCC1] | (400 MHz, CDCl₃): δ 1.60-1.65 (m, 2H), 1.78-1.84 (m, 4H), 1.93-1.95 (m, 2H), 2.02-2.05 (m, 2H), 2.79-2.83 (m, 2H), 3.37-3.41 (m, 3H), 3.52 (s, 3H), 4.47 (s, 2H), 6.52-6.54 (m, 1H), 6.98-7.02 (m, 2H), 7.80 (s, 1H). |
| 234 | 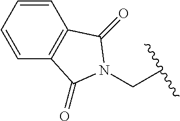 [SMILES: *CN1C(=O)c2ccccc2C1=O] | 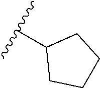 [SMILES: *C1CCCC1] | (400 MHz, CDCl₃): δ 1.22-1.52 (m, 4H), 1.60-1.74 (m, 4H), 2.97 (quint, J = 8.4 Hz, 1H), 3.60 (s, 3H), 4.94 (s, 2H), 7.76 (s, 1H), 7.85-7.87 (m, 2H), 7.99-8.00 (m, H). |
| 235 | 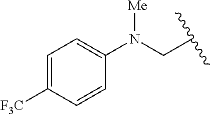 [SMILES: *CN(C)c1ccc(C(F)(F)F)cc1] | 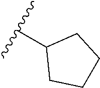 [SMILES: *C1CCCC1] | (400 MHz, CDCl₃): δ 1.58-1.89 (m, 8H), 3.17 (s, 3H), 3.25-3.34 (m, 1H), 3.51 (s, 3H), 4.63 (s, 2H), 6.83 (d, J = 8.4 Hz, 2H), 7.51 (d, J = 8.4 Hz, 2H), 7.79 (s, 1H). |

### Example 236

### 3-Methyl-2-(4-methylphenyl)-7-(tetrahydro-2H-pyran-4-yl)imidazo[5,1-f][1,2,4]triazin-4(3H)-one

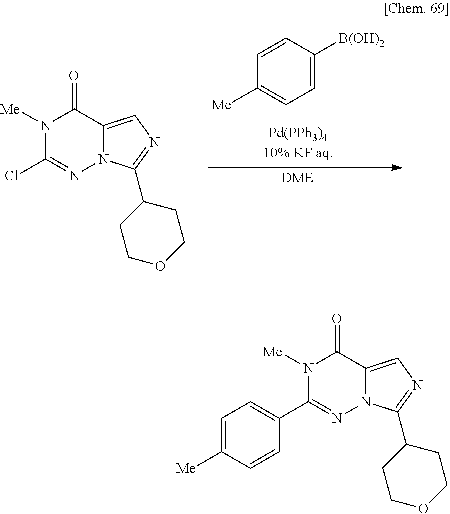 [SMILES: Cn1c(Cl)nn2c(C3CCOCC3)ncc2c1=O ; Cc1ccc(B(O)O)cc1 ; "DME" ; Cc1ccc(-c2nn3c(C4CCOCC4)ncc3c(=O)n2C)cc1]

To a mixture of 2-chloro-3-methyl-7-(tetrahydro-2H-pyran-4-yl)imidazo[5,1-f][1,2,4]triazin-4(3H)-one (50 mg, 0.186 mmol), 4-methylphenylbronic acid (64 mg, 0.372 mmol) and 10% KF aq. (0.35 mL) in DME (1.0 mL) was added tetrakis(triphenylphosphine)palladium (42 mg, 0.028 mmol) under nitrogen atmosphere. The mixture was heated for 1 h at 120° C. under microwave irradiation. The reaction was quenched with water and extracted with ethyl acetate. The combined organic extracts were washed with brine, dried over sodium sulfate and filtered. After concentration under reduced pressure, the residue was purified by silica gel column chromatography (hexane/EtOAc) to give a crude solid. The solid was washed with iPr₂O/IPA to give the titled compound (11 mg, yield 18%). LC-MS (m/z)=325 [M+H]+.

### Example 237

### 3-[(4,4-Difluorocyclohexyl)methyl]-2-methyl-7-(tetrahydro-2H-pyran-4-yl)imidazo[5,1-f][1,2,4]triazin-4(3H)-one

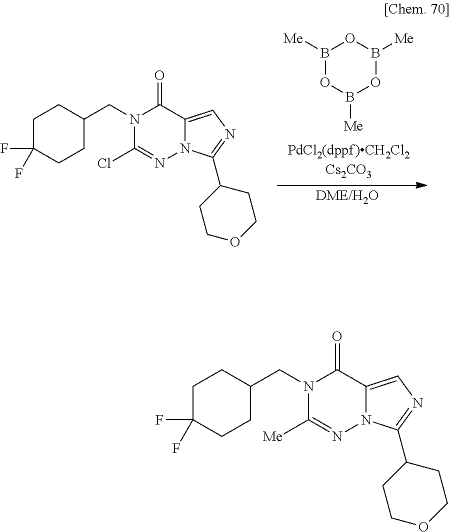 [SMILES: O=c1c2cnc(C3CCOCC3)n2nc(Cl)n1CC1CCC(F)(F)CC1 ; CB1OB(C)OB(C)O1 ; "DME/H2O" ; Cc1nn2c(C3CCOCC3)ncc2c(=O)n1CC1CCC(F)(F)CC1]

To a mixture of 2-chloro-3-[(4,4-difluorocyclohexyl)methyl]-7-(tetrahydro-2H-pyran-4-yl)imidazo[5,1-f][1,2,4]triazin-4(3H)-one (130 mg, 0.34 mmol), trimethylboroxine (105 mg, 0.84 mmol) and Cs₂CO₃ (329 mg, 1.01 mmol) in DME (2.0 mL) and H₂O (0.8 mL) was added [1,1′-bis(diphenylphosphino)ferrocene]palladium (II) chloride dichoromethane adduct (41 mg, 0.051 mmol) under nitrogen atmosphere. The mixture was heated for 1 h at 120° C. under microwave irradiation. The reaction was quenched with water, and extracted with ethyl acetate. The combined organic extracts were washed with brine, dried over sodium sulfate and filtered. After concentration under reduced pressure, the residue was purified by silica gel column chromatography (hexane/EtOAc) to give the titled compound (91 mg, yield 73%).

¹H-NMR (400 MHz, CDCl₃): δ 1.37-1.52 (m, 2H), 1.56-1.97 (m, 7H), 2.02-2.20 (m, 4H), 2.48 (s, 3H), 3.37-3.47 (m, 1H), 3.59 (td, J=11.7, 2.0 Hz, 2H), 3.87 (d, J=7.3 Hz, 2H), 4.05-4.13 (m, 2H), 7.78 (s, 1H).

### Example 238

### 3-Benzyl-2-methyl-7-(tetrahydro-2H-pyran-4-yl)imidazo[5,1-f][1,2,4]triazin-4(3H)-one

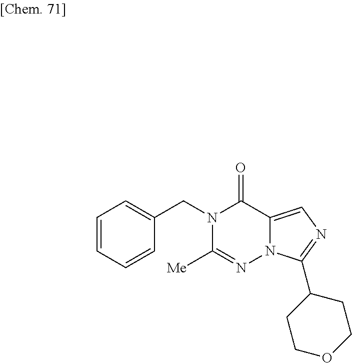 [SMILES: Cc1nn2c(C3CCOCC3)ncc2c(=O)n1Cc1ccccc1]

The titled compound was synthesized in a similar manner to Example 237.

¹H-NMR (300 MHz, CDCl₃): δ 1.87-1.92 (m, 2H), 2.02-2.16 (m, 2H), 2.37 (s, 3H), 3.36-3.46 (m, 1H), 3.52-3.61 (m, 2H), 4.05-4.10 (m, 2H), 5.22 (s, 2H), 7.18-7.36 (m, 5H), 7.84 (s, 1H).

### Example 239

### 3-[(4,4-Difluorocyclohexyl)methyl]-2-ethyl-7-(tetrahydro-2H-pyran-4-yl)imidazo[5,1-f][1,2,4]triazin-4(3H)-one

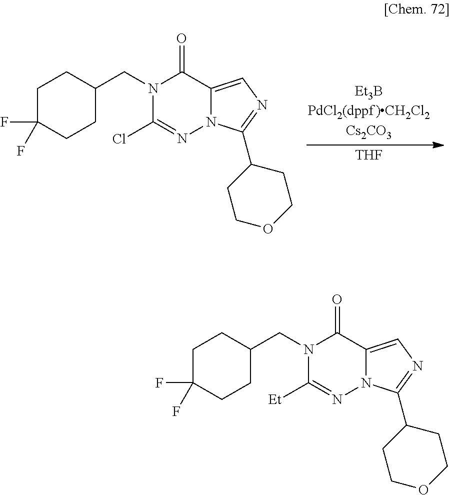 [SMILES: O=c1c2cnc(C3CCOCC3)n2nc(Cl)n1CC1CCC(F)(F)CC1 ; "THF" ; CCc1nn2c(C3CCOCC3)ncc2c(=O)n1CC1CCC(F)(F)CC1]

To a mixture of 2-chloro-3-[(4,4-difluorocyclohexyl)methyl]-7-(tetrahydro-2H-pyran-4-yl)imidazo[5,1-f][1,2,4]triazin-4(3H)-one (50 mg, 0.13 mmol), Cs₂CO₃ (127 mg, 0.39 mmol) and [1,1′-bis(diphenylphosphino)ferrocene]palladium (II) chloride dichoromethane adduct (21 mg, 0.026 mmol) in THF (1 mL) was added triethylborane (ca. 1 mol/l in THF, 0.26 ml, 0.26 mmol). The mixture was heated overnight at 60° C. under nitrogen atmosphere. The reaction was quenched with water, and extracted with ethyl acetate. The combined organic extracts were washed with brine, dried over sodium sulfate and filtered. After concentration under reduced pressure, the residue was purified by amino silica gel column chromatography (hexane/EtOAc) to give the titled compound (23 mg, yield 46%) as a white solid.

LC-MS (m/z)=381 [M+H]⁺. ¹H-NMR (400 MHz, CDCl₃): δ 1.37 (t, J=7.2 Hz, 3H), 1.41-1.51 (m, 2H), 1.59-1.71 (m, 1H), 1.71-1.81 (m, 3H), 1.81-1.98 (m, 3H), 2.04-2.20 (m, 4H), 2.75 (q, J=7.3 Hz, 2H), 3.38-3.49 (m, 1H), 3.55-3.65 (m, 2H), 3.84-3.92 (m, 2H), 4.06-4.14 (m, 2H), 7.79 (s, 1H).

### Example 240

### 2-[(E)-2-(4-Fluorophenyl)ethenyl]-3-methyl-7-(tetrahydro-2H-pyran-4-yl)imidazo[5,1-f][1,2,4]triazin-4(3H)-one

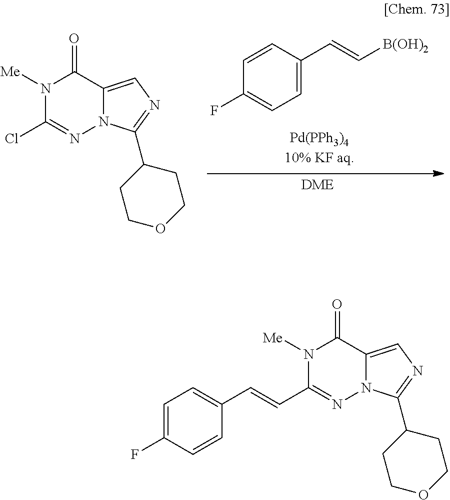 [SMILES: Cn1c(Cl)nn2c(C3CCOCC3)ncc2c1=O ; OB(O)/C=C/c1ccc(F)cc1 ; Cn1c(/C=C/c2ccc(F)cc2)nn2c(C3CCOCC3)ncc2c1=O]

To a solution of 2-chloro-3-methyl-7-(tetrahydro-2H-pyran-4-yl)imidazo[5,1-f][1,2,4]triazin-4(3H)-one (0.1 g, 0.373 mmol) and [(E)-2-(4-fluorophenyl)ethenyl]boronic acid (124 mg, 0.746 mmol) in DME (1.5 mL) and 10% KF aq. (0.5 mL) was added tetrakis(triphenylphosphine)palladium (64.7 mg, 0.056 mmol). The mixture was heated for 1 hour at 120° C. under nitrogen atmosphere and microwave irradiation. Upon completion, the reaction mixture was cooled and partitioned between ethyl acetate and brine. The aqueous layer was extracted with EtOAc, and the combined organic phases were dried with sodium sulfate and concentrated to dryness. The residue was purified by amino silica gel column chromatography (hexane/EtOAc) to give the titled compound (53 mg, yield 40%) as a white solid.

LC-MS (m/z)=355 [M+H]⁺. ¹H-NMR (400 MHz, CDCl₃): δ 1.91-1.96 (m, 2H), 2.05-2.15 (m, 2H), 3.45-3.54 (m, 1H), 3.56-3.63 (m, 5H), 4.06-4.10 (m, 2H), 6.78 (d, J=15.6 Hz, 1H), 7.08-7.13 (m, 2H), 7.54-7.59 (m, 3H), 7.79 (s, 1H).

### Example 241

### 2-[2-(4-Fluorophenyl)ethyl]-3-methyl-7-(tetrahydro-2H-pyran-4-yl)imidazo[5,1-f][1,2,4]triazin-4(3H)-one

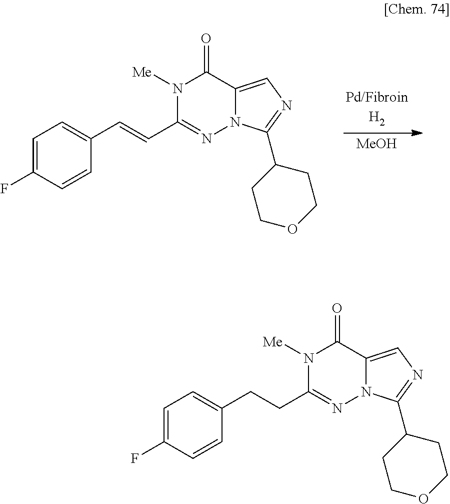 [SMILES: "MeOH" ; Cn1c(/C=C/c2ccc(F)cc2)nn2c(C3CCOCC3)ncc2c1=O ; Cn1c(CCc2ccc(F)cc2)nn2c(C3CCOCC3)ncc2c1=O]

To a solution of 2-[(E)-2-(4-fluorophenyl)ethenyl]-3-methyl-7-(tetrahydro-2H-pyran-4-yl)imidazo[5,1-f][1,2,4]triazin-4(3H)-one (32.9 mg, 0.093 mmol) in MeOH (1.0 mL) was added Pd/Fibroin (150 mg). The mixture was stirred for 4 hours at room temperature under H₂ atmosphere. Upon completion, the reaction mixture was filtered through a Celite filter, and the filtrate was concentrated to dryness. The residue was purified by amino silica gel column chromatography (hexane/EtOAc) to give the titled compound (27 mg, yield 82%) as a white solid.

LC-MS (m/z)=357 [M+H]⁺. ¹H-NMR (400 MHz, CDCl₃): δ 1.82-1.86 (m, 2H), 1.99-2.10 (m, 2H), 2.97-3.01 (m, 2H), 3.07-3.10 (m, 2H), 3.33-3.40 (m, 1H), 3.45 (s, 3H), 3.52-3.59 (m, 2H), 4.04-4.08 (m, 2H), 6.96-7.01 (m, 2H), 7.18-7.21 (m, 2H), 7.76 (s, 1H).

### Example 242

### 2-[(4-Methoxyphenyl)ethynyl]-3-methyl-7-(tetrahydro-2H-pyran-4-yl)imidazo[5,1-f][1,2,4]triazin-4(3H)-one

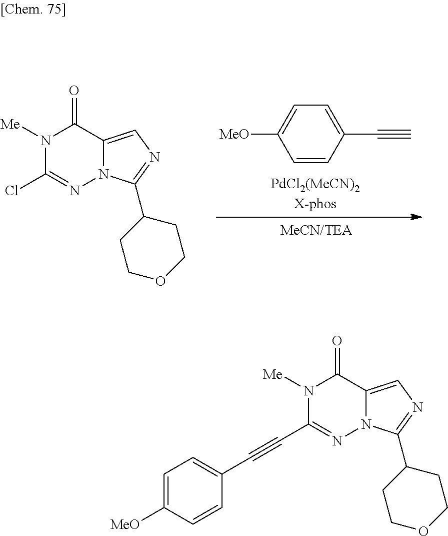

To a solution of 2-chloro-3-methyl-7-(tetrahydro-2H-pyran-4-yl)imidazo[5,1-f][1,2,4]triazin-4(3H)-one (50 mg, 0.187 mmol) and 1-ethynyl-4-methoxybenzene (87.4 μL, 0.674 mmol) in MeCN (0.3 mL) and TEA (0.3 mL) was added PdCl₂(MeCN)₂ (9.7 mg, 0.037 mmol) and x-phos (35.7 mg, 0.075 mmol). The mixture was heated overnight at 80° C. under nitrogen atmosphere. Upon completion, the reaction mixture was cooled and partitioned between ethyl acetate and brine. The aqueous layer was extracted with EtOAc, and the combined organic phases were dried with sodium sulfate and concentrated to dryness. The residue was purified by amino silica gel column chromatography (hexane/EtOAc) to give the titled compound (26 mg, yield 38%) as a white solid.

LC-MS (m/z)=365 [M+H]⁺.

### Example 243

### 2-[2-(4-Methoxyphenyl)ethyl]-3-methyl-7-(tetrahydro-2H-pyran-4-yl)imidazo[5,1-f][1,2,4]triazin-4(3H)-one

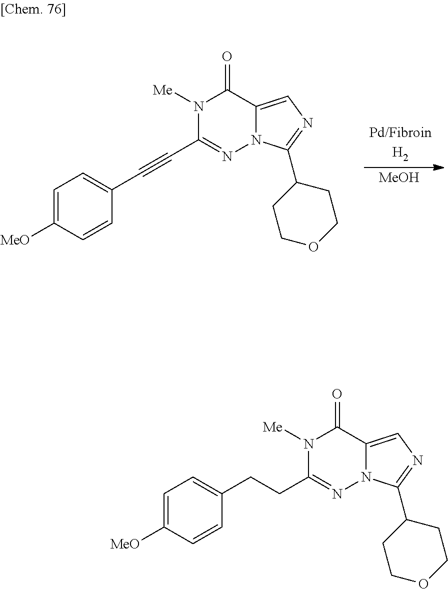 [SMILES: "MeOH" ; COc1ccc(C#Cc2nn3c(C4CCOCC4)ncc3c(=O)n2C)cc1 ; COc1ccc(CCc2nn3c(C4CCOCC4)ncc3c(=O)n2C)cc1]

To a solution of 2-[(4-methoxyphenyl)ethynyl]-3-methyl-7-(tetrahydro-2H-pyran-4-yl)imidazo[5,1-f][1,2,4]triazin-4(3H)-one (25.7 mg) in MeOH (1.0 mL) was added Pd/Fibroin (151 mg). The mixture was stirred for 4 hours at room temperature under H₂ atmosphere. Upon completion, the reaction mixture was filtered through a Celite filter, and the filtrate was concentrated to dryness. The residue was purified by amino silica gel column chromatography (hexane/EtOAc) to give the titled compound (15 mg, yield 58%) as a yellow solid.

LC-MS (m/z)=369 [M+H]⁺. ¹H-NMR (400 MHz, CDCl₃): δ 1.84-1.87 (m, 2H), 2.00-2.10 (m, 2H), 2.96-2.99 (m, 2H), 3.03-3.07 (m, 2H), 3.35-3.44 (m, 4H), 3.53-3.60 (m, 2H), 3.77 (s, 3H), 4.05-4.09 (m, 2H), 6.83 (d, J=8.8 Hz, 2H), 7.15 (d, J=8.8 Hz, 2H), 7.76 (s, 1H).

### Example 244

### 3-Methyl-2-[2-(pyridin-2-yl)ethyl]-7-(tetrahydro-2H-pyran-4-yl)imidazo[5,1-f][1,2,4]triazin-4(3H)-one

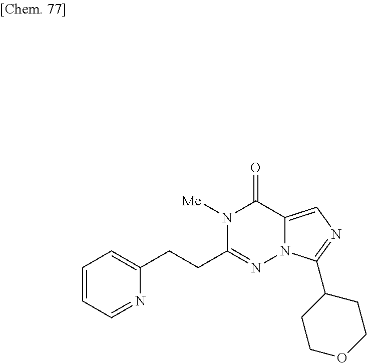 [SMILES: Cn1c(CCc2ccccn2)nn2c(C3CCOCC3)ncc2c1=O]

The titled compound was synthesized in a similar manner to Example 243.

¹H-NMR (400 MHz, CDCl₃): δ 1.74-1.78 (m, 2H), 1.94-2.04 (m, 2H), 3.25-3.33 (m, 5H), 3.49-3.55 (m, 5H), 4.02-4.05 (m, 2H), 7.11-7.14 (m, 1H), 7.24-7.26 (m, 1H), 7.59-7.63 (m, 1H), 7.74 (s, 1H), 8.50-8.51 (m, 1H).

### Example 245

### 3-Methyl-7-(tetrahydro-2H-pyran-4-yl)-2-{[trans-4-(trifluoromethyl)cyclohexyl]-methoxy}imidazo[5,1-f][1,2,4]triazin-4(3H)-one

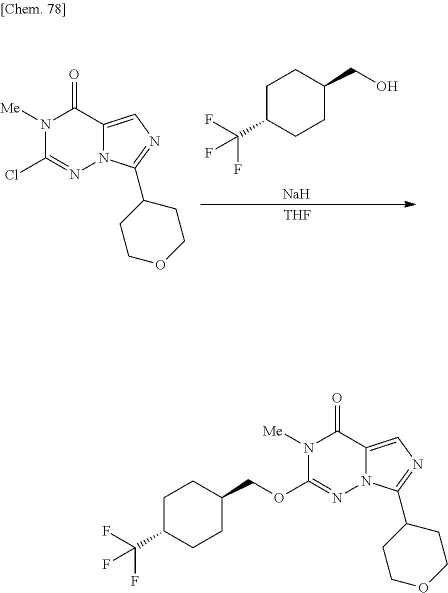

To a solution of [trans-4-(trifluoromethyl)cyclohexyl]methanol (1.017 g, 5.58 mmol) in THF (10 mL), sodium hydride (55% oil suspension, 292 mg, 6.7 mmol) was added at 0° C. The reaction mixture was stirred for 10 min at room temperature. After 2-chloro-3-methyl-7-(tetrahydro-2H-pyran-4-yl)imidazo[5,1-f][1,2,4]triazin-4(3H)-one (1 g, 3.72 mmol) was added, the mixture was stirred for 2 h at room temperature. Upon completion, the reaction mixture was quenched with H₂O (10 mL) and saturated NaCl aq. (10 mL). The aqueous layer was extracted with EtOAc (20 mL+10 mL×2) and the combined organic layer was concentrated in vacuo. The residue was purified by silica gel column chromatography (hexane/EtOAc) to give the crude product (947 mg). The crude was recrystallized with (IPA/heptane=1/3) to give the titled compound (839 mg, 2.025 mmol, 54%).

LC-MS (m/z)=415 [M+H]⁺. ¹H-NMR (400 MHz, CDCl₃): δ 1.12-1.26 (m, 2H), 1.32-1.48 (m, 2H), 1.84-1.95 (m, 3H), 1.97-2.17 (m, 7H), 3.29-3.39 (m, 1H), 3.41 (s, 3H), 3.54-3.63 (m, 2H), 4.05-4.13 (m, 2H), 4.21 (d, J=6.3 Hz, 2H), 7.78 (s, 1H).

The compounds of Examples 246 to 253 were synthesized in a similar manner to Example 1, 2, 3 or 245.

[Chem. 79]

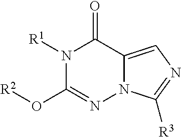

| No. | R¹— | R²— | R³— | ¹H-NMR |
|---|---|---|---|---|
| 246 | Me— | 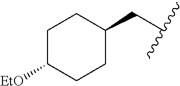 [SMILES: *C[C@H]1CC[C@H](OCC)CC1] | 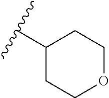 [SMILES: *C1CCOCC1] | (400 MHz, CDCl₃): δ 1.10-1.37 (m, 7H), 1.78-1.99 (m, 5H), 2.03- 2.20 (m, 4H), 3.18-3.28 (m, 1H), 3.29-3.39 (m, 1H), 3.41 (s, 3H), 3.51-3.63 (m, 4H), 4.05-4.13 (m, 2H), 4.18 (d, J = 6.1 Hz, 2H), |
| | | | | 7.78 (s, 1H). |
| 247 | Me— | 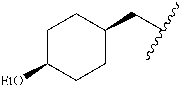 [SMILES: *C[C@H]1CC[C@@H](OCC)CC1] | 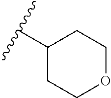 [SMILES: *C1CCOCC1] | (400 MHz, CDCl₃): δ 1.21 (t, J = 7.1 Hz, 3H), 1.41-1.67 (m, 6H), 1.86-1.99 (m, 5H), 2.02-2.17 (m, 2H), 3.30-3.40 (m, 1H), 3.41 (s, 3H), 3.47 (q, J = 7.1 Hz, 2H), 3.54-3.66 (m, 3H), 4.05-4.14 (m, |
| | | | | 2H), 4.21 (d, J = 6.6 Hz, 2H), |
| | | | | 7.77 (s, 1H). |
| 248 | Me— | 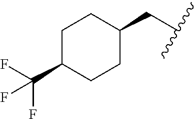 [SMILES: *C[C@H]1CC[C@@H](C(F)(F)F)CC1] | 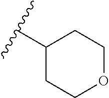 [SMILES: *C1CCOCC1] | (400 MHz, CDCl₃): δ 1.63-1.83 (m, 8H), 1.87-1.95 (m, 2H), 2.03- 2.29 (m, 4H), 3.31-3.40 (m, 1H), 3.41 (s, 3H), 3.54-3.64 (m, 2H), 4.06-4.14 (m, 2H), 4.36 (d, J = 7.1 Hz, 2H), 7.79 (s, 1H). |
| 249 | Et— | 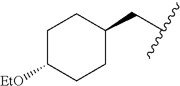 [SMILES: *C[C@H]1CC[C@H](OCC)CC1] | 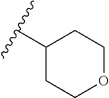 [SMILES: *C1CCOCC1] | (400 MHz, CDCl₃): δ 1.09-1.37 (m, 10H), 1.80-1.98 (m, 5H), 2.03-2.21 (m, 4H), 3.18-3.28 (m, 1H), 3.28-3.39 (m, 1H), 3.50-3.64 (m, 4H), 3.98-4.14 (m, 4H), 4.19 (d, J = 5.9 Hz, 2H), 7.77 (s, 1H). |
| 250 | Et— | 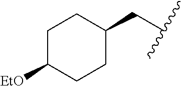 [SMILES: *C[C@H]1CC[C@@H](OCC)CC1] | 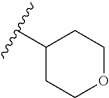 [SMILES: *C1CCOCC1] | (400 MHz, CDCl₃): δ 1.21 (t, J = 7.0 Hz, 3H), 1.28 (t, J = 7.1 Hz, 3H), 1.43-1.64 (m, 6H), 1.85-1.99 (m, 5H), 2.03-2.17 (m, 2H), 3.29- 3.40 (m, 1H), 3.47 (q, J = 7.0 Hz, 2H), 3.54-3.64 (m, 3H), 4.00-4.14 |
| | | | | (m, 4H), 4.22 (d, J = 6.1 Hz, 2H), |
| | | | | 7.77 (s, 1H). |
| 251 | Et— | 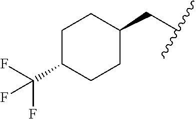 [SMILES: *C[C@H]1CC[C@H](C(F)(F)F)CC1] | 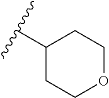 [SMILES: *C1CCOCC1] | (400 MHz, CDCl₃): δ 1.12-1.25 (m, 2H), 1.28 (t, J = 7.1 Hz, 3H), 1.34-1.48 (m, 2H), 1.83-1.95 (m, 3H), 1.96-2.18 (m, 7H), 3.28-3.39 (m, 1H), 3.53-3.63 (m, 2H), 4.03 (t, J = 7.1 Hz, 2H), 4.06-4.14 (m, 2H), 4.21 (d, J = 6.1 Hz, 2H), 7.77 (s, 1H). |
| 252 | Et— | 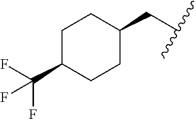 [SMILES: *C[C@H]1CC[C@@H](C(F)(F)F)CC1] | 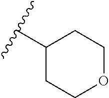 [SMILES: *C1CCOCC1] | (400 MHz, CDCl₃): δ 1.27 (t, J = 7.1 Hz, 3H), 1.63-1.83 (m, 8H), 1.86-1.96 (m, 2H), 2.03-2.30 (m, 4H), 3.30-3.41 (m, 1H), 3.55-3.64 (m, 2H), 4.04 (q, J = 7.1 Hz, 2H), 4.07-4.14 (m, 2H), 4.36 (d, J = 7.1 Hz, 2H), 7.78 (s, 1H). |
| 253 | 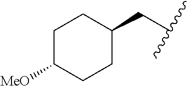 [SMILES: *C[C@H]1CC[C@H](OC)CC1] | Me— | 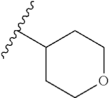 [SMILES: *C1CCOCC1] | (400 MHz, CDCl₃): δ 1.02-1.22 (m, 4H), 1.66-1.79 (m, 3H), 1.88- 1.99 (m, 2H), 2.04-2.20 (m, 4H), 3.04-3.15 (m, 1H), 3.31-3.42 (m, 1H), 3.34 (s, 3H), 3.53-3.64 (m, 2H), 3.84 (d, J = 6.8 Hz, 2H), 4.06 (s, 3H), 4.07-4.14 (m, 2H), |
| | | | | 7.77 (s, 1H). |

### Example 254

### 3-Methyl-2-[(trans-4-methylcyclohexyl)oxy]-7-(tetrahydro-2H-pyran-4-yl)imidazo[5,1-f][1,2,4]triazin-4(3H)-one

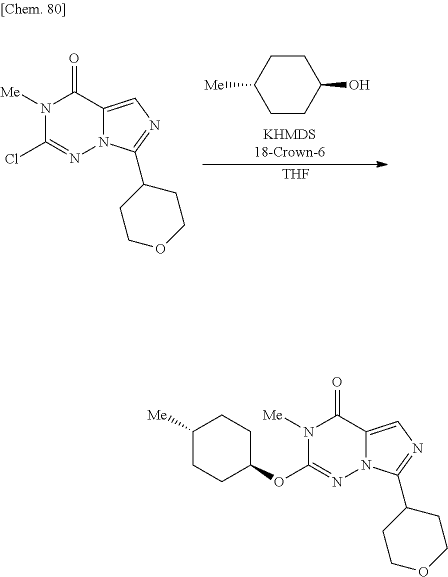 [SMILES: Cn1c(Cl)nn2c(C3CCOCC3)ncc2c1=O ; C[C@H]1CC[C@H](O)CC1 ; Cn1c(O[C@H]2CC[C@H](C)CC2)nn2c(C3CCOCC3)ncc2c1=O ; "THF"]

To a solution of trans-4-methylcyclohexanol (76 mg, 0.67 mmol) and 18-crown-6 (212 mg, 0.804 mmol) in THF (2.7 mL), potassium hexamethyldisilazide (160 mg, 0.804 mmol) was added at 0° C. Then, 2-chloro-3-methyl-7-(tetrahydro-2H-pyran-4-yl)imidazo[5,1-f][1,2,4]triazin-4(3H)-one (90 mg, 0.335 mmol) was added and the mixture was stirred for 15 min at same temperature. Upon completion, the reaction mixture was quenched with H₂O and saturated NaCl aq. The aqueous layer was extracted with EtOAc and the combined organic layer was concentrated in vacuo. The residue was purified by silica gel column chromatography (hexane/EtOAc) to give the titled compound (51 mg, 0.147 mmol, 44%).

LC-MS (m/z)=347 [M+H]⁺. ¹H-NMR (400 MHz, CDCl₃): δ 0.96 (d, J=6.6 Hz, 3H), 1.06-1.19 (m, 2H), 1.41-1.59 (m, 3H), 1.80-1.98 (m, 4H), 2.04-2.16 (m, 2H), 2.19-2.29 (m, 2H), 3.28-3.36 (m, 1H), 3.37 (s, 3H), 3.59 (td, J=11.6, 2.3 Hz, 2H), 4.07-4.14 (m, 2H), 4.80-4.90 (m, 1H), 7.77 (s, 1H).

The compounds of Examples 255 to 258 were synthesized in a similar manner to Example 1, 2 or 254.

[Chem. 81]

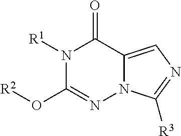

| No. | R¹— | R²— | R³— | 1H-NMR |
|---|---|---|---|---|
| 255 | Me— | 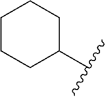 [SMILES: *C1CCCCC1] | 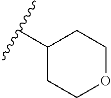 [SMILES: *C1CCOCC1] | (400 MHz, CDCl₃): δ 1.39-1.63 (m, 4H), 1.66-1.84 (m, 4H), 1.88- 1.96 (m, 2H), 1.97-2.17 (m, 4H), 3.27-3.37 (m, 1H), 3.40 (s, 3H), 3.52-3.63 (m, 2H), 4.05-4.15 (m, 2H), 4.97-5.06 (m, 1H), 7.77 (s, 1H). |
| 256 | Me— | 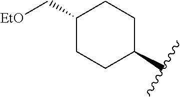 [SMILES: *[C@H]1CC[C@H](COCC)CC1] | 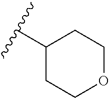 [SMILES: *C1CCOCC1] | (400 MHz, CDCl₃): δ 1.09-1.28 (m, 5H), 1.46-1.60 (m, 2H), 1.60- 1.74 (m, 1H), 1.87-2.02 (m, 4H), 2.03-2.17 (m, 2H), 2.24-2.35 (m, 2H), 3.24-3.36 (m, 3H), 3.38 (s, 3H), 3.49 (q, J = 7.0 Hz, 2H), 3.53-3.64 (m, 2H), 4.05-4.14 (m, |
| | | | | 2H), 4.80-4.92 (m, 1H), 7.77 (s, |
| | | | | 1H). |
| 257 | Et— | 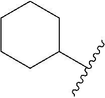 [SMILES: *C1CCCCC1] | 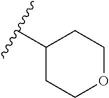 [SMILES: *C1CCOCC1] | (400 MHz, CDCl₃): δ 1.27 (t, J = 7.1 Hz, 3H), 1.42-1.61 (m, 4H), 1.68-1.85 (m, 4H), 1.87-1.96 (m, 2H), 1.96-2.17 (m, 4H), 3.27-3.37 (m, 1H), 3.58 (td, J = 11.7, 2.2 Hz, 2H), 4.00-4.14 (m, 4H), 4.99- 5.07 (m, 1H), 7.76 (s, 1H). |
| 258 | Et— | 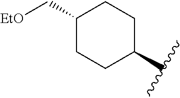 [SMILES: *[C@H]1CC[C@H](COCC)CC1] | 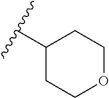 [SMILES: *C1CCOCC1] | (400 MHz, CDCl₃): δ 1.10-1.33 (m, 8H), 1.46-1.63 (m, 2H), 1.63- 1.75 (m, 1H), 1.86-2.04 (m, 4H), 2.04-2.19 (m, 2H), 2.24-2.36 (m, 2H), 3.25-3.38 (m, 3H), 3.44-3.54 (m, 2H), 3.54-3.66 (m, 2H), 3.98- 4.07 (m, 2H), 4.07-4.17 (m, 2H), |
| | | | | 4.80-4.96 (m, 1H), 7.76 (s, 1H). |

### Example 259

### 3-Ethyl-2-[(4-fluorophenoxy)methyl]-7-(tetrahydro-2H-pyran-4-yl)imidazo[5,1-f][1,2,4]triazin-4(3H)-one

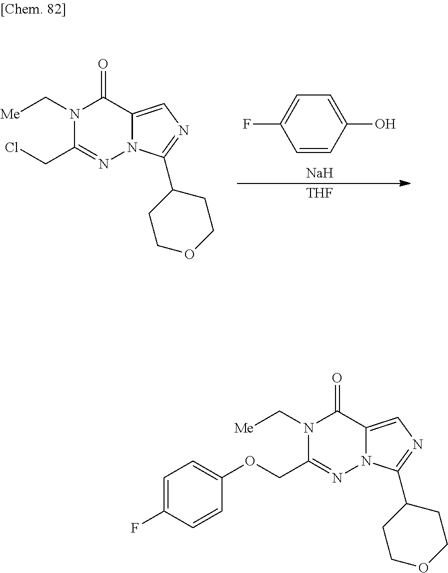

To a solution of 4-fluorophenol (17 mg, 0.152 mmol) in THF (1.0 mL), sodium hydride (55% oil suspension, 6.6 mg, 0.152 mmol) was added at 0° C. The reaction mixture was stirred for 10 min at the same temperature. After 2-(chloromethyl)-3-ethyl-7-(tetrahydro-2H-pyran-4-yl)imidazo[5,1-f][1,2,4]triazin-4(3H)-one (30 mg, 0.101 mmol) was added, the mixture was stirred for 17 h at room temperature. To complete the reaction, THF (4 mL), 4-fluorophenol (17 mg, 0.152 mmol) and sodium hydride (55% oil suspension, 7 mg, 0.161 mmol) was added and the mixture was stirred for 7 h at same temperature. Upon the completion, the mixture was quenched with H₂O and saturated NaCl aq. The aqueous layer was extracted with EtOAc and the combined organic layer was dried over Na₂SO₄ and concentrated in vacuo. The residue was purified by silica gel column chromatography (hexane/EtOAc) to give the titled compound (23 mg, yield 62%) as a white solid.

LC-MS (m/z)=373 [M+H]⁺. ¹H-NMR (400 MHz, CDCl₃): δ 1.39 (t, J=7.0 Hz, 3H), 1.85-1.97 (m, 2H), 2.04-2.18 (m, 2H), 3.36-3.50 (m, 1H), 3.53-3.66 (m, 2H), 4.04-4.22 (m, 4H), 5.02 (s, 2H), 6.94-7.09 (m, 4H), 7.83 (s, 1H).

### Example 260

### 2-{[(4,4-Difluorocyclohexyl)oxy]methyl}-3-ethyl-7-(tetrahydro-2H-pyran-4-yl)imidazo[5,1-f][1,2,4]triazin-4(3H)-one

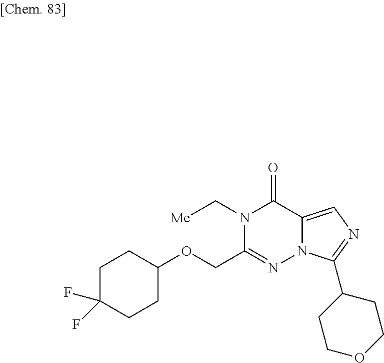 [SMILES: CCn1c(COC2CCC(F)(F)CC2)nn2c(C3CCOCC3)ncc2c1=O]

The titled compound was synthesized in a similar manner to Example 259.

LC-MS (m/z)=397 [M+H]⁺. ¹H-NMR (400 MHz, CDCl₃): δ 1.36 (t, J=7.1 Hz, 3H), 1.82-1.98 (m, 8H), 1.99-2.19 (m, 4H), 3.36-3.51 (m, 1H), 3.55-3.67 (m, 2H), 3.69-3.79 (m, 1H), 4.06-4.23 (m, 4H), 4.51 (s, 2H), 7.81 (s, 1H).

### Example 261

### 2-[(4,4-Difluorocyclohexyl)methoxy]-7-(tetrahydro-2H-pyran-4-yl)imidazo[5,1-f][1,2,4]triazin-4(3H)-one

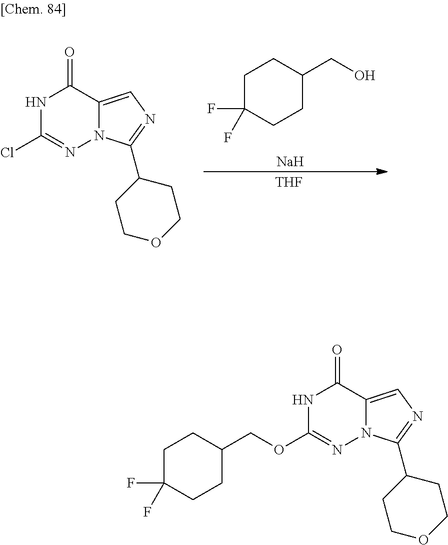

To a solution of (4,4-difluorocyclohexyl)methanol (347 mg, 2.3 mmol) in THF (50 mL) was added NaH (60% in oil, 93 mg, 2.3 mmol) at 0° C., then 2-chloro-7-(tetrahydro-2H-pyran-4-yl)imidazo[5,1-f][1,2,4]triazin-4(3H)-one (100 mg, 0.29 mmol) was added. The mixture was heated to 80° C. for 3 d. The solvent was evaporated under vacuum. The residue was dissolved in EtOAc (30 mL), and then washed with water (30 mL×3). The organic layer was dried over anhydrous Na₂SO₄, evaporated under vacuum to give crude product, which was used without further purification (105 mg, yield 98%).

LC-MS (m/z)=369 [M+H]⁺.

The compounds of Examples 262 to 277 were synthesized in a similar manner to Example 115 or Reference Example 7.

[Chem. 85]

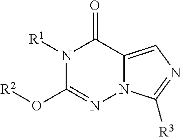

| No. | R¹— | R²— | R³— | ¹H NMR |
|---|---|---|---|---|
| 262 | 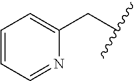 [SMILES: *Cc1ccccn1] | Me— | 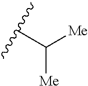 [SMILES: *C(C)C] | (400 MHz, CDCl₃): δ 1.41 (d, J = 6.8 Hz, 6H), 3.44-3.51 (m, 1H), 3.98 (s, 3H), 5.30 (s, 2H), 7.17- 7.22 (m, 2H), 7.63-7.67 (m, 1H), 7.82 (s, 1H), 8.52 (d, J = 4.8 Hz, 1H). |
| 263 | 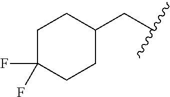 [SMILES: *CC1CCC(F)(F)CC1] | Me— | 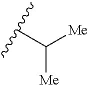 [SMILES: *C(C)C] | (400 MHz, CDCl₃): δ 1.32-1.39 (m, 8H), 1.53-1.68 (m, 4H), 1.79 (s, 1H), 2.01-2.04 (m, 2H), 3.34- 3.41 (m, 1H), 3.81 (d, J = 7.2 Hz, 2H), 3.40 (s, 3H), 7.70 (s, 1H). |
| 264 | 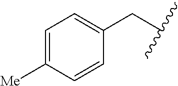 [SMILES: *Cc1ccc(C)cc1] | Me— | 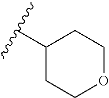 [SMILES: *C1CCOCC1] | (400 MHz, CDCl₃): δ 1.89-1.93 (m, 2H), 2.05-2.16 (m, 2H), 2.33 (s, 3H), 3.32-3.40 (m, 1H), 3.54- 3.61 (m, 2H), 4.04 (s, 3H), 4.07- 4.11 (m, 2H), 5.12 (s, 2H), 7.13 (d, J = 8.0 Hz, 2H), 7.28 (d, J = 8.4 Hz, 2H), 7.80 (s, 1H). |
| 265 | 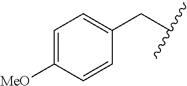 [SMILES: *Cc1ccc(OC)cc1] | Me— | 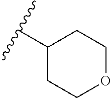 [SMILES: *C1CCOCC1] | (400 MHz, MeOD): δ 1.89-1.93 (m, 2H), 1.96-2.07 (m, 2H), 3.43- 3.51 (m, 1H), 3.56-3.63 (m, 2H), 3.77 (s, 3H), 4.03-4.06 (m, 2H), 4.08 (s, 3H), 5.10 (s, 2H), 6.86- 6.88 (d, J = 8.8 Hz, 2H), 7.29- 7.32 (d, J = 8.8 Hz, 2H), 7.73 (s, |
| | | | | 1H). |
| 266 | 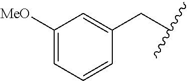 [SMILES: *Cc1cccc(OC)c1] | Me— | 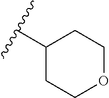 [SMILES: *C1CCOCC1] | (400 MHz, CDCl₃): δ 1.88-1.90 (m, 2H), 2.14-2.26 (m, 2H), 3.51 (t, J = 12.0, 1.2 Hz, 3H), 3.80 (s, 3H), 4.10 (s, 4H), 4.12-4.13 (m, 1H), 5.14 (s, 2H), 6.84 (dd, J = 8.4, 2.4 Hz, 1H), 6.90 (s, 1H), |
| | | | | 6.94 (d, J = 7.6 Hz, 1H), 7.26 (t, |
| | | | | J = 8.0 Hz, 1H), 8.01 (s, 1H). |
| 267 | 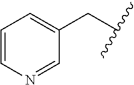 [SMILES: *Cc1cccnc1] | Me— | 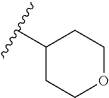 [SMILES: *C1CCOCC1] | (400 MHz, CDCl₃): δ 1.89-1.93 (m, 2H), 2.06-2.16 (m, 2H), 3.33- 3.42 (m, 1H), 3.55-3.61 (m, 2H), 4.07 (s, 3H), 4.09-4.11 (m, 2H), 5.17 (s, 2H), 7.28 (d, J = 4.8 Hz, 1H), 7.77-7.79 (m, 1H), 7.84 (s, |
| | | | | 1H), 8.55-8.57 (m, 1H), 8.67-8.68 |
| | | | | (m, 1H). |
| 268 | 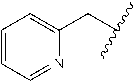 [SMILES: *Cc1ccccn1] | Me— | 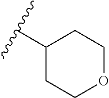 [SMILES: *C1CCOCC1] | (400 MHz, CDCl₃): δ 1.94-1.98 (m, 2H), 2.09-2.20 (m, 2H), 3.37- 3.45 (m, 1H), 3.57-3.63 (m, 2H), 3.99 (s, 3H), 4.10-4.13 (m, 2H), 5.31 (s, 2H), 7.18-7.23 (m, 2H), 7.64-7.69 (m, 1H), 7.83 (s, 1H), 8.52 (d, J = 4.4 Hz, 1H). |
| 269 | 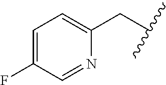 [SMILES: *Cc1ccc(F)cn1] | Me— | 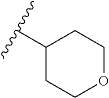 [SMILES: *C1CCOCC1] | (400 MHz, MeOD): δ 1.93-1.96 (m, 2H), 2.00-2.10 (m, 2H), 3.47- 3.55 (m, 1H), 3.58-3.65 (m, 2H), 4.01(s, 3H), 4.05-4.08 (m, 2H), 5.31(s, 2H), 7.44 (dd, J = 8.4, 4.4 Hz, 1H), 7.59 (td, J = 8.4, 2.8 Hz, 1H), 7.74 (s, 1H), 8.33 (d, J = 2.8 |
| | | | | Hz, 1H). |
| 270 | ⁿPr— | Me— | 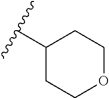 [SMILES: *C1CCOCC1] | (400 MHz, CDCl₃): δ 0.96 (t, J = 7.4 Hz, 3H), 1.63-1.72 (m, 2H), 1.91-1.95 (m, 2H), 2.07-2.17 (m, 2H), 3.33-3.41 (m, 1H), 3.56-3.62 (m, 2H), 3.87-3.95 (m, 2H), 4.06 (s, 3H), 4.08-4.12 (m, 2H), 7.77 |
| | | | | (s, 1H). |
| 271 | 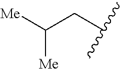 [SMILES: *CC(C)C] | Me— | 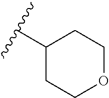 [SMILES: *C1CCOCC1] | (400 MHz, MeOD): δ 0.93 (d, J = 6.8 Hz, 6H), 1.91-1.94 (m, 2H), 1.97-2.13 (m, 3H), 3.44-3.52 (m, 1H), 3.58 (td, J = 12.0, 2.0 Hz, 2H), 3.82 (d, J = 8.0 Hz, 2H), 4.03-4.07 (m, 2H), 4.10 (s, 3H), |
| | | | | 7.67 (s, 1H). |
| 272 | 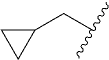 [SMILES: *CC1CC1] | Me— | 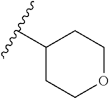 [SMILES: *C1CCOCC1] | (400 MHz, CDCl₃): δ 0.40-0.46 (m, 2H), 0.48-0.54 (m, 2H), 1.16- 1.23 (m, 1H), 1.92-1.96 (m, 2H), 2.08-2.18 (m, 2H), 3.34-3.42 (m, 1H), 3.56-3.62 (m, 2H), 3.86 (d, J = 6.8 Hz, 2H), 4.07 (s, 3H), 4.09- |
| | | | | 4.12 (m, 2H), 7.78 (s, 1H). |
| 273 | 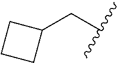 [SMILES: *CC1CCC1] | Me— | 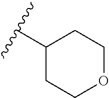 [SMILES: *C1CCOCC1] | (400 MHz, CDCl₃): δ 1.78-1.87 (m, 4H), 1.91-1.95 (m, 2H), 1.98- 2.05 (m, 2H), 2.06-2.16 (m, 2H), 2.62-2.70 (m, 1H), 3.32-3.40 (m, 1H), 3.55-3.61 (m, 2H), 4.01 (d, J = 7.6 Hz, 2H), 4.05 (s, 3H), 4.07- |
| | | | | 4.11 (m, 2H), 7.76 (s, 1H). |
| 274 | 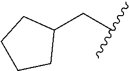 [SMILES: *CC1CCCC1] | Me— | 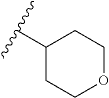 [SMILES: *C1CCOCC1] | (400 MHz, MeOD): δ 1.27-1.38 (m, 2H), 1.52-1.59 (m, 2H), 1.59- 1.70 (m, 4H), 1.91-1.94 (m, 2H), 1.94-2.07 (m, 2H), 2.28-2.36 (m, 1H), 3.44-3.51 (m, 1H), 3.58 (td, J = 12.0, 2.4 Hz, 2H), 3.94 (d, J = 7.6 Hz, 2H), 4.04-4.07 (m, 2H), |
| | | | | 4.10 (s, 3H), 7.69 (s, 1H). |
| 275 | 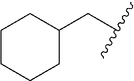 [SMILES: *CC1CCCCC1] | Me— | 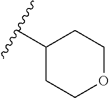 [SMILES: *C1CCOCC1] | (400 MHz, CDCl₃): δ 0.99-1.08 (m, 2H), 1.15-1.27 (m, 3H), 1.66- 1.78 (m, 6H), 1.93-1.96 (m, 2H), 2.06-2.18 (m, 2H), 3.34-3.42 (m, 1H), 3.57-3.63 (m, 2H), 3.82 (d, J = 7.2 Hz, 2H), 4.06 (s, 3H), 4.09- |
| | | | | 4.13 (m, 2H), 7.78 (s, 1H). |
| 276 | 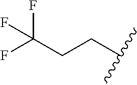 [SMILES: *CCC(F)(F)F] | Me— | 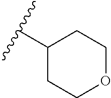 [SMILES: *C1CCOCC1] | (400 MHz, MeOD): δ 1.91-1.94 (m, 2H), 1.97-2.08 (m, 2H), 2.54- 2.66 (m, 2H), 3.45-3.53 (m, 1H), 3.61 (td, J = 12.0, 2.0 Hz, 2H), 4.04-4.05 (m, 2H), 4.13 (s, 3H), 4.25 (t, J = 7.2 Hz, 2H), 7.72 (s, |
| | | | | 1H). |
| 277 | 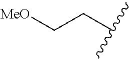 [SMILES: *CCOC] | 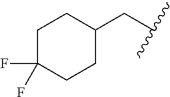 [SMILES: *CC1CCC(F)(F)CC1] | 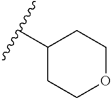 [SMILES: *C1CCOCC1] | (400 MHz, MeOD): δ 1.35-1.44 (m, 2H), 1.70-2.02 (m, 12H), 3.23 (s, 3H), 3.31-3.39 (m, 1H), 3.49- 3.53 (m, 3H), 3.93-3.95 (m, 2H), 4.09 (t, J = 5.6 Hz, 2H), 4.23 (d, J = 6.0 Hz, 2H), 7.58 (s, 1H) |

The compounds of Examples 278 to 298 were synthesized in a similar manner to Example 1, 2, 3 or 245.

[Chem. 86]

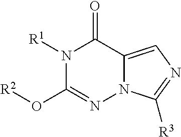

| No. | R¹— | R²— | R³— | ¹H NMR |
|---|---|---|---|---|
| 278 | 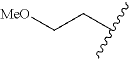 [SMILES: *CCOC] | 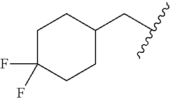 [SMILES: *CC1CCC(F)(F)CC1] | 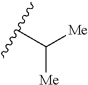 [SMILES: *C(C)C] | (400 MHz, CDCl₃): δ 1.39 (d, J = 6.8 Hz, 6H), 1.47-1.54 (m, 2H), 1.71-1.79 (m, 2H), 1.92- 1.96 (m, 3H), 2.18-2.19 (m, 2H), 3.34 (s, 3H), 3.39-3.45 (m, 1H), 3.61 (t, J = 5.6 Hz, 2H), 4.20 (t, J = 5.6 Hz, 2H), 4.25 (d, J = 6.0 |
| | | | | Hz, 2H), 7.78 (s, 1H). |
| 279 | 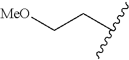 [SMILES: *CCOC] | 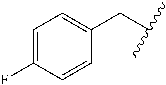 [SMILES: *Cc1ccc(F)cc1] | 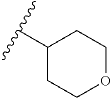 [SMILES: *C1CCOCC1] | (400 MHz, CDCl₃): δ 1.85-1.88 (m, 2H), 2.04-2.21 (m, 2H), 3.31 (s, 3H), 3.33-3.37 (m, 1H), 3.58- 3.61 (m, 4H), 4.09-4.12 (m, 2H), 4.21 (t, J = 4.0 Hz, 2H), 5.37 (s, 2H), 7.10 (t, J = 8.8 Hz, 2H), 7.43-7.47 (m, 2H), 7.77 (s, 1H). |
| 280 | 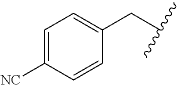 [SMILES: *Cc1ccc(C#N)cc1] | Me— | 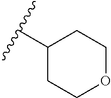 [SMILES: *C1CCOCC1] | (400 MHz, MeOD): δ 1.91-1.94 (m, 2H), 1.98-2.08 (m, 2H), 3.46-3.53 (m, 1H), 3.57-3.63 (td, J = 11.6, 2.0 Hz, 2H), 4.04-4.05 (m, 2H), 4.06 (s, 3H), 5.24 (s, 2H), 7.52 (d, J = 8.0 Hz, 2H), 7.71 (d, J = 8.0 Hz, 2H), 7.76 (s, |
| | | | | 1H). |
| 281 | 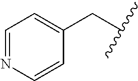 [SMILES: *Cc1ccncc1] | Me— | 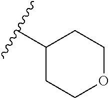 [SMILES: *C1CCOCC1] | (400 MHz, CDCl₃): δ 1.91-1.95 (m, 2H), 2.08-2.18 (m, 2H), 3.34-3.42 (m, 1H), 3.56-3.62 (m, 2H), 4.04 (s, 3H), 4.09-4.12 (m, 2H), 5.16 (s, 2H), 7.23 (d, J = 6.0 Hz, 2H), 7.86 (s, 1H), 8.58 (d, J = 5.6 Hz, 2H). |
| 282 | Me— | 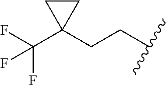 [SMILES: *CCC1(C(F)(F)F)CC1] | 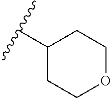 [SMILES: *C1CCOCC1] | (400 MHz, CDCl₃): δ 0.73 (s, 2H), 1.12 (t, J = 6.0 Hz, 2H), 1.90-1.93 (m, 2H), 2.02-2.15 (m, 4H), 3.31-3.38 (m, 1H), 3.40 (s, 3H), 3.55-3.62 (m, 2H), 4.08- 4.12 (m, 2H), 4.55 (t, J = 7.0 Hz, 2H), 7.79 (s, 1H). |
| 283 | Et— | 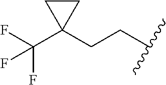 [SMILES: *CCC1(C(F)(F)F)CC1] | 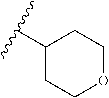 [SMILES: *C1CCOCC1] | (400 MHz, CDCl₃): δ 0.73 (s, 2H), 1.12 (t, J = 5.8 Hz, 2H), 1.26 (t, J = 7.2 Hz, 3H), 1.90- 1.93 (m, 2H), 2.05-2.14 (m, 4H), 3.31-3.38 (m, 1H), 3.55-3.62 (m, 2H), 4.03 (d, J = 7.0 Hz, 2H), 4.08-4.12 (m, 2H), 4.55 (t, J = |
| | | | | 7.0 Hz, 2H), 7.78 (s, 1H). |
| 284 | Me— | 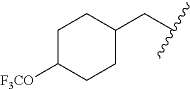 [SMILES: *CC1CCC(OC(F)(F)F)CC1] | 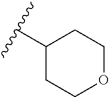 [SMILES: *C1CCOCC1] | (400 MHz, CDCl₃): δ 1.20-1.30 (m, 2H), 1.53-1.63 (m, 2H), 1.71-1.79 (m, 1H), 1.89-2.22 (m, 8H), 3.31-3.38 (m, 1H), 3.41 (s, 3H), 3.59 (tt, J = 2.4, 11.6 Hz, 2H), 4.10 (d, J = 11.6 Hz, 2H), 4.13-4.18 (m, 0.64H), 4.21 (dd, |
| | | | | J = 6.0, 12.0 Hz, 2H), 4.58 (br, |
| | | | | 0.36H), 7.78 (s, 1H). |
| 285 | Et— | 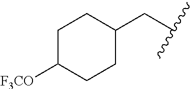 [SMILES: *CC1CCC(OC(F)(F)F)CC1] | 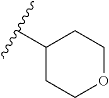 [SMILES: *C1CCOCC1] | (400 MHz, CDCl₃): δ 1.22-1.32 (m, 5H), 1.53-1.61 (m, 2H), 1.61-1.76 (m, 1H), 1.82-1.93 (m, 2H), 1.93-2.07 (m, 2H), 2.08- 2.17(m, 3H), 2.19-2.23 (m, 1H), 3.32-3.38 (m, 1H), 3.56-3.62 (m, |
| | | | | 2H), 4.03-4.06 (m, 2H). 4.06- |
| | | | | 4.16 (m, 2H), 4.16-4.22 (m, 2H), |
| | | | | 4.22-4.59 (m, 1H), 7.78 (s, 1H). |
| 286 | Me— | 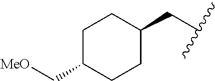 [SMILES: *C[C@H]1CC[C@H](COC)CC1] | 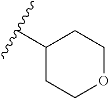 [SMILES: *C1CCOCC1] | (400 MHz, CDCl₃): δ 0.97-1.23 (m, 4H), 1.54-1.66 (m, 1H), 1.77-1.96 (m, 7H), 2.03-2.16 (m, 2H), 3.23 (d, J = 6.3 Hz, 2H), 3.29-3.39 (m, 4H), 3.41 (s, 3H), 3.59 (td, J = 11.6, 2.3 Hz, 2H), 4.06-4.13 (m, 2H), 4.18 (d, J = |
| | | | | 6.1 Hz, 2H), 7.77 (s, 1H). |
| 287 | Et— | 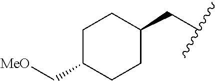 [SMILES: *C[C@H]1CC[C@H](COC)CC1] | 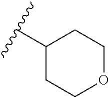 [SMILES: *C1CCOCC1] | (400 MHz, CDCl₃): δ 0.98-1.31 (m, 7H), 1.54-1.67 (m, 1H), 1.78-1.96 (m, 7H), 2.03-2.17 (m, 2H), 3.23 (d, J = 6.3 Hz, 2H), 3.29-3.39 (m, 4H), 3.59 (td, J = 11.7, 2.2 Hz, 2H), 4.00-4.14 (m, 4H), 4.19 (d, J = 5.9 Hz, 2H), |
| | | | | 7.77 (s, 1H). |
| 288 | Me— | 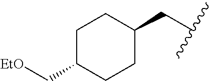 [SMILES: *C[C@H]1CC[C@H](COCC)CC1] | 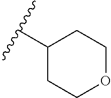 [SMILES: *C1CCOCC1] | (400 MHz, CDCl₃): δ 0.94-1.08 (m, 2H), 1.08-1.23 (m, 5H), 1.53-1.65 (m, 1H), 1.77-1.96 (m, 7H), 2.02-2.15 (m, 2H), 3.25 (d, J = 6.3 Hz, 2H), 3.29-3.42 (m, 4H), 3.47 (q, J = 7.0 Hz, 2H), 3.58 (td, J = 11.6, 2.2 Hz, 2H), |
| | | | | 4.03-4.12 (m, 2H), 4.17 (d, J = |
| | | | | 6.1 Hz, 2H), 7.76 (s, 1H). |
| 289 | Et— | 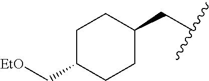 [SMILES: *C[C@H]1CC[C@H](COCC)CC1] | 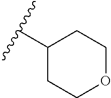 [SMILES: *C1CCOCC1] | (400 MHz, CDCl₃): δ 0.97-1.30 (m, 10H), 1.54-1.65 (m, 1H), 1.79-1.97 (m, 7H), 2.03-2.16 (m, 2H), 3.26 (d, J = 6.3 Hz, 2H), 3.30-3.39 (m, 1H), 3.48 (q, J = 7.1 Hz, 2H), 3.59 (td, J = 11.7, 2.2 Hz, 2H), 4.00-4.13 (m, 4H), |
| | | | | 4.19 (d, J = 5.9 Hz, 2H), 7.77 (s, |
| | | | | 1H). |
| 290 | Me— | 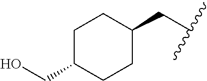 [SMILES: *C[C@H]1CC[C@H](CO)CC1] | 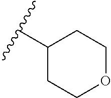 [SMILES: *C1CCOCC1] | (400 MHz, CDCl₃): δ 0.98-1.24 (m, 4H), 1.39-1.60 (m, 2H), 1.78-1.99 (m, 7H), 2.03-2.17 (m, 2H), 3.30-3.40 (m, 1H), 3.41 (s, 3H), 3.48-3.54 (m, 2H), 3.59 (td, J = 11.6, 2.2 Hz, 2H), 4.06-4.14 (m, 2H), 4.19 (d, J = 6.1 Hz, |
| | | | | 2H), 7.78 (s, 1H). |
| 291 | 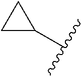 [SMILES: *C1CC1] | 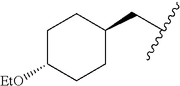 [SMILES: *C[C@H]1CC[C@H](OCC)CC1] | 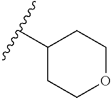 [SMILES: *C1CCOCC1] | (400 MHz, CDCl₃): δ 0.78-0.92 (m, 2H), 1.11-1.39 (m, 9H), 1.79-1.99 (m, 5H), 2.02-2.20 (m, 4H), 2.65-2.73 (m, 1H), 3.17- 3.36 (m, 2H), 3.50-3.61 (m, 4H), 4.04-4.11 (m, 2H), 4.16 (d, J = 6.1 Hz, 2H), 7.73 (s, 1H). |
| 292 | Me— | 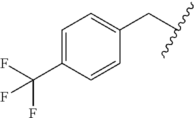 [SMILES: *Cc1ccc(C(F)(F)F)cc1] | 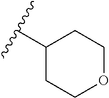 [SMILES: *C1CCOCC1] | (400 MHz, CDCl₃): δ 1.80-1.88 (m, 2H), 2.02-2.14 (m, 2H), 3.28-3.37 (m, 1H), 3.45 (s, 3H), 3.58 (td, J = 11.7, 2.2 Hz, 2H), 4.06-4.13 (m, 2H), 5.46 (s, 2H), 7.58 (d, J = 8.1 Hz, 2H), 7.70 (d, J = 8.1 Hz, 2H), 7.79 (s, 1H). |
| 293 | Me— | 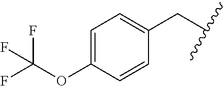 [SMILES: *Cc1ccc(OC(F)(F)F)cc1] | 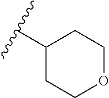 [SMILES: *C1CCOCC1] | (400 MHz, CDCl₃): δ 1.82-1.91 (m, 2H), 2.03-2.16 (m, 2H), 3.29-3.39 (m, 1H), 3.44 (s, 3H), 3.54-3.63 (m, 2H), 4.06-4.15 (m, 2H), 5.41 (s, 2H), 7.25-7.30 (m, 2H), 7.47-7.53 (m, 2H), 7.78 (s, 1H). |
| 294 | ¹³CD₃— | 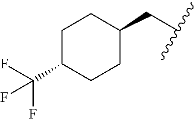 [SMILES: *C[C@H]1CC[C@H](C(F)(F)F)CC1] | 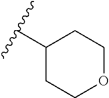 [SMILES: *C1CCOCC1] | (400 MHz, CDCl₃): δ 1.12-1.26 (m, 2H), 1.33-1.48 (m, 2H), 1.83-1.96 (m, 3H), 1.97-2.17 (m, 7H), 3.30-3.39 (m, 1H), 3.59 (td, J = 11.6, 2.3 Hz, 2H), 4.05-4.13 (m, 2H), 4.21 (d, J = 6.1 Hz, 2H), 7.78 (s, 1H). |
| 295 | Me— | 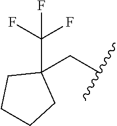 [SMILES: *CC1(C(F)(F)F)CCCC1] | 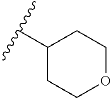 [SMILES: *C1CCOCC1] | (400 MHz, CDCl₃): δ 1.70-1.95 (m, 8H), 1.99-2.16 (m, 4H), 3.31-3.42 (m, 4H), 3.60 (td, J = 11.7, 2.2 Hz, 2H), 4.06-4.14 (m, 2H), 4.33 (s, 2H), 7.79 (s, 1H). |
| 296 | Et— | 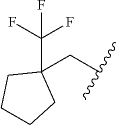 [SMILES: *CC1(C(F)(F)F)CCCC1] | 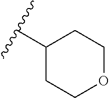 [SMILES: *C1CCOCC1] | (400 MHz, CDCl₃): δ 1.27 (t, J = 7.1 Hz, 3H), 1.69-1.95 (m, 8H), 1.99-2.16 (m, 4H), 3.30-3.40 (m, 1H), 3.60 (td, J = 11.6, 2.2 Hz, 2H), 3.99-4.15 (m, 4H), 4.33 (s, 2H), 7.79 (s, 1H). |
| 297 | Me— | 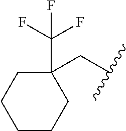 [SMILES: *CC1(C(F)(F)F)CCCCC1] | 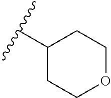 [SMILES: *C1CCOCC1] | (400 MHz, CDCl₃): δ 1.28-1.53 (m, 3H), 1.68-1.82 (m, 5H), 1.82-1.96 (m, 4H), 2.04-2.18 (m, 2H), 3.32-3.42 (m, 4H), 3.60 (td, J = 11.6, 2.3 Hz, 2H), 4.07-4.15 (m, 2H), 4.53 (s, 2H), 7.80 (s, 1H). |
| 298 | Me— | 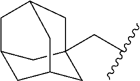 [SMILES: *CC12CC3CC(CC(C3)C1)C2] | 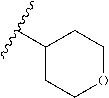 [SMILES: *C1CCOCC1] | (400 MHz, CDCl₃): δ 1.59-1.63 (m, 2H), 1.65-1.74 (m, 7H), 1.76-1.84 (m, 3H), 1.88-1.96 (m, 2H), 2.03-2.16 (m, 5H), 3.32- 3.41 (m, 1H), 3.43 (s, 3H), 3.60 (td, J = 11.6, 2.3 Hz, 2H), 3.92 (s, 2H), 4.06-4.13 (m, 2H), 7.77 |
| | | | | (s, 1H). |

The compounds of Examples 299 to 323 were synthesized in a similar manner to Example 1, 2 or 254.

[Chem. 87]

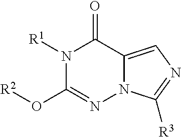

| No. | R¹— | R²— | R³— | ¹H-NMR |
|---|---|---|---|---|
| 299 | Me— | 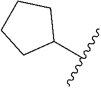 [SMILES: *C1CCCC1] | 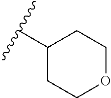 [SMILES: *C1CCOCC1] | (400 MHz, CDCl₃): δ 1.66-2.20 (m, 12H), 3.28-3.40 (m, 4H), 3.58 (td, J = 11.6, 2.3 Hz, 2H), 4.04-4.15 (m, 2H), 5.31-5.41 (m, 1H), 7.76 (s, 1H). |
| 300 | Et— | 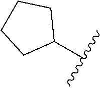 [SMILES: *C1CCCC1] | 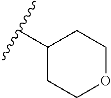 [SMILES: *C1CCOCC1] | (400 MHz, CDCl₃): δ 1.24 (t, J = 7.1 Hz, 3H), 1.67-2.20 (m, 12H), 3.28-3.40 (m, 1H), 3.58 (td, J = 11.6, 2.3 Hz, 2H), 4.01 (q, J = 7.1 Hz, 2H), 4.06-4.14 (m, 2H), 5.33-5.43 (m, 1H), 7.76 (s, 1H). |
| 301 | Me— | 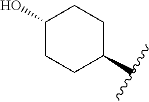 [SMILES: *[C@H]1CC[C@H](O)CC1] | 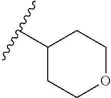 [SMILES: *C1CCOCC1] | (400 MHz, CDCl₃): δ 1.50-1.78 (m, 5H), 1.87-1.96 (m, 2H), 1.97-2.15 (m, 4H), 2.18-2.29 (m, 2H), 3.27-3.36 (m, 1H), 3.38 (s, 3H), 3.57 (td, J = 11.7, 2.2 Hz, 2H), 3.85-3.95 (m, 1H), 4.05-4.14 (m, 2H), 4.98- |
| | | | | 5.07 (m, 1H), 7.77 (s, 1H). |
| 302 | Me— | 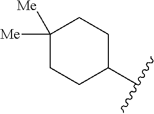 [SMILES: *C1CCC(C)(C)CC1] | 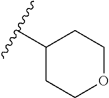 [SMILES: *C1CCOCC1] | (400 MHz, CDCl₃): δ 0.99 (s, 3H), 1.00 (s, 3H), 1.29-1.39 (m, 2H), 1.48-1.58 (m, 2H), 1.77-2.01 (m, 6H), 2.04-2.16 (m, 2H), 3.27-3.38 (m, 1H), 3.40 (s, 3H), 3.58 (td, J = 11.7, 2.2 Hz, 2H), 4.05-4.13 (m, |
| | | | | 2H), 4.96-5.05 (m, 1H), 7.77 |
| | | | | (s, 1H). |
| 303 | Me— | 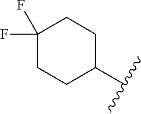 [SMILES: *C1CCC(F)(F)CC1] | 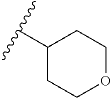 [SMILES: *C1CCOCC1] | (400 MHz, CDCl₃): δ 1.85-1.93 (m, 2H), 2.01-2.24 (m, 10H), 3.26-3.36 (m, 1H), 3.41 (s, 3H), 3.57 (td, J = 11.6, 2.4 Hz, 2H), 4.06-4.13 (m, 2H), 5.16- 5.27 (m, 1H), 7.79 (s, 1H). |
| 304 | Et— | 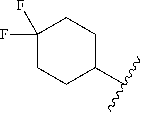 [SMILES: *C1CCC(F)(F)CC1] | 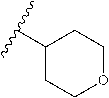 [SMILES: *C1CCOCC1] | (400 MHz, CDCl₃): δ 1.28 (t, J = 7.1 Hz, 3H), 1.85-1.94 (m, 2H), 2.00-2.25 (m, 10H), 3.25- 3.36 (m, 1H), 3.57 (td, J = 11.7, 2.2 Hz, 2H), 3.98-4.14 (m, 4H), 5.16-5.26 (m, 1H), 7.78 (s, 1H). |
| 305 | Me— | 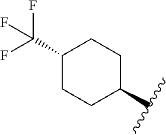 [SMILES: *[C@H]1CC[C@H](C(F)(F)F)CC1] | 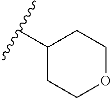 [SMILES: *C1CCOCC1] | (400 MHz, CDCl₃): δ 1.46-1.64 (m, 4H), 1.85-1.98 (m, 2H), 2.01-2.22 (m, 5H), 2.35-2.48 (m, 2H), 3.25-3.42 (m, 4H), 3.53-3.66 (m, 2H), 4.04-4.16 (m, 2H), 4.82-4.97 (m, 1H), 7.78 (s, 1H). |
| 306 | Et— | 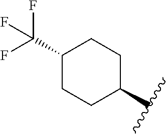 [SMILES: *[C@H]1CC[C@H](C(F)(F)F)CC1] | 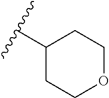 [SMILES: *C1CCOCC1] | (400 MHz, CDCl₃): δ 1.25 (t, J = 7.1 Hz, 3H), 1.50-1.63 (m, 4H), 1.86-1.96 (m, 2H), 2.02- 2.19 (m, 5H), 2.35-2.46 (m, 2H), 3.26-3.37 (m, 1H), 3.59 (td, J = 11.6, 2.2 Hz, 2H), 4.02 (q, J = 7.1 Hz, 2H), 4.07-4.15 (m, 2H), 4.84-4.96 (m, 1H), 7.78 (s, 1H). |
| 307 | Me— | 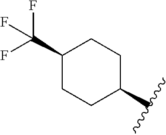 [SMILES: *[C@H]1CC[C@@H](C(F)(F)F)CC1] | 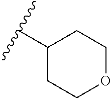 [SMILES: *C1CCOCC1] | (400 MHz, CDCl₃): δ 1.47-1.61 (m, 4H), 1.87-1.96 (m, 2H), 2.03-2.18 (m, 5H), 2.36-2.47 (m, 2H), 3.27-3.42 (m, 4H), 3.59 (td, J = 11.6, 2.1 Hz, 2H), 4.06-4.15 (m, 2H), 4.83-4.94 (m, 1H), 7.78 (s, 1H). |
| 308 | Et— | 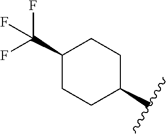 [SMILES: *[C@H]1CC[C@@H](C(F)(F)F)CC1] | 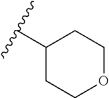 [SMILES: *C1CCOCC1] | (400 MHz, CDCl₃): δ 1.25 (t, J = 7.1 Hz, 3H), 1.50-1.62 (m, 4H), 1.86-1.96 (m, 2H), 2.03- 2.19 (m, 5H), 2.35-2.46 (m, 2H), 3.25-3.37 (m, 1H), 3.59 (td, J = 11.6, 2.2 Hz, 2H), 4.02 (q, J = 7.1 Hz, 2H), 4.07-4.15 (m, 2H), 4.83-4.97 (m, 1H), 7.78 (s, 1H). |
| 309 | Me— | 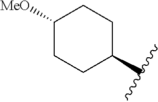 [SMILES: *[C@H]1CC[C@H](OC)CC1] | 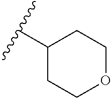 [SMILES: *C1CCOCC1] | (400 MHz, CDCl₃): δ 1.54-1.65 (m, 2H), 1.67-1.78 (m, 2H), 1.87-2.23 (m, 8H), 3.26-3.42 (m, 8H), 3.57 (td, J = 11.6, 2.1 Hz, 2H), 4.05-4.13 (m, 2H), 5.01-5.10 (m, 1H), 7.77 (s, 1H). |
| 310 | Et— | 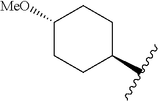 [SMILES: *[C@H]1CC[C@H](OC)CC1] | 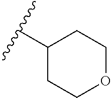 [SMILES: *C1CCOCC1] | (400 MHz, CDCl₃): δ 1.27 (t, J = 7.1 Hz, 3H), 1.55-1.67 (m, 2H), 1.68-1.80 (m, 2H), 1.87- 2.23 (m, 8H), 3.27-3.43 (m, 5H), 3.57 (td, J = 11.6, 2.1 Hz, 2H), 3.98-4.14 (m, 4H), 5.02- 5.11 (m, 1H), 7.77 (s, 1H). |
| 311 | 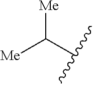 [SMILES: *C(C)C] | 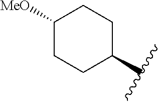 [SMILES: *[C@H]1CC[C@H](OC)CC1] | 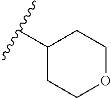 [SMILES: *C1CCOCC1] | (400 MHz, CDCl₃): δ 1.46 (d, J = 7.1 Hz, 6H), 1.55-1.66 (m, 2H), 1.68-1.81 (m, 2H), 1.87- 1.95 (m, 2H), 1.96-2.26 (m, 6H), 3.24-3.43 (m, 5H), 3.57 (td, J = 11.6, 2.3 Hz, 2H), 4.05- 4.13 (m, 2H), 5.02-5.12 (m, |
| | | | | 1H), 5.29-5.44 (m, 1H), 7.75 |
| | | | | (s, 1H). |
| 312 | Me— | 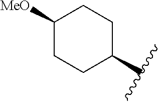 [SMILES: *[C@H]1CC[C@@H](OC)CC1] | 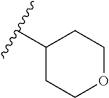 [SMILES: *C1CCOCC1] | (400 MHz, CDCl₃): δ 1.72-1.95 (m, 8H), 2.03-2.17 (m, 4H), 3.26-3.43 (m, 8H), 3.57 (td, J = 11.6, 2.1 Hz, 2H), 4.05-4.13 (m, 2H), 5.04-5.13 (m, 1H), 7.77 (s, 1H). |
| 313 | Et— | 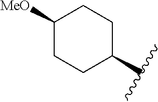 [SMILES: *[C@H]1CC[C@@H](OC)CC1] | 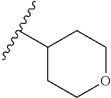 [SMILES: *C1CCOCC1] | (400 MHz, CDCl₃): δ 1.28 (t, J = 7.1 Hz, 3H), 1.72-1.95 (m, 8H), 2.04-2.18 (m, 4H), 3.26- 3.40 (m, 5H), 3.57 (td, J = 11.7, 2.2 Hz, 2H), 3.99-4.14 (m, 4H), 5.05-5.14 (m, 1H), 7.77 (s, 1H). |
| 314 | Me— | 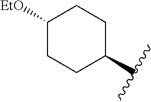 [SMILES: *[C@H]1CC[C@H](OCC)CC1] | 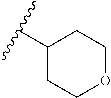 [SMILES: *C1CCOCC1] | (400 MHz, CDCl₃): δ 1.22 (t, J = 7.1 Hz, 3H), 1.53-1.64 (m, 2H), 1.65-1.77 (m, 2H), 1.87- 2.25 (m, 8H), 3.25-3.62 (m, 9H), 4.05-4.14 (m, 2H), 4.99- 5.08 (m, 1H), 7.77 (s, 1H). |
| 315 | Et— | 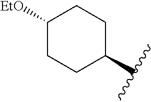 [SMILES: *[C@H]1CC[C@H](OCC)CC1] | 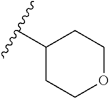 [SMILES: *C1CCOCC1] | (400 MHz, CDCl₃): δ 1.16-1.35 (m, 6H), 1.53-1.78 (m, 4H), 1.85-2.26 (m, 8H), 3.23-3.36 (m, 1H), 3.41-3.61 (m, 5H), 3.95-4.15 (m, 4H), 4.99-5.11 (m, 1H), 7.76 (s, 1H). |
| 316 | Me— | 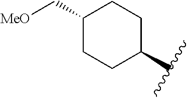 [SMILES: *[C@H]1CC[C@H](COC)CC1] | 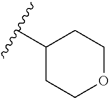 [SMILES: *C1CCOCC1] | (400 MHz, CDCl₃): δ 1.12-1.34 (m, 2H), 1.38-1.58 (m, 2H), 1.62-2.36 (m, 9H), 3.16-3.39 (m, 9H), 3.58 (td, J = 11.6, 2.3 Hz, 2H), 4.03-4.16 (m, 2H), 4.79-4.95 (m, 1H), 7.77 (s, 1H). |
| 317 | Et— | 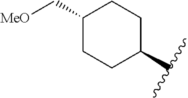 [SMILES: *[C@H]1CC[C@H](COC)CC1] | 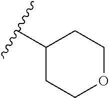 [SMILES: *C1CCOCC1] | (400 MHz, CDCl₃): δ 1.14-1.27 (m, 5H), 1.47-1.59 (m, 2H), 1.64-1.72 (m, 1H), 1.87-2.00 (m, 4H), 2.04-2.16 (m, 2H), 2.25-2.34 (m, 2H), 3.25-3.37 (m, 6H), 3.58 (td, J = 11.6, 2.3 Hz, 2H), 4.02 (q, J = 7.1 Hz, 2H), 4.07-4.13 (m, 2H), 4.81- 4.93 (m, 1H), 7.76 (s, 1H). |
| 318 | Me— | 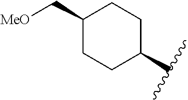 [SMILES: *[C@H]1CC[C@@H](COC)CC1] | 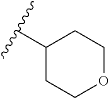 [SMILES: *C1CCOCC1] | (400 MHz, CDCl₃): δ 1.29-1.42 (m, 2H), 1.67-1.77 (m, 5H), 1.86-1.95 (m, 2H), 2.03-2.21 (m, 4H), 3.24-3.37 (m, 6H), 3.42 (s, 3H), 3.57 (td, J = 11.7, 2.2 Hz, 2H), 4.05-4.13 (m, 2H), 5.25-5.31 (m, 1H), 7.77 (s, 1H). |
| 319 | Et— | 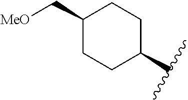 [SMILES: *[C@H]1CC[C@@H](COC)CC1] | 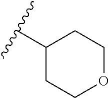 [SMILES: *C1CCOCC1] | (400 MHz, CDCl₃): δ 1.23-1.42 (m, 5H), 1.63-1.80 (m, 5H), 1.85-1.97 (m, 2H), 2.03-2.25 (m, 4H), 3.23-3.38 (m, 6H), 3.53-3.62 (m, 2H), 4.00-4.15 (m, 4H), 5.23-5.32 (m, 1H), 7.77 (s, 1H). |
| 320 | Me— | 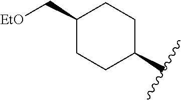 [SMILES: *[C@H]1CC[C@@H](COCC)CC1] | 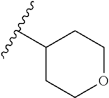 [SMILES: *C1CCOCC1] | (400 MHz, CDCl₃): δ 1.21 (t, J = 7.0 Hz, 3H), 1.28-1.41 (m, 2H), 1.66-1.80 (m, 5H), 1.86- 1.95 (m, 2H), 2.04-2.22 (m, 4H), 3.27-3.37 (m, 3H), 3.42 (s, 3H), 3.49 (q, J = 7.0 Hz, 2H), 3.55-3.61 (m, 2H), 4.05- 4.13 (m, 2H), 5.25-5.31 (m, 1H), 7.77 (s, 1H). |
| 321 | Et— | 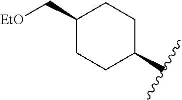 [SMILES: *[C@H]1CC[C@@H](COCC)CC1] | 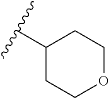 [SMILES: *C1CCOCC1] | (400 MHz, CDCl₃): δ 1.21 (t, J = 7.1 Hz, 3H), 1.27-1.43 (m, 5H), 1.65-1.81 (m, 5H), 1.86- 1.95 (m, 2H), 2.04-2.25 (m, 4H), 3.27-3.37 (m, 3H), 3.49 (q, J = 7.1 Hz, 2H), 3.53-3.62 (m, 2H), 4.00-4.15 (m, 4H), 5.23-5.32 (m, 1H), 7.77 (s, 1H). |
| 322 | Me— | 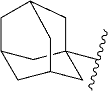 [SMILES: *C12CC3CC(CC(C3)C1)C2] | 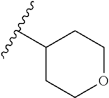 [SMILES: *C1CCOCC1] | |
| 323 | Me— | 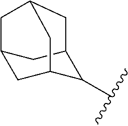 [SMILES: *C1C2CC3CC(C2)CC1C3] | 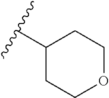 [SMILES: *C1CCOCC1] | |

### Example 324

### 3-Methyl-7-(tetrahydro-2H-pyran-4-yl)-2-{[trans-4-(trifluoromethyl)cyclohexyl]-methoxy}imidazo[5,1-f][1,2,4]triazin-4(3H)-one

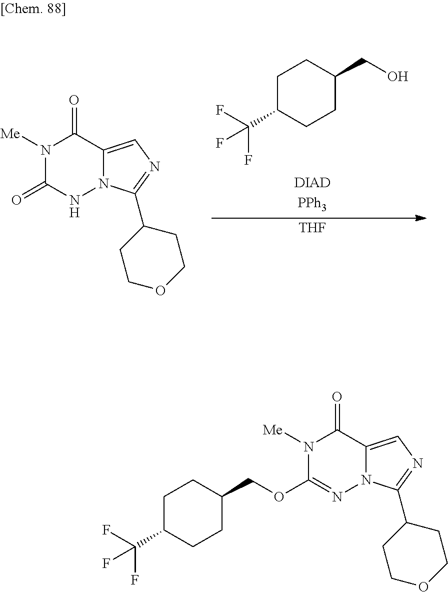 [SMILES: Cn1c(=O)[nH]n2c(C3CCOCC3)ncc2c1=O ; OC[C@H]1CC[C@H](C(F)(F)F)CC1 ; Cn1c(OC[C@H]2CC[C@H](C(F)(F)F)CC2)nn2c(C3CCOCC3)ncc2c1=O ; "THF"]

The titled compound was also prepared according to the Preparation Method 4. [trans-4-(Trifluoromethyl)cyclohexyl]methanol can be synthesized according to the procedure described in US2011/53974, WO2014/179564 or WO2014/123882. To a mixture of [trans-4-(trifluoromethyl)cyclohexyl]methanol (1.30 g, 7.14 mmol), 3-methyl-7-(tetrahydro-2H-pyran-4-yl)imidazo[5,1-f][1,2,4]triazine-2,4(1H,3H)-dione (1.49 g, 5.95 mmol) and triphenylphosphine (2.34 g, 8.93 mmol) in THF (15 ml) was added dropwise diisopropyl azodicarboxylate (1.76 ml, 8.93 mmol) and the mixture was stirred for 2 h at room temperature. Upon completion, the mixture was evaporated. After toluene (10 mL) was added to the residue, the resulting precipitate was filtered. MgCl₂ (1.7 g, 17.86 g) was added to the filtrate and the mixture was stirred for 3 h at 60° C. After filtration, brine and EtOAc were added to the filtrate. The organic layer was extracted and dried over Na₂SO₄. The obtained solid was recrystallized with IPA/ⁿHeptane (1/3) twice to give the titled compound (1.67 g, yield 67%).

LC-MS (m/z)=415 [M+H]⁺. ¹H-NMR (400 MHz, CDCl₃): δ 1.12-1.26 (m, 2H), 1.32-1.48 (m, 2H), 1.84-1.95 (m, 3H), 1.97-2.17 (m, 7H), 3.29-3.39 (m, 1H), 3.41 (s, 3H), 3.54-3.63 (m, 2H), 4.05-4.13 (m, 2H), 4.21 (d, J=6.3 Hz, 2H), 7.78 (s, 1H).

### Example 325

### 3-Methyl-2-{[trans-4-(trifluoromethyl)cyclohexyl]methoxy}imidazo[5,1-f][1,2,4]triazin-4(3H)-one

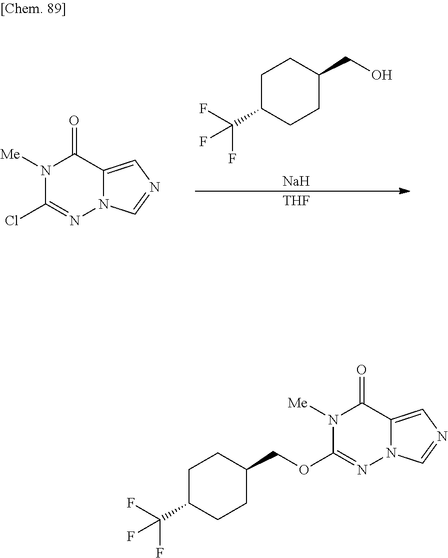

To the mixture of [trans-4-(trifluoromethyl)cyclohexyl]methanol (1.81 g, 9.96 mmol) in THF (15 mL) was added sodium hydride (239 mg, 9.96 mmol) at room temperature, then the mixture was stirred at same temperature for 0.5 h. 2-chloro-3-methylimidazo[5,1-f][1,2,4]triazin-4(3H)-one (0.92 g, 4.98 mmol) was added, then the mixture was stirred for 0.5 h. Water (1 mL) was added to the mixture and it was extracted with EtOAc (50 mL×2) and combined organic layers were dried over anhydrous Na₂SO₄ and concentrated pressure. The crude residue was purified by silica gel column chromatography using PE/EtOAc=1/1 as eluent to afford the titled compound (1.2 g, yield 73%).

LC-MS (m/z)=331 [M+H]⁺. ¹H-NMR (400 MHz, CDCl₃): δ 1.11-1.20 (m, 2H), 1.36-1.46 (m, 2H), 1.85-1.94 (m, 1H), 1.99-2.08 (m, 5H), 3.43 (s, 3H), 4.21 (d, J=6.0 Hz, 2H), 7.85 (s, 1H), 7.95 (s, 1H).

### Example 326

### 7-(4-Hydroxytetrahydro-2H-pyran-4-yl)-3-methyl-2-{[trans-4-(trifluoromethyl)-cyclohexyl]methoxy}imidazo[5,1-f][1,2,4]triazin-4(3H)-one

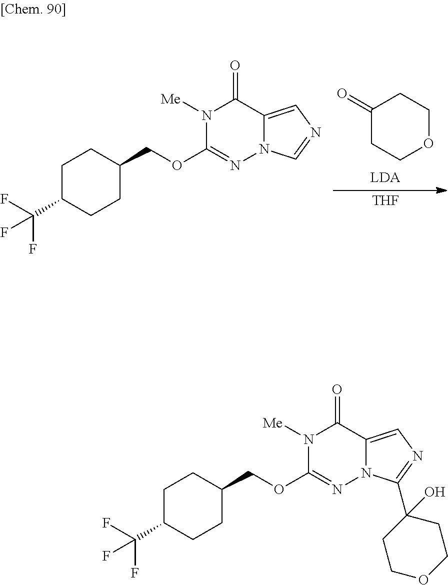 [SMILES: Cn1c(OC[C@H]2CC[C@H](C(F)(F)F)CC2)nn2cncc2c1=O ; O=C1CCOCC1 ; Cn1c(OC[C@H]2CC[C@H](C(F)(F)F)CC2)nn2c(C3(O)CCOCC3)ncc2c1=O ; "THF"]

To the mixture of 3-methyl-2-{[trans-4-(trifluoromethyl)cyclohexyl]methoxy}imidazo[5,1-f][1,2,4]triazin-4(3H)-one (330 mg, 1 mmol) in THF (4 mL) was added lithium diisopropylamide (128 mg, 1.20 mmol) at −78° C., then it was stirred at −78° C. for 0.4 h. tetrahydro-4H-pyran-4-one (150 mg, 1.50 mmol) in THF (4 mL) was added, then the mixture was stirred at −78° C. for 0.5 h. Aq. NH₄Cl (2 mL) was added and the mixture was extracted with EtOAc (30 mL×2). The combined organic layers were dried over anhydrous Na₂SO₄ and concentrated pressure, then purified by prep-HPLC (CH₃NH₂:H₂O=30:70) to give the titled compound (0.19 g, yield 44%).

LC-MS (m/z)=431 [M+H]⁺. ¹H-NMR (400 MHz, CDCl₃): δ 1.15-1.24 (m, 2H), 1.36-1.46 (m, 2H), 1.86-1.94 (m, 3H), 2.00-2.09 (m, 5H), 2.48-2.55 (m, 2H), 3.44 (s, 3H), 3.85-3.90 (m, 2H), 3.96-4.02 (m, 2H), 4.14 (s, 1H), 4.19 (d, J=6.0 Hz, 2H), 7.78 (s, 1H).

### Example 327

### 7-(1-Hydroxycyclohexyl)-3-methyl-2-{[trans-4-(trifluoromethyl)cyclohexyl]-methoxy}imidazo[5,1-f][1,2,4]triazin-4(3H)-one

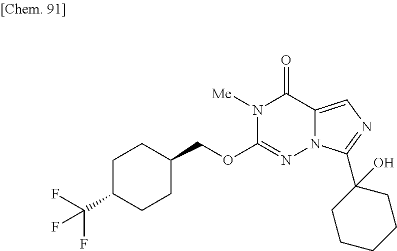 [SMILES: Cn1c(OC[C@H]2CC[C@H](C(F)(F)F)CC2)nn2c(C3(O)CCCCC3)ncc2c1=O]

The titled compound was synthesized in a similar manner to Example 326.

LC-MS (m/z)=429 [M+H]⁺. ¹H-NMR (400 MHz, CDCl₃): δ 1.14-1.23 (m, 2H), 1.33-1.46 (m, 3H), 1.63-1.73 (m, 3H), 1.78-2.22 (m, 12H), 3.43 (s, 3H), 4.03 (s, 1H), 4.19 (d, J=6.0 Hz, 2H), 7.78 (s, 1H).

### Example 328

### 7-(3,6-Dihydro-2H-pyran-4-yl)-3-methyl-2-{[trans-4-(trifluoromethyl)-cyclohexyl]methoxy}imidazo[5,1-f][1,2,4]triazin-4(3H)-one

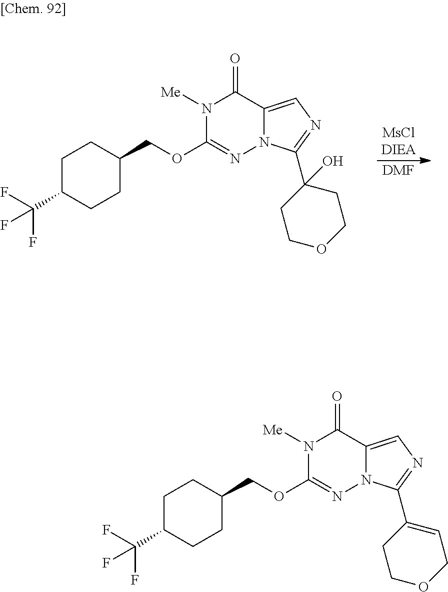

To the mixture of 7-(4-hydroxytetrahydro-2H-pyran-4-yl)-3-methyl-2-{[trans-4-(trifluoromethyl)-cyclohexyl]methoxy}imidazo[5,1-f][1,2,4]triazin-4(3H)-one (21.5 mg, 0.05 mmol) in DMF (1 mL) was added methanesulfonyl chloride (14.3 mg, 0.125 mmol) and DIEA (14.3 mg, 0.125 mmol) at room temperature, then the mixture was stirred for 16 h. The combined organic layers was filtered and purified by prep-TLC (PE/EtOAc=3:1) to give the titled compound (4.2 mg, yield 20%) as white solid.

LC-MS (m/z)=413 [M+H]⁺. ¹H-NMR (400 MHz, CDCl₃): δ 1.15-1.24 (m, 2H), 1.36-1.46 (m, 2H), 1.99-2.09 (m, 6H), 2.79-2.81 (m, 2H), 3.44 (s, 3H), 3.95-3.97 (m, 2H), 4.20 (d, J=6.0 Hz, 2H), 4.41-4.43 (m, 2H), 7.12-7.13 (m, 1H),7.86 (s, 1H).

### Example 329

### 7-(Cyclohex-1-en-1-yl)-3-methyl-2-{[trans-4-(trifluoromethyl)cyclohexyl]-methoxy}imidazo[5,1-f][1,2,4]triazin-4(3H)-one

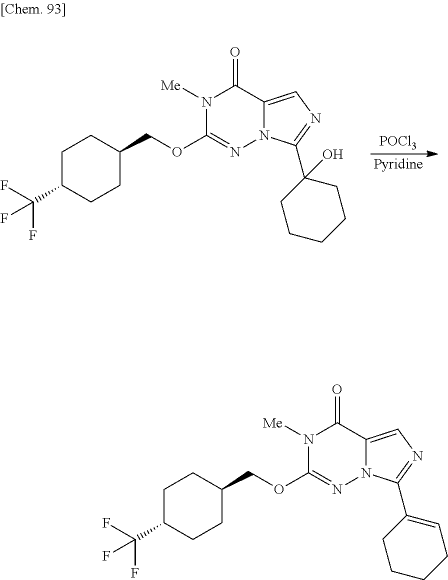 [SMILES: Cn1c(OC[C@H]2CC[C@H](C(F)(F)F)CC2)nn2c(C3(O)CCCCC3)ncc2c1=O ; "POCl3" ; "Pyridine" ; Cn1c(OC[C@H]2CC[C@H](C(F)(F)F)CC2)nn2c(C3=CCCCC3)ncc2c1=O]

To the mixture of 7-(1-hydroxycyclohexyl)-3-methyl-2-{[trans-4-(trifluoromethyl)-cyclohexyl]-methoxy}imidazo[5,1-f][1,2,4]triazin-4(3H)-one (0.15 g, 0.35 mmol) in pyridine (2 mL) was added phosphoryl chloride (321 mg, 2.10 mmol), then the mixture was stirred at room temperature for 16 h. The reaction was quenched with water and extracted with DCM. The organic layer was dried and the solvent was removed to give the titled compound (17.2 mg, yield 12%) as white solid.

LC-MS (m/z)=411 [M+H]⁺. ¹H-NMR (400 MHz, CDCl₃): δ 1.15-1.24 (m, 2H), 1.36-1.46 (m, 2H), 1.68-1.74 (m, 2H), 1.77-1.83 (m, 2H), 1.88-1.95 (m, 1H), 1.99-2.08 (m, 5H), 2.29-2.33 (m, 2H), 2.64-2.67 (m, 2H), 3.42 (s, 3H), 4.21 (d, J=6.0 Hz, 2H), 7.10-7.12 (m, 1H), 7.84 (s, 1H).

### Example 330

### 2-{(4,4-Difluorocyclohexyl)methoxy]-3-ethylimidazo[5,1-f][1,2,4]triazin-4(3H)-one

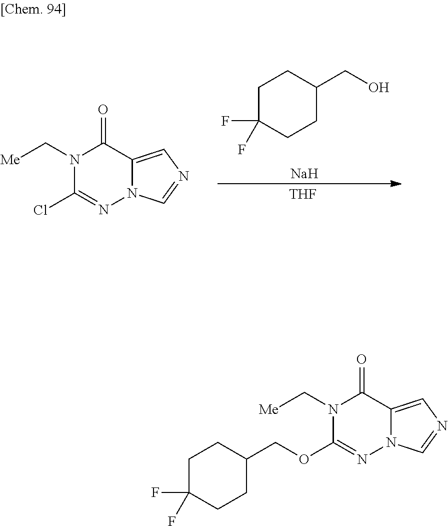

To a solution of (4,4-difluorocyclohexyl)methanol (1.65 g, 11 mmol) in THF (25 mL) was added NaH (528 mg, 22 mmol) at 0° C. and stirred at same temperature for 0.5 h. Then 2-chloro-3-ethylimidazo[1,5-f][1,2,4]triazin-4(3H)-one (1.45 g,7.5 mmol) was added. Then the reaction mixture was stirred at 0° C. for 1 h. Upon the completion, the reaction was quenched with water, then extracted with EtOAc (30 mL×2) and concentrated in vacuo to give the titled compound (1.5 g, yield 66%).

LC-MS (m/z)=313 [M+H]⁺. ¹H-NMR (400 MHz, DMSO-d₆): δ 1.27-1.30 (m, 4H), 1.44-1.54 (m, 2H), 1.71-1.95 (m, 3H), 2.07-2.23 (m, 3H), 4.06 (q, J=7.2 Hz, 2H), 4.26 (q, J=6.4 Hz, 2H), 7.83 (s, 1H), 7.94 (s, 1H).

### Example 331

### 7-Bromo-2-[(4,4-difluorocyclohexyl)methoxy]-3-ethylimidazo[5,1-f][1,2,4]triazin-4(3H)-one

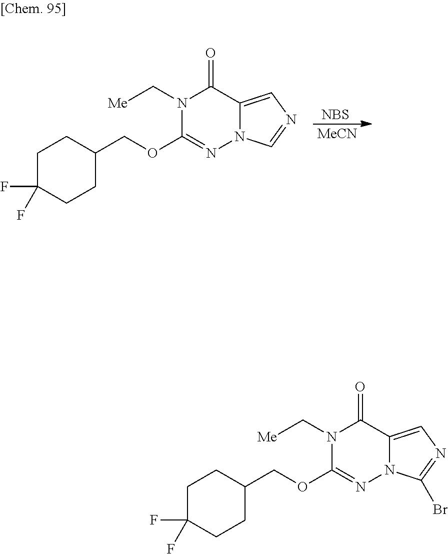

To a solution of 2-[(4,4-difluorocyclohexyl)methoxy]-3-ethylimidazo[5,1-f][1,2,4]triazin-4(3H)-one (1.4 g, 4.5 mmol) in MeCN (20 mL) was added NBS (880 mg, 4.95 mmol) at 0° C. and stirred at same temperature for 1 h. Upon the completion, the reaction was concentrated in vacuo and purified by silica gel chromatography (eluted with PE:EtOAc=5:1) to give the titled compound (900 mg, yield 51%).

LC-MS (m/z)=393 [M+H]⁺. ¹H-NMR (400 MHz, DMSO-d₆): δ 1.25-1.31 (m, 4H), 1.43-1.52 (m, 2H), 1.74-1.93 (m, 4H), 2.15-2.18 (m, 2H), 4.02 (q, J=7.2 Hz, 2H), 4.23 (d, J=6.4 Hz, 2H), 7.80 (s, 1H).

### Example 332

### 2-[(4,4-Difluorocyclohexyl)methoxy]-3-ethyl-7-(pyrrolidin-1-yl)imidazo[5,1-f][1,2,4]triazin-4(3H)-one

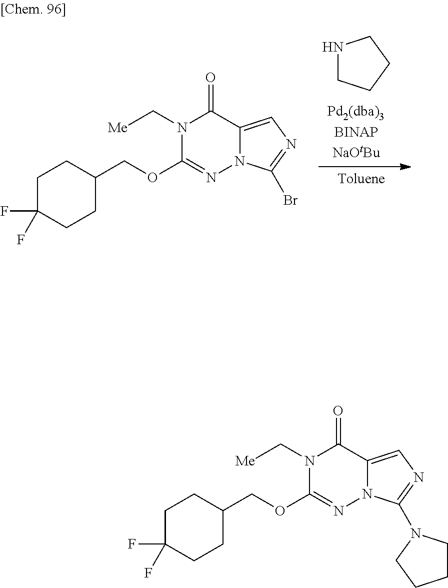 [SMILES: CCn1c(OCC2CCC(F)(F)CC2)nn2c(Br)ncc2c1=O ; C1CCNC1 ; CCn1c(OCC2CCC(F)(F)CC2)nn2c(N3CCCC3)ncc2c1=O]

To a solution of 7-bromo-2-[(4,4-difluorocyclohexyl)methoxy]-3-ethylimidazo[5,1-f][1,2,4]triazin-4(3H)-one (98 mg, 0.25 mmol) in toluene (2 mL) was added pyrrolidine (36 mg, 0.5 mmol), tBuONa (48 mg, 0.5 mmol), Pd₂(dba)₃ (35 mg, 0.038 mmol) and BINAP (47 mg, 0.075 mmol). Then the reaction was stirred at 100° C. for 16 h. Upon the completion, the reaction was concentrated in vacuo and purified by silica gel chromatography (eluted with PE:EtOAc=5:1) and prep-HPLC (MeCN/H₂O=0˜100%) to give the titled compound (55 mg, yield 58%).

LC-MS (m/z)=382 [M+H]⁺. ¹H-NMR (400 MHz, DMSO-d₆): δ 1.21-1.26 (m, 4H), 1.43-1.52 (m, 2H), 1.72-1.84 (m, 2H), 1.87-1.97 (m, 6H), 2.15-2.21 (m, 2H), 3.66-3.70 (m, 4H), 3.94 (q, J=7.2 Hz, 2H), 4.17 (d, J=6.4 Hz, 2H), 7.53 (s, 1H).

### Test Example 1

### In Vitro HTRF PDE1 Inhibition Assay

An exemplary procedure for the in vitro Homogenous Time Resolved Fluorescence assay, which can be used to determine the inhibitory action of compounds of the present invention toward PDE1 or its isoforms, follows.

The HTRF PDE1 assay utilized the HTRF (Homogenous Time Resolved Fluorescence) technology, which is based on the competition between unlabeled cyclic nucleotide and cyclic nucleotide labeled with XL665 for the binding to cyclic nucleotide-specific antibody labeled with cryptate. The HTRF signal is thus inversely proportional to the concentration of cyclic nucleotide being measured. Since phosphodiesterases break down cyclic nucleotides, the HTRF signal was used to determine PDE activity.

The Cisbio cGMP HTRF assay kit (Cat no: 62GM2PEC) was utilized. Cyclic GMP was diluted to 200 nM in HTRF assay buffer (1 mM CaCl₂, 10 mM MgCl₂, 10 mM Tris-HCl, 0.1% BSA, pH 7.4). 10 μl of compound or DMSO was diluted in 200 nM cyclic GMP solution and added to wells of a 96 well white plate to give 100 nM cyclic GMP in 1% DMSO final concentration. PDE (1A3, 1B or 1C) was diluted to 2× working concentration in HTRF assay buffer with 2 μg/ml Calmodulin, and 10 μl was added to initiate the reaction. The plate was then incubated for 45 minutes at 27° C. d2-Labelled cyclic GMP and anti-cGMP cryptate were diluted in 50 mM phosphate buffer, 0.8 M KF, 1% Triton X100, 0.2% BSA, pH 7.0. Following incubation 10 μl d2-cGMP, then 10 μl anti cGMP cryptate were added to each well and the plate was incubated for 60 minutes at room temperature. The plate was then read on Perkin Elmer EnVision at 2 different FRET readings ex/em 340 nm/665 nm and 340 nm/615 nm.

Data obtained from the HTRF assay for selected compounds of the invention are listed in Tables below. Compounds having an IC₅₀ of <1 μM are denoted as +++. Compounds having an IC₅₀ of 1-10 μM are denoted as ++. Compounds having an IC₅₀ of 10-100 μM are denoted as +.

TABLE 1
Results of in vitro PDE1B HTRF Assay

| Example | IC₅₀ |
|---|---|
| No. | (μM) |
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | +++ |
| 16 | +++ |
| 17 | +++ |
| 18 | +++ |
| 19 | +++ |
| 20 | +++ |
| 21 | +++ |
| 22 | ++ |
| 23 | +++ |
| 24 | +++ |
| 25 | +++ |
| 26 | +++ |
| 27 | +++ |
| 28 | +++ |
| 29 | ++ |
| 30 | +++ |
| 31 | +++ |
| 33 | +++ |
| 34 | +++ |
| 35 | +++ |
| 36 | +++ |
| 37 | +++ |
| 39 | +++ |
| 40 | +++ |
| 41 | +++ |
| 42 | +++ |
| 43 | +++ |
| 44 | +++ |
| 45 | +++ |
| 46 | +++ |
| 47 | +++ |
| 48 | +++ |
| 50 | +++ |
| 51 | ++ |
| 52 | +++ |
| 53 | +++ |
| 54 | +++ |
| 55 | +++ |
| 56 | +++ |
| 57 | +++ |
| 58 | +++ |
| 59 | +++ |
| 60 | +++ |
| 61 | +++ |
| 62 | +++ |
| 63 | +++ |
| 64 | +++ |
| 65 | +++ |
| 66 | +++ |
| 67 | +++ |
| 68 | +++ |
| 69 | +++ |
| 70 | +++ |
| 71 | +++ |
| 72 | +++ |
| 73 | +++ |
| 74 | +++ |
| 75 | +++ |
| 76 | +++ |
| 77 | +++ |
| 78 | +++ |
| 79 | +++ |
| 80 | ++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | +++ |
| 96 | +++ |
| 97 | +++ |
| 98 | +++ |
| 99 | +++ |
| 100 | +++ |
| 101 | +++ |
| 102 | +++ |
| 103 | +++ |
| 104 | +++ |
| 105 | +++ |
| 107 | +++ |
| 108 | +++ |
| 109 | +++ |
| 111 | +++ |
| 112 | +++ |
| 114 | +++ |
| 115 | +++ |
| 116 | +++ |
| 117 | +++ |
| 118 | +++ |
| 119 | +++ |
| 121 | +++ |
| 122 | +++ |
| 123 | +++ |
| 124 | +++ |
| 125 | +++ |
| 126 | +++ |
| 127 | +++ |
| 128 | +++ |
| 129 | +++ |
| 130 | +++ |
| 131 | ++ |
| 132 | +++ |
| 133 | +++ |
| 134 | +++ |
| 135 | +++ |
| 136 | +++ |
| 137 | +++ |
| 138 | +++ |
| 139 | +++ |
| 140 | +++ |
| 141 | +++ |
| 142 | +++ |
| 143 | +++ |
| 144 | +++ |
| 145 | +++ |
| 146 | +++ |
| 147 | +++ |
| 148 | +++ |
| 149 | +++ |
| 150 | +++ |
| 151 | +++ |
| 152 | +++ |
| 153 | +++ |
| 154 | ++ |
| 155 | +++ |
| 156 | +++ |
| 157 | +++ |
| 158 | +++ |
| 159 | +++ |
| 160 | +++ |
| 161 | +++ |
| 162 | ++ |
| 163 | ++ |
| 164 | +++ |
| 165 | +++ |
| 166 | +++ |
| 167 | ++ |
| 168 | +++ |
| 169 | +++ |
| 170 | +++ |
| 171 | +++ |
| 172 | +++ |
| 173 | ++ |
| 174 | +++ |
| 175 | +++ |
| 176 | +++ |
| 177 | +++ |
| 178 | +++ |
| 179 | +++ |
| 180 | +++ |
| 181 | +++ |
| 182 | +++ |
| 183 | +++ |
| 184 | +++ |
| 185 | +++ |
| 186 | +++ |
| 187 | +++ |
| 188 | +++ |
| 189 | +++ |
| 190 | +++ |
| 191 | +++ |
| 192 | +++ |
| 193 | +++ |
| 194 | ++ |
| 195 | +++ |
| 196 | +++ |
| 197 | +++ |
| 198 | +++ |
| 199 | +++ |
| 200 | +++ |
| 202 | +++ |
| 203 | +++ |
| 204 | +++ |
| 205 | +++ |
| 206 | ++ |
| 207 | + |
| 208 | ++ |
| 209 | + |
| 210 | + |
| 211 | + |
| 212 | + |
| 213 | ++ |
| 214 | + |
| 215 | + |
| 216 | +++ |
| 217 | ++ |
| 218 | +++ |
| 219 | +++ |
| 220 | + |
| 221 | ++ |
| 222 | + |
| 223 | ++ |
| 225 | ++ |
| 226 | +++ |
| 227 | ++ |
| 228 | ++ |
| 229 | +++ |
| 230 | +++ |
| 231 | +++ |
| 232 | ++ |
| 233 | +++ |
| 234 | +++ |
| 235 | +++ |
| 236 | +++ |
| 237 | +++ |
| 238 | +++ |
| 239 | +++ |
| 240 | +++ |
| 241 | +++ |
| 243 | +++ |
| 244 | +++ |
| 245 | +++ |
| 246 | +++ |
| 247 | +++ |
| 248 | +++ |
| 249 | +++ |
| 250 | +++ |
| 251 | +++ |
| 252 | +++ |
| 253 | +++ |
| 254 | +++ |
| 255 | +++ |
| 256 | +++ |
| 258 | +++ |
| 259 | +++ |
| 260 | +++ |
| 262 | +++ |
| 263 | +++ |
| 264 | +++ |
| 265 | +++ |
| 266 | +++ |
| 267 | ++ |
| 268 | ++ |
| 269 | +++ |
| 270 | +++ |
| 271 | +++ |
| 272 | +++ |
| 273 | +++ |
| 274 | +++ |
| 275 | +++ |
| 276 | ++ |
| 277 | ++ |
| 278 | +++ |
| 279 | +++ |
| 280 | + |
| 281 | ++ |
| 282 | +++ |
| 283 | +++ |
| 284 | +++ |
| 285 | +++ |
| 286 | +++ |
| 287 | +++ |
| 288 | +++ |
| 289 | +++ |
| 290 | +++ |
| 291 | +++ |
| 292 | +++ |
| 293 | +++ |
| 295 | +++ |
| 296 | +++ |
| 297 | +++ |
| 298 | +++ |
| 299 | +++ |
| 300 | +++ |
| 301 | +++ |
| 302 | +++ |
| 303 | +++ |
| 304 | +++ |
| 305 | +++ |
| 306 | +++ |
| 307 | +++ |
| 308 | +++ |
| 309 | +++ |
| 310 | +++ |
| 311 | +++ |
| 312 | +++ |
| 313 | +++ |
| 314 | +++ |
| 315 | +++ |
| 316 | +++ |
| 317 | +++ |
| 318 | +++ |
| 319 | +++ |
| 320 | +++ |
| 321 | +++ |
| 326 | ++ |
| 327 | ++ |
| 328 | +++ |
| 329 | ++ |
| 332 | + |

TABLE 2
Results of in vitro PDE1A3 HTRF Assay

| Example | IC₅₀ |
|---|---|
| No. | (μM) |
| 1 | +++ |
| 6 | +++ |
| 91 | +++ |
| 101 | +++ |
| 109 | +++ |
| 115 | +++ |
| 116 | +++ |
| 149 | +++ |
| 200 | +++ |
| 238 | +++ |
| 239 | +++ |
| 245 | +++ |
| 246 | +++ |
| 247 | +++ |
| 249 | +++ |
| 251 | +++ |
| 253 | +++ |
| 255 | +++ |
| 256 | +++ |
| 258 | +++ |
| 283 | +++ |
| 288 | +++ |
| 302 | +++ |
| 304 | +++ |
| 306 | +++ |
| 309 | +++ |
| 310 | +++ |
| 312 | +++ |
| 313 | +++ |
| 315 | +++ |
| 317 | +++ |
| 318 | +++ |

TABLE 3
Results of in vitro PDE1C HTRF Assay

| Example | IC₅₀ |
|---|---|
| No. | (μM) |
| 1 | +++ |
| 6 | ++ |
| 91 | +++ |
| 101 | +++ |
| 109 | +++ |
| 115 | +++ |
| 116 | +++ |
| 149 | +++ |
| 200 | +++ |
| 238 | +++ |
| 239 | +++ |
| 245 | +++ |
| 246 | +++ |
| 247 | +++ |
| 249 | +++ |
| 251 | +++ |
| 253 | +++ |
| 255 | +++ |
| 256 | +++ |
| 258 | +++ |
| 283 | +++ |
| 288 | +++ |
| 302 | +++ |
| 304 | +++ |
| 306 | +++ |
| 309 | ++ |
| 310 | +++ |
| 312 | +++ |
| 313 | +++ |
| 315 | +++ |
| 317 | +++ |
| 318 | +++ |

### Test Example 2

### In Vitro SPA PDE1 Inhibition Assay

The commercially available Scintillation Proximity Assay (SPA) system (Perkinelmer) was used for PDE1 inhibition assays. SPA Yttrium Silicate beads preferentially binds to non-cyclic GMP compared to cyclic GMP (cGMP) in the presence of zinc sulphate, which enables to measure the amount of enzyme reaction product ([³H]-GMP) by scintillation counting.

For PDE1 inhibition assay, 10 μl of the test compounds in 5% dimethyl sulfoxide diluted in SPA assay buffer (1 mM CaCl₂, 10 mM MgSO₄, 10 mM Tris-HCl, pH 7.4) were placed in a 96-well white microtiter plate, and 20 μl of [³H]-cGMP solution (100 nM final concentration, Perkinelmer) in SPA assay buffer was added. Assays were initiated by adding 20 μl of PDE (1A3, 1B or 1C) enzyme solution including calmodulin (final concentration 1 μg/ml). After 30 min incubation at 27° C., the reaction was stopped by the addition of 50 μl of a suspension of 10 mg/ml SPA beads in 18 mM ZnSO₄. The microtiter plates were settled for 60 min, and then were measured in a TopCount NXT scintillation counter (PerkinElmer). IC₅₀ values were determined from sigmoidal curves by plotting the percentage of PDE activity versus log compound concentration. IC₅₀ was defined as the concentration of the test compounds required to inhibit the cyclic nucleotide hydrolyzing activities of tested PDEs by 50%.

Data obtained from the SPA assay for selected compounds of the invention are listed in Tables below.

TABLE 4
Results of in vitro PDE1B SPA Assay

| Example | IC₅₀ |
|---|---|
| No. | (μM) |
| 1 | 0.088 |
| 6 | 0.469 |
| 91 | 0.028 |
| 101 | 0.096 |
| 109 | 0.028 |
| 115 | 0.033 |
| 116 | 0.084 |
| 149 | 0.051 |
| 200 | 0.109 |
| 238 | 0.145 |
| 239 | 0.079 |
| 245 | 0.036 |
| 246 | 0.064 |
| 288 | 0.023 |
| 317 | 0.027 |

TABLE 5
Results of in vitro PDE1A3 SPA Assay

| Example | IC₅₀ |
|---|---|
| No. | (μM) |
| 1 | 0.220 |
| 6 | 1.43 |
| 91 | 0.074 |
| 101 | 0.121 |
| 109 | 0.023 |
| 115 | 0.034 |
| 116 | 0.207 |
| 149 | 0.144 |
| 200 | 0.133 |
| 238 | 0.137 |
| 239 | 0.065 |
| 245 | 0.113 |
| 246 | 0.289 |
| 288 | 0.080 |
| 317 | 0.042 |

TABLE 6
Results of in vitro PDE1C SPA Assay

| Example | IC₅₀ |
|---|---|
| No. | (μM) |
| 1 | 0.665 |
| 6 | 4.88 |
| 91 | 0.199 |
| 101 | 0.192 |
| 109 | 0.057 |
| 115 | 0.065 |
| 116 | 0.771 |
| 149 | 0.425 |
| 200 | 0.024 |
| 238 | 0.027 |
| 239 | 0.125 |
| 245 | 0.547 |
| 246 | 0.788 |
| 288 | 0.276 |
| 317 | 0.194 |

### Test Example 3

### Evaluation of Bioavailability

PK Test in Rats

This test can be used to assess pharmacokinetics of the compounds of the present invention. Each compound of the invention was administered to 7 week-old SD rats intravenously with a solution in saline: 0.1N HCl=9:1 or orally with a 0.5% suspension in methyl cellulose, and each blood sample was collected at an appropriate interval as follows:

5, 15, and 30 minutes, and 1, 2, 4, 6, and 24 hours after intravenous administration

15 and 30 minutes, and 1, 2, 4, 6, and 24 hours after oral administration

The plasma was obtained from each blood, and the concentration of each compound in the plasma was measured with an LC-MS to calculate each pharmacokinetic parameter based on the concentration change.

### Test Example 4

### Evaluation of Drug Transportation to Brain

Test for Drug Transportation to Brain in Rats

This test can be used to assess transportation of the compounds of the present invention to the brain. Each compound of the invention was orally administered to 7 week-old SD rats with a 0.5% suspension in methyl cellulose, and each plasma and brain were collected one hour after administration to measure the concentration of each compound in the plasma and the brain with an LC-MS.

Each protein binding ratio of the compounds in the plasma and the brain was measured by the equilibrium dialysis method.

The obtained concentrations and protein binding ratios of the compounds in the plasma and the brain can be applied to the following equation to calculate Kp, uu, brain (i.e., non-binding drug concentration ratio between brain and plasma).

Kp, uu, brain=[Compound concentration in brain×(100−Protein binding ratio in brain (%))/100]/[Compound concentration in plasma×(100−Protein binding ratio in plasma (%))/100]

Results of Test Examples 3 and 4 are shown in the Table below.

TABLE 7

| Example | | |
|---|---|---|
| No. | Bioavailability (%) | Kp, uu, brain |
| 1 | 23.3 | 0.69 |
| 109 | — | 1.02 |
| 115 | 13.4 | 0.96 |
| 149 | 55.6 | 0.30 |
| 245 | 30.4 | 0.72 |
| 246 | 51.6 | 0.26 |
| 256 | 14.3 | — |
| 288 | 54.5 | 0.61 |

### Test Example 5

### Evaluation of the Effects of Compounds on Cognitive Dysfunction in Rats Repeatedly Treated with Phencyclidine

Since an N-Methyl-D-aspartate receptor antagonist phencyclidine (PCP) has been known to cause schizophrenia-like symptoms including cognitive dysfunction in humans, PCP-treated animals have been widely used as a schizophrenic animal model. Using rats to which PCP was repeatedly administered as a schizophrenic animal model, the improvement in cognitive dysfunction with the compounds of the invention was evaluated in the novel object recognition test. Rats were habituated to the test environment which is a box (50 cm×50 cm×35 cm) with no object inside for 1 hour per day for 3 days. On the test day, rats were again put into the test box with no object inside to habituate themselves for three minutes. The rats were then put into the test box with two identical objects inside and were made to explore inside for three minutes for a training trial. After one minute, they were put into the test box with one of the objects (i.e., the familiar object) that was the same as used in the training trial and another object (i.e., a novel object) inside and were made to explore inside for three minutes for a test trial. Cognitive function was evaluated on the basis of preference for the novel object in the test trial utilizing rats' habit of exploring a novel object longer than a familiar object. Discrimination Index was calculated from the following equation as an index for preference of a novel object.

Discrimination Index=(Time to explore novel object−Time to explore familiar object)/(Time to explore novel object+Time to explore familiar object)

Discrimination Indices were significantly decreased when PCP 3 mg/kg i.p. was administered to rats twice a day for 10 days, compared to the case where the vehicle was administered to rats, which indicated that cognitive function was impaired. When the compound of Example 1, 115, 149, 245 or 288 (1 or 3 mg/kg) was orally administered to the rats to which PCP was administered for 10 days beforehand 60 minutes before the start of the novel object recognition test, a significant increase of Discrimination Index was observed, compared to the PCP+vehicle-administered group (FIG. 1, 2, 3, 4 or 5).

While we have described a number of embodiments of the present invention, it is apparent that the examples as described herein may be altered to provide other embodiments that utilize the compounds and methods of the present invention. Therefore, it will be appreciated that the scope of the present invention is to be defined by the appended claims rather than by the specific embodiments that have been represented by way of example.

1. A compound of formula I: 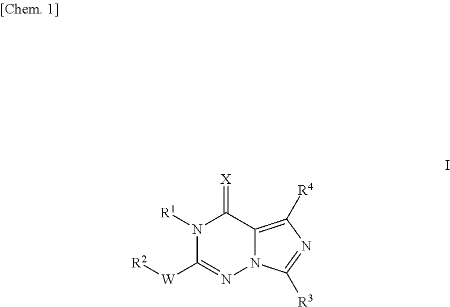
or a pharmaceutically acceptable salt thereof, wherein:
R¹ is selected from
(i) a hydrogen,
(ii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy),
(c) a hydroxyl,
(d) a phenoxy (said group being optionally substituted with the same or different 1 to 4 halogen, C₁₋₆ alkyl, or C₁₋₆ alkoxy), and
(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and
(iii) a C₃₋₁₀ cycloalkyl; a phenyl; a 5-6 membered monocyclic heteroaryl; a 4-8 membered saturated or partially unsaturated monocyclic heterocyclyl; a C₃₋₁₀ cycloalkyl-C₁₋₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;
wherein each of said groups in the aforesaid (iii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl);
X is O or S;
W is a covalent bond, —C≡CH—, —CH═CH—, —O—, or —N(R⁵)—;
R⁵ is a hydrogen or a C₁₋₆ alkyl;
R² is selected from
(i) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy),
(c) a hydroxyl,
(d) a phenoxy (said group being optionally substituted with the same or different 1 to 4 halogen, C₁₋₆ alkyl, or C₁₋₆ alkoxy), and
(e) an amino (said group being optionally substituted with the same or different 1 to 2 C₁₋₆ alkyl, C₃₋₈ cycloalkyl, or phenyl (said phenyl being optionally substituted with the same or different 1 to 4 halogen, C₁₋₆ alkyl, C₁₋₆ alkoxy, or trifluoromethyl)), and
(ii) a C₃₋₁₀ cycloalkyl; a phenyl; a 5-6 membered heteroaryl; a 4-8 membered saturated or partially unsaturated heterocyclyl; a C₃₋₁₀ cycloalkyl-C₁₋₄ alkyl; a phenyl-C₁₋₄ alkyl; a 5 or 6-membered heteroaryl-C₁₋₄ alkyl; or a 4-8 membered saturated or partially unsaturated heterocyclyl-C₁₋₄ alkyl;
wherein each of said groups in the aforesaid (ii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl);
alternatively, R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, hydroxy, or aminocarbonyl (said amino being optionally substituted with the same or different 1 to 2 C₁₋₆ alkyl)),
(c) a C₁₋₆ alkoxy (said group being optionally substituted with the same or different 1 to 3 halogen, C₁₋₆ alkoxy, or hydroxy),
(d) a phenyl (said group being optionally substituted with the same or different 1 to 4 halogen, cyano, C₁₋₆ alkyl, or C₁₋₆ alkoxy),
(e) a 5-6 membered monocyclic heteroaryl (said group being optionally substituted with the same or different 1 to 4 halogen, cyano, C₁₋₆ alkyl, or C₁₋₆ alkoxy),
(f) a 5-6 membered monocyclic heteroaryloxy (said group being optionally substituted with the same or different 1 to 4 halogen, cyano, C₁₋₆ alkyl, or C₁₋₆ alkoxy),
(g) a C₁₋₆ alkylcarbonyl (said alkyl being optionally substituted with the same or different 1 to 3 halogen, C₁₋₆ alkoxy, or amino (said amino being optionally substituted with the same or different 1 to 2 C₁₋₆ alkyl)), and
(h) an aminocarbonyl (said amino being optionally substituted with the same or different 1 to 2 C₁₋₆ alkyl);
R³ is selected from
(i) a hydrogen,
(ii) a halogen,
(iii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen),
(c) a hydroxy, and
(d) an oxo), or
(iv) a C₃₋₈ cycloalkyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo),
(v) a C₂₋₆ alkenyl (said group being optionally substituted with the same or different 1 to 4 halogen),
(vi) a C₃₋₈ cycloalkenyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) an oxo), and
(vii) 4-8 membered saturated or partially unsaturated monocyclic heterocyclyl (said group being optionally substituted with the same or different 1 to 4 group(s) selected from
(a) a halogen,
(b) a C₁₋₆ alkyl (said group being optionally substituted with the same or different 1 to 3 halogen),
(c) a C₁₋₆ alkoxy (said group being optionally substituted with the same or different 1 to 3 halogen),
(d) a hydroxy, and
(e) a cyano);
R⁴ is selected from
(i) a hydrogen,
(ii) a halogen,
(iii) a C₁₋₆ alkyl (said group being optionally substituted with the same or different 1 to 3 halogen), or
(iv) a cyano;
provided that when R¹ is a hydrogen, then W is —O— or —N(R⁵)— and R⁴ is a hydrogen.

2. The compound of claim 1, or a pharmaceutically acceptable salt thereof, wherein R¹ is selected from
(i) a hydrogen,
(ii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy), and
(c) a hydroxyl), and
(iii) a C₃₋₁₀ cycloalkyl; a C₃₋₁₀ cycloalkyl-C₁₋₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;
wherein each of said groups in the aforesaid (iii) is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy), and
(g) a 5-6 membered monocyclic heteroaryl (said group being optionally substituted with the same or different 1 to 4 halogen, cyano, C₁₋₆ alkyl, or C₁₋₆ alkoxy).

3. The compound of claim 1, or a pharmaceutically acceptable salt thereof, wherein R¹ is selected from
(i) a hydrogen,
(ii) a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy), and
(c) a hydroxyl),
(iii) a C₃₋₈ cycloalkyl (said group being optionally substituted with the same or different 1 to 4 halogen, C₁₋₆ alkyl, or C₁₋₆ alkoxy), and
(iv) C₃₋₈ cycloalkyl-C₁₋₄ alkyl (said group being optionally substituted with the same or different 1 to 4 halogen, C₁₋₆ alkyl, or C₁₋₆ alkoxy);
W is —O—, or —N(R⁵)—;
R⁵ is a hydrogen, or a C₁₋₆ alkyl; and
R² is a C₃₋₁₀ cycloalkyl-C₁₋₄ alkyl; a phenyl-C₁₋₄ alkyl; a 5 or 6-membered monocyclic heteroaryl-C₁₋₄ alkyl; a 4-8 membered saturated or partially unsaturated monocyclic heterocyclyl-C₁₋₄ alkyl; a C₃₋₁₀ cycloalkyl; or a 4-8 membered saturated or partially unsaturated monocyclic heterocyclyl;
wherein each of said groups in R² is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy),
(g) a 5-6 membered monocyclic heteroaryl (said group being optionally substituted with the same or different 1 to 4 halogen, cyano, C₁₋₆ alkyl, or C₁₋₆ alkoxy), and
(h) an aminocarbonyl (said amino being optionally substituted with the same or different 1 to 2 C₁₋₆ alkyl);
alternatively, R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, hydroxyl, or aminocarbonyl (said amino being optionally substituted with the same or different 1 to 2 C₁₋₆ alkyl)),
(c) a C₁₋₆ alkoxy (said group being optionally substituted with the same or different 1 to 3 halogen, C₁₋₆ alkoxy, or hydroxy),
(d) a phenyl (said group being optionally substituted with the same or different 1 to 4 halogen, cyano, C₁₋₆ alkyl, or C₁₋₆ alkoxy),
(e) a 5-6 membered monocyclic heteroaryl (said group being optionally substituted with the same or different 1 to 4 halogen, cyano, C₁₋₆ alkyl, or C₁₋₆ alkoxy),
(f) a 5-6 membered monocyclic heteroaryloxy (said group being optionally substituted with the same or different 1 to 4 halogen, cyano, C₁₋₆ alkyl, or C₁₋₆ alkoxy),
(g) a C₁₋₆ alkylcarbonyl (said alkyl being optionally substituted with the same or different 1 to 3 halogen, C₁₋₆ alkoxy, or amino (said amino being optionally substituted with the same or different 1 to 2 C₁₋₆ alkyl)), and
(h) an aminocarbonyl (said amino being optionally substituted with the same or different 1 to 2 C₁₋₆ alkyl).

4. The compound of claim 1, or a pharmaceutically acceptable salt thereof, wherein R¹ is a C₁₋₆ alkyl (said group being optionally substituted with the same or different 1 to 3 halogen, or C₁₋₆ alkoxy); and
R² is a C₃₋₁₀ cycloalkyl-C₁₋₄ alkyl; a phenyl-C₁₋₄ alkyl; a 5 or 6-membered monocyclic heteroaryl-C₁₋₄ alkyl; or a C₃₋₁₀ cycloalkyl;
wherein each of said groups in R² is optionally substituted with the same or different 1 to 4 group(s) selected from
(a) a halogen,
(b) a C₁₋₆ alkyl (said group being optionally substituted with the same or different 1 to 3 halogen, hydroxy or C₁₋₆ alkoxy),
(c) a C₁₋₆ alkoxy (said group being optionally substituted with the same or different 1 to 3 halogen, hydroxy or C₁₋₆ alkoxy),
(d) a cyano,
(e) a phenyl (said group being optionally substituted with the same or different 1 to 4 halogen, cyano, C₁₋₆ alkyl, or C₁₋₆ alkoxy), and
(f) a 5-6 membered monocyclic heteroaryl (said group being optionally substituted with the same or different 1 to 4 halogen, cyano, C₁₋₆ alkyl, or C₁₋₆ alkoxy).

5. The compound of claim 4, or a pharmaceutically acceptable salt thereof, wherein R² is a C₃₋₈ cycloalkyl-C₁₋₄ alkyl or a C₃₋₈ cycloalkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, hydroxy or C₁₋₆ alkoxy), and
(c) a C₁₋₆ alkoxy (said group being optionally substituted with the same or different 1 to 3 halogen, hydroxy or C₁₋₆ alkoxy).

6. The compound of claim 1, or a pharmaceutically acceptable salt thereof, wherein R¹ is a C₃₋₁₀ cycloalkyl; a C₃₋₁₀ cycloalkyl-C₁₋₄ alkyl; a phenyl-C₁₋₄ alkyl; a 5 or 6-membered monocyclic heteroaryl-C₁₋₄ alkyl; or a 4-8 membered saturated or partially unsaturated monocyclic heterocyclyl-C₁₋₄ alkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a hydroxy,
(e) a cyano,
(f) a phenyl (said group being optionally substituted with the same or different 1 to 4 halogen, cyano, C₁₋₆ alkyl, or C₁₋₆ alkoxy), and
(g) a 5-6 membered monocyclic heteroaryl (said group being optionally substituted with the same or different 1 to 4 halogen, cyano, C₁₋₆ alkyl, or C₁₋₆ alkoxy);
W is —O— or —N(R⁵)—;
R⁵ is a hydrogen or a C₁₋₆ alkyl; and
R² is a C₁₋₆ alkyl (said group being optionally substituted with the same or different 1 to 3 group(s) selected from
(a) a halogen,
(b) a C₁₋₆ alkoxy (said group being optionally substituted with the same or different 1 to 3 halogen or hydroxy), and
(c) a hydroxy);
alternatively, R² and R⁵ may be taken together with the nitrogen atom to which they attach to form a 4-8 membered saturated or partially unsaturated monocyclic heterocyclic ring; said ring is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy),
(c) a C₁₋₆ alkoxy (said group being optionally substituted with the same or different 1 to 3 halogen, C₁₋₆ alkoxy, or hydroxy),
(d) a phenyl (said group being optionally substituted with the same or different 1 to 4 halogen, cyano, C₁₋₆ alkyl, or C₁₋₆ alkoxy), and
(e) a 5-6 membered monocyclic heteroaryl (said group being optionally substituted with the same or different 1 to 4 halogen, cyano, C₁₋₆ alkyl, or C₁₋₆ alkoxy).

7. The compound of claim 6, or a pharmaceutically acceptable salt thereof, wherein R¹ is a C₃₋₈ cycloalkyl-C₁₋₄ alkyl; or a phenyl-C₁₋₄ alkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, C₁₋₆ alkoxy, or hydroxy), and
(c) a C₁₋₆ alkoxy (said group being optionally substituted with the same or different 1 to 3 halogen or hydroxy).

8. The compound of claim 1, or a pharmaceutically acceptable salt thereof, wherein X is O.

9. The compound of claim 1, or a pharmaceutically acceptable salt thereof, wherein W is —O—.

10. The compound of claim 1, or a pharmaceutically acceptable salt thereof, wherein R³ is selected from
(i) a C₁₋₆ alkyl (said group being optionally substituted with the same or different 1 to 3 halogen, hydroxy or C₁₋₆ alkoxy),
(ii) a C₃₋₈ cycloalkyl (said group being optionally substituted with the same or different 1 to 4 halogen, C₁₋₆ alkyl, hydroxy or C₁₋₆ alkoxy), and
(iii) a 4-8 membered saturated or partially unsaturated monocyclic heterocyclyl (said group being optionally substituted with the same or different 1 to 4 halogen, hydroxy, C₁₋₆ alkyl, or C₁₋₆ alkoxy).

11. The compound of claim 10, or a pharmaceutically acceptable salt thereof, wherein R³ is a C₁₋₆ alkyl (said group being optionally substituted with the same or different 1 to 3 halogen, hydroxy or C₁₋₆ alkoxy), a tetrahydropyranyl (said group being optionally substituted with the same or different 1 to 4 halogen, hydroxy, or C₁₋₆ alkoxy), or a dihydropyranyl.

12. The compound of claim 1, or a pharmaceutically acceptable salt thereof, wherein R⁴ is selected from
(i) a hydrogen,
(ii) a halogen, or
(iii) a C₁₋₆ alkyl (said group being optionally substituted with the same or different 1 to 3 halogen).

13. The compound of claim 1, or a pharmaceutically acceptable salt thereof, wherein R⁴ is a hydrogen.

14. The compound of claim 1, or a pharmaceutically acceptable salt thereof, wherein R¹ is a C₁₋₆ alkyl (said group being optionally substituted with the same or different 1 to 3 halogen, or C₁₋₆ alkoxy);
R² is a C₃₋₈ cycloalkyl-C₁₋₄ alkyl or a C₃₋₈ cycloalkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, hydroxy or C₁₋₆ alkoxy), and
(c) a C₁₋₆ alkoxy (said group being optionally substituted with the same or different 1 to 3 halogen, hydroxy or C₁₋₆ alkoxy);
X is O; and W is —O—.

15. The compound of claim 1, having the structure of formula Ia: 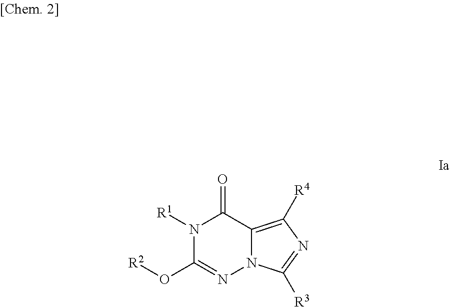
or a pharmaceutically acceptable salt thereof, wherein:
R¹ is a C₁₋₆ alkyl (said group being optionally substituted with the same or different 1 to 3 halogen or C₁₋₆ alkoxy);
R² is a C₃₋₈ cycloalkyl-C₁₋₄ alkyl or a C₃₋₈ cycloalkyl;
wherein each of said groups is optionally substituted with the same or different 1 to 4 group(s) selected from
(a) a halogen,
(b) a C₁₋₆ alkyl (said group being optionally substituted with the same or different 1 to 3 halogen, hydroxy or C₁₋₆ alkoxy), and
(c) a C₁₋₆ alkoxy (said group being optionally substituted with the same or different 1 to 3 halogen, hydroxy or C₁₋₆ alkoxy);
R³ is selected from
(i) a C₁₋₆ alkyl (said group being optionally substituted with the same or different 1 to 3 halogen, hydroxy or C₁₋₆ alkoxy),
(ii) a C₃₋₈ cycloalkyl (said group being optionally substituted with the same or different 1 to 4 halogen, C₁₋₆ alkyl, hydroxy or C₁₋₆ alkoxy), and
(iii) a 4-8 membered saturated or partially unsaturated monocyclic heterocyclyl (said group being optionally substituted with the same or different 1 to 4 halogen, hydroxy, C₁₋₆ alkyl, or C₁₋₆ alkoxy); and
R⁴ is selected from
(i) a hydrogen,
(ii) a halogen, or
(iii) a C₁₋₆ alkyl (said group being optionally substituted with the same or different 1 to 3 halogen).

16. The compound of claim 1, or a pharmaceutically acceptable salt thereof, wherein R³ is a tetrahydropyranyl (said group being optionally substituted with hydroxy), or a dihydropyranyl; and R⁴ is a hydrogen.

17. The compound of claim 1, selected from the group consisting of:
2-[(4,4-difluorocyclohexyl)methoxy]-3-ethyl-7-(tetrahydro-2H-pyran-4-yl)imidazo[5,1-f][1,2,4]triazin-4(3H)-one;
3-(4-chlorobenzyl)-2-methoxy-7-(tetrahydro-2H-pyran-4-yl)imidazo[5,1-f][1,2,4]triazin-4(3H)-one;
3-[(4,4-difluorocyclohexyl)methyl]-2-methoxy-7-(tetrahydro-2H-pyran-4-yl)imidazo[5,1-f][1,2,4]triazin-4(3H)-one;
5-chloro-2-[(4,4-difluorocyclohexyl)methoxy]-3-ethyl-7-(tetrahydro-2H-pyran-4-yl)-imidazo[5,1-f][1,2,4]triazin-4(3H)-one;
2-{[(5-chloropyridin-2-yl)methyl]amino}-3-methyl-7-(propan-2-yl)imidazo[5,1-f][1,2,4]triazin-4(3H)-one;
3-methyl-7-(tetrahydro-2H-pyran-4-yl)-2-{[trans-4-(trifluoromethyl)cyclohexyl]methoxy}-imidazo[5,1-f][1,2,4]triazin-4(3H)-one;
2-[(trans-4-ethoxycyclohexyl)methoxy]-3-methyl-7-(tetrahydro-2H-pyran-4-yl)imidazo[5,1-f][1,2,4]triazin-4(3H)-one;
3-methyl-7-(tetrahydro-2H-pyran-4-yl)-2-{[cis-4-(trifluoromethyl)cyclohexyl]methoxy}-imidazo[5,1-f][1,2,4]triazin-4(3H)-one;
2-(cyclohexyloxy)-3-methyl-7-(tetrahydro-2H-pyran-4-yl)imidazo[5,1-f][1,2,4]triazin-4(3H)-one;
2-{[trans-4-(ethoxymethyl)cyclohexyl]oxy}-3-methyl-7-(tetrahydro-2H-pyran-4-yl)-imidazo-[5,1-f][1,2,4]triazin-4(3H)-one;
2-{[trans-4-(ethoxymethyl)cyclohexyl]methoxy}-3-methyl-7-(tetrahydro-2H-pyran-4-yl)imidazo[5,1-f][1,2,4]triazin-4(3H)-one;
3-(¹³C,²H₃)methyl-7-(tetrahydro-2H-pyran-4-yl)-2-{[trans-4-(trifluoromethyl)cyclohexyl]-methoxy}imidazo[5,1-f][1,2,4]triazin-4(3H)-one;
2-[(4,4-difluorocyclohexyl)oxy]-3-ethyl-7-(tetrahydro-2H-pyran-4-yl)imidazo[5,1-f][1,2,4]triazin-4(3H)-one;
3-methyl-7-(tetrahydro-2H-pyran-4-yl)-2-{[trans-4-(trifluoromethyl)cyclohexyl]oxy}-imidazo[5,1-f][1,2,4]triazin-4(3H)-one;
3-ethyl-2-{[trans-4-(methoxymethyl)cyclohexyl]oxy}-7-(tetrahydro-2H-pyran-4-yl)-imidazo[5,1-f][1,2,4]triazin-4(3H)-one;
7-(4-hydroxytetrahydro-2H-pyran-4-yl)-3-methyl -2-{[trans-4-(trifluoromethyl)cyclohexyl]-methoxy}imidazo[5,1-f][1,2,4]triazin-4(3H)-one; and
7-(3,6-dihydro-2H-pyran-4-yl)-3-methyl-2-{[trans-4-(trifluoromethyl)cyclohexyl]methoxy}-imidazo[5,1-f][1,2,4]triazin-4(3H)-one; or a pharmaceutically acceptable salt thereof.

18. A composition comprising the compound according to claim 1 and a pharmaceutically acceptable carrier, adjuvant, or vehicle.

19. A method of inhibiting PDE1 in a patient in need thereof, comprising administering to said patient the composition according to claim 18.

20. A method of inhibiting PDE1 in a biological sample, comprising contacting the biological sample with the compound according to claim 1.

21. A method for treating a neurological or psychiatric disorder in a patient in need thereof, comprising administering to said patient the composition according to claim 18.

22. The method according to claim 21, wherein the neurological or psychiatric disorder is Alzheimer's Disease, Parkinson's Disease, depression, cognitive impairment, stroke, schizophrenia, Down Syndrome, or Fetal Alcohol Syndrome.

23. The method according to claim 21, wherein the neurological or psychiatric disorder involves a deficiency in one or more cognitive domain as defined by DSM-5.
